(19) United States
(12) Patent Application Publication
(10) Pub. No.: US 2019/0000923 A1
(43) Pub. Date: Jan. 3, 2019

(54) METHODS OF TREATING OR AMELIORATING METABOLIC DISORDERS USING GROWTH DIFFERENTIATION FACTOR 15 (GDF-15)

(71) Applicant: NOVARTIS AG, Basel (CH)

(72) Inventors: William CHUTKOW, Needham, MA (US); John Richard Neville HADCOCK, Dedham, MA (US); Kurt Alex HELDWEIN, Belmont, MA (US); Aimee Richardson USERA, Winchester, MA (US)

(21) Appl. No.: 16/064,054

(22) PCT Filed: Dec. 20, 2016
(86) PCT No.: PCT/IB2016/057839

§ 371 Date: Jun. 20, 2018

Related U.S. Application Data
(60) Provisional application No. 62/270,967, filed on Dec. 22, 2015.

(51) Int. Cl. A61K 38/19; A61P 1/16; A61K 47/54; A61K 47/60

(52) U.S. Cl. CPC A61K 38/19; A61K 47/60; A61K 47/542; A61P 1/16

(57) ABSTRACT

The disclosure relates to the treatment of non-alcoholic fatty liver disease (NAFLD) and non-alcoholic steatohepatitis (NASH), as well as end-stage liver disease, hepatic steatosis (fatty liver), liver fibrosis, liver inflammation, liver cirrhosis, primary biliary cirrhosis (PBC), and hepatocellular carcinoma (HCC), by administering to a subject in need a GDF15 protein or a functional variant, mutation, fusion, or conjugate thereof, and to pharmaceutical compositions that contain the same.

### CROSS-REFERENCE TO RELATED APPLICATIONS

This application claims the benefit of U.S. Provisional Application No. 62/270,967 filed on Dec. 22, 2015, which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Dec. 16, 2016, is named PAT057168-WO-PCT_SL.txt and is 920,254 bytes in size.

### FIELD

This invention relates to the treatment of non-alcoholic fatty liver disease (NAFLD) and non-alcoholic steatohepatitis (NASH), as well as related conditions that include but are not limited to alcoholic steatohepatitis (ASH), end-stage liver disease, hepatic steatosis (fatty liver), liver fibrosis, liver inflammation, liver cirrhosis, primary biliary cirrhosis (PBC), and hepatocellular carcinoma (HCC).

### BACKGROUND

Non-alcoholic fatty liver disease (NAFLD) is a disorder affecting as many as 1 in 3-5 adults and 1 in 10 children in the United States, and refers to conditions where there is an accumulation of excess fat in the liver of people who drink little or no alcohol. The most common form of NAFLD is a non-serious condition called hepatic steatosis (fatty liver), in which fat accumulates in the liver cells; although not a physiologically normal condition, hepatic steatosis by itself likely does not damage the liver.

NAFLD most often presents itself in individuals with a constellation of risk factors termed “metabolic syndrome,” which is characterized by elevated fasting plasma glucose (FPG) with or without intolerance to post-prandial glucose, being overweight or obese, high blood lipids such as cholesterol and triglycerides (TGs) and low high-density lipoprotein cholesterol (HDL-C) levels, and high blood pressure. Not all NAFLD patients have all the manifestations of the metabolic syndrome.

Obesity is thought to be the most common cause of NAFLD and some experts estimate that about two-thirds of obese adults and one-half of obese children may have hepatic steatosis. The majority of individuals with NAFLD have no symptoms and a normal physical examination (although the liver may be slightly enlarged); children may exhibit symptoms such as abdominal pain and fatigue, and may show patchy dark skin discoloration (acanthosis nigricans). A diagnosis of NAFLD is usually first suspected in an overweight or obese person who is found to have mild elevations in their liver blood tests during routine testing; NAFLD can be present with normal liver blood tests, however, or incidentally detected on imaging investigations such as abdominal ultrasound or CT scan. It is confirmed by imaging studies, most commonly a liver ultrasound or magnetic resonance imaging (MRI), and exclusion of other causes.

Some people with NAFLD may develop a more serious condition called non-alcoholic steatohepatitis (NASH): about 2-5 percent of adult Americans and up to 20 percent of those who are obese may suffer from NASH. In NASH, fat accumulation in the liver is associated with inflammation and different degrees of scarring. NASH is a potentially serious condition that carries a substantial risk of progression to end-stage liver disease, cirrhosis and hepatocellular carcinoma. Some patients who develop cirrhosis are at risk of liver failure and may eventually require a liver transplant.

NAFLD may be differentiated from NASH by the NAFLD Activity Score (NAS), the sum of the histopathology scores of a liver biopsy for steatosis (0 to 3), lobular inflammation (0 to 2), and hepatocellular ballooning (0 to 2). A NAS of <3 corresponds to NAFLD, 3-4 corresponds to borderline NASH, and >5 corresponds to NASH. The biopsy is also scored for fibrosis (0 to 4).

There are no drugs currently approved to prevent or treat NAFLD or NASH. A number of pharmacological interventions have been tried in NAFLD/NASH but with overall limited benefit.

### SUMMARY

The present invention relates to methods for treating non-alcoholic fatty liver disease (NAFLD) and non-alcoholic steatohepatitis (NASH), as well as related conditions that include but are not limited to alcoholic steatohepatitis (ASH), said method comprising administering to the subject in need thereof an effective amount of a GDF15 fusion polypeptide or GDF15 conjugate, e.g., a GDF15 fatty acid conjugate (usually in the form of a pharmaceutical composition) as described herein. In some aspects, the invention relates to methods for treating non-alcoholic fatty liver disease (NAFLD) and non-alcoholic steatohepatitis (NASH), as well as end-stage liver disease, hepatic steatosis (fatty liver), liver fibrosis, liver inflammation, liver cirrhosis, primary biliary cirrhosis (PBC), and hepatocellular carcinoma (HCC) in a subject in need thereof, said method comprising administering to the subject in need thereof an effective amount of a GDF15 fusion polypeptide (usually in the form of a pharmaceutical composition) as described herein.

In some embodiments, the methods of the invention comprise a portion of the wild type GDF15 full length protein, e.g., having NCBI reference sequence number NP_004855.2, and encoded by the polynucleotide sequence which has NCBI reference sequence number NM_004864.2, and found in such published patent applications as, e.g., WO97/00958, assigned to St. Vincents Hospital. By way of non-limiting example, in some embodiments, the methods of the invention comprise the mature GDF15 protein, i.e., amino acid residues 198-308 of the wild type GDF15 full length protein. In other embodiments, the methods of the invention comprise smaller fragments, domains, and/or regions of full length GDF15 protein.

In some embodiments, the methods of the invention comprise variants or mutations of the GDF15 protein sequence, e.g., biologically active GDF15 variants, and can include truncated versions of the GDF15 protein (in which residues from the C- and/or N-terminal regions have been eliminated, thereby shortening/truncating the protein), as well as variants with one or more point substitutions, deletions, and/or site-specific incorporation of amino acids at positions of interest (e.g., with conservative amino acid residues, with non-conservative residues, or with non-natural amino acid residues such as pyrrolysine). The terms “variant” and “mutant” are used interchangeably and are further defined herein.

In some embodiments, the methods of the invention comprise GDF15 fusion protein sequences, such as Fc fusions, or serum albumin (SA) fusions. The terms “fusion protein,” “fusion polypeptide,” and “fusions” are used interchangeably and are further defined herein. In still other embodiments, the methods of the invention comprise conjugations of GDF15 and fatty acids. Said conjugates and fusions may be intended to extend the half-life of the GDF15 moiety, in addition to serving as therapeutic agents for the conditions listed herein. In some embodiments, the conjugates and fusions used in the methods of the inventions comprise wild type GDF15; in other embodiments, the conjugates and fusions comprise variant GDF15 sequences relative to the wild type full length or mature protein.

Representative examples of said GDF15 variants, conjugates, and fusions are described, e.g., in PCT Publications WO13/148117 and WO14/120619 and all related patent family members (including but not limited to U.S. Pat. No. 9,161,966B1); and in PCT Publications WO2012/138919, WO13/113008, and WO15/017710, and all related patent family members. In all cases, representative examples of said GDF15 variants, conjugates, and fusions may be found in any related applications, issued patents, and family members of the above, both in the US and in the rest of the world. The contents of all of the above, as well as of any related applications, issued patents, and family members, are hereby incorporated herein by reference in their entirety. Specific embodiments can be found in the following table (Table 1):

TABLE 1
GDF15 Variants and fusion proteins

| | SEQ ID | |
|---|---|---|
| Description | NO: | SEQUENCE |
| Full-length | 2 | MPGQELRTVN GSQMLLVLLV LSWLPHGGAL SLAEASRASF PGPSELHSED |
| human GDF15 | | SRFRELRKRY EDLLTRLRAN QSWEDSNTDL VPAPAVRILT PEVRLGSGGH |
| | | LHLRISRAAL PEGLPEASRL HRALFRLSPT ASRSWDVTRP LRRQLSLARP |
| | | QAPALHLRLS PPPSQSDQLL AESSSARPQL ELHLRPQAAR GRRRARARNG |
| | | DHCPLGPGRC CRLHTVRASL EDLGWADWVL SPREVQVTMC IGACPSQFRA |
| | | ANMHAQIKTS LHRLKPDTVP APCCVPASYN PMVLIQKTDT GVSLQTYDDL |
| | | LAKDCHCI |
| Full-length | 3 | MKWVTFISLLFLFSSAYSRGVFRRDAHKSEVAHRFKDLGEENFKALVLIAFAQYLQQCPF |
| human serum | | EDHVKLVNEVTEFAKTCVADESAENCDKSLHTLFGDKLCTVATLRETYGEMADCCAKQEP |
| albumin | | ERNECFLQHKDDNPNLPRLVRPEVDVMCTAFHDNEETFLKKYLYEIARRHPYFYAPELLF |
| | | FAKRYKAAFTECCQAADKAACLLPKLDELRDEGKASSAKQRLKCASLQKFGERAFKAWAV |
| | | ARLSQRFPKAEFAEVSKLVTDLTKVHTECCHGDLLECADDRADLAKYICENQDSISSKLK |
| | | ECCEKPLLEKSHCIAEVENDEMPADLPSLAADFVESKDVCKNYAEAKDVFLGMFLYEYAR |
| | | RHPDYSVVLLLRLAKTYETTLEKCCAAADPHECYAKVFDEFKPLVEEPQNLIKQNCELFE |
| | | QLGEYKFQNALLVRYTKKVPQVSTPTLVEVSRNLGKVGSKCCKHPEAKRMPCAEDYLSVV |
| | | LNQLCVLHEKTPVSDRVTKCCTESLVNRRPCFSALEVDETYVPKEFNAETFTFHADICTL |
| | | SEKERQIKKQTALVELVKHKPKATKEQLKAVMDDFAAFVEKCCKADDKETCFAEEGKKLV |
| | | AASQAALGL |
| Mature | 4 | DAHKSE VAHRFKDLGE ENFKALVLIA FAQYLQQCPF EDHVKLVNEV |
| HSA(25-609) | | TEFAKTCVAD ESAENCDKSL HTLFGDKLCT VATLRETYGE MADCCAKQEP |
| | | ERNECFLQHK DDNPNLPRLV RPEVDVMCTA FHDNEETFLK KYLYEIARRH |
| | | PYFYAPELLF FAKRYKAAFT ECCQAADKAA CLLPKLDELR DEGKASSAKQ |
| | | RLKCASLQKF GERAFKAWAV ARLSQRFPKA EFAEVSKLVT DLTKVHTECC |
| | | HGDLLECADD RADLAKYICE NQDSISSKLK ECCEKPLLEK SHCIAEVEND |
| | | EMPADLPSLA ADFVESKDVC KNYAEAKDVF LGMFLYEYAR RHPDYSVVLL |
| | | LRLAKTYETT LEKCCAAADP HECYAKVFDE FKPLVEEPQN LIKQNCELFE |
| | | QLGEYKFQNA LLVRYTKKVP QVSTPTLVEV SRNLGKVGSK CCKHPEAKRM |
| | | PCAEDYLSVV LNQLCVLHEK TPVSDRVTKC CTESLVNRRP CFSALEVDET |
| | | YVPKEFNAET FTFHADICTL SEKERQIKKQ TALVELVKHK PKATKEQLKA |
| | | VMDDFAAFVE KCCKADDKET CFAEEGKKLV AASQAALGL |
| Mature | 5 | ARNGDHCPLG PGRCCRLHTV RASLEDLGWA DWVLSPREVQ VTMCIGACPS |
| GDF15(197- | | QFRAANMHAQ IKTSLHRLKP DTVPAPCCVP ASYNPMVLIQ KTDTGVSLQT |
| 308) | | YDDLLAKDCH CI |
| Mature | 6 | HHHHHHARNG DHCPLGPGRC CRLHTVRASL EDLGWADWVL SPREVQVTMC |
| 6xHis- | | IGACPSQFRA ANMHAQIKTS LHRLKPDTVP APCCVPASYN PMVLIQKTDT |
| GDF15(197- | | GVSLQTYDDL LAKDCHCI |
| 308) | | |
| Mature | 7 | HHHHHHHHGG SENLYFQGAR NGDHCPLGPG RCCRLHTVRA SLEDLGWADW |
| 6xHis-TEV- | | VLSPREVQVT MCIGACPSQF RAANMHAQIK TSLHRLKPDT VPAPCCVPAS |
| GDF15(197- | | YNPMVLIQKT DTGVSLQTYD DLLAKDCHCI |
| 308) | | |
| Mature M- | 8 | MHHHHHHQKP VGVEEPVYDT AGRPLFGNPS EVHPQSTLKL PHDRGEDDIE |
| 6xHis- | | TTLRDLPRKG DCRSGNHLGP VSGIYIKPGP VYYQDYTGPV YHRAPLEFFD |
| GDF15(197- | | ETQFEETTKR IGRVTGSDGK LYHIYVEVDG EILLKQAKRG TPRTLKWTRN |
| 308) | | TTNCPLWVTS CARNGDHCPL GPGRCCRLHT VRASLEDLGW ADWVLSPREV |
| | | QVTMCIGACP SQFRAANMHA QIKTSLHRLK PDTVPAPCCV |
| | | PASYNPMVLI QKTDTGVSLQ TYDDLLAKDC HCI |
| Full-length | 9 | EAHKSEIAHR YNALGEQHFK GLVLIAFSQY LQKASYDEHA KLVQEVTDFA |
| Murine serum | | KTCVADESAA NCDKSLHTLF GDKLCAIPNL RENYGELADC CTKQEPERNE |
| albumin MSA- | | CFLQHKDDNP SLPPFERPEA EAMCTSFKEN PTTFMGHYLH EVARRHPYFY |
| GDF15 fusion | | APELLYYAEQ YNEILTQCCA EADKESCLTP KLDGVKEKAL VSSVRQRMKC |
| protein(197- | | SSMQKFGERA FKAWAVARLS QTFPNADFAE ITKLATDLTK VNKECCHGDL |
| 308) | | LECADDRAEL AKYMCENQAT ISSKLQTCCD KPLLKKAHCL SEVEHDTMPA |
| | | DLPAIAADFV EDQEVCKNYA EAKDVFLGTF LYEYSRRHPD YSVSLLLRLA |
| | | KKYEATLEKC CAEANPPACY GTVLAEFQPL VEEPKNLVKT NCDLYEKLGE |
| | | YGFQNAILVR YTQKAPQVST PTLVEAARNL GRVGTKCCTL PEDQRLPCVE |
| | | DYLSAILNRV CLLHEKTPVS EHVTKCCSGS LVERRPCFSA LTVDETYVPK |
| | | EFKAETFTFH SDICTLPEKE KQIKKQTALA ELVKHKPKAT AEQLKTVMDD |
| | | FAQFLDTCCK AADKDTCFST EGPNLVTRAK DALAGGGGSG GGGSGGGGSA |
| | | RNGDHCPLGP GRCCRLHTVR ASLEDLGWAD WVLSPREVQV TMCIGACPSQ |
| | | FRAANMHAQI KTSLHRLKPD TVPAPCCVPA SYNPMVLIQK TDTGVSLQTY |
| | | DDLLAKDCHC I |
| Full-length | 10 | EAHKSEIAHR YNALGEQHFK GLVLIAFSQY LQKASYDEHA KLVQEVTDFA |
| Murine serum | | KTCVADESAA NCDKSLHTLF GDKLCAIPNL RENYGELADC CTKQEPERNE |
| albumin MSA- | | CFLQHKDDNP SLPPFERPEA EAMCTSFKEN PTTFMGHYLH EVARRHPYFY |
| GDF15 fusion | | APELLYYAEQ YNEILTQCCA EADKESCLTP KLDGVKEKAL VSSVRQRMKC |
| protein(211- | | SSMQKFGERA FKAWAVARLS QTFPNADFAE ITKLATDLTK VNKECCHGDL |
| 308) | | LECADDRAEL AKYMCENQAT ISSKLQTCCD KPLLKKAHCL SEVEHDTMPA |
| | | DLPAIAADFV EDQEVCKNYA EAKDVFLGTF LYEYSRRHPD YSVSLLLRLA |
| | | KKYEATLEKC CAEANPPACY GTVLAEFQPL VEEPKNLVKT NCDLYEKLGE |
| | | YGFQNAILVR YTQKAPQVST PTLVEAARNL GRVGTKCCTL PEDQRLPCVE |
| | | DYLSAILNRV CLLHEKTPVS EHVTKCCSGS LVERRPCFSA LTVDETYVPK |
| | | EFKAETFTFH SDICTLPEKE KQIKKQTALA ELVKHKPKAT AEQLKTVMDD |
| | | FAQFLDTCCK AADKDTCFST EGPNLVTRAK DALAGGGGSG GGGSGGGGSC |
| | | RLHTVRASLE DLGWADWVLS PREVQVTMCI GACPSQFRAA NMHAQIKTSL |
| | | HRLKPDTVPA PCCVPASYNP MVLIQKTDTG VSLQTYDDLL AKDCHCI |
| HSA(25- | 11 | DAHKSEVAHR FKDLGEENFK ALVLIAFAQY LQQSPFEDHV KLVNEVTEFA |
| 609)(C34S)(N503Q)- | | KTCVADESAE NCDKSLHTLF GDKLCTVATL RETYGEMADC CAKQEPERNE |
| GDF15(211- | | CFLQHKDDNP NLPRLVRPEV DVMCTAFHDN EETFLKKYLY EIARRHPYFY |
| 308) | | APELLFFAKR YKAAFTECCQ AADKAACLLP KLDELRDEGK ASSAKQRLKC |
| | | ASLQKFGERA FKAWAVARLS QRFPKAEFAE VSKLVTDLTK VHTECCHGDL |
| | | LECADDRADL AKYICENQDS ISSKLKECCE KPLLEKSHCI AEVENDEMPA |
| | | DLPSLAADFV ESKDVCKNYA EAKDVFLGMF LYEYARRHPD YSVVLLLRLA |
| | | KTYETTLEKC CAAADPHECY AKVFDEFKPL VEEPQNLIKQ NCELFEQLGE |
| | | YKFQNALLVR YTKKVPQVST PTLVEVSRNL GKVGSKCCKH PEAKRMPCAE |
| | | DYLSVVLNQL CVLHEKTPVS DRVTKCCTES LVNRRPCFSA LEVDETYVPK |
| | | EFQAETFTFH ADICTLSEKE RQIKKQTALV ELVKHKPKAT KEQLKAVMDD |
| | | FAAFVEKCCK ADDKETCFAE EGKKLVAASQ AALGLGGGGS GGGGSGGGGS |
| | | CRLHTVRASL EDLGWADWVL SPREVQVTMC IGACPSQFRA ANMHAQIKTS |
| | | LHRLKPDTVP APCCVPASYN PMVLIQKTDT GVSLQTYDDL LAKDCHCI |
| MSA- | 12 | EAHKSEIAHR YNALGEQHFK GLVLIAFSQY LQKASYDEHA KLVQEVTDFA |
| GDF15(197- | | KTCVADESAA NCDKSLHTLF GDKLCAIPNL RENYGELADC CTKQEPERNE |
| 308)(C203S) | | CFLQHKDDNP SLPPFERPEA EAMCTSFKEN PTTFMGHYLH EVARRHPYFY |
| (C210S) | | APELLYYAEQ YNEILTQCCA EADKESCLTP KLDGVKEKAL VSSVRQRMKC |
| | | SSMQKFGERA FKAWAVARLS QTFPNADFAE ITKLATDLTK VNKECCHGDL |
| | | LECADDRAEL AKYMCENQAT ISSKLQTCCD KPLLKKAHCL SEVEHDTMPA |
| | | DLPAIAADFV EDQEVCKNYA EAKDVFLGTF LYEYSRRHPD YSVSLLLRLA |
| | | KKYEATLEKC CAEANPPACY GTVLAEFQPL VEEPKNLVKT NCDLYEKLGE |
| | | YGFQNAILVR YTQKAPQVST PTLVEAARNL GRVGTKCCTL PEDQRLPCVE |
| | | DYLSAILNRV CLLHEKTPVS EHVTKCCSGS LVERRPCFSA LTVDETYVPK |
| | | EFKAETFTFH SDICTLPEKE KQIKKQTALA ELVKHKPKAT AEQLKTVMDD |
| | | FAQFLDTCCK AADKDTCFST EGPNLVTRAK DALAGGGGSG GGGSGGGGSA |
| | | RNGDHSPLGP GRSCRLHTVR ASLEDLGWAD WVLSPREVQV TMCIGACPSQ |
| | | FRAANMHAQI KTSLHRLKPD TVPAPCCVPA SYNPMVLIQK TDTGVSLQTY |
| | | DDLLAKDCHC I |
| MSA- | 13 | EAHKSEIAHR YNALGEQHFK GLVLIAFSQY LQKASYDEHA KLVQEVTDFA |
| GDF15(197- | | KTCVADESAA NCDKSLHTLF GDKLCAIPNL RENYGELADC CTKQEPERNE |
| 308)(C273S) | | CFLQHKDDNP SLPPFERPEA EAMCTSFKEN PTTFMGHYLH EVARRHPYFY |
| | | APELLYYAEQ YNEILTQCCA EADKESCLTP KLDGVKEKAL VSSVRQRMKC |
| | | SSMQKFGERA FKAWAVARLS QTFPNADFAE ITKLATDLTK VNKECCHGDL |
| | | LECADDRAEL AKYMCENQAT ISSKLQTCCD KPLLKKAHCL SEVEHDTMPA |
| | | DLPAIAADFV EDQEVCKNYA EAKDVFLGTF LYEYSRRHPD YSVSLLLRLA |
| | | KKYEATLEKC CAEANPPACY GTVLAEFQPL VEEPKNLVKT NCDLYEKLGE |
| | | YGFQNAILVR YTQKAPQVST PTLVEAARNL GRVGTKCCTL PEDQRLPCVE |
| | | DYLSAILNRV CLLHEKTPVS EHVTKCCSGS LVERRPCFSA LTVDETYVPK |
| | | EFKAETFTFH SDICTLPEKE KQIKKQTALA ELVKHKPKAT AEQLKTVMDD |
| | | FAQFLDTCCK AADKDTCFST EGPNLVTRAK DALAGGGGSG GGGSGGGGSA |
| | | RNGDHCPLGP GRCCRLHTVR ASLEDLGWAD WVLSPREVQV TMCIGACPSQ |
| | | FRAANMHAQI KTSLHRLKPD TVPAPSCVPA SYNPMVLIQK TDTGVSLQTY |
| | | DDLLAKDCHC I |
| HSA-(G4S)3- | 14 | DAHKSEVAHR FKDLGEENFK ALVLIAFAQY LQQCPFEDHV KLVNEVTEFA |
| GDF15(197- | | KTCVADESAE NCDKSLHTLF GDKLCTVATL RETYGEMADC CAKQEPERNE |
| 308) | | CFLQHKDDNP NLPRLVRPEV DVMCTAFHDN EETFLKKYLY EIARRHPYFY |
| | | APELLFFAKR YKAAFTECCQ AADKAACLLP KLDELRDEGK ASSAKQRLKC |
| | | ASLQKFGERA FKAWAVARLS QRFPKAEFAE VSKLVTDLTK VHTECCHGDL |
| | | LECADDRADL AKYICENQDS ISSKLKECCE KPLLEKSHCI AEVENDEMPA |
| | | DLPSLAADFV ESKDVCKNYA EAKDVFLGMF LYEYARRHPD YSVVLLLRLA |
| | | KTYETTLEKC CAAADPHECY AKVFDEFKPL VEEPQNLIKQ NCELFEQLGE |
| | | YKFQNALLVR YTKKVPQVST PTLVEVSRNL GKVGSKCCKH PEAKRMPCAE |
| | | DYLSVVLNQL CVLHEKTPVS DRVTKCCTES LVNRRPCFSA LEVDETYVPK |
| | | EFNAETFTFH ADICTLSEKE RQIKKQTALV ELVKHKPKAT KEQLKAVMDD |
| | | FAAFVEKCCK ADDKETCFAE EGKKLVAASQ AALGLGGGGS GGGGSGGGGS |
| | | ARNGDHCPLG PGRCCRLHTV RASLEDLGWA DWVLSPREVQ VTMCIGACPS |
| | | QFRAANMHAQ IKTSLHRLKP DTVPAPCCVP ASYNPMVLIQ KTDTGVSLQT |
| | | YDDLLAKDCH CI |
| HSA-GGGS- | 15 | DAHKSEVAHR FKDLGEENFK ALVLIAFAQY LQQCPFEDHV KLVNEVTEFA |
| GDF15(197- | | KTCVADESAE NCDKSLHTLF GDKLCTVATL RETYGEMADC CAKQEPERNE |
| 308) | | CFLQHKDDNP NLPRLVRPEV DVMCTAFHDN EETFLKKYLY EIARRHPYFY |
| | | APELLFFAKR YKAAFTECCQ AADKAACLLP KLDELRDEGK ASSAKQRLKC |
| | | ASLQKFGERA FKAWAVARLS QRFPKAEFAE VSKLVTDLTK VHTECCHGDL |
| | | LECADDRADL AKYICENQDS ISSKLKECCE KPLLEKSHCI AEVENDEMPA |
| | | DLPSLAADFV ESKDVCKNYA EAKDVFLGMF LYEYARRHPD YSVVLLLRLA |
| | | KTYETTLEKC CAAADPHECY AKVFDEFKPL VEEPQNLIKQ NCELFEQLGE |
| | | YKFQNALLVR YTKKVPQVST PTLVEVSRNL GKVGSKCCKH PEAKRMPCAE |
| | | DYLSVVLNQL CVLHEKTPVS DRVTKCCTES LVNRRPCFSA LEVDETYVPK |
| | | EFNAETFTFH ADICTLSEKE RQIKKQTALV ELVKHKPKAT KEQLKAVMDD |
| | | FAAFVEKCCK ADDKETCFAE EGKKLVAASQ AALGLGGGGS ARNGDHCPLG |
| | | PGRCCRLHTV RASLEDLGWA DWVLSPREVQ VTMCIGACPS QFRAANMHAQ |
| | | IKTSLHRLKP DTVPAPCCVP ASYNPMVLIQ KTDTGVSLQT YDDLLAKDCH CI |
| HSA-GPPGS- | 16 | DAHKSEVAHR FKDLGEENFK ALVLIAFAQY LQQCPFEDHV KLVNEVTEFA |
| GDF15(197- | | KTCVADESAE NCDKSLHTLF GDKLCTVATL RETYGEMADC CAKQEPERNE |
| 308) | | CFLQHKDDNP NLPRLVRPEV DVMCTAFHDN EETFLKKYLY EIARRHPYFY |
| | | APELLFFAKR YKAAFTECCQ AADKAACLLP KLDELRDEGK ASSAKQRLKC |
| | | ASLQKFGERA FKAWAVARLS QRFPKAEFAE VSKLVTDLTK VHTECCHGDL |
| | | LECADDRADL AKYICENQDS ISSKLKECCE KPLLEKSHCI AEVENDEMPA |
| | | DLPSLAADFV ESKDVCKNYA EAKDVFLGMF LYEYARRHPD YSVVLLLRLA |
| | | KTYETTLEKC CAAADPHECY AKVFDEFKPL VEEPQNLIKQ NCELFEQLGE |
| | | YKFQNALLVR YTKKVPQVST PTLVEVSRNL GKVGSKCCKH PEAKRMPCAE |
| | | DYLSVVLNQL CVLHEKTPVS DRVTKCCTES LVNRRPCFSA LEVDETYVPK |
| | | EFNAETFTFH ADICTLSEKE RQIKKQTALV ELVKHKPKAT KEQLKAVMDD |
| | | FAAFVEKCCK ADDKETCFAE EGKKLVAASQ AALGLGPPGS ARNGDHCPLG |
| | | PGRCCRLHTV RASLEDLGWA DWVLSPREVQ VTMCIGACPS QFRAANMHAQ |
| | | IKTSLHRLKP DTVPAPCCVP ASYNPMVLIQ KTDTGVSLQT YDDLLAKDCH CI |
| HSA- | 17 | DAHKSEVAHR FKDLGEENFK ALVLIAFAQY LQQCPFEDHV KLVNEVTEFA |
| GDF15(197- | | KTCVADESAE NCDKSLHTLF GDKLCTVATL RETYGEMADC CAKQEPERNE |
| 308) | | CFLQHKDDNP NLPRLVRPEV DVMCTAFHDN EETFLKKYLY EIARRHPYFY |
| | | APELLFFAKR YKAAFTECCQ AADKAACLLP KLDELRDEGK ASSAKQRLKC |
| | | ASLQKFGERA FKAWAVARLS QRFPKAEFAE VSKLVTDLTK VHTECCHGDL |
| | | LECADDRADL AKYICENQDS ISSKLKECCE KPLLEKSHCI AEVENDEMPA |
| | | DLPSLAADFV ESKDVCKNYA EAKDVFLGMF LYEYARRHPD YSVVLLLRLA |
| | | KTYETTLEKC CAAADPHECY AKVFDEFKPL VEEPQNLIKQ NCELFEQLGE |
| | | YKFQNALLVR YTKKVPQVST PTLVEVSRNL GKVGSKCCKH PEAKRMPCAE |
| | | DYLSVVLNQL CVLHEKTPVS DRVTKCCTES LVNRRPCFSA LEVDETYVPK |
| | | EFNAETFTFH ADICTLSEKE RQIKKQTALV ELVKHKPKAT KEQLKAVMDD |
| | | FAAFVEKCCK ADDKETCFAE EGKKLVAASQ AALGLARNGD HCPLGPGRCC |
| | | RLHTVRASLE DLGWADWVLS PREVQVTMCI GACPSQFRAA NMHAQIKTSL |
| | | HRLKPDTVPA PCCVPASYNP MVLIQKTDTG VSLQTYDDLL AKDCHCI |
| MSA(Domain1)- | 18 | EAHKSEIAHR YNDLGEQHFK GLVLIAFSQY LQKCSYDEHA KLVQEVTDFA |
| (G4S)3- | | KTCVADESAA NCDKSLHTLF GDKLCAIPNL RENYGELADC CTKQEPERNE |
| GDF15(197- | | CFLQHKDDNP SLPPFERPEA EAMCTSFKEN PTTFMGHYLH EVARRHPYFY |
| 308) | | APELLYYAEQ YNEILTQCCA EADKESCLTP KLDGVKEKAL VSSVRQRGGG |
| | | GSGGGGSGGG GSARNGDHCP LGPGRCCRLH TVRASLEDLG WADWVLSPRE |
| | | VQVTMCIGAC PSQFRAANMH AQIKTSLHRL KPDTVPAPCC VPASYNPMVL |
| | | IQKTDTGVSL QTYDDLLAKD CHCI |
| Fc-(G4S)3- | 19 | DKTHTCPPCP APELLGGPSV FLFPPKPKDT LMISRTPEVT CVVVDVSHED |
| GDF15(197- | | PEVKFNWYVD GVEVHNAKTK PREEQYNSTY RVVSVLTVLH QDWLNGKEYK |
| 308) | | CKVSNKALPA PIEKTISKAK GQPREPQVYT LPPSRDELTK NQVSLTCLVK |
| | | GFYPSDIAVE WESNGQPENN YKTTPPVLDS DGSFFLYSKL TVDKSRWQQG |
| | | NVFSCSVMHE ALHNHYTQKS LSLSPGKGGG GSGGGGSGGG GSARNGDHCP |
| | | LGPGRCCRLH TVRASLEDLG WADWVLSPRE VQVTMCIGAC PSQFRAANMH |
| | | AQIKTSLHRL KPDTVPAPCC VPASYNPMVL IQKTDTGVSL QTYDDLLAKD CHCI |
| HSA- | 20 | DAHKSEVAHR FKDLGEENFK ALVLIAFAQY LQQCPFEDHV KLVNEVTEFA |
| GDF15(197- | | KTCVADESAE NCDKSLHTLF GDKLCTVATL RETYGEMADC CAKQEPERNE |
| 308)(R198H) | | CFLQHKDDNP NLPRLVRPEV DVMCTAFHDN EETFLKKYLY EIARRHPYFY |
| | | APELLFFAKR YKAAFTECCQ AADKAACLLP KLDELRDEGK ASSAKQRLKC |
| | | ASLQKFGERA FKAWAVARLS QRFPKAEFAE VSKLVTDLTK VHTECCHGDL |
| | | LECADDRADL AKYICENQDS ISSKLKECCE KPLLEKSHCI AEVENDEMPA |
| | | DLPSLAADFV ESKDVCKNYA EAKDVFLGMF LYEYARRHPD YSVVLLLRLA |
| | | KTYETTLEKC CAAADPHECY AKVFDEFKPL VEEPQNLIKQ NCELFEQLGE |
| | | YKFQNALLVR YTKKVPQVST PTLVEVSRNL GKVGSKCCKH PEAKRMPCAE |
| | | DYLSVVLNQL CVLHEKTPVS DRVTKCCTES LVNRRPCFSA LEVDETYVPK |
| | | EFNAETFTFH ADICTLSEKE RQIKKQTALV ELVKHKPKAT KEQLKAVMDD |
| | | FAAFVEKCCK ADDKETCFAE EGKKLVAASQ AALGLGGGGS GGGGSGGGGS |
| | | AHNGDHCPLG PGRCCRLHTV RASLEDLGWA DWVLSPREVQ VTMCIGACPS |
| | | QFRAANMHAQ IKTSLHRLKP DTVPAPCCVP ASYNPMVLIQ KTDTGVSLQT |
| | | YDDLLAKDCH CI |
| HSA- | 21 | DAHKSEVAHR FKDLGEENFK ALVLIAFAQY LQQCPFEDHV KLVNEVTEFA |
| GDF15(197- | | KTCVADESAE NCDKSLHTLF GDKLCTVATL RETYGEMADC CAKQEPERNE |
| 308)(R198H) | | CFLQHKDDNP NLPRLVRPEV DVMCTAFHDN EETFLKKYLY EIARRHPYFY |
| (N199A) | | APELLFFAKR YKAAFTECCQ AADKAACLLP KLDELRDEGK ASSAKQRLKC |
| | | ASLQKFGERA FKAWAVARLS QRFPKAEFAE VSKLVTDLTK VHTECCHGDL |
| | | LECADDRADL AKYICENQDS ISSKLKECCE KPLLEKSHCI AEVENDEMPA |
| | | DLPSLAADFV ESKDVCKNYA EAKDVFLGMF LYEYARRHPD YSVVLLLRLA |
| | | KTYETTLEKC CAAADPHECY AKVFDEFKPL VEEPQNLIKQ NCELFEQLGE |
| | | YKFQNALLVR YTKKVPQVST PTLVEVSRNL GKVGSKCCKH PEAKRMPCAE |
| | | DYLSVVLNQL CVLHEKTPVS DRVTKCCTES LVNRRPCFSA LEVDETYVPK |
| | | EFNAETFTFH ADICTLSEKE RQIKKQTALV ELVKHKPKAT KEQLKAVMDD |
| | | FAAFVEKCCK ADDKETCFAE EGKKLVAASQ AALGLGGGGS GGGGSGGGGS |
| | | AHAGDHCPLG PGRCCRLHTV RASLEDLGWA DWVLSPREVQ VTMCIGACPS |
| | | QFRAANMHAQ IKTSLHRLKP DTVPAPCCVP ASYNPMVLIQ KTDTGVSLQT |
| | | YDDLLAKDCH CI |
| HSA- | 22 | DAHKSEVAHR FKDLGEENFK ALVLIAFAQY LQQCPFEDHV KLVNEVTEFA |
| GDF15(197- | | KTCVADESAE NCDKSLHTLF GDKLCTVATL RETYGEMADC CAKQEPERNE |
| 308)(N199E) | | CFLQHKDDNP NLPRLVRPEV DVMCTAFHDN EETFLKKYLY EIARRHPYFY |
| | | APELLFFAKR YKAAFTECCQ AADKAACLLP KLDELRDEGK ASSAKQRLKC |
| | | ASLQKFGERA FKAWAVARLS QRFPKAEFAE VSKLVTDLTK VHTECCHGDL |
| | | LECADDRADL AKYICENQDS ISSKLKECCE KPLLEKSHCI AEVENDEMPA |
| | | DLPSLAADFV ESKDVCKNYA EAKDVFLGMF LYEYARRHPD YSVVLLLRLA |
| | | KTYETTLEKC CAAADPHECY AKVFDEFKPL VEEPQNLIKQ NCELFEQLGE |
| | | YKFQNALLVR YTKKVPQVST PTLVEVSRNL GKVGSKCCKH PEAKRMPCAE |
| | | DYLSVVLNQL CVLHEKTPVS DRVTKCCTES LVNRRPCFSA LEVDETYVPK |
| | | EFNAETFTFH ADICTLSEKE RQIKKQTALV ELVKHKPKAT KEQLKAVMDD |
| | | FAAFVEKCCK ADDKETCFAE EGKKLVAASQ AALGLGGGGS GGGGSGGGGS |
| | | AREGDHCPLG PGRCCRLHTV RASLEDLGWA DWVLSPREVQ VTMCIGACPS |
| | | QFRAANMHAQ IKTSLHRLKP DTVPAPCCVP ASYNPMVLIQ KTDTGVSLQT |
| | | YDDLLAKDCH CI |
| MSA-GDF15- | 23 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(197- | | MCTSFKENPTTFMGHYLHEVARRHPYFYAPELLYYAEQYNEILTQCCAEADKESCLTPKLD |
| 308)(Q247R) | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVQVTMCIGACPSRFRAANMHAQIKTSLHRLKPDT |
| | | VPAPCCVPASYNPMVLIQKTDTGVSLQTYDDLLAKDCHCI |
| MSA-GDF15- | 24 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(197- | | MCTSFKENPTTFMGHYLHEVARRHPYFYAPELLYYAEQYNEILTQCCAEADKESCLTPKLD |
| 308)(S278R) | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVQVTMCIGACPSQFRAANMHAQIKTSLHRLKPDT |
| | | VPAPCCVPARYNPMVLIQKTDTGVSLQTYDDLLAKDCHCI |
| MSA-GDF15- | 25 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(197- | | MCTSFKENPTTFMGHYLHEVARRHPYFYAPELLYYAEQYNEILTQCCAEADKESCLTPKLD |
| 308)(D289R) | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVQVTMCIGACPSQFRAANMHAQIKTSLHRLKPDT |
| | | VPAPCCVPASYNPMVLIQKTRTGVSLQTYDDLLAKDCHCI |
| MSA-GDF15- | 26 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(197- | | MCTSFKENPTTFMGHYLHEVARRHPYFYAPELLYYAEQYNEILTQCCAEADKESCLTPKLD |
| 308)(L294R) | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVQVTMCIGACPSQFRAANMHAQIKTSLHRLKPDT |
| | | VPAPCCVPASYNPMVLIQKTDTGVSRQTYDDLLAKDCHCI |
| MSA-GDF15- | 27 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| (T215R) | | MCTSFKENPTTFMGHYLHEVARRHPYFYAPELLYYAEQYNEILTQCCAEADKESCLTPKLD |
| | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHRVRASLEDLGWADWVLSPREVQVTMCIGACPSQFRAANMHAQIKTSLHRLKPDT |
| | | VPAPCCVPASYNPMVLIQKTDTGVSLQTYDDLLAKDCHCI |
| MSA-GDF15- | 28 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(E221R) | | MCTSFKENPTTFMGHYLHEVARRHPYFYAPELLYYAEQYNEILTQCCAEADKESCLTPKLD |
| | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLRDLGWADWVLSPREVQVTMCIGACPSQFRAANMHAQIKTSLHRLKPDT |
| | | VPAPCCVPASYNPMVLIQKTDTGVSLQTYDDLLAKDCHCI |
| MSA-GDF15- | 29 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(W228A) | | MCTSFKENPTTFMGHYLHEVARRHPYFYAPELLYYAEQYNEILTQCCAEADKESCLTPKLD |
| | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADRVLSPREVQVTMCIGACPSQFRAANMHAQIKTSLHRLKPDT |
| | | VPAPCCVPASYNPMVLIQKTDTGVSLQTYDDLLAKDCHCI |
| MSA-GDF15- | 30 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(S231R) | | MCTSFKENPTTFMGHYLHEVARRHPYFYAPELLYYAEQYNEILTQCCAEADKESCLTPKLD |
| | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLRPREVQVTMCIGACPSQFRAANMHAQIKTSLHRLKPDT |
| | | VPAPCCVPASYNPMVLIQKTDTGVSLQTYDDLLAKDCHCI |
| MSA-GDF15- | 31 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(Q236R) | | MCTSFKENPTTFMGHYLHEVARRHPYFYAPELLYYAEQYNEILTQCCAEADKESCLTPKLD |
| | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVRVTMCIGACPSQFRAANMHAQIKTSLHRLKPDT |
| | | VPAPCCVPASYNPMVLIQKTDTGVSLQTYDDLLAKDCHCI |
| MSA-GDF15- | 32 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(M253R) | | MCTSFKENPTTFMGHYLHEVARRHPYFYAPELLYYAEQYNEILTQCCAEADKESCLTPKLD |
| | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVQVTMCIGACPSQFRAANRHAQIKTSLHRLKPDT |
| | | VPAPCCVPASYNPMVLIQKTDTGVSLQTYDDLLAKDCHCI |
| MSA-GDF15- | 33 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(I285R) | | MCTSFKENPTTFMGHYLHEVARRHPYFYAPELLYYAEQYNEILTQCCAEADKESCLTPKLD |
| | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVQVTMCIGACPSQFRAANMHAQIKTSLHRLKPDT |
| | | VPAPCCVPASYNPMVLRQKTDTGVSLQTYDDLLAKDCHCI |
| MSA-GDF15- | 34 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(I285A) | | MCTSFKENPTTFMGHYLHEVARRHPYFYAPELLYYAEQYNEILTQCCAEADKESCLTPKLD |
| | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVQVTMCIGACPSQFRAANMHAQIKTSLHRLKPDT |
| | | VPAPCCVPASYNPMVLAQKTDTGVSLQTYDDLLAKDCHCI |
| MSA-GDF15- | 35 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(Q286R) | | MCTSFKENPTTFMGHYLHEVARRHPYFYAPELLYYAEQYNEILTQCCAEADKESCLTPKLD |
| | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVQVTMCIGACPSQFRAANMHAQIKTSLHRLKPDT |
| | | VPAPCCVPASYNPMVLIRKTDTGVSLQTYDDLLAKDCHCI |
| MSA-GDF15- | 36 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(V292R) | | MCTSFKENPTTFMGHYLHEVARRHPYFYAPELLYYAEQYNEILTQCCAEADKESCLTPKLD |
| | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVQVTMCIGACPSQFRAANMHAQIKTSLHRLKPDT |
| | | VPAPCCVPASYNPMVLIQKTDTGRSLQTYDDLLAKDCHCI |
| MSA-GDF15- | 37 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(L294A) | | MCTSFKENPTTFMGHYLHEVARRHPYFYAPELLYYAEQYNEILTQCCAEADKESCLTPKLD |
| | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVQVTMCIGACPSQFRAANMHAQIKTSLHRLKPDT |
| | | VPAPCCVPASYNPMVLIQKTDTGVSAQTYDDLLAKDCHCI |
| MSA-GDF15- | 38 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(I285A) | | MCTSFKENPTTFMGHYLHEVARRHPYFYAPELLYYAEQYNEILTQCCAEADKESCLTPKLD |
| (L294A) | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVQVTMCIGACPSQFRAANMHAQIKTSLHRLKPDT |
| | | VPAPCCVPASYNPMVLAQKTDTGVSAQTYDDLLAKDCHCI |
| MSA-GDF15- | 39 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(Q295R) | | MCTSFKENPTTFMGHYLHEVARRHPYFYAPELLYYAEQYNEILTQCCAEADKESCLTPKLD |
| | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVQVTMCIGACPSQFRAANMHAQIKTSLHRLKPDT |
| | | VPAPCCVPASYNPMVLIQKTDTGVSLRTYDDLLAKDCHCI |
| MSA-GDF15- | 40 | EAHKSEIAHRYNALGEQHFKGLVLIAFSQYLQKASYDEHAKLVQEVTDFAKTCVADESAAN |
| (G4S)3- | | CDKSLHTLFGDKLCAIPNLRENYGELADCCTKQEPERNECFLQHKDDNPSLPPFERPEAEA |
| GDF15(T296R) | | MCTSFKENPTTFMGHYLHEVARRHPYFYAPELLYYAEQYNEILTQCCAEADKESCLTPKLD |
| | | GVKEKALVSSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDTMPADLPAI |
| | | AADFVEDQEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQLKTVMD |
| | | DFAQFLDTCCKAADKDTCFSTEGPNLVTRAKDALAGGGGSGGGGSGGGGSARNGDHCPLGP |
| | | GRCCRLHTVRASLEDLGWADWVLSPREVQVTMCIGACPSQFRAANMHAQIKTSLHRLKPDT |
| | | VPAPCCVPASYNPMVLIQKTDTGVSLQRYDDLLAKDCHCI |
| hGDF15-AHA- | 41 | AHAGDHCPLGPGRCCRLHTVRASLEDLGWADWVLSPREVQVTMCIGACPSQFRAANMHAQI |
| PEG24-FA, or | | KTSLHRLKPDTVPAPCCVPASYNPMVLIQKTDTGVSLQTYDDLLAKDCHCI |
| AHA-(200- | | |
| 308)-hGDF15 | | |
| GDF15 mutein | 42 | ARNGDHCPLGPGRCCRLHTVRASLEDLGWADWVLSPREVQVTMCIGACPSQFRAANMHAQ |
| v1 K69Q, | | IKTSLHRLQPDTVPAPCCVPASYNPMVLIQRTDTGVSLQTYDDLLARDCHCI |
| K107R | | |
| GDF15 mutein | 43 | ARNGDHCPLGPGRCCRLHTVRASLEDLGWADWVLSPREVQVTMCIGACPSQFRAANMHAQ |
| v2 K62Q, | | IQTSLHRLKPDTVPAPCCVPASYNPMVLIQRTDTGVSLQTYDDLLARDCHCI |
| K91R, K107R | | |
| GDF15 mutein | 44 | ARNGDHCPLGPGRCCRLHTVRASLEDLGWADWVLSPREVQVTMCIGACPSQFRAANMHAQ |
| v3 K62Q, | | IQTSLHRLQPDTVPAPCCVPASYNPMVLIQKTDTGVSLQTYDDLLARDCHCI |
| K69Q, K107R | | |
| GDF15 mutein | 45 | ARNGDHCPLGPGRCCRLHTVRASLEDLGWADWVLSPREVQVTMCIGACPSQFRAANMHAQ |
| v4 K62Q, | | IQTSLHRLQPDTVPAPCCVPASYNPMVLIQRTDTGVSLQTYDDLLAKDCHCI |
| K69Q, K91R | | |
| GDF15 mutein | 46 | ARNGDHCPLGPGRCCRLHTVRASLEDLGWADWVLSPREVQVTMCIGACPSQFRAANMHAQ |
| v5 K91R, | | IKTSLHRLKPDTVPAPCCVPASYNPMVLIQRTDTGVSLQTYDDLLARDCHCI |
| K107R | | |
| GDF15 mutein | 47 | ARNGDHCPLGPGRCCRLHTVRASLEDLGWADWVLSPREVQVTMCIGACPSQFRAANMHAQ |
| v6 K69Q, | | IKTSLHRLQPDTVPAPCCVPASYNPMVLIQKTDTGVSLQTYDDLLARDCHCI |
| K107R | | |
| GDF15 mutein | 48 | ARNGDHCPLGPGRCCRLHTVRASLEDLGWADWVLSPREVQVTMCIGACPSQFRAANMHAQ |
| v7 K69Q, | | IKTSLHRLQPDTVPAPCCVPASYNPMVLIQRTDTGVSLQTYDDLLAKDCHCI |
| K91R | | |
| GDF15 mutein | 49 | ARNGDHCPLGPGRCCRLQSLRASLEDLGWADWVLSPREVQVTMCIGACPSQFRAANMHAQ |
| v8 H18Q, | | IQTSLHRLQPDTVPAPCCVPASYNPMVLIQRTDTGVSLQTYDDLLARDCHCI |
| T19S, V20L, | | |
| K62Q, K69Q, | | |
| K91R, K107R | | |
| GDF15 mutein | 50 | ARNGDHCPLGPGRCCRLQSLRASLEDLGWADWVLSPREVQVTMCIGACPSQFRAANMHAQ |
| v9 H18Q, | | IQTSLHRLKPDTVPAPCCVPASYNPMVLIQRTDTGVSLQTYDDLLARDCHCI |
| T19S, V20L, | | |
| K62Q, K91R, | | |
| K107R | | |
| GDF15 mutein | 51 | ARNGDHCPLGPGRCCRLQSLRASLEDLGWADWVLSPREVQVTMCIGACPSQFRAANMHAQ |
| v10 H18Q, | | IQTSLHRLQPDTVPAPCCVPASYNPMVLIQKTDTGVSLQTYDDLLARDCHCI |
| T19S, V20L, | | |
| K62Q, K69Q, | | |
| K107R | | |
| GDF15 mutein | 52 | ARNGDHCPLGPGRCCRLQSLRASLEDLGWADWVLSPREVQVTMCIGACPSQFRAANMHAQ |
| v11 H18Q, | | IQTSLHRLQPDTVPAPCCVPASYNPMVLIQRTDTGVSLQTYDDLLAKDCHCI |
| T19S, V20L, | | |
| K62Q, K69Q, | | |
| K91R | | |
| GDF15 mutein | 53 | PARNGDHCPLGPGRCCRLHTVRASLEDLGWADWVLSPREVQVTMCIGACPSQFRAANMHA |
| v12 NPro- | | QIKTSLHRLKPDTVPAPCCVPASYNPMVLIQKTDTGVSLQTYDDLLAKDCHCI |
| GDF15 | | |
| GDF15 mutein | 54 | PARNGDHCPLGPGRCCRLHTVRASLEDLGWADWVLSPREVQVTMCIGACPSQFRAANMHA |
| v13 NPro, | | QIKTSLHRLQPDTVPAPCCVPASYNPMVLIQRTDTGVSLQTYDDLLARDCHCI |
| K70Q, K92R | | |
| GDF15 mutein | 55 | PARNGDHCPLGPGRCCRLHTVRASLEDLGWADWVLSPREVQVTMCIGACPSQFRAANMHA |
| v14 NPro, | | QIQTSLHRLKPDTVPAPCCVPASYNPMVLIQRTDTGVSLQTYDDLLARDCHCI |
| K63Q, K92R, | | |
| K108R | | |
| GDF15 mutein | 56 | PARNGDHCPLGPGRCCRLHTVRASLEDLGWADWVLSPREVQVTMCIGACPSQFRAANMHA |
| v15 NPro, | | QIQTSLHRLQPDTVPAPCCVPASYNPMVLIQKTDTGVSLQTYDDLLARDCHCI |
| K63Q, K70Q, | | |
| K108R | | |
| GDF15 mutein | 57 | PARNGDHCPLGPGRCCRLHTVRASLEDLGWADWVLSPREVQVTMCIGACPSQFRAANMHA |
| v16 NPro, | | QIQTSLHRLQPDTVPAPCCVPASYNPMVLIQRTDTGVSLQTYDDLLAKDCHCI |
| K63Q, K70Q, | | |
| K92R | | |
| GDF15 mutein | 58 | PARNGDHCPLGPGRCCRLHTVRASLEDLGWADWVLSPREVQVTMCIGACPSQFRAANMHA |
| v17 NPro, | | QIKTSLHRLKPDTVPAPCCVPASYNPMVLIQRTDTGVSLQTYDDLLARDCHCI |
| K92R, K108R | | |
| GDF15 mutein | 59 | PARNGDHCPLGPGRCCRLHTVRASLEDLGWADWVLSPREVQVTMCIGACPSQFRAANMHA |
| v18 NPro, | | QIKTSLHRLQPDTVPAPCCVPASYNPMVLIQKTDTGVSLQTYDDLLARDCHCI |
| K70Q, K108R | | |
| GDF15 mutein | 60 | PARNGDHCPLGPGRCCRLHTVRASLEDLGWADWVLSPREVQVTMCIGACPSQFRAANMHA |
| v19 NPro, | | QIKTSLHRLQPDTVPAPCCVPASYNPMVLIQRTDTGVSLQTYDDLLAKDCHCI |
| K70Q, K92R | | |
| GDF15 mutein | 61 | PARNGDHCPLGPGRCCRLQSLRASLEDLGWADWVLSPREVQVTMCIGACPSQFRAANMHA |
| v20 NPro, | | QIQTSLHRLQPDTVPAPCCVPASYNPMVLIQRTDTGVSLQTYDDLLARDCHCI |
| H19Q, T20S, | | |
| V21L, K63Q, | | |
| K70Q, K92R, | | |
| K108R | | |
| GDF15 mutein | 62 | PARNGDHCPLGPGRCCRLQSLRASLEDLGWADWVLSPREVQVTMCIGACPSQFRAANMHA |
| v21 NPro, | | QIQTSLHRLKPDTVPAPCCVPASYNPMVLIQRTDTGVSLQTYDDLLARDCHCI |
| H19Q, T20S, | | |
| V21L, K63Q, | | |
| K92R, K108R | | |
| GDF15 mutein | 63 | PARNGDHCPLGPGRCCRLQSLRASLEDLGWADWVLSPREVQVTMCIGACPSQFRAANMHA |
| v22 NPro, | | QIQTSLHRLQPDTVPAPCCVPASYNPMVLIQKTDTGVSLQTYDDLLARDCHCI |
| H19Q, T20S, | | |
| V21L, K63Q, | | |
| K70Q, K108R | | |
| GDF15 mutein | 64 | PARNGDHCPLGPGRCCRLQSLRASLEDLGWADWVLSPREVQVTMCIGACPSQFRAANMHA |
| v23 NPro, | | QIQTSLHRLQPDTVPAPCCVPASYNPMVLIQRTDTGVSLQTYDDLLAKDCHCI |
| H19Q, T20S, | | |
| V21L, K63Q, | | |
| K70Q, K92R | | |
| Fusion | 65 | MDMRVPAQLLGLLLLWLRGARCDAHKSEVAHRFKDLGEENFKALVLIAFAQYLQQCPFED |
| molecule HSA | | HVKLVNEVTEFAKTCVADESAENCDKSLHTLFGDKLCTVATLRETYGEMADCCAKQEPER |
| with IgK | | NECFLQHKDDNPNLPRLVRPEVDVMCTAFHDNEETFLKKYLYEIARRHPYFYAPELLFFA |
| signal | | KRYKAAFTECCQAADKAACLLPKLDELRDEGKASSAKQRLKCASLQKFGERAFKAWAVAR |
| sequence | | LSQRFPKAEFAEVSKLVTDLTKVHTECCHGDLLECADDRADLAKYICENQDSISSKLKEC |
| fused to N- | | CEKPLLEKSHCIAEVENDEMPADLPSLAADFVESKDVCKNYAEAKDVFLGMFLYEYARRH |
| terminus of | | PDYSVVLLLRLAKTYETTLEKCCAAADPHECYAKVFDEFKPLVEEPQNLIKQNCELFEQL |
| mature human | | GEYKFQNALLVRYTKKVPQVSTPTLVEVSRNLGKVGSKCCKHPEAKRMPCAEDYLSVVLN |
| GDF15 (wild- | | QLCVLHEKTPVSDRVTKCCTESLVNRRPCFSALEVDETYVPKEFNAETFTFHADICTLSE |
| type) | | KERQIKKQTALVELVKHKPKATKEQLKAVMDDFAAFVEKCCKADDKETCFAEEGKKLVAA |
| through a | | SQAALGLGGGGSGGGGSIEGRARNGDHCPLGPGRCCRLHTVRASLEDLGWADWVLSPREV |
| protease- | | QVTMCIGACPSQFRAANMHAQIKTSLHRLKPDTVPAPCCVPASYNPMVLIQKTDTGVSLQ |
| sensitive | | TYDDLLAKDCHCI |
| cleavable | | |
| linker | | |
| Fusion | 66 | DAHKSEVAHRFKDLGEENFKALVLIAFAQYLQQCPFEDHVKLVNEVTEFAKTCVADESAE |
| molecule HSA | | NCDKSLHTLFGDKLCTVATLRETYGEMADCCAKQEPERNECFLQHKDDNPNLPRLVRPEV |
| fused to N- | | DVMCTAFHDNEETFLKKYLYEIARRHPYFYAPELLFFAKRYKAAFTECCQAADKAACLLP |
| terminus of | | KLDELRDEGKASSAKQRLKCASLQKFGERAFKAWAVARLSQRFPKAEFAEVSKLVTDLTK |
| mature human | | VHTECCHGDLLECADDRADLAKYICENQDSISSKLKECCEKPLLEKSHCIAEVENDEMPA |
| GDF15 (wild- | | DLPSLAADFVESKDVCKNYAEAKDVFLGMFLYEYARRHPDYSVVLLLRLAKTYETTLEKC |
| type) | | CAAADPHECYAKVFDEFKPLVEEPQNLIKQNCELFEQLGEYKFQNALLVRYTKKVPQVST |
| through a | | PTLVEVSRNLGKVGSKCCKHPEAKRMPCAEDYLSVVLNQLCVLHEKTPVSDRVTKCCTES |
| protease- | | LVNRRPCFSALEVDETYVPKEFNAETFTFHADICTLSEKERQIKKQTALVELVKHKPKAT |
| sensitive | | KEQLKAVMDDFAAFVEKCCKADDKETCFAEEGKKLVAASQAALGLGGGGSGGGGSIEGRA |
| cleavable | | RNGDHCPLGPGRCCRLHTVRASLEDLGWADWVLSPREVQVTMCIGACPSQFRAANMHAQI |
| linker | | KTSLHRLKPDTVPAPCCVPASYNPMVLIQKTDTGVSLQTYDDLLAKDCHCI |
| Fusion | 67 | MDMRVPAQLLGLLLLWLRGARCDAHKSEVAHRFKDLGEENFKALVLIAFAQYLQQCPFED |
| molecule HSA | | HVKLVNEVTEFAKTCVADESAENCDKSLHTLFGDKLCTVATLRETYGEMADCCAKQEPER |
| with IgK | | NECFLQHKDDNPNLPRLVRPEVDVMCTAFHDNEETFLKKYLYEIARRHPYFYAPELLFFA |
| signal | | KRYKAAFTECCQAADKAACLLPKLDELRDEGKASSAKQRLKCASLQKFGERAFKAWAVAR |
| sequence | | LSQRFPKAEFAEVSKLVTDLTKVHTECCHGDLLECADDRADLAKYICENQDSISSKLKEC |
| fused to N- | | CEKPLLEKSHCIAEVENDEMPADLPSLAADFVESKDVCKNYAEAKDVFLGMFLYEYARRH |
| terminus of | | PDYSVVLLLRLAKTYETTLEKCCAAADPHECYAKVFDEFKPLVEEPQNLIKQNCELFEQL |
| mature human | | GEYKFQNALLVRYTKKVPQVSTPTLVEVSRNLGKVGSKCCKHPEAKRMPCAEDYLSVVLN |
| GDF15 (wild- | | QLCVLHEKTPVSDRVTKCCTESLVNRRPCFSALEVDETYVPKEFNAETFTFHADICTLSE |
| type) | | KERQIKKQTALVELVKHKPKATKEQLKAVMDDFAAFVEKCCKADDKETCFAEEGKKLVAA |
| through a | | SQAALGLGGGGSGGGGSGGGGSARNGDHCPLGPGRCCRLHTVRASLEDLGWADWVLSPRE |
| non- | | VQVTMCIGACPSQFRAANMHAQIKTSLHRLKPDTVPAPCCVPASYNPMVLIQKTDTGVSL |
| cleavable | | QTYDDLLAKDCHCI |
| linker | | |
| Fusion | 68 | DAHKSEVAHRFKDLGEENFKALVLIAFAQYLQQCPFEDHVKLVNEVTEFAKTCVADESAE |
| molecule HSA | | NCDKSLHTLFGDKLCTVATLRETYGEMADCCAKQEPERNECFLQHKDDNPNLPRLVRPEV |
| fused to N- | | DVMCTAFHDNEETFLKKYLYEIARRHPYFYAPELLFFAKRYKAAFTECCQAADKAACLLP |
| terminus of | | KLDELRDEGKASSAKQRLKCASLQKFGERAFKAWAVARLSQRFPKAEFAEVSKLVTDLTK |
| mature human | | VHTECCHGDLLECADDRADLAKYICENQDSISSKLKECCEKPLLEKSHCIAEVENDEMPA |
| GDF15 (wild- | | DLPSLAADFVESKDVCKNYAEAKDVFLGMFLYEYARRHPDYSVVLLLRLAKTYETTLEKC |
| type) | | CAAADPHECYAKVFDEFKPLVEEPQNLIKQNCELFEQLGEYKFQNALLVRYTKKVPQVST |
| through a | | PTLVEVSRNLGKVGSKCCKHPEAKRMPCAEDYLSVVLNQLCVLHEKTPVSDRVTKCCTES |
| non- | | LVNRRPCFSALEVDETYVPKEFNAETFTFHADICTLSEKERQIKKQTALVELVKHKPKAT |
| cleavable | | KEQLKAVMDDFAAFVEKCCKADDKETCFAEEGKKLVAASQAALGLGGGGSGGGGSGGGGS |
| linker | | ARNGDHCPLGPGRCCRLHTVRASLEDLGWADWVLSPREVQVTMCIGACPSQFRAANMHAQ |
| | | IKTSLHRLKPDTVPAPCCVPASYNPMVLIQKTDTGVSLQTYDDLLAKDCHCI |
| Mature human | 69 | ARNGDHCPLGPGRCCRLHTVRASLEDLGAADWVLSPREVQVTMCIGACPSQFRAANMHAQ |
| GDF15 | | IKTSLHRLKPDTVPAPCCVPASYNPMVLIQKTDTGVSLQTYDDLLAKDCHCI |
| Alanine | | |
| mutant w29 | | |
| Mature human | 70 | ARNGDHCPLGPGRCCRLHTVRASLEDLGWADAVLSPREVQVTMCIGACPSQFRAANMHAQ |
| GDF15 | | IKTSLHRLKPDTVPAPCCVPASYNPMVLIQKTDTGVSLQTYDDLLAKDCHCI |
| Alanine | | |
| mutant w32 | | |
| Mature human | 71 | ARNGDHCPLGPGRCCRLHTVRASLEDLGWADWVLSPREVQVTMCIGACPSQARAANMHAQ |
| GDF15 | | IKTSLHRLKPDTVPAPCCVPASYNPMVLIQKTDTGVSLQTYDDLLAKDCHCI |
| Alanine | | |
| mutant w52 | | |
| Mature human | 72 | ARNGDHCPLGPGRCCRLHTVRASLEDLGWADWVLSPREVQVTMCIGACPSQFRAANMHAQ |
| GDF15 | | IKTSAHRLKPDTVPAPCCVPASYNPMVLIQKTDTGVSLQTYDDLLAKDCHCI |
| Alanine | | |
| mutant w65 | | |
| Mature human | 73 | ARNGDHCPLGPGRCCRLHTVRASLEDLGWADWVLSPREVQVTMCIGACPSQFRAANMHAQ |
| GDF15 | | IKTSLHRAKPDTVPAPCCVPASYNPMVLIQKTDTGVSLQTYDDLLAKDCHCI |
| Alanine | | |
| mutant w68 | | |
| Mature human | 74 | ARNGDHCPLGPGRCCRLHTVRASLEDLGWADWVLSPREVQVTMCIGACPSQFRAANMHAQ |
| GDF15 | | IKTSLHRLKPDTVPAPCCVPASYNPMVLAQKTDTGVSLQTYDDLLAKDCHCI |
| Alanine | | |
| mutant w89 | | |
| Mature human | 75 | ARNGDHCPLGPGRCCRLHTVRASLAALGWAAWVLSPREVQVTMCIGACPSQFRAANMHAQ |
| GDF15 | | IKTSLHRLKPDTVPAPCCVPASYNPMVLIQKTDTGVSLQTYDDLLAKDCHCI |
| Alanine | | |
| mutant w113 | | |
| Mature human | 76 | ARNGDHCPLGPGRCCRLHTVRASLAALGWAAWVLSPRAVQVTMCIGACPSQFRAANMHAQ |
| GDF15 | | IKTSLHRLKPDTVPAPCCVPASYNPMVLIQKTDTGVSLQTYDDLLAKDCHCI |
| Alanine | | |
| mutant w114 | | |
| Mature human | 77 | ARNGDHCPLGPGRCCRLHTVRASLEDLGWADWVLSPREVQVTMCIGACPSQFRAANMHAQ |
| GDF15 | | IKTSLHRLKPDTVPAPCCVPASYNPMVLIQKTDTGVSLQTYAALLAKACHCI |
| Alanine | | |
| mutant w115 | | |
| Mature human | 78 | ARNGDHCPLGPGRCCRLHTVRASLEDLGWADWVLSPREVQVTMCIGACPSQFRAANMHAQ |
| GDF15 | | IKTSLHRLKPDTVPAPCCVPASYNPMVLIQKTATGVSLQTYAALLAKACHCI |
| Alanine | | |
| mutant w116 | | |
| Mature human | 79 | ARNGDHCPLGPGRCCRLHTVRASLEDLGWADWVLSPREVQVTMCIGACPSQFRAANMHAQ |
| GDF15 | | IKTSLHRLKPDTVPAPCCVPASYNPMVLIQKTATGVSLQTYDDLLAKDCHCI |
| Alanine | | |
| mutant w117 | | |
| Mature human | 80 | ARNGTHCPLGPGRCCRLHTVRASLEDLGWADWVLSPREVQVTMCIGACPSQFRAANMHAQ |
| GDF15 N- | | IKTSLHRLKPDTVPAPCCVPASYNPMVLIQKTDTGVSLQTYDDLLAKDCHCI |
| glycosylation | | |
| mutant | | |
| w118 | | |
| Mature human | 81 | ARNGDHCPLGPGRCCRNHTVRASLEDLGWADWVLSPREVQVTMCIGACPSQFRAANMHAQ |
| GDF15 N- | | IKTSLHRLKPDTVPAPCCVPASYNPMVLIQKTDTGVSLQTYDDLLAKDCHCI |
| glycosylation | | |
| mutant | | |
| w119 | | |
| Mature human | 82 | ARNGDHCPLGPGRCCRLHTVNASLEDLGWADWVLSPREVQVTMCIGACPSQFRAANMHAQ |
| GDF15 N- | | IKTSLHRLKPDTVPAPCCVPASYNPMVLIQKTDTGVSLQTYDDLLAKDCHCI |
| glycosylation | | |
| mutant | | |
| w120 | | |
| Mature human | 83 | ARNGDHCPLGPGRCCRLHTVRASLEDLGWADWNLSPREVQVTMCIGACPSQFRAANMHAQ |
| GDF15 N- | | IKTSLHRLKPDTVPAPCCVPASYNPMVLIQKTDTGVSLQTYDDLLAKDCHCI |
| glycosylation | | |
| mutant | | |
| w121 | | |
| Mature human | 84 | ARNGDHCPLGPGRCCRLHTVRASLEDLGWADWVLSPREVNVTMCIGACPSQFRAANMHAQ |
| GDF15 N- | | IKTSLHRLKPDTVPAPCCVPASYNPMVLIQKTDTGVSLQTYDDLLAKDCHCI |
| glycosylation | | |
| mutant | | |
| w122 | | |
| Mature human | 85 | ARNGDHCPLGPGRCCRLHTVRASLEDLGWADWVLSPREVQVTMCIGACPSQFRAANMTAQ |
| GDF15 N- | | IKTSLHRLKPDTVPAPCCVPASYNPMVLIQKTDTGVSLQTYDDLLAKDCHCI |
| glycosylation | | |
| mutant | | |
| w123 | | |
| Mature human | 86 | ARNGDHCPLGPGRCCRLHTVRASLEDLGWADWVLSPREVQVTMCIGACPSQFRAANMHAQ |
| GDF15 N- | | NKTSLHRLKPDTVPAPCCVPASYNPMVLIQKTDTGVSLQTYDDLLAKDCHCI |
| glycosylation | | |
| mutant | | |
| w124 | | |
| Mature human | 87 | ARNGDHCPLGPGRCCRLHTVRASLEDLGWADWVLSPREVQVTMCIGACPSQFRAANMHAQ |
| GDF15 N- | | INTSLHRLKPDTVPAPCCVPASYNPMVLIQKTDTGVSLQTYDDLLAKDCHCI |
| glycosylation | | |
| mutant | | |
| w125 | | |
| Mature human | 88 | ARNGDHCPLGPGRCCRLHTVRASLEDLGWADWVLSPREVQVTMCIGACPSQFRAANMHAQ |
| GDF15 N- | | IKTSLHRLKNDTVPAPCCVPASYNPMVLIQKTDTGVSLQTYDDLLAKDCHCI |
| glycosylation | | |
| mutant | | |
| w126 | | |
| Mature human | 89 | ARNGDHCPLGPGRCCRLHTVRASLEDLGWADWVLSPREVQVTMCIGACPSQFRAANMHAQ |
| GDF15 N- | | IKTSLHRLKPDTVPAPCCVNASYNPMVLIQKTDTGVSLQTYDDLLAKDCHCI |
| glycosylation | | |
| mutant | | |
| w127 | | |
| Mature human | 90 | ARNGDHCPLGPGRCCRLHTVRASLEDLGWADWVLSPREVQVTMCIGACPSQFRAANMHAQ |
| GDF15 N- | | IKTSLHRLKPDTVPAPCCVPASYNPMVLINKTDTGVSLQTYDDLLAKDCHCI |
| glycosylation | | |
| mutant | | |
| w128 | | |
| Mature human | 91 | ARNGDHCPLGPGRCCRLHTVRASLEDLGWADWVLSPREVQVTMCIGACPSQFRAANMHAQ |
| GDF15 N- | | IKTSLHRLKPDTVPAPCCVPASYNPMVLIQKNDTGVSLQTYDDLLAKDCHCI |
| glycosylation | | |
| mutant | | |
| w129 | | |
| Mature human | 92 | ARNGDHCPLGPGRCCRLHTVRASLEDLGWADWVLSPREVQVTMCIGACPSQFRAANMHAQ |
| GDF15 N- | | IKTSLHRLKPDTVPAPCCVPASYNPMVLIQKTDTNVSLQTYDDLLAKDCHCI |
| glycosylation | | |
| mutant | | |
| w130 | | |
| Mature human | 93 | ARNGDHCPLGPGRCCRLHTVRASLEDLGWADWVLSPREVQVTMCIGACPSQFRAANMHAQ |
| GDF15 N- | | IKTSLHRLKPDTVPAPCCVPASYNPMVLIQKTDTGVSNQTYDDLLAKDCHCI |
| glycosylation | | |
| mutant | | |
| w131 | | |
| Mature human | 94 | ARNGTHCPLGPGRCCRLHTVRASLEDLGWADWVLSPREVQVTMCIGACPSQFRAANMHAQ |
| GDF15 N- | | IKTSLHRLKPDTVPAPCCVPASYNPMVLINKTDTGVSLQTYDDLLAKDCHCI |
| glycosylation | | |
| mutant | | |
| w132 | | |
| Mature human | 95 | ARNGTHCPLGPGRCCRLHTVRASLEDLGWADWVLSPREVQVTMCIGACPSQFRAANMHAQ |
| GDF15 N- | | IKTSLHRLKPDTVPAPCCVPASYNPMVLIQKTDTNVSLQTYDDLLAKDCHCI |
| glycosylation | | |
| mutant | | |
| w133 | | |
| Mature human | 96 | ARNGDHCPLGPGRCCRLHTVRASLEDLGWADWVLSPREVNVTMCIGACPSQFRAANMHAQ |
| GDF15 N- | | IKTSLHRLKPDTVPAPCCVPASYNPMVLINKTDTGVSLQTYDDLLAKDCHCI |
| glycosylation | | |
| mutant | | |
| w134 | | |
| Mature human | 97 | ARNGDHCPLGPGRCCRLHTVRASLEDLGWADWVLSPREVNVTMCIGACPSQFRAANMHAQ |
| GDF15 N- | | IKTSLHRLKPDTVPAPCCVPASYNPMVLIQKTDTNVSLQTYDDLLAKDCHCI |
| glycosylation | | |
| mutant | | |
| w135 | | |
| Mature human | 98 | ARNGDHCPLGPGRCCRLHTVRASLEDLGWADWVLSPREVQVTMCIGACPSQFRAANMHAQ |
| GDF15 N- | | IKTSLHRLKNDTVPAPCCVPASYNPMVLINKTDTGVSLQTYDDLLAKDCHCI |
| glycosylation | | |
| mutant | | |
| w136 | | |
| Mature human | 99 | ARNGDHCPLGPGRCCRLHTVRASLEDLGWADWVLSPREVQVTMCIGACPSQFRAANMHAQ |
| GDF15 N- | | IKTSLHRLKNDTVPAPCCVPASYNPMVLIQKTDTNVSLQTYDDLLAKDCHCI |
| glycosylation | | |
| mutant | | |
| w137 | | |
| Mature human | 100 | ARNGDHCPLGPGRCCRLHTVRASLEDLGWADWVLSPREVQVTMCIGACPSQFRAANMHAQ |
| GDF15 N- | | IKTSLHRLKPDTVPAPCCVPASYNPMVLINKTDTNVSLQTYDDLLAKDCHCI |
| glycosylation | | |
| mutant | | |
| w138 | | |
| Mature human | 101 | ARNGDHCPLGPGRCCRLHTVRASLEDLGWADWVLSPREVQVTMCIGACPSQFRAANMHAQ |
| GDF15 N- | | IKTSLHRLKPDTVPAPCCVPASYNPMVLINKTDTGVSNQTYDDLLAKDCHCI |
| glycosylation | | |
| mutant | | |
| w139 | | |
| Mature human | 102 | ARNGDHCPLGPGRCCRLHTVRASLEDLGWADWVLSPREVQVTMCIGACPSQFRAANMHAQ |
| GDF15 N- | | IKTSLHRLKPDTVPAPCCVPASYNPMVLIQKTDTNVSNQTYDDLLAKDCHCI |
| glycosylation | | |
| mutant | | |
| w140 | | |
| Mature human | 103 | MEWSWVFLFFLSVTTGVHSARNGDHCPLGPGRCCRLHTVRASLEDLGWADWVLSPREVQV |
| GDF15 with a | | TMCIGACPSQFRAANMHAQIKTSLHRLKPDTVPAPCCVPASYNPMVLIQKTDTGVSLQTY |
| VH21 signal | | DDLLAKDCHCI |
| sequence | | |
| Mature human | 104 | MEWSWVFLFFLSVTTGVHSARNGDDCPLGPGRCCRLHTVRASLEDLGWADWVLSPREVQV |
| GDF15 H6D | | TMCIGACPSQFRAANMHAQIKTSLHRLKPDTVPAPCCVPASYNPMVLIQKTDTGVSLQTY |
| variant with | | DDLLAKDCHCI |
| a VH21 | | |
| signal | | |
| sequence | | |
| Mature human | 105 | ARNGDDCPLGPGRCCRLHTVRASLEDLGWADWVLSPREVQVTMCIGACPSQFRAANMHAQ |
| GDF15 H6D | | IKTSLHRLKPDTVPAPCCVPASYNPMVLIQKTDTGVSLQTYDDLLAKDCHCI |
| variant | | |
| Mature human | 106 | MEWSWVFLFFLSVTTGVHSARQGDHCPLGPGRCCRLHTVRASLEDLGWADWVLSPREVQV |
| GDF15 N3Q | | TMCIGACPSQFRAANMHAQIKTSLHRLKPDTVPAPCCVPASYNPMVLIQKTDTGVSLQTY |
| variant with | | DDLLAKDCHCI |
| a VH21 | | |
| signal | | |
| sequence | | |
| Mature human | 107 | ARQGDHCPLGPGRCCRLHTVRASLEDLGWADWVLSPREVQVTMCIGACPSQFRAANMHAQ |
| GDF15 N3Q | | IKTSLHRLKPDTVPAPCCVPASYNPMVLIQKTDTGVSLQTYDDLLAKDCHCI |
| variant | | |
| DhCpmFc(+) | 108 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGK |
| DhCpmFc(−)- | 109 | APELLGGPSVFLFPPKPKDTLMISRTPEVTCVVVDVSHEDPEVKFNWYVDGVEVHNAKTK |
| (G4S)4-GDF15 | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGSGGGGSGGGGSGGGGSARNG |
| | | DHCPLGPGRCCRLHTVRASLEDLGWADWVLSPREVQVTMCIGACPSQFRAANMHAQIKTS |
| | | LHRLKPDTVPAPCCVPASYNPMVLIQKTDTGVSLQTYDDLLAKDCHCI |
| DhCpmFc(+) | 110 | MEWSWVFLFFLSVTTGVHSAPELLGGPSVFLFPPKPKDTLMISRTPEVTCVVVDVSHEDP |
| with a VH21 | | EVKFNWYVDGVEVHNAKTKPREEQYNSTYRVVSVLTVLHQDWLNGKEYKCKVSNKALPAP |
| signal | | IEKTISKAKGQPREPQVYTLPPSRKEMTKNQVSLTCLVKGFYPSDIAVEWESNGQPENNY |
| sequence | | KTTPPVLKSDGSFFLYSKLTVDKSRWQQGNVFSCSVMHEALHNHYTQKSLSLSPGK |
| DhCpmFc(−)- | 111 | MEWSWVFLFFLSVTTGVHSAPELLGGPSVFLFPPKPKDTLMISRTPEVTCVVVDVSHEDP |
| (G4S)4-GDF15 | | EVKFNWYVDGVEVHNAKTKPREEQYNSTYRVVSVLTVLHQDWLNGKEYKCKVSNKALPAP |
| with a VH21 | | IEKTISKAKGQPREPQVYTLPPSREEMTKNQVSLTCLVKGFYPSDIAVEWESNGQPENNY |
| signal | | DTTPPVLDSDGSFFLYSDLTVDKSRWQQGNVFSCSVMHEALHNHYTQKSLSLSPGGGGGS |
| sequence | | GGGGSGGGGSGGGGSARNGDHCPLGPGRCCRLHTVRASLEDLGWADWVLSPREVQVTMCI |
| | | GACPSQFRAANMHAQIKTSLHRLKPDTVPAPCCVPASYNPMVLIQKTDTGVSLQTYDDLL |
| | | AKDCHCI |
| DhCpmFc(−) | 112 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGK |
| DhCpmFc(+)- | 113 | APELLGGPSVFLFPPKPKDTLMISRTPEVTCVVVDVSHEDPEVKFNWYVDGVEVHNAKTK |
| (G4S)4-GDF15 | | PREEQYNSTYRVVSVLTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGGGGGSGGGGSGGGGSGGGGSARNG |
| | | DHCPLGPGRCCRLHTVRASLEDLGWADWVLSPREVQVTMCIGACPSQFRAANMHAQIKTS |
| | | LHRLKPDTVPAPCCVPASYNPMVLIQKTDTGVSLQTYDDLLAKDCHCI |
| DhCpmFc(−) | 114 | MEWSWVFLFFLSVTTGVHSAPELLGGPSVFLFPPKPKDTLMISRTPEVTCVVVDVSHEDP |
| with a VH21 | | EVKFNWYVDGVEVHNAKTKPREEQYNSTYRVVSVLTVLHQDWLNGKEYKCKVSNKALPAP |
| signal | | IEKTISKAKGQPREPQVYTLPPSREEMTKNQVSLTCLVKGFYPSDIAVEWESNGQPENNY |
| sequence | | DTTPPVLDSDGSFFLYSDLTVDKSRWQQGNVFSCSVMHEALHNHYTQKSLSLSPGK |
| DhCpmFc(+)- | 115 | MEWSWVFLFFLSVTTGVHSAPELLGGPSVFLFPPKPKDTLMISRTPEVTCVVVDVSHEDP |
| (G4S)4-GDF15 | | EVKFNWYVDGVEVHNAKTKPREEQYNSTYRVVSVLTVLHQDWLNGKEYKCKVSNKALPAP |
| with a VH21 | | IEKTISKAKGQPREPQVYTLPPSRKEMTKNQVSLTCLVKGFYPSDIAVEWESNGQPENNY |
| signal | | KTTPPVLKSDGSFFLYSKLTVDKSRWQQGNVFSCSVMHEALHNHYTQKSLSLSPGGGGGS |
| sequence | | GGGGSGGGGSGGGGSARNGDHCPLGPGRCCRLHTVRASLEDLGWADWVLSPREVQVTMCI |
| | | GACPSQFRAANMHAQIKTSLHRLKPDTVPAPCCVPASYNPMVLIQKTDTGVSLQTYDDLL |
| | | AKDCHCI |
| DhCpmFc(−)- | 116 | APELLGGPSVFLFPPKPKDTLMISRTPEVTCVVVDVSHEDPEVKFNWYVDGVEVHNAKTK |
| (G4S)4- | | PREEQYNSTYRVVSVLTVLHQDWLNGKEYKCKVSNKALPAPIEKTISKAKGQPREPQVYT |
| GDF15(H6D) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGSGGGGSGGGGSGGGGSARNG |
| | | DDCPLGPGRCCRLHTVRASLEDLGWADWVLSPREVQVTMCIGACPSQFRAANMHAQIKTS |
| | | LHRLKPDTVPAPCCVPASYNPMVLIQKTDTGVSLQTYDDLLAKDCHCI |
| DhCpmFc(−)- | 117 | MEWSWVFLFFLSVTTGVHSAPELLGGPSVFLFPPKPKDTLMISRTPEVTCVVVDVSHEDP |
| (G4S)4- | | EVKFNWYVDGVEVHNAKTKPREEQYNSTYRVVSVLTVLHQDWLNGKEYKCKVSNKALPAP |
| GDF15(H6D) | | IEKTISKAKGQPREPQVYTLPPSREEMTKNQVSLTCLVKGFYPSDIAVEWESNGQPENNY |
| with a VH21 | | DTTPPVLDSDGSFFLYSDLTVDKSRWQQGNVFSCSVMHEALHNHYTQKSLSLSPGGGGGS |
| signal | | GGGGSGGGGSGGGGSARNGDDCPLGPGRCCRLHTVRASLEDLGWADWVLSPREVQVTMCI |
| sequence | | GACPSQFRAANMHAQIKTSLHRLKPDTVPAPCCVPASYNPMVLIQKTDTGVSLQTYDDLL |
| | | AKDCHCI |
| DhCpmFc(+)- | 118 | APELLGGPSVFLFPPKPKDTLMISRTPEVTCVVVDVSHEDPEVKFNWYVDGVEVHNAKTK |
| (G4S)4- | | PREEQYNSTYRVVSVLTVLHQDWLNGKEYKCKVSNKALPAPIEKTISKAKGQPREPQVYT |
| GDF15(H6D) | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGGGGGSGGGGSGGGGSGGGGSARNG |
| | | DDCPLGPGRCCRLHTVRASLEDLGWADWVLSPREVQVTMCIGACPSQFRAANMHAQIKTS |
| | | LHRLKPDTVPAPCCVPASYNPMVLIQKTDTGVSLQTYDDLLAKDCHCI |
| DhCpmFc(+)- | 119 | MEWSWVFLFFLSVTTGVHSAPELLGGPSVFLFPPKPKDTLMISRTPEVTCVVVDVSHEDP |
| (G4S)4- | | EVKFNWYVDGVEVHNAKTKPREEQYNSTYRVVSVLTVLHQDWLNGKEYKCKVSNKALPAP |
| GDF15(H6D) | | IEKTISKAKGQPREPQVYTLPPSRKEMTKNQVSLTCLVKGFYPSDIAVEWESNGQPENNY |
| with a VH21 | | KTTPPVLKSDGSFFLYSKLTVDKSRWQQGNVFSCSVMHEALHNHYTQKSLSLSPGGGGGS |
| signal | | GGGGSGGGGSGGGGSARNGDDCPLGPGRCCRLHTVRASLEDLGWADWVLSPREVQVTMCI |
| sequence | | GACPSQFRAANMHAQIKTSLHRLKPDTVPAPCCVPASYNPMVLIQKTDTGVSLQTYDDLL |
| | | AKDCHCI |
| DhCpmFc(+)- | 120 | APELLGGPSVFLFPPKPKDTLMISRTPEVTCVVVDVSHEDPEVKFNWYVDGVEVHNAKTK |
| (G4S)4- | | PREEQYNSTYRVVSVLTVLHQDWLNGKEYKCKVSNKALPAPIEKTISKAKGQPREPQVYT |
| GDF15(N3Q) | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGGGGGSGGGGSGGGGSGGGGSARQG |
| | | DHCPLGPGRCCRLHTVRASLEDLGWADWVLSPREVQVTMCIGACPSQFRAANMHAQIKTS |
| | | LHRLKPDTVPAPCCVPASYNPMVLIQKTDTGVSLQTYDDLLAKDCHCI |
| DhCpmFc(+)- | 121 | MEWSWVFLFFLSVTTGVHSAPELLGGPSVFLFPPKPKDTLMISRTPEVTCVVVDVSHEDP |
| (G4S)4- | | EVKFNWYVDGVEVHNAKTKPREEQYNSTYRVVSVLTVLHQDWLNGKEYKCKVSNKALPAP |
| GDF15(N3Q) | | IEKTISKAKGQPREPQVYTLPPSRKEMTKNQVSLTCLVKGFYPSDIAVEWESNGQPENNY |
| with a VH21 | | KTTPPVLKSDGSFFLYSKLTVDKSRWQQGNVFSCSVMHEALHNHYTQKSLSLSPGGGGGS |
| signal | | GGGGSGGGGSGGGGSARQGDHCPLGPGRCCRLHTVRASLEDLGWADWVLSPREVQVTMCI |
| sequence | | GACPSQFRAANMHAQIKTSLHRLKPDTVPAPCCVPASYNPMVLIQKTDTGVSLQTYDDLL |
| | | AKDCHCI |
| DhCpmFc(+)- | 122 | APELLGGPSVFLFPPKPKDTLMISRTPEVTCVVVDVSHEDPEVKFNWYVDGVEVHNAKTK |
| GDF15 | | PREEQYNSTYRVVSVLTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGARNGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| DhCpmFc(+)- | 123 | MEWSWVFLFFLSVTTGVHSAPELLGGPSVFLFPPKPKDTLMISRTPEVTCVVVDVSHEDP |
| GDF15 with a | | EVKFNWYVDGVEVHNAKTKPREEQYNSTYRVVSVLTVLHQDWLNGKEYKCKVSNKALPAP |
| VH21 signal | | IEKTISKAKGQPREPQVYTLPPSRKEMTKNQVSLTCLVKGFYPSDIAVEWESNGQPENNY |
| sequence | | KTTPPVLKSDGSFFLYSKLTVDKSRWQQGNVFSCSVMHEALHNHYTQKSLSLSPGARNGD |
| | | HCPLGPGRCCRLHTVRASLEDLGWADWVLSPREVQVTMCIGACPSQFRAANMHAQIKTSL |
| | | HRLKPDTVPAPCCVPASYNPMVLIQKTDTGVSLQTYDDLLAKDCHCI |
| DhCpmFc(+)- | 124 | APELLGGPSVFLFPPKPKDTLMISRTPEVTCVVVDVSHEDPEVKFNWYVDGVEVHNAKTK |
| G4-GDF15 | | PREEQYNSTYRVVSVLTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGGGGGARNGDHCPLGPGRCCRLHTV |
| | | RASLEDLGWADWVLSPREVQVTMCIGACPSQFRAANMHAQIKTSLHRLKPDTVPAPCCVP |
| | | ASYNPMVLIQKTDTGVSLQTYDDLLAKDCHCI |
| DhCpmFc(+)- | 125 | MEWSWVFLFFLSVTTGVHSAPELLGGPSVFLFPPKPKDTLMISRTPEVTCVVVDVSHEDP |
| G4-GDF15 | | EVKFNWYVDGVEVHNAKTKPREEQYNSTYRVVSVLTVLHQDWLNGKEYKCKVSNKALPAP |
| with a VH21 | | IEKTISKAKGQPREPQVYTLPPSRKEMTKNQVSLTCLVKGFYPSDIAVEWESNGQPENNY |
| signal | | KTTPPVLKSDGSFFLYSKLTVDKSRWQQGNVFSCSVMHEALHNHYTQKSLSLSPGGGGGA |
| sequence | | RNGDHCPLGPGRCCRLHTVRASLEDLGWADWVLSPREVQVTMCIGACPSQFRAANMHAQI |
| | | KTSLHRLKPDTVPAPCCVPASYNPMVLIQKTDTGVSLQTYDDLLAKDCHCI |
| DhCpmFc(+)- | 126 | APELLGGPSVFLFPPKPKDTLMISRTPEVTCVVVDVSHEDPEVKFNWYVDGVEVHNAKTK |
| (G4S)2-GDF15 | | PREEQYNSTYRVVSVLTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGGGGGSGGGGSARNGDHCPLGPGRC |
| | | CRLHTVRASLEDLGWADWVLSPREVQVTMCIGACPSQFRAANMHAQIKTSLHRLKPDTVP |
| | | APCCVPASYNPMVLIQKTDTGVSLQTYDDLLAKDCHCI |
| DhCpmFc(+)- | 127 | MEWSWVFLFFLSVTTGVHSAPELLGGPSVFLFPPKPKDTLMISRTPEVTCVVVDVSHEDP |
| (G4S)2-GDF15 | | EVKFNWYVDGVEVHNAKTKPREEQYNSTYRVVSVLTVLHQDWLNGKEYKCKVSNKALPAP |
| with a VH21 | | IEKTISKAKGQPREPQVYTLPPSRKEMTKNQVSLTCLVKGFYPSDIAVEWESNGQPENNY |
| signal | | KTTPPVLKSDGSFFLYSKLTVDKSRWQQGNVFSCSVMHEALHNHYTQKSLSLSPGGGGGS |
| sequence | | GGGGSARNGDHCPLGPGRCCRLHTVRASLEDLGWADWVLSPREVQVTMCIGACPSQFRAA |
| | | NMHAQIKTSLHRLKPDTVPAPCCVPASYNPMVLIQKTDTGVSLQTYDDLLAKDCHCI |
| DhCpmFc(+)- | 128 | APELLGGPSVFLFPPKPKDTLMISRTPEVTCVVVDVSHEDPEVKFNWYVDGVEVHNAKTK |
| (G4Q)2-GDF15 | | PREEQYNSTYRVVSVLTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGGGGGQGGGGQGGGGQGGGGQARNG |
| | | DHCPLGPGRCCRLHTVRASLEDLGWADWVLSPREVQVTMCIGACPSQFRAANMHAQIKTS |
| | | LHRLKPDTVPAPCCVPASYNPMVLIQKTDTGVSLQTYDDLLAKDCHCI |
| DhCpmFc(+)- | 129 | MEWSWVFLFFLSVTTGVHSAPELLGGPSVFLFPPKPKDTLMISRTPEVTCVVVDVSHEDP |
| (G4Q)2-GDF15 | | EVKFNWYVDGVEVHNAKTKPREEQYNSTYRVVSVLTVLHQDWLNGKEYKCKVSNKALPAP |
| with a VH21 | | IEKTISKAKGQPREPQVYTLPPSRKEMTKNQVSLTCLVKGFYPSDIAVEWESNGQPENNY |
| signal | | KTTPPVLKSDGSFFLYSKLTVDKSRWQQGNVFSCSVMHEALHNHYTQKSLSLSPGGGGGQ |
| sequence | | GGGGQGGGGQGGGGQARNGDHCPLGPGRCCRLHTVRASLEDLGWADWVLSPREVQVTMCI |
| | | GACPSQFRAANMHAQIKTSLHRLKPDTVPAPCCVPASYNPMVLIQKTDTGVSLQTYDDLL |
| | | AKDCHCI |
| DhCpmFc(−) | 130 | APELLGGPSVFLFPPKPKDTLMISRTPEVTCVVVDVSHEDPEVKFNWYVDGVEVHNAKTK |
| (L351C) | | PREEQYNSTYRVVSVLTVLHQDWLNGKEYKCKVSNKALPAPIEKTISKAKGQPREPQVYT |
| | | CPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGK |
| DhCpmFc(+)(L351C)- | 131 | APELLGGPSVFLFPPKPKDTLMISRTPEVTCVVVDVSHEDPEVKFNWYVDGVEVHNAKTK |
| G4- | | PREEQYNSTYRVVSVLTVLHQDWLNGKEYKCKVSNKALPAPIEKTISKAKGQPREPQVYT |
| GDF15 | | CPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGGGGGARNGDHCPLGPGRCCRLHTV |
| | | RASLEDLGWADWVLSPREVQVTMCIGACPSQFRAANMHAQIKTSLHRLKPDTVPAPCCVP |
| | | ASYNPMVLIQKTDTGVSLQTYDDLLAKDCHCI |
| DhCpmFc(−) | 132 | MEWSWVFLFFLSVTTGVHSAPELLGGPSVFLFPPKPKDTLMISRTPEVTCVVVDVSHEDP |
| (L351C) | | EVKFNWYVDGVEVHNAKTKPREEQYNSTYRVVSVLTVLHQDWLNGKEYKCKVSNKALPAP |
| with a VH21 | | IEKTISKAKGQPREPQVYTCPPSREEMTKNQVSLTCLVKGFYPSDIAVEWESNGQPENNY |
| signal | | DTTPPVLDSDGSFFLYSDLTVDKSRWQQGNVFSCSVMHEALHNHYTQKSLSLSPGK |
| sequence | | |
| DhCpmFc(+)(L351C)- | 133 | MEWSWVFLFFLSVTTGVHSAPELLGGPSVFLFPPKPKDTLMISRTPEVTCVVVDVSHEDP |
| G4- | | EVKFNWYVDGVEVHNAKTKPREEQYNSTYRVVSVLTVLHQDWLNGKEYKCKVSNKALPAP |
| GDF15 with a | | IEKTISKAKGQPREPQVYTCPPSRKEMTKNQVSLTCLVKGFYPSDIAVEWESNGQPENNY |
| VH21 signal | | KTTPPVLKSDGSFFLYSKLTVDKSRWQQGNVFSCSVMHEALHNHYTQKSLSLSPGGGGGA |
| sequence | | RNGDHCPLGPGRCCRLHTVRASLEDLGWADWVLSPREVQVTMCIGACPSQFRAANMHAQI |
| | | KTSLHRLKPDTVPAPCCVPASYNPMVLIQKTDTGVSLQTYDDLLAKDCHCI |
| DhCpmFc(−) | 134 | APELLGGPSVFLFPPKPKDTLMISRTPEVTCVVVDVSHEDPEVKFNWYVDGVEVHNAKTK |
| (Y349C) | | PREEQYNSTYRVVSVLTVLHQDWLNGKEYKCKVSNKALPAPIEKTISKAKGQPREPQVCT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGK |
| DhCpmFc(+)(S354C)- | 135 | APELLGGPSVFLFPPKPKDTLMISRTPEVTCVVVDVSHEDPEVKFNWYVDGVEVHNAKTK |
| G4- | | PREEQYNSTYRVVSVLTVLHQDWLNGKEYKCKVSNKALPAPIEKTISKAKGQPREPQVYT |
| GDF15 | | LPPCRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGGGGGARNGDHCPLGPGRCCRLHTV |
| | | RASLEDLGWADWVLSPREVQVTMCIGACPSQFRAANMHAQIKTSLHRLKPDTVPAPCCVP |
| | | ASYNPMVLIQKTDTGVSLQTYDDLLAKDCHCI |
| DhCpmFc(−) | 136 | MEWSWVFLFFLSVTTGVHSAPELLGGPSVFLFPPKPKDTLMISRTPEVTCVVVDVSHEDP |
| (Y349C) | | EVKFNWYVDGVEVHNAKTKPREEQYNSTYRVVSVLTVLHQDWLNGKEYKCKVSNKALPAP |
| with a VH21 | | IEKTISKAKGQPREPQVCTLPPSREEMTKNQVSLTCLVKGFYPSDIAVEWESNGQPENNY |
| signal | | DTTPPVLDSDGSFFLYSDLTVDKSRWQQGNVFSCSVMHEALHNHYTQKSLSLSPGK |
| sequence | | |
| DhCpmFc(+)(S354C)- | 137 | MEWSWVFLFFLSVTTGVHSAPELLGGPSVFLFPPKPKDTLMISRTPEVTCVVVDVSHEDP |
| G4- | | EVKFNWYVDGVEVHNAKTKPREEQYNSTYRVVSVLTVLHQDWLNGKEYKCKVSNKALPAP |
| GDF15 with a | | IEKTISKAKGQPREPQVYTLPPCRKEMTKNQVSLTCLVKGFYPSDIAVEWESNGQPENNY |
| VH21 signal | | KTTPPVLKSDGSFFLYSKLTVDKSRWQQGNVFSCSVMHEALHNHYTQKSLSLSPGGGGGA |
| sequence | | RNGDHCPLGPGRCCRLHTVRASLEDLGWADWVLSPREVQVTMCIGACPSQFRAANMHAQI |
| | | KTSLHRLKPDTVPAPCCVPASYNPMVLIQKTDTGVSLQTYDDLLAKDCHCI |
| CpmFc(+) | 138 | DKTHTCPPCPAPELLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKALPAPIEKTISKAK |
| | | GQPREPQVYTLPPSRKEMTKNQVSLTCLVKGFYPSDIAVEWESNGQPENNYKTTPPVLKS |
| | | DGSFFLYSKLTVDKSRWQQGNVFSCSVMHEALHNHYTQKSLSLSPGK |
| CpmFc(−)- | 139 | DKTHTCPPCPAPELLGGPSVFLFPPKPKDTLMISRTPEVTCVVVDVSHEDPEVKFNWYVD |
| (G4S)4-GDF15 | | GVEVHNAKTKPREEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPENNYDTTPPVLDS |
| | | DGSFFLYSDLTVDKSRWQQGNVFSCSVMHEALHNHYTQKSLSLSPGGGGGSGGGGSGGGG |
| | | SGGGGSARNGDHCPLGPGRCCRLHTVRASLEDLGWADWVLSPREVQVTMCIGACPSQFRA |
| | | ANMHAQIKTSLHRLKPDTVPAPCCVPASYNPMVLIQKTDTGVSLQTYDDLLAKDCHCI |
| CpmFc(+) | 140 | MEWSWVFLFFLSVTTGVHSDKTHTCPPCPAPELLGGPSVFLFPPKPKDTLMISRTPEVTC |
| with a VH21 | | VVVDVSHEDPEVKFNWYVDGVEVHNAKTKPREEQYNSTYRVVSVLTVLHQDWLNGKEYKC |
| signal | | KVSNKALPAPIEKTISKAKGQPREPQVYTLPPSRKEMTKNQVSLTCLVKGFYPSDIAVEW |
| sequence | | ESNGQPENNYKTTPPVLKSDGSFFLYSKLTVDKSRWQQGNVFSCSVMHEALHNHYTQKSL |
| | | SLSPGK |
| CpmFc(−)- | 141 | MEWSWVFLFFLSVTTGVHSDKTHTCPPCPAPELLGGPSVFLFPPKPKDTLMISRTPEVTC |
| (G4S)4-GDF15 | | VVVDVSHEDPEVKFNWYVDGVEVHNAKTKPREEQYNSTYRVVSVLTVLHQDWLNGKEYKC |
| with a VH21 | | KVSNKALPAPIEKTISKAKGQPREPQVYTLPPSREEMTKNQVSLTCLVKGFYPSDIAVEW |
| signal | | ESNGQPENNYDTTPPVLDSDGSFFLYSDLTVDKSRWQQGNVFSCSVMHEALHNHYTQKSL |
| sequence | | SLSPGGGGGSGGGGSGGGGSGGGGSARNGDHCPLGPGRCCRLHTVRASLEDLGWADWVLS |
| | | PREVQVTMCIGACPSQFRAANMHAQIKTSLHRLKPDTVPAPCCVPASYNPMVLIQKTDTG |
| | | VSLQTYDDLLAKDCHCI |
| Fc-(G4S)8- | 142 | GGGERKSSVECPPCPAPPVAGPSVFLFPPKPKDTLMISRTPEVTCVVVDVSHEDPEVQFN |
| Fc-GS(G4S)4- | | WYVDGVEVHNAKTKPREEQFNSTFRVVSVLTVVHQDWLNGKEYKCKVSNKGLPAPIEKTI |
| GDF15 | | SKTKGQPREPQVYTLPPSREEMTKNQVSLTCLVKGFYPSDIAVEWESNGQPENNYKTTPP |
| | | MLDSDGSFFLYSKLTVDKSRWQQGNVFSCSVMHEALHNHYTQKSLSLSPGGGGGSGGGGS |
| | | GGGGSGGGGSGGGGSGGGGSGGGGSGGGGSERKSSVECPPCPAPPVAGPSVFLFPPKPKD |
| | | TLMISRTPEVTCVVVDVSHEDPEVQFNWYVDGVEVHNAKTKPREEQFNSTFRVVSVLTVV |
| | | HQDWLNGKEYKCKVSNKGLPAPIEKTISKTKGQPREPQVYTLPPSREEMTKNQVSLTCLV |
| | | KGFYPSDIAVEWESNGQPENNYKTTPPMLDSDGSFFLYSKLTVDKSRWQQGNVFSCSVMH |
| | | EALHNHYTQKSLSLSPGSGGGGSGGGGSGGGGSGGGGSARNGDHCPLGPGRCCRLHTVRA |
| | | SLEDLGWADWVLSPREVQVTMCIGACPSQFRAANMHAQIKTSLHRLKPDTVPAPCCVPAS |
| | | YNPMVLIQKTDTGVSLQTYDDLLAKDCHCI |
| Fc-(G4S)8- | 143 | MEWSWVFLFFLSVTTGVHSGGGERKSSVECPPCPAPPVAGPSVFLFPPKPKDTLMISRTP |
| Fc-GS(G4S)4- | | EVTCVVVDVSHEDPEVQFNWYVDGVEVHNAKTKPREEQFNSTFRVVSVLTVVHQDWLNGK |
| GDF15 with a | | EYKCKVSNKGLPAPIEKTISKTKGQPREPQVYTLPPSREEMTKNQVSLTCLVKGFYPSDI |
| VH21 signal | | AVEWESNGQPENNYKTTPPMLDSDGSFFLYSKLTVDKSRWQQGNVFSCSVMHEALHNHYT |
| sequence | | QKSLSLSPGGGGGSGGGGSGGGGSGGGGSGGGGSGGGGSGGGGSGGGGSERKSSVECPPC |
| | | PAPPVAGPSVFLFPPKPKDTLMISRTPEVTCVVVDVSHEDPEVQFNWYVDGVEVHNAKTK |
| | | PREEQFNSTFRVVSVLTVVHQDWLNGKEYKCKVSNKGLPAPIEKTISKTKGQPREPQVYT |
| | | LPPSREEMTKNQVSLTCLVKGFYPSDIAVEWESNGQPENNYKTTPPMLDSDGSFFLYSKL |
| | | TVDKSRWQQGNVFSCSVMHEALHNHYTQKSLSLSPGSGGGGSGGGGSGGGGSGGGGSARN |
| | | GDHCPLGPGRCCRLHTVRASLEDLGWADWVLSPREVQVTMCIGACPSQFRAANMHAQIKT |
| | | SLHRLKPDTVPAPCCVPASYNPMVLIQKTDTGVSLQTYDDLLAKDCHCI |
| Fc-(G4S)3- | 144 | GGGERKSSVECPPCPAPPVAGPSVFLFPPKPKDTLMISRTPEVTCVVVDVSHEDPEVQFN |
| Fc-GS(G4S)4- | | WYVDGVEVHNAKTKPREEQFNSTFRVVSVLTVVHQDWLNGKEYKCKVSNKGLPAPIEKTI |
| GDF15 | | SKTKGQPREPQVYTLPPSREEMTKNQVSLTCLVKGFYPSDIAVEWESNGQPENNYKTTPP |
| | | MLDSDGSFFLYSKLTVDKSRWQQGNVFSCSVMHEALHNHYTQKSLSLSPGGGGGSGGGGS |
| | | GGGGSERKSSVECPPCPAPPVAGPSVFLFPPKPKDTLMISRTPEVTCVVVDVSHEDPEVQ |
| | | FNWYVDGVEVHNAKTKPREEQFNSTFRVVSVLTVVHQDWLNGKEYKCKVSNKGLPAPIEK |
| | | TISKTKGQPREPQVYTLPPSREEMTKNQVSLTCLVKGFYPSDIAVEWESNGQPENNYKTT |
| | | PPMLDSDGSFFLYSKLTVDKSRWQQGNVFSCSVMHEALHNHYTQKSLSLSPGSGGGGSGG |
| | | GGSGGGGSGGGGSARNGDHCPLGPGRCCRLHTVRASLEDLGWADWVLSPREVQVTMCIGA |
| | | CPSQFRAANMHAQIKTSLHRLKPDTVPAPCCVPASYNPMVLIQKTDTGVSLQTYDDLLAK |
| | | DCHCI |
| Fc-(G4S)3- | 145 | MEWSWVFLFFLSVTTGVHSGGGERKSSVECPPCPAPPVAGPSVFLFPPKPKDTLMISRTP |
| Fc-GS(G4S)4- | | EVTCVVVDVSHEDPEVQFNWYVDGVEVHNAKTKPREEQFNSTFRVVSVLTVVHQDWLNGK |
| GDF15 with a | | EYKCKVSNKGLPAPIEKTISKTKGQPREPQVYTLPPSREEMTKNQVSLTCLVKGFYPSDI |
| VH21 signal | | AVEWESNGQPENNYKTTPPMLDSDGSFFLYSKLTVDKSRWQQGNVFSCSVMHEALHNHYT |
| sequence | | QKSLSLSPGGGGGSGGGGSGGGGSERKSSVECPPCPAPPVAGPSVFLFPPKPKDTLMISR |
| | | TPEVTCVVVDVSHEDPEVQFNWYVDGVEVHNAKTKPREEQFNSTFRVVSVLTVVHQDWLN |
| | | GKEYKCKVSNKGLPAPIEKTISKTKGQPREPQVYTLPPSREEMTKNQVSLTCLVKGFYPS |
| | | DIAVEWESNGQPENNYKTTPPMLDSDGSFFLYSKLTVDKSRWQQGNVFSCSVMHEALHNH |
| | | YTQKSLSLSPGSGGGGSGGGGSGGGGSGGGGSARNGDHCPLGPGRCCRLHTVRASLEDLG |
| | | WADWVLSPREVQVTMCIGACPSQFRAANMHAQIKTSLHRLKPDTVPAPCCVPASYNPMVL |
| | | IQKTDTGVSLQTYDDLLAKDCHCI |
| Fc-(G4S)5- | 146 | GGGERKSSVECPPCPAPPVAGPSVFLFPPKPKDTLMISRTPEVTCVVVDVSHEDPEVQFN |
| Fc-GS(G4S)4- | | WYVDGVEVHNAKTKPREEQFNSTFRVVSVLTVVHQDWLNGKEYKCKVSNKGLPAPIEKTI |
| GDF15 | | SKTKGQPREPQVYTLPPSREEMTKNQVSLTCLVKGFYPSDIAVEWESNGQPENNYKTTPP |
| | | MLDSDGSFFLYSKLTVDKSRWQQGNVFSCSVMHEALHNHYTQKSLSLSPGGGGGSGGGGS |
| | | GGGGSGGGGSGGGGSERKSSVECPPCPAPPVAGPSVFLFPPKPKDTLMISRTPEVTCVVV |
| | | DVSHEDPEVQFNWYVDGVEVHNAKTKPREEQFNSTFRVVSVLTVVHQDWLNGKEYKCKVS |
| | | NKGLPAPIEKTISKTKGQPREPQVYTLPPSREEMTKNQVSLTCLVKGFYPSDIAVEWESN |
| | | GQPENNYKTTPPMLDSDGSFFLYSKLTVDKSRWQQGNVFSCSVMHEALHNHYTQKSLSLS |
| | | PGSGGGGSGGGGSGGGGSGGGGSARNGDHCPLGPGRCCRLHTVRASLEDLGWADWVLSPR |
| | | EVQVTMCIGACPSQFRAANMHAQIKTSLHRLKPDTVPAPCCVPASYNPMVLIQKTDTGVS |
| | | LQTYDDLLAKDCHCI |
| Fc-(G4S)5- | 147 | MEWSWVFLFFLSVTTGVHSGGGERKSSVECPPCPAPPVAGPSVFLFPPKPKDTLMISRTP |
| Fc-GS(G4S)4- | | EVTCVVVDVSHEDPEVQFNWYVDGVEVHNAKTKPREEQFNSTFRVVSVLTVVHQDWLNGK |
| GDF15 with a | | EYKCKVSNKGLPAPIEKTISKTKGQPREPQVYTLPPSREEMTKNQVSLTCLVKGFYPSDI |
| VH21 signal | | AVEWESNGQPENNYKTTPPMLDSDGSFFLYSKLTVDKSRWQQGNVFSCSVMHEALHNHYT |
| sequence | | QKSLSLSPGGGGGSGGGGSGGGGSGGGGSGGGGSERKSSVECPPCPAPPVAGPSVFLFPP |
| | | KPKDTLMISRTPEVTCVVVDVSHEDPEVQFNWYVDGVEVHNAKTKPREEQFNSTFRVVSV |
| | | LTVVHQDWLNGKEYKCKVSNKGLPAPIEKTISKTKGQPREPQVYTLPPSREEMTKNQVSL |
| | | TCLVKGFYPSDIAVEWESNGQPENNYKTTPPMLDSDGSFFLYSKLTVDKSRWQQGNVFSC |
| | | SVMHEALHNHYTQKSLSLSPGSGGGGSGGGGSGGGGSGGGGSARNGDHCPLGPGRCCRLH |
| | | TVRASLEDLGWADWVLSPREVQVTMCIGACPSQFRAANMHAQIKTSLHRLKPDTVPAPCC |
| | | VPASYNPMVLIQKTDTGVSLQTYDDLLAKDCHCI |
| Mature human | 148 | ARDGDHCPLGPGRCCRLHTVRASLEDLGWADWVLSPREVQVTMCIGACPSQFRAANMHAQ |
| GDF15(N3D) | | IKTSLHRLKPDTVPAPCCVPASYNPMVLIQKTDTGVSLQTYDDLLAKDCHCI |
| Mature human | 149 | GDHCPLGPGRCCRLHTVRASLEDLGWADWVLSPREVQVTMCIGACPSQFRAANMHAQIKT |
| GDF15(Ndel3) | | SLHRLKPDTVPAPCCVPASYNPMVLIQKTDTGVSLQTYDDLLAKDCHCI |
| Sequence for | 150 | APELLGGPSVFLFPPKPKDTLMISRTPEVTCVVVDVSHEDPEVKFNWYVDGVEVHNAKTK |
| dimer of | | PREEQYNSTYRVVSVLTVLHQDWLNGKEYKCKVSNKALPAPIEKTISKAKGQPREPQVTT |
| DhMonoFc- | | LPPSREEMTKNQVSLTCLVKGFYPSDIAVEWESNGQPENNYDTTPPVLDSDGSFFLYSDL |
| GDF15 | | TVDKSRWQQGNVFSCSVMHEALHNHYTQKSLSLSPGARNGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| Sequence for | 151 | APELLGGPSVFLFPPKPKDTLMISRTPEVTCVVVDVSHEDPEVKFNWYVDGVEVHNAKTK |
| dimer of | | PREEQYNSTYRVVSVLTVLHQDWLNGKEYKCKVSNKALPAPIEKTISKAKGQPREPQVTT |
| DhMonoFc- | | LPPSREEMTKNQVSLTCLVKGFYPSDIAVEWESNGQPENNYDTTPPVLDSDGSFFLYSDL |
| (G4S)4-GDF15 | | TVDKSRWQQGNVFSCSVMHEALHNHYTQKSLSLSPGGGGGSGGGGSGGGGSGGGGSARNG |
| | | DHCPLGPGRCCRLHTVRASLEDLGWADWVLSPREVQVTMCIGACPSQFRAANMHAQIKTS |
| | | LHRLKPDTVPAPCCVPASYNPMVLIQKTDTGVSLQTYDDLLAKDCHCI |
| Sequence for | 152 | APELLGGPSVFLFPPKPKDTLMISRTPEVTCVVVDVSHEDPEVKFNWYVDGVEVHNAKTK |
| dimer of | | PREEQYNSTYRVVSVLTVLHQDWLNGKEYKCKVSNKALPAPIEKTISKAKGQPREPQVTT |
| DhMonoFc- | | LPPSREEMTKNQVSLTCLVKGFYPSDIAVEWESNGQPENNYDTTPPVLDSDGSFFLYSDL |
| (G4S)4- | | TVDKSRWQQGNVFSCSVMHEALHNHYTQKSLSLSPGGGGGSGGGGSGGGGSGGGGSARNG |
| GDF15(H6D) | | DDCPLGPGRCCRLHTVRASLEDLGWADWVLSPREVQVTMCIGACPSQFRAANMHAQIKTS |
| | | LHRLKPDTVPAPCCVPASYNPMVLIQKTDTGVSLQTYDDLLAKDCHCI |
| Sequence for | 153 | GGGERKSSVECPPCPAPPVAGPSVFLFPPKPKDTLMISRTPEVTCVVVDVSHEDPEVQFN |
| dimer of | | WYVDGVEVHNAKTKPREEQFNSTFRVVSVLTVVHQDWLNGKEYKCKVSNKGLPAPIEKTI |
| GGGFc- | | SKTKGQPREPQVYTLPPSREEMTKNQVSLTCLVKGFYPSDIAVEWESNGQPENNYKTTPP |
| (G4S)4-Fc- | | MLDSDGSFFLYSKLTVDKSRWQQGNVFSCSVMHEALHNHYTQKSLSLSPGGGGGSGGGGS |
| S(G4S)4- | | GGGGSGGGGSERKSSVECPPCPAPPVAGPSVFLFPPKPKDTLMISRTPEVTCVVVDVSHE |
| GDF15 | | DPEVQFNWYVDGVEVHNAKTKPREEQFNSTFRVVSVLTVVHQDWLNGKEYKCKVSNKGLP |
| | | APIEKTISKTKGQPREPQVYTLPPSREEMTKNQVSLTCLVKGFYPSDIAVEWESNGQPEN |
| | | NYKTTPPMLDSDGSFFLYSKLTVDKSRWQQGNVFSCSVMHEALHNHYTQKSLSLSPGSGG |
| | | GGSGGGGSGGGGSGGGGSARNGDHCPLGPGRCCRLHTVRASLEDLGWADWVLSPREVQVT |
| | | MCIGACPSQFRAANMHAQIKTSLHRLKPDTVPAPCCVPASYNPMVLIQKTDTGVSLQTYD |
| | | DLLAKDCHCI |
| Sequence for | 154 | GGGAPPVAGPSVFLFPPKPKDTLMISRTPEVTCVVVDVSHEDPEVQFNWYVDGVEVHNAK |
| dimer of | | TKPREEQFNSTFRVVSVLTVVHQDWLNGKEYKCKVSNKGLPAPIEKTISKTKGQPREPQV |
| GGGDhFc- | | YTLPPSREEMTKNQVSLTCLVKGFYPSDIAVEWESNGQPENNYKTTPPMLDSDGSFFLYS |
| (G4S)-5- | | KLTVDKSRWQQGNVFSCSVMHEALHNHYTQKSLSLSPGGGGGSGGGGSGGGGSGGGGSGG |
| DhFc- | | GGSAPPVAGPSVFLFPPKPKDTLMISRTPEVTCVVVDVSHEDPEVQFNWYVDGVEVHNAK |
| S(G4S)4- | | TKPREEQFNSTFRVVSVLTVVHQDWLNGKEYKCKVSNKGLPAPIEKTISKTKGQPREPQV |
| GDF15 | | YTLPPSREEMTKNQVSLTCLVKGFYPSDIAVEWESNGQPENNYKTTPPMLDSDGSFFLYS |
| | | KLTVDKSRWQQGNVFSCSVMHEALHNHYTQKSLSLSPGSGGGGSGGGGSGGGGSGGGGSA |
| | | RNGDHCPLGPGRCCRLHTVRASLEDLGWADWVLSPREVQVTMCIGACPSQFRAANMHAQI |
| | | KTSLHRLKPDTVPAPCCVPASYNPMVLIQKTDTGVSLQTYDDLLAKDCHCI |
| DhholeFc | 155 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPSRDELTKNQVSLSCAVKGFYPSDIAVEWESNGQPENNYKTTPPVLDSDGSFFLVSKL |
| | | TVDKSRWQQGNVFSCSVMHEALHNHYTQKSLSLSPGK |
| DhknobFc- | 156 | APELLGGPSVFLFPPKPKDTLMISRTPEVTCVVVDVSHEDPEVKFNWYVDGVEVHNAKTK |
| (G4S)4-GDF15 | | PREEQYNSTYRVVSVLTVLHQDWLNGKEYKCKVSNKALPAPIEKTISKAKGQPREPQVYT |
| | | LPPSRDELTKNQVSLWCLVKGFYPSDIAVEWESNGQPENNYKTTPPVLDSDGSFFLYSKL |
| | | TVDKSRWQQGNVFSCSVMHEALHNHYTQKSLSLSPGGGGGSGGGGSGGGGSGGGGSARNG |
| | | DHCPLGPGRCCRLHTVRASLEDLGWADWVLSPREVQVTMCIGACPSQFRAANMHAQIKTS |
| | | LHRLKPDTVPAPCCVPASYNPMVLIQKTDTGVSLQTYDDLLAKDCHCI |
| DhknobFc | 157 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPSRDELTKNQVSLWCLVKGFYPSDIAVEWESNGQPENNYKTTPPVLDSDGSFFLYSKL |
| | | TVDKSRWQQGNVFSCSVMHEALHNHYTQKSLSLSPG |
| DhholeFc | 158 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPSRDELTKNQVSLSCAVKGFYPSDIAVEWESNGQPENNYKTTPPVLDSDGSFFLVSKL |
| | | TVDKSRWQQGNVFSCSVMHEALHNHYTQKSLSLSPG |
| DhCpmFc(−) | 159 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGK |
| DhCpmFc(+)- | 160 | APELLGGPSVFLFPPKPKDTLMISRTPEVTCVVVDVSHEDPEVKFNWYVDGVEVHNAKTK |
| (1K)-GDF15 | | PREEQYNSTYRVVSVLTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGGSGSATGGSGSVASSGSGSATHLA |
| | | RNGDHCPLGPGRCCRLHTVRASLEDLGWADWVLSPREVQVTMCIGACPSQFRAANMHAQI |
| | | KTSLHRLKPDTVPAPCCVPASYNPMVLIQKTDTGVSLQTYDDLLAKDCHCI |
| DhCpmFc(+) | 161 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPG |
| DhCpmFc(−) | 162 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPG |
| DhCpmFc(+) | 163 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGK |
| DhCpmFc(−)- | 164 | APELLGGPSVFLFPPKPKDTLMISRTPEVTCVVVDVSHEDPEVKFNWYVDGVEVHNAKTK |
| GDF15 | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGARNGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| DhCpmFc(−) | 165 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPG |
| DhCpmFc(+) | 166 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPG |
| DhCpmFc(−)- | 167 | APELLGGPSVFLFPPKPKDTLMISRTPEVTCVVVDVSHEDPEVKFNWYVDGVEVHNAKTK |
| GDF15(N3D) | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGARDGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| DhCpmFc(−)- | 168 | APELLGGPSVFLFPPKPKDTLMISRTPEVTCVVVDVSHEDPEVKFNWYVDGVEVHNAKTK |
| GDF15(Nde13) | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGDHCPLGPGRCCRLHTVRASLEDL |
| | | GWADWVLSPREVQVTMCIGACPSQFRAANMHAQIKTSLHRLKPDTVPAPCCVPASYNPMV |
| | | LIQKTDTGVSLQTYDDLLAKDCHCI |
| DhCpmFc(−)- | 169 | APELLGGPSVFLFPPKPKDTLMISRTPEVTCVVVDVSHEDPEVKFNWYVDGVEVHNAKTK |
| G4- | | PREEQYNSTYRVVSVLTVLHQDWLNGKEYKCKVSNKALPAPIEKTISKAKGQPREPQVYT |
| GDF15(N3D) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGARDGDHCPLGPGRCCRLHTV |
| | | RASLEDLGWADWVLSPREVQVTMCIGACPSQFRAANMHAQIKTSLHRLKPDTVPAPCCVP |
| | | ASYNPMVLIQKTDTGVSLQTYDDLLAKDCHCI |
| DhCpmFc(−)- | 170 | APELLGGPSVFLFPPKPKDTLMISRTPEVTCVVVDVSHEDPEVKFNWYVDGVEVHNAKTK |
| G4S-GDF15 | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGSARNGDHCPLGPGRCCRLHT |
| | | VRASLEDLGWADWVLSPREVQVTMCIGACPSQFRAANMHAQIKTSLHRLKPDTVPAPCCV |
| | | PASYNPMVLIQKTDTGVSLQTYDDLLAKDCHCI |
| DhCpmFc(−)- | 171 | APELLGGPSVFLFPPKPKDTLMISRTPEVTCVVVDVSHEDPEVKFNWYVDGVEVHNAKTK |
| (G4S)2-GDF15 | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGSGGGGSARNGDHCPLGPGRC |
| | | CRLHTVRASLEDLGWADWVLSPREVQVTMCIGACPSQFRAANMHAQIKTSLHRLKPDTVP |
| | | APCCVPASYNPMVLIQKTDTGVSLQTYDDLLAKDCHCI |
| DhCpmFc(−)- | 172 | APELLGGPSVFLFPPKPKDTLMISRTPEVTCVVVDVSHEDPEVKFNWYVDGVEVHNAKTK |
| (G4S)2- | | PREEQYNSTYRVVSVLTVLHQDWLNGKEYKCKVSNKALPAPIEKTISKAKGQPREPQVYT |
| GDF15(N3D) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGSGGGGSARDGDHCPLGPGRC |
| | | CRLHTVRASLEDLGWADWVLSPREVQVTMCIGACPSQFRAANMHAQIKTSLHRLKPDTVP |
| | | APCCVPASYNPMVLIQKTDTGVSLQTYDDLLAKDCHCI |
| DhCpmFc(−)- | 173 | APELLGGPSVFLFPPKPKDTLMISRTPEVTCVVVDVSHEDPEVKFNWYVDGVEVHNAKTK |
| G4P-GDF15 | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGPARNGDHCPLGPGRCCRLHT |
| | | VRASLEDLGWADWVLSPREVQVTMCIGACPSQFRAANMHAQIKTSLHRLKPDTVPAPCCV |
| | | PASYNPMVLIQKTDTGVSLQTYDDLLAKDCHCI |
| DhCpmFc(−)- | 174 | APELLGGPSVFLFPPKPKDTLMISRTPEVTCVVVDVSHEDPEVKFNWYVDGVEVHNAKTK |
| (G4P)2-GDF15 | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGPGGGGPARNGDHCPLGPGRC |
| | | CRLHTVRASLEDLGWADWVLSPREVQVTMCIGACPSQFRAANMHAQIKTSLHRLKPDTVP |
| | | APCCVPASYNPMVLIQKTDTGVSLQTYDDLLAKDCHCI |
| DhCpmFc(−)- | 175 | APELLGGPSVFLFPPKPKDTLMISRTPEVTCVVVDVSHEDPEVKFNWYVDGVEVHNAKTK |
| G4Q-GDF15 | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGQARNGDHCPLGPGRCCRLHT |
| | | VRASLEDLGWADWVLSPREVQVTMCIGACPSQFRAANMHAQIKTSLHRLKPDTVPAPCCV |
| | | PASYNPMVLIQKTDTGVSLQTYDDLLAKDCHCI |
| DhCpmFc(−)- | 176 | APELLGGPSVFLFPPKPKDTLMISRTPEVTCVVVDVSHEDPEVKFNWYVDGVEVHNAKTK |
| (G4Q)2-GDF15 | | PREEQYNSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGQGGGGQARNGDHCPLGPGRC |
| | | CRLHTVRASLEDLGWADWVLSPREVQVTMCIGACPSQFRAANMHAQIKTSLHRLKPDTVP |
| | | APCCVPASYNPMVLIQKTDTGVSLQTYDDLLAKDCHCI |
| DhCpmFc(−)- | 177 | APELLGGPSVFLFPPKPKDTLMISRTPEVTCVVVDVSHEDPEVKFNWYVDGVEVHNAKTK |
| (G4Q)2- | | PREEQYNSTYRVVSVLTVLHQDWLNGKEYKCKVSNKALPAPIEKTISKAKGQPREPQVYT |
| GDF15(ND3) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGQGGGGQARDGDHCPLGPGRC |
| | | CRLHTVRASLEDLGWADWVLSPREVQVTMCIGACPSQFRAANMHAQIKTSLHRLKPDTVP |
| | | APCCVPASYNPMVLIQKTDTGVSLQTYDDLLAKDCHCI |
| DhCpmFc(−)- | 178 | APELLGGPSVFLFPPKPKDTLMISRTPEVTCVVVDVSHEDPEVKFNWYVDGVEVHNAKTK |
| (G4Q)2- | | PREEQYNSTYRVVSVLTVLHQDWLNGKEYKCKVSNKALPAPIEKTISKAKGQPREPQVYT |
| GDF15(Ndel3) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGQGGGGQGDHCPLGPGRCCRL |
| | | HTVRASLEDLGWADWVLSPREVQVTMCIGACPSQFRAANMHAQIKTSLHRLKPDTVPAPC |
| | | CVPASYNPMVLIQKTDTGVSLQTYDDLLAKDCHCI |
| DhCpmFc(−) | 179 | APELLGGPSVFLFPPKPKDTLMISRTPEVTCVVVDVSHEDPEVKFNWYVDGVEVHNAKTK |
| (Y349C) | | PREEQYNSTYRVVSVLTVLHQDWLNGKEYKCKVSNKALPAPIEKTISKAKGQPREPQVCT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGK |
| DhCpmFc(+)(S354C)- | 180 | APELLGGPSVFLFPPKPKDTLMISRTPEVTCVVVDVSHEDPEVKFNWYVDGVEVHNAKTK |
| GDF15(N3D) | | PREEQYNSTYRVVSVLTVLHQDWLNGKEYKCKVSNKALPAPIEKTISKAKGQPREPQVYT |
| | | LPPCRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGARDGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| DhCpmFc(+)(S354C) | 181 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPCRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPG |
| DhCpmFc(−) | 182 | APELLGGPSVFLFPPKPKDTLMISRTPEVTCVVVDVSHEDPEVKFNWYVDGVEVHNAKTK |
| (Y349C) | | PREEQYNSTYRVVSVLTVLHQDWLNGKEYKCKVSNKALPAPIEKTISKAKGQPREPQVCT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPG |
| DhCpmFc(+)(S354C) | 183 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPCRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGK |
| DhCpmFc(−) | 184 | APELLGGPSVFLFPPKPKDTLMISRTPEVTCVVVDVSHEDPEVKFNWYVDGVEVHNAKTK |
| (Y349C)- | | PREEQYNSTYRVVSVLTVLHQDWLNGKEYKCKVSNKALPAPIEKTISKAKGQPREPQVCT |
| GDF15 | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGARNGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| DhCpmFc(−) | 185 | APELLGGPSVFLFPPKPKDTLMISRTPEVTCVVVDVSHEDPEVKFNWYVDGVEVHNAKTK |
| (Y349C) | | PREEQYNSTYRVVSVLTVLHQDWLNGKEYKCKVSNKALPAPIEKTISKAKGQPREPQVCT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPG |
| DhCpmFc(+)(S354C) | 186 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | LPPCRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPG |
| DhCpmFc(−) | 187 | APELLGGPSVFLFPPKPKDTLMISRTPEVTCVVVDVSHEDPEVKFNWYVDGVEVHNAKTK |
| (Y349C)- | | PREEQYNSTYRVVSVLTVLHQDWLNGKEYKCKVSNKALPAPIEKTISKAKGQPREPQVCT |
| GDF15(N3D) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGARDGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| DhCpmFc(−) | 188 | APELLGGPSVFLFPPKPKDTLMISRTPEVTCVVVDVSHEDPEVKFNWYVDGVEVHNAKTK |
| (Y349C)- | | PREEQYNSTYRVVSVLTVLHQDWLNGKEYKCKVSNKALPAPIEKTISKAKGQPREPQVCT |
| GDF15(Nde13) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGDHCPLGPGRCCRLHTVRASLEDL |
| | | GWADWVLSPREVQVTMCIGACPSQFRAANMHAQIKTSLHRLKPDTVPAPCCVPASYNPMV |
| | | LIQKTDTGVSLQTYDDLLAKDCHCI |
| DhCpmFc(−) | 189 | APELLGGPSVFLFPPKPKDTLMISRTPEVTCVVVDVSHEDPEVKFNWYVDGVEVHNAKTK |
| (Y349C)-G4- | | PREEQYNSTYRVVSVLTVLHQDWLNGKEYKCKVSNKALPAPIEKTISKAKGQPREPQVCT |
| GDF15(N3D) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGARDGDHCPLGPGRCCRLHTV |
| | | RASLEDLGWADWVLSPREVQVTMCIGACPSQFRAANMHAQIKTSLHRLKPDTVPAPCCVP |
| | | ASYNPMVLIQKTDTGVSLQTYDDLLAKDCHCI |
| DhCpmFc(−) | 190 | APELLGGPSVFLFPPKPKDTLMISRTPEVTCVVVDVSHEDPEVKFNWYVDGVEVHNAKTK |
| (Y349C)- | | PREEQYNSTYRVVSVLTVLHQDWLNGKEYKCKVSNKALPAPIEKTISKAKGQPREPQVCT |
| (G4S)2- | | LPPSREEMTKNQVSLTCLVKGFYPSDIAVEWESNGQPENNYDTTPPVLDSDGSFFLYSDL |
| GDF15(N3D) | | TVDKSRWQQGNVFSCSVMHEALHNHYTQKSLSLSPGGGGGSGGGGSARDGDHCPLGPGRC |
| | | CRLHTVRASLEDLGWADWVLSPREVQVTMCIGACPSQFRAANMHAQIKTSLHRLKPDTVP |
| | | APCCVPASYNPMVLIQKTDTGVSLQTYDDLLAKDCHCI |
| DhCpmFc(−) | 191 | APELLGGPSVFLFPPKPKDTLMISRTPEVTCVVVDVSHEDPEVKFNWYVDGVEVHNAKTK |
| (Y349C)- | | PREEQYNSTYRVVSVLTVLHQDWLNGKEYKCKVSNKALPAPIEKTISKAKGQPREPQVCT |
| (G4Q)2- | | LPPSREEMTKNQVSLTCLVKGFYPSDIAVEWESNGQPENNYDTTPPVLDSDGSFFLYSDL |
| GDF15(N3D) | | TVDKSRWQQGNVFSCSVMHEALHNHYTQKSLSLSPGGGGGQGGGGQARDGDHCPLGPGRC |
| | | CRLHTVRASLEDLGWADWVLSPREVQVTMCIGACPSQFRAANMHAQIKTSLHRLKPDTVP |
| | | APCCVPASYNPMVLIQKTDTGVSLQTYDDLLAKDCHCI |
| DhCpmFc(+)(L351C) | 192 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | CPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGK |
| DhCpmFc(−) | 193 | APELLGGPSVFLFPPKPKDTLMISRTPEVTCVVVDVSHEDPEVKFNWYVDGVEVHNAKTK |
| (L351C)- | | PREEQYNSTYRVVSVLTVLHQDWLNGKEYKCKVSNKALPAPIEKTISKAKGQPREPQVYT |
| (G4S)2-GDF15 | | CPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGSGGGGSARNGDHCPLGPGRC |
| | | CRLHTVRASLEDLGWADWVLSPREVQVTMCIGACPSQFRAANMHAQIKTSLHRLKPDTVP |
| | | APCCVPASYNPMVLIQKTDTGVSLQTYDDLLAKDCHCI |
| DhCpmFc(−) | 194 | APELLGGPSVFLFPPKPKDTLMISRTPEVTCVVVDVSHEDPEVKFNWYVDGVEVHNAKTK |
| (L351C) | | PREEQYNSTYRVVSVLTVLHQDWLNGKEYKCKVSNKALPAPIEKTISKAKGQPREPQVYT |
| | | CPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPG |
| DhCpmFc(+)(L351C) | 195 | APELLGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVYT |
| | | CPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPG |
| HSA-(G4S)4- | 196 | DAHKSEVAHRFKDLGEENFKALVLIAFAQYLQQCPFEDHVKLVNEVTEFAKTCVADESAE |
| GDF15 | | NCDKSLHTLFGDKLCTVATLRETYGEMADCCAKQEPERNECFLQHKDDNPNLPRLVRPEV |
| | | DVMCTAFHDNEETFLKKYLYEIARRHPYFYAPELLFFAKRYKAAFTECCQAADKAACLLP |
| | | KLDELRDEGKASSAKQRLKCASLQKFGERAFKAWAVARLSQRFPKAEFAEVSKLVTDLTK |
| | | VHTECCHGDLLECADDRADLAKYICENQDSISSKLKECCEKPLLEKSHCIAEVENDEMPA |
| | | DLPSLAADFVESKDVCKNYAEAKDVFLGMFLYEYARRHPDYSVVLLLRLAKTYETTLEKC |
| | | CAAADPHECYAKVFDEFKPLVEEPQNLIKQNCELFEQLGEYKFQNALLVRYTKKVPQVST |
| | | PTLVEVSRNLGKVGSKCCKHPEAKRMPCAEDYLSVVLNQLCVLHEKTPVSDRVTKCCTES |
| | | LVNRRPCFSALEVDETYVPKEFNAETFTFHADICTLSEKERQIKKQTALVELVKHKPKAT |
| | | KEQLKAVMDDFAAFVEKCCKADDKETCFAEEGKKLVAASQAALGLGGGGSGGGGSGGGGS |
| | | GGGGSARNGDHCPLGPGRCCRLHTVRASLEDLGWADWVLSPREVQVTMCIGACPSQFRAA |
| | | NMHAQIKTSLHRLKPDTVPAPCCVPASYNPMVLIQKTDTGVSLQTYDDLLAKDCHCI |
| HSA- | 197 | DAHKSEVAHRFKDLGEENFKALVLIAFAQYLQQCPFEDHVKLVNEVTEFAKTCVADESAE |
| GSPAPAPGS- | | NCDKSLHTLFGDKLCTVATLRETYGEMADCCAKQEPERNECFLQHKDDNPNLPRLVRPEV |
| GDF15 | | DVMCTAFHDNEETFLKKYLYEIARRHPYFYAPELLFFAKRYKAAFTECCQAADKAACLLP |
| | | KLDELRDEGKASSAKQRLKCASLQKFGERAFKAWAVARLSQRFPKAEFAEVSKLVTDLTK |
| | | VHTECCHGDLLECADDRADLAKYICENQDSISSKLKECCEKPLLEKSHCIAEVENDEMPA |
| | | DLPSLAADFVESKDVCKNYAEAKDVFLGMFLYEYARRHPDYSVVLLLRLAKTYETTLEKC |
| | | CAAADPHECYAKVFDEFKPLVEEPQNLIKQNCELFEQLGEYKFQNALLVRYTKKVPQVST |
| | | PTLVEVSRNLGKVGSKCCKHPEAKRMPCAEDYLSVVLNQLCVLHEKTPVSDRVTKCCTES |
| | | LVNRRPCFSALEVDETYVPKEFNAETFTFHADICTLSEKERQIKKQTALVELVKHKPKAT |
| | | KEQLKAVMDDFAAFVEKCCKADDKETCFAEEGKKLVAASQAALGLGSPAPAPGSARNGDH |
| | | CPLGPGRCCRLHTVRASLEDLGWADWVLSPREVQVTMCIGACPSQFRAANMHAQIKTSLH |
| | | RLKPDTVPAPCCVPASYNPMVLIQKTDTGVSLQTYDDLLAKDCHCI |
| HSA- | 198 | DAHKSEVAHRFKDLGEENFKALVLIAFAQYLQQCPFEDHVKLVNEVTEFAKTCVADESAE |
| GS(PAPAP)2GS- | | NCDKSLHTLFGDKLCTVATLRETYGEMADCCAKQEPERNECFLQHKDDNPNLPRLVRPEV |
| GDF15 | | DVMCTAFHDNEETFLKKYLYEIARRHPYFYAPELLFFAKRYKAAFTECCQAADKAACLLP |
| | | KLDELRDEGKASSAKQRLKCASLQKFGERAFKAWAVARLSQRFPKAEFAEVSKLVTDLTK |
| | | VHTECCHGDLLECADDRADLAKYICENQDSISSKLKECCEKPLLEKSHCIAEVENDEMPA |
| | | DLPSLAADFVESKDVCKNYAEAKDVFLGMFLYEYARRHPDYSVVLLLRLAKTYETTLEKC |
| | | CAAADPHECYAKVFDEFKPLVEEPQNLIKQNCELFEQLGEYKFQNALLVRYTKKVPQVST |
| | | PTLVEVSRNLGKVGSKCCKHPEAKRMPCAEDYLSVVLNQLCVLHEKTPVSDRVTKCCTES |
| | | LVNRRPCFSALEVDETYVPKEFNAETFTFHADICTLSEKERQIKKQTALVELVKHKPKAT |
| | | KEQLKAVMDDFAAFVEKCCKADDKETCFAEEGKKLVAASQAALGLGSPAPAPPAPAPGSA |
| | | RNGDHCPLGPGRCCRLHTVRASLEDLGWADWVLSPREVQVTMCIGACPSQFRAANMHAQI |
| | | KTSLHRLKPDTVPAPCCVPASYNPMVLIQKTDTGVSLQTYDDLLAKDCHCI |
| HSA- | 199 | DAHKSEVAHRFKDLGEENFKALVLIAFAQYLQQCPFEDHVKLVNEVTEFAKTCVADESAE |
| GSAAQAAQQGS- | | NCDKSLHTLFGDKLCTVATLRETYGEMADCCAKQEPERNECFLQHKDDNPNLPRLVRPEV |
| GDF15 | | DVMCTAFHDNEETFLKKYLYEIARRHPYFYAPELLFFAKRYKAAFTECCQAADKAACLLP |
| | | KLDELRDEGKASSAKQRLKCASLQKFGERAFKAWAVARLSQRFPKAEFAEVSKLVTDLTK |
| | | VHTECCHGDLLECADDRADLAKYICENQDSISSKLKECCEKPLLEKSHCIAEVENDEMPA |
| | | DLPSLAADFVESKDVCKNYAEAKDVFLGMFLYEYARRHPDYSVVLLLRLAKTYETTLEKC |
| | | CAAADPHECYAKVFDEFKPLVEEPQNLIKQNCELFEQLGEYKFQNALLVRYTKKVPQVST |
| | | PTLVEVSRNLGKVGSKCCKHPEAKRMPCAEDYLSVVLNQLCVLHEKTPVSDRVTKCCTES |
| | | LVNRRPCFSALEVDETYVPKEFNAETFTFHADICTLSEKERQIKKQTALVELVKHKPKAT |
| | | KEQLKAVMDDFAAFVEKCCKADDKETCFAEEGKKLVAASQAALGLGSAAQAAQQGSARNG |
| | | DHCPLGPGRCCRLHTVRASLEDLGWADWVLSPREVQVTMCIGACPSQFRAANMHAQIKTS |
| | | LHRLKPDTVPAPCCVPASYNPMVLIQKTDTGVSLQTYDDLLAKDCHCI |
| HSA- | 200 | DAHKSEVAHRFKDLGEENFKALVLIAFAQYLQQCPFEDHVKLVNEVTEFAKTCVADESAE |
| GS(AAQAAQQ)2GS- | | NCDKSLHTLFGDKLCTVATLRETYGEMADCCAKQEPERNECFLQHKDDNPNLPRLVRPEV |
| GDF15 | | DVMCTAFHDNEETFLKKYLYEIARRHPYFYAPELLFFAKRYKAAFTECCQAADKAACLLP |
| | | KLDELRDEGKASSAKQRLKCASLQKFGERAFKAWAVARLSQRFPKAEFAEVSKLVTDLTK |
| | | VHTECCHGDLLECADDRADLAKYICENQDSISSKLKECCEKPLLEKSHCIAEVENDEMPA |
| | | DLPSLAADFVESKDVCKNYAEAKDVFLGMFLYEYARRHPDYSVVLLLRLAKTYETTLEKC |
| | | CAAADPHECYAKVFDEFKPLVEEPQNLIKQNCELFEQLGEYKFQNALLVRYTKKVPQVST |
| | | PTLVEVSRNLGKVGSKCCKHPEAKRMPCAEDYLSVVLNQLCVLHEKTPVSDRVTKCCTES |
| | | LVNRRPCFSALEVDETYVPKEFNAETFTFHADICTLSEKERQIKKQTALVELVKHKPKAT |
| | | KEQLKAVMDDFAAFVEKCCKADDKETCFAEEGKKLVAASQAALGLGSAAQAAQQAAQAAQ |
| | | QGSARNGDHCPLGPGRCCRLHTVRASLEDLGWADWVLSPREVQVTMCIGACPSQFRAANM |
| | | HAQIKTSLHRLKPDTVPAPCCVPASYNPMVLIQKTDTGVSLQTYDDLLAKDCHCI |
| HSA- | 201 | DAHKSEVAHRFKDLGEENFKALVLIAFAQYLQQCPFEDHVKLVNEVTEFAKTCVADESAE |
| GGNAEAAAKEAA | | NCDKSLHTLFGDKLCTVATLRETYGEMADCCAKQEPERNECFLQHKDDNPNLPRLVRPEV |
| AKEAAAKAGG- | | DVMCTAFHDNEETFLKKYLYEIARRHPYFYAPELLFFAKRYKAAFTECCQAADKAACLLP |
| GDF15 | | KLDELRDEGKASSAKQRLKCASLQKFGERAFKAWAVARLSQRFPKAEFAEVSKLVTDLTK |
| | | VHTECCHGDLLECADDRADLAKYICENQDSISSKLKECCEKPLLEKSHCIAEVENDEMPA |
| | | DLPSLAADFVESKDVCKNYAEAKDVFLGMFLYEYARRHPDYSVVLLLRLAKTYETTLEKC |
| | | CAAADPHECYAKVFDEFKPLVEEPQNLIKQNCELFEQLGEYKFQNALLVRYTKKVPQVST |
| | | PTLVEVSRNLGKVGSKCCKHPEAKRMPCAEDYLSVVLNQLCVLHEKTPVSDRVTKCCTES |
| | | LVNRRPCFSALEVDETYVPKEFNAETFTFHADICTLSEKERQIKKQTALVELVKHKPKAT |
| | | KEQLKAVMDDFAAFVEKCCKADDKETCFAEEGKKLVAASQAALGLGGNAEAAAKEAAAKE |
| | | AAAKEAAAKAGGARNGDHCPLGPGRCCRLHTVRASLEDLGWADWVLSPREVQVTMCIGAC |
| | | PSQFRAANMHAQIKTSLHRLKPDTVPAPCCVPASYNPMVLIQKTDTGVSLQTYDDLLAKD |
| | | CHCI |
| HSA-(G4S)6- | 202 | DAHKSEVAHRFKDLGEENFKALVLIAFAQYLQQCPFEDHVKLVNEVTEFADAHKSEVAHR |
| GDF15 | | FKDLGEENFKALVLIAFAQYLQQCPFEDHVKLVNEVTEFAKTCVADESAENCDKSLHTLF |
| | | GDKLCTVATLRETYGEMADCCAKQEPERNECFLQHKDDNPNLPRLVRPEVDVMCTAFHDN |
| | | EETFLKKYLYEIARRHPYFYAPELLFFAKRYKAAFTECCQAADKAACLLPKLDELRDEGK |
| | | ASSAKQRLKCASLQKFGERAFKAWAVARLSQRFPKAEFAEVSKLVTDLTKVHTECCHGDL |
| | | LECADDRADLAKYICENQDSISSKLKECCEKPLLEKSHCIAEVENDEMPADLPSLAADFV |
| | | ESKDVCKNYAEAKDVFLGMFLYEYARRHPDYSVVLLLRLAKTYETTLEKCCAAADPHECY |
| | | AKVFDEFKPLVEEPQNLIKQNCELFEQLGEYKFQNALLVRYTKKVPQVSTPTLVEVSRNL |
| | | GKVGSKCCKHPEAKRMPCAEDYLSVVLNQLCVLHEKTPVSDRVTKCCTESLVNRRPCFSA |
| | | LEVDETYVPKEFNAETFTFHADICTLSEKERQIKKQTALVELVKHKPKATKEQLKAVMDD |
| | | FAAFVEKCCKADDKETCFAEEGKKLVAASQAALGLGGGGSGGGGSGGGGSGGGGSGGGGS |
| | | GGGGSARNGDHCPLGPGRCCRLHTVRASLEDLGWADWVLSPREVQVTMCIGACPSQFRAA |
| | | NMHAQIKTSLHRLKPDTVPAPCCVPASYNPMVLIQKTDTGVSLQTYDDLLAKDCHCI |
| HSA- | 203 | DAHKSEVAHRFKDLGEENFKALVLIAFAQYLQQCPFEDHVKLVNEVTEFAKTCVADESAE |
| GS(AAQAAQQ)2GS- | | NCDKSLHTLFGDKLCTVATLRETYGEMADCCAKQEPERNECFLQHKDDNPNLPRLVRPEV |
| GDF15(N3D) | | DVMCTAFHDNEETFLKKYLYEIARRHPYFYAPELLFFAKRYKAAFTECCQAADKAACLLP |
| | | KLDELRDEGKASSAKQRLKCASLQKFGERAFKAWAVARLSQRFPKAEFAEVSKLVTDLTK |
| | | VHTECCHGDLLECADDRADLAKYICENQDSISSKLKECCEKPLLEKSHCIAEVENDEMPA |
| | | DLPSLAADFVESKDVCKNYAEAKDVFLGMFLYEYARRHPDYSVVLLLRLAKTYETTLEKC |
| | | CAAADPHECYAKVFDEFKPLVEEPQNLIKQNCELFEQLGEYKFQNALLVRYTKKVPQVST |
| | | PTLVEVSRNLGKVGSKCCKHPEAKRMPCAEDYLSVVLNQLCVLHEKTPVSDRVTKCCTES |
| | | LVNRRPCFSALEVDETYVPKEFNAETFTFHADICTLSEKERQIKKQTALVELVKHKPKAT |
| | | KEQLKAVMDDFAAFVEKCCKADDKETCFAEEGKKLVAASQAALGLGSAAQAAQQAAQAAQ |
| | | QGSARDGDHCPLGPGRCCRLHTVRASLEDLGWADWVLSPREVQVTMCIGACPSQFRAANM |
| | | HAQIKTSLHRLKPDTVPAPCCVPASYNPMVLIQKTDTGVSLQTYDDLLAKDCHCI |
| DhCpmFc(+)(N297G) | 204 | APELLGGPSVFLFPPKPKDTLMISRTPEVTCVVVDVSHEDPEVKFNWYVDGVEVHNAKTK |
| | | PREEQYGSTYRVVSVLTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGK |
| DhCpmFc(−) | 205 | APELLGGPSVFLFPPKPKDTLMISRTPEVTCVVVDVSHEDPEVKFNWYVDGVEVHNAKTK |
| (N297G)- | | PREEQYGSTYRVVSVLTVLHQDWLNGKEYKCKVSNKALPAPIEKTISKAKGQPREPQVYT |
| GDF15(Ndel3) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGDHCPLGPGRCCRLHTVRASLEDL |
| | | GWADWVLSPREVQVTMCIGACPSQFRAANMHAQIKTSLHRLKPDTVPAPCCVPASYNPMV |
| | | LIQKTDTGVSLQTYDDLLAKDCHCI |
| DhCpmFc(−) | 206 | APELLGGPSVFLFPPKPKDTLMISRTPEVTCVVVDVSHEDPEVKFNWYVDGVEVHNAKTK |
| (N297G) | | PREEQYGSTYRVVSVLTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPG |
| DhCpmFc(+)(N297G) | 207 | APELLGGPSVFLFPPKPKDTLMISRTPEVTCVVVDVSHEDPEVKFNWYVDGVEVHNAKTK |
| | | PREEQYGSTYRVVSVLTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPG |
| DhCpmFc(−) | 208 | APELLGGPSVFLFPPKPKDTLMISRTPEVTCVVVDVSHEDPEVKFNWYVDGVEVHNAKTK |
| (N297G)- | | PREEQYGSTYRVVSVLTVLHQDWLNGKEYKCKVSNKALPAPIEKTISKAKGQPREPQVYT |
| GDF15(ND3) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGARDGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| DhCpmFc(−) | 209 | APELLGGPSVFLFPPKPKDTLMISRTPEVTCVVVDVSHEDPEVKFNWYVDGVEVHNAKTK |
| (N297G)-G4- | | PREEQYGSTYRVVSVLTVLHQDWLNGKEYKCKVSNKALPAPIEKTISKAKGQPREPQVYT |
| GDF15(N3D) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGGGGARDGDHCPLGPGRCCRLHTV |
| | | RASLEDLGWADWVLSPREVQVTMCIGACPSQFRAANMHAQIKTSLHRLKPDTVPAPCCVP |
| | | ASYNPMVLIQKTDTGVSLQTYDDLLAKDCHCI |
| DhCpmFc(+)(N297G) | 210 | APELLGGPSVFLFPPKPKDTLMISRTPEVTCVVVDVSHEDPEVKFNWYVDGVEVHNAKTK |
| (S354C) | | PREEQYGSTYRVVSVLTVLHQDWLNGKEYKCKVSNKALPAPIEKTISKAKGQPREPQVYT |
| | | LPPCRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGK |
| DhCpmFc(−) | 211 | APELLGGPSVFLFPPKPKDTLMISRTPEVTCVVVDVSHEDPEVKFNWYVDGVEVHNAKTK |
| (N297G)(Y349C)- | | PREEQYGSTYRVVSVLTVLHQDWLNGKEYKCKVSNKALPAPIEKTISKAKGQPREPQVCT |
| GDF15(Ndel3) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGDHCPLGPGRCCRLHTVRASLEDL |
| | | GWADWVLSPREVQVTMCIGACPSQFRAANMHAQIKTSLHRLKPDTVPAPCCVPASYNPMV |
| | | LIQKTDTGVSLQTYDDLLAKDCHCI |
| DhCpmFc(−) | 212 | APELLGGPSVFLFPPKPKDTLMISRTPEVTCVVVDVSHEDPEVKFNWYVDGVEVHNAKTK |
| (N297G)(Y349C) | | PREEQYGSTYRVVSVLTVLHQDWLNGKEYKCKVSNKALPAPIEKTISKAKGQPREPQVCT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPG |
| DhCpmFc(+)(N297G) | 213 | APELLGGPSVFLFPPKPKDTLMISRTPEVTCVVVDVSHEDPEVKFNWYVDGVEVHNAKTK |
| (S354C) | | PREEQYGSTYRVVSVLTVLHQDWLNGKEYKCKVSNKALPAPIEKTISKAKGQPREPQVYT |
| | | LPPCRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPG |
| DhCpmFc(−) | 214 | APELLGGPSVFLFPPKPKDTLMISRTPEVTCVVVDVSHEDPEVKFNWYVDGVEVHNAKTK |
| (N297G)(Y349C)- | | PREEQYGSTYRVVSVLTVLHQDWLNGKEYKCKVSNKALPAPIEKTISKAKGQPREPQVCT |
| GDF15(N3D) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGARDGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| DhCpmFc(+)(N297G) | 215 | APELLGGPSVFLFPPKPKDTLMISRTPEVTCVVVDVSHEDPEVKFNWYVDGVEVHNAKTK |
| (L351C) | | PREEQYGSTYRVVSVLTVLHQDWLNGKEYKCKVSNKALPAPIEKTISKAKGQPREPQVYT |
| | | CPPSREEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGK |
| DhCpmFc(−) | 216 | APELLGGPSVFLFPPKPKDTLMISRTPEVTCVVVDVSHEDPEVKFNWYVDGVEVHNAKTK |
| (N297G)(L351C)- | | PREEQYGSTYRVVSVLTVLHQDWLNGKEYKCKVSNKALPAPIEKTISKAKGQPREPQVYT |
| GDF15(Ndel3) | | CPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGARNGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| DhCpmFc(−) | 217 | APELLGGPSVFLFPPKPKDTLMISRTPEVTCVVVDVSHEDPEVKFNWYVDGVEVHNAKTK |
| (N297G)(L351C) | | PREEQYGSTYRVVSVLTVLHQDWLNGKEYKCKVSNKALPAPIEKTISKAKGQPREPQVYT |
| | | CPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPG |
| DhCpmFc(+)(N297G) | 218 | APELLGGPSVFLFPPKPKDTLMISRTPEVTCVVVDVSHEDPEVKFNWYVDGVEVHNAKTK |
| (L351C) | | PREEQYGSTYRVVSVLTVLHQDWLNGKEYKCKVSNKALPAPIEKTISKAKGQPREPQVYT |
| | | CPPSREEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPG |
| DhCpmFc(−) | 219 | APELLGGPSVFLFPPKPKDTLMISRTPEVTCVVVDVSHEDPEVKFNWYVDGVEVHNAKTK |
| (N297G)(L351C)- | | PREEQYGSTYRVVSVLTVLHQDWLNGKEYKCKVSNKALPAPIEKTISKAKGQPREPQVYT |
| GDF15(N3D) | | CPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGARDGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| DhCpmFc(+)(N297G) | 220 | APELLGGPSVFLFPPKPKDTLMISRTPEVTCVVVDVSHEDPEVKFNWYVDGVEVHNCKTK |
| (L306C) | | PREEQYGSTYRVVSVCTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGK |
| DhCpmFc(−) | 221 | APELLGGPSVFLFPPKPKDTLMISRTPEVTCVVVDVSHEDPEVKFNWYVDGVEVHNCKTK |
| (N297G)(A287C)- | | PREEQYGSTYRVVSVCTVLHQDWLNGKEYKCKVSNKALPAPIEKTISKAKGQPREPQVYT |
| GDF15(Ndel3) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGGDHCPLGPGRCCRLHTVRASLEDL |
| | | GWADWVLSPREVQVTMCIGACPSQFRAANMHAQIKTSLHRLKPDTVPAPCCVPASYNPMV |
| | | LIQKTDTGVSLQTYDDLLAKDCHCI |
| DhCpmFc(−) | 222 | APELLGGPSVFLFPPKPKDTLMISRTPEVTCVVVDVSHEDPEVKFNWYVDGVEVHNCKTK |
| (N297G)(A287C) | | PREEQYGSTYRVVSVCTVLHQDWLNGKEYKCKVSNKALPAPIEKTISKAKGQPREPQVYT |
| | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPG |
| DhCpmFc(+)(N297G) | 223 | APELLGGPSVFLFPPKPKDTLMISRTPEVTCVVVDVSHEDPEVKFNWYVDGVEVHNCKTK |
| (L306C) | | PREEQYGSTYRVVSVCTVLHQDWLNGKEYKCKVSNKALPAPIEKTISKAKGQPREPQVYT |
| | | LPPSRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPG |
| DhCpmFc(−) | 224 | APELLGGPSVFLFPPKPKDTLMISRTPEVTCVVVDVSHEDPEVKFNWYVDGVEVHNCKTK |
| (N297G)(A287C)- | | PREEQYGSTYRVVSVCTVLHQDWLNGKEYKCKVSNKALPAPIEKTISKAKGQPREPQVYT |
| GDF15(N3D) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPGARDGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| DhCpmFc(+)(N297G) | 225 | APELLGGPSVFLFPPKPKDTLMISRTPEVTCVVVDVSHEDPEVKFNWYVDGVEVHNCKTK |
| (L306C) | | PREEQYGSTYRVVSVCTVLHQDWLNGKEYKCKVSNKALPAPIEKTISKAKGQPREPQVYT |
| (S354C) | | LPPCRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPGK |
| DhCpmFc(−) | 226 | APELLGGPSVFLFPPKPKDTLMISRTPEVTCVVVDVSHEDPEVKFNWYVDGVEVHNCKTK |
| (N297G)(A287C) | | PREEQYGSTYRVVSVCTVLHQDWLNGKEYKCKVSNKALPAPIEKTISKAKGQPREPQVCT |
| (Y349C)- | | LPPSREEMTKNQVSLTCLVKGFYPSDIAVEWESNGQPENNYDTTPPVLDSDGSFFLYSDL |
| GDF15(Ndel3) | | TVDKSRWQQGNVFSCSVMHEALHNHYTQKSLSLSPGGDHCPLGPGRCCRLHTVRASLEDL |
| | | GWADWVLSPREVQVTMCIGACPSQFRAANMHAQIKTSLHRLKPDTVPAPCCVPASYNPMV |
| | | LIQKTDTGVSLQTYDDLLAKDCHCI |
| DhCpmFc(−) | 227 | APELLGGPSVFLFPPKPKDTLMISRTPEVTCVVVDVSHEDPEVKFNWYVDGVEVHNCKTK |
| (N297G)(A287C) | | PREEQYGSTYRVVSVCTVLHQDWLNGKEYKCKVSNKALPAPIEKTISKAKGQPREPQVCT |
| (S354C) | | LPPSREEMTKNQVSLTCLVKGFYPSDIAVEWESNGQPENNYDTTPPVLDSDGSFFLYSDL |
| | | TVDKSRWQQGNVFSCSVMHEALHNHYTQKSLSLSPG |
| DhCpmFc(+)(N297G) | 228 | APELLGGPSVFLFPPKPKDTLMISRTPEVTCVVVDVSHEDPEVKFNWYVDGVEVHNCKTK |
| (L306C) | | PREEQYGSTYRVVSVCTVLHQDWLNGKEYKCKVSNKALPAPIEKTISKAKGQPREPQVYT |
| (Y349C) | | LPPCRKEMTKNQVSLTCLVKGFYPSDIAVEWESNGQPENNYKTTPPVLKSDGSFFLYSKL |
| | | TVDKSRWQQGNVFSCSVMHEALHNHYTQKSLSLSPG |
| DhCpmFc(−) | 229 | APELLGGPSVFLFPPKPKDTLMISRTPEVTCVVVDVSHEDPEVKFNWYVDGVEVHNCKTK |
| (N297G)(A287C) | | PREEQYGSTYRVVSVCTVLHQDWLNGKEYKCKVSNKALPAPIEKTISKAKGQPREPQVCT |
| (Y349C)- | | LPPSREEMTKNQVSLTCLVKGFYPSDIAVEWESNGQPENNYDTTPPVLDSDGSFFLYSDL |
| GDF15(N3D) | | TVDKSRWQQGNVFSCSVMHEALHNHYTQKSLSLSPGARDGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| Dh2CpmFc(+) | 230 | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRKE |
| | | MTKNQVSLTCLVKGFYPSDIAVEWESNGQPENNYKTTPPVLKSDGSFFLYSKLTVDKSRW |
| | | QQGNVFSCSVMHEALHNHYTQKSLSLSPGK |
| Dh2CpmFc(−)- | 231 | PSVFLFPPKPKDTLMISRTPEVTCVVVDVSHEDPEVKFNWYVDGVEVHNAKTKPREEQYN |
| GDF15(Ndel3) | | STYRVVSVLTVLHQDWLNGKEYKCKVSNKALPAPIEKTISKAKGQPREPQVYTLPPSREE |
| | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPGGDHCPLGPGRCCRLHTVRASLEDLGWADWVL |
| | | SPREVQVTMCIGACPSQFRAANMHAQIKTSLHRLKPDTVPAPCCVPASYNPMVLIQKTDT |
| | | GVSLQTYDDLLAKDCHCI |
| Dh2CpmFc(−) | 232 | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSREE |
| | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPG |
| Dh2CpmFc(+) | 233 | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRKE |
| | | MTKNQVSLTCLVKGFYPSDIAVEWESNGQPENNYKTTPPVLKSDGSFFLYSKLTVDKSRW |
| | | QQGNVFSCSVMHEALHNHYTQKSLSLSPG |
| Dh2CpmFc(−)- | 234 | PSVFLFPPKPKDTLMISRTPEVTCVVVDVSHEDPEVKFNWYVDGVEVHNAKTKPREEQYN |
| GDF15(N3D) | | STYRVVSVLTVLHQDWLNGKEYKCKVSNKALPAPIEKTISKAKGQPREPQVYTLPPSREE |
| | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPGARDGDHCPLGPGRCCRLHTVRASLEDLGWAD |
| | | WVLSPREVQVTMCIGACPSQFRAANMHAQIKTSLHRLKPDTVPAPCCVPASYNPMVLIQK |
| | | TDTGVSLQTYDDLLAKDCHCI |
| Dh2CpmFc(+)(S354C) | 235 | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPCRKE |
| | | MTKNQVSLTCLVKGFYPSDIAVEWESNGQPENNYKTTPPVLKSDGSFFLYSKLTVDKSRW |
| | | QQGNVFSCSVMHEALHNHYTQKSLSLSPGK |
| Dh2CpmFc(−) | 236 | PSVFLFPPKPKDTLMISRTPEVTCVVVDVSHEDPEVKFNWYVDGVEVHNAKTKPREEQYN |
| (Y349C)- | | STYRVVSVLTVLHQDWLNGKEYKCKVSNKALPAPIEKTISKAKGQPREPQVCTLPPSREE |
| GDF15(Ndel3) | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPGGDHCPLGPGRCCRLHTVRASLEDLGWADWVL |
| | | SPREVQVTMCIGACPSQFRAANMHAQIKTSLHRLKPDTVPAPCCVPASYNPMVLIQKTDT |
| | | GVSLQTYDDLLAKDCHCI |
| Dh2CpmFc(−) | 237 | PSVFLFPPKPKDTLMISRTPEVTCVVVDVSHEDPEVKFNWYVDGVEVHNAKTKPREEQYN |
| (Y349C) | | STYRVVSVLTVLHQDWLNGKEYKCKVSNKALPAPIEKTISKAKGQPREPQVCTLPPSREE |
| | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPG |
| Dh2CpmFc(+) | 238 | PSVFLFPPKPKDTLMISRTPEVTCVVVDVSHEDPEVKFNWYVDGVEVHNAKTKPREEQYN |
| (S354C) | | STYRVVSVLTVLHQDWLNGKEYKCKVSNKALPAPIEKTISKAKGQPREPQVYTLPPCRKE |
| | | MTKNQVSLTCLVKGFYPSDIAVEWESNGQPENNYKTTPPVLKSDGSFFLYSKLTVDKSRW |
| | | QQGNVFSCSVMHEALHNHYTQKSLSLSPG |
| Dh2CpmFc(−) | 239 | PSVFLFPPKPKDTLMISRTPEVTCVVVDVSHEDPEVKFNWYVDGVEVHNAKTKPREEQYN |
| (Y349C)- | | STYRVVSVLTVLHQDWLNGKEYKCKVSNKALPAPIEKTISKAKGQPREPQVCTLPPSREE |
| GDF15(N3D) | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPGARDGDHCPLGPGRCCRLHTVRASLEDLGWAD |
| | | WVLSPREVQVTMCIGACPSQFRAANMHAQIKTSLHRLKPDTVPAPCCVPASYNPMVLIQK |
| | | TDTGVSLQTYDDLLAKDCHCI |
| CpmFc(+)(N297G) | 240 | DKTHTCPPCPAPELLGGPSVFLFPPKPKDTLMISRTPEVTCVVVDVSHEDPEVKFNWYVD |
| | | GVEVHNAKTKPREEQYGSTYRVVSVLTVLHQDWLNGKEYKCKVSNKALPAPIEKTISKAK |
| | | GQPREPQVYTLPPSRKEMTKNQVSLTCLVKGFYPSDIAVEWESNGQPENNYKTTPPVLKS |
| | | DGSFFLYSKLTVDKSRWQQGNVFSCSVMHEALHNHYTQKSLSLSPGK |
| CpmFc(−) | 241 | DKTHTCPPCPAPELLGGPSVFLFPPKPKDTLMISRTPEVTCVVVDVSHEDPEVKFNWYVD |
| (N297G)- | | GVEVHNAKTKPREEQYGSTYRVVSVLTVLHQDWLNGKEYKCKVSNKALPAPIEKTISKAK |
| GDF15(Ndel3) | | GQPREPQVYTLPPSREEMTKNQVSLTCLVKGFYPSDIAVEWESNGQPENNYDTTPPVLDS |
| | | DGSFFLYSDLTVDKSRWQQGNVFSCSVMHEALHNHYTQKSLSLSPGGDHCPLGPGRCCRL |
| | | HTVRASLEDLGWADWVLSPREVQVTMCIGACPSQFRAANMHAQIKTSLHRLKPDTVPAPC |
| | | CVPASYNPMVLIQKTDTGVSLQTYDDLLAKDCHCI |
| CpmFc(−) | 242 | DKTHTCPPCPAPELLGGPSVFLFPPKPKDTLMISRTPEVTCVVVDVSHEDPEVKFNWYVD |
| (N297G) | | GVEVHNAKTKPREEQYGSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPENNYDTTPPVLDS |
| | | DGSFFLYSDLTVDKSRWQQGNVFSCSVMHEALHNHYTQKSLSLSPG |
| CpmFc(+)(N297G) | 243 | DKTHTCPPCPAPELLGGPSVFLFPPKPKDTLMISRTPEVTCVVVDVSHEDPEVKFNWYVD |
| | | GVEVHNAKTKPREEQYGSTYRVVSVLTVLHQDWLNGKEYKCKVSNKALPAPIEKTISKAK |
| | | GQPREPQVYTLPPSRKEMTKNQVSLTCLVKGFYPSDIAVEWESNGQPENNYKTTPPVLKS |
| | | DGSFFLYSKLTVDKSRWQQGNVFSCSVMHEALHNHYTQKSLSLSPG |
| CpmFc(−) | 244 | DKTHTCPPCPAPELLGGPSVFLFPPKPKDTLMISRTPEVTCVVVDVSHEDPEVKFNWYVD |
| (N297G)- | | GVEVHNAKTKPREEQYGSTYRVVSVLTVLHQDWLNGKEYKCKVSNKALPAPIEKTISKAK |
| GDF15(N3D) | | GQPREPQVYTLPPSREEMTKNQVSLTCLVKGFYPSDIAVEWESNGQPENNYDTTPPVLDS |
| | | DGSFFLYSDLTVDKSRWQQGNVFSCSVMHEALHNHYTQKSLSLSPGARDGDHCPLGPGRC |
| | | CRLHTVRASLEDLGWADWVLSPREVQVTMCIGACPSQFRAANMHAQIKTSLHRLKPDTVP |
| | | APCCVPASYNPMVLIQKTDTGVSLQTYDDLLAKDCHCI |
| Dh2CpmFc(+)(N297G) | 245 | PSVFLFPPKPKDTLMISRTPEVTCVVVDVSHEDPEVKFNWYVDGVEVHNAKTKPREEQYG |
| | | STYRVVSVLTVLHQDWLNGKEYKCKVSNKALPAPIEKTISKAKGQPREPQVYTLPPSRKE |
| | | MTKNQVSLTCLVKGFYPSDIAVEWESNGQPENNYKTTPPVLKSDGSFFLYSKLTVDKSRW |
| | | QQGNVFSCSVMHEALHNHYTQKSLSLSPGK |
| Dh2CpmFc(−) | 246 | PSVFLFPPKPKDTLMISRTPEVTCVVVDVSHEDPEVKFNWYVDGVEVHNAKTKPREEQYG |
| (N297G)- | | STYRVVSVLTVLHQDWLNGKEYKCKVSNKALPAPIEKTISKAKGQPREPQVYTLPPSREE |
| GDF15(Ndel3) | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPGGDHCPLGPGRCCRLHTVRASLEDLGWADWVL |
| | | SPREVQVTMCIGACPSQFRAANMHAQIKTSLHRLKPDTVPAPCCVPASYNPMVLIQKTDT |
| | | GVSLQTYDDLLAKDCHCI |
| Dh2CpmFc(−) | 247 | PSVFLFPPKPKDTLMISRTPEVTCVVVDVSHEDPEVKFNWYVDGVEVHNAKTKPREEQYG |
| (N297G) | | STYRVVSVLTVLHQDWLNGKEYKCKVSNKALPAPIEKTISKAKGQPREPQVYTLPPSREE |
| | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPG |
| Dh2CpmFc(+) | 248 | PSVFLFPPKPKDTLMISRTPEVTCVVVDVSHEDPEVKFNWYVDGVEVHNAKTKPREEQYG |
| (N297G) | | STYRVVSVLTVLHQDWLNGKEYKCKVSNKALPAPIEKTISKAKGQPREPQVYTLPPSRKE |
| | | MTKNQVSLTCLVKGFYPSDIAVEWESNGQPENNYKTTPPVLKSDGSFFLYSKLTVDKSRW |
| | | QQGNVFSCSVMHEALHNHYTQKSLSLSPG |
| Dh2CpmFc(−) | 249 | PSVFLFPPKPKDTLMISRTPEVTCVVVDVSHEDPEVKFNWYVDGVEVHNAKTKPREEQYG |
| (N297G)- | | STYRVVSVLTVLHQDWLNGKEYKCKVSNKALPAPIEKTISKAKGQPREPQVYTLPPSREE |
| GDF15(N3D) | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPGARDGDHCPLGPGRCCRLHTVRASLEDLGWAD |
| | | WVLSPREVQVTMCIGACPSQFRAANMHAQIKTSLHRLKPDTVPAPCCVPASYNPMVLIQK |
| | | TDTGVSLQTYDDLLAKDCHCI |
| Dh2CpmFc(+) | 250 | PSVFLFPPKPKDTLMISRTPEVTCVVVDVSHEDPEVKFNWYVDGVEVHNAKTKPREEQYG |
| (N297G)(S354C) | | STYRVVSVLTVLHQDWLNGKEYKCKVSNKALPAPIEKTISKAKGQPREPQVYTLPPCRKE |
| | | MTKNQVSLTCLVKGFYPSDIAVEWESNGQPENNYKTTPPVLKSDGSFFLYSKLTVDKSRW |
| | | QQGNVFSCSVMHEALHNHYTQKSLSLSPGK |
| Dh2CpmFc(−) | 251 | PSVFLFPPKPKDTLMISRTPEVTCVVVDVSHEDPEVKFNWYVDGVEVHNAKTKPREEQYG |
| (N297G)(Y349C)- | | STYRVVSVLTVLHQDWLNGKEYKCKVSNKALPAPIEKTISKAKGQPREPQVCTLPPSREE |
| GDF15(Ndel3) | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPGGDHCPLGPGRCCRLHTVRASLEDLGWADWVL |
| | | SPREVQVTMCIGACPSQFRAANMHAQIKTSLHRLKPDTVPAPCCVPASYNPMVLIQKTDT |
| | | GVSLQTYDDLLAKDCHCI |
| Dh2CpmFc(−) | 252 | PSVFLFPPKPKDTLMISRTPEVTCVVVDVSHEDPEVKFNWYVDGVEVHNAKTKPREEQYG |
| (N297G)(Y349C) | | STYRVVSVLTVLHQDWLNGKEYKCKVSNKALPAPIEKTISKAKGQPREPQVCTLPPSREE |
| | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPG |
| Dh2CpmFc(+) | 253 | PSVFLFPPKPKDTLMISRTPEVTCVVVDVSHEDPEVKFNWYVDGVEVHNAKTKPREEQYG |
| (N297G)(S354C) | | STYRVVSVLTVLHQDWLNGKEYKCKVSNKALPAPIEKTISKAKGQPREPQVYTLPPCRKE |
| | | MTKNQVSLTCLVKGFYPSDIAVEWESNGQPENNYKTTPPVLKSDGSFFLYSKLTVDKSRW |
| | | QQGNVFSCSVMHEALHNHYTQKSLSLSPG |
| Dh2CpmFc(−) | 254 | PSVFLFPPKPKDTLMISRTPEVTCVVVDVSHEDPEVKFNWYVDGVEVHNAKTKPREEQYG |
| (N297G)(Y349C)- | | STYRVVSVLTVLHQDWLNGKEYKCKVSNKALPAPIEKTISKAKGQPREPQVCTLPPSREE |
| GDF15(N3D) | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPGARDGDHCPLGPGRCCRLHTVRASLEDLGWAD |
| | | WVLSPREVQVTMCIGACPSQFRAANMHAQIKTSLHRLKPDTVPAPCCVPASYNPMVLIQK |
| | | TDTGVSLQTYDDLLAKDCHCI |
| Dh2CpmFc(+) | 255 | PSVFLFPPKPKDTLMISRTPEVTCVVVDVSHEDPEVKFNWYVDGVEVHNCKTKPREEQYG |
| (N297G)(L306C) | | TYRVVSVCTVLHQDWLNGKEYKCKVSNKALPAPIEKTISKAKGQPREPQVYTLPPSRKE |
| | | MTKNQVSLTCLVKGFYPSDIAVEWESNGQPENNYKTTPPVLKSDGSFFLYSKLTVDKSRW |
| | | QQGNVFSCSVMHEALHNHYTQKSLSLSPGK |
| Dh2CpmFc(−) | 256 | PSVFLFPPKPKDTLMISRTPEVTCVVVDVSHEDPEVKFNWYVDGVEVHNCKTKPREEQYG |
| (N297G)(A287C)- | | STYRVVSVCTVLHQDWLNGKEYKCKVSNKALPAPIEKTISKAKGQPREPQVYTLPPSREE |
| GDF15(Ndel3) | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPGGDHCPLGPGRCCRLHTVRASLEDLGWADWVL |
| | | SPREVQVTMCIGACPSQFRAANMHAQIKTSLHRLKPDTVPAPCCVPASYNPMVLIQKTDT |
| | | GVSLQTYDDLLAKDCHCI |
| Dh2CpmFc(−) | 257 | PSVFLFPPKPKDTLMISRTPEVTCVVVDVSHEDPEVKFNWYVDGVEVHNCKTKPREEQYG |
| (N297G)(A287C) | | STYRVVSVCTVLHQDWLNGKEYKCKVSNKALPAPIEKTISKAKGQPREPQVYTLPPSREE |
| | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPG |
| Dh2CpmFc(+) | 258 | PSVFLFPPKPKDTLMISRTPEVTCVVVDVSHEDPEVKFNWYVDGVEVHNCKTKPREEQYG |
| (N297G)(L306C) | | STYRVVSVCTVLHQDWLNGKEYKCKVSNKALPAPIEKTISKAKGQPREPQVYTLPPSRKE |
| | | MTKNQVSLTCLVKGFYPSDIAVEWESNGQPENNYKTTPPVLKSDGSFFLYSKLTVDKSRW |
| | | QQGNVFSCSVMHEALHNHYTQKSLSLSPG |
| Dh2CpmFc(−) | 259 | PSVFLFPPKPKDTLMISRTPEVTCVVVDVSHEDPEVKFNWYVDGVEVHNCKTKPREEQYG |
| (N297G)(A287C)- | | STYRVVSVCTVLHQDWLNGKEYKCKVSNKALPAPIEKTISKAKGQPREPQVYTLPPSREE |
| GDF15(N3D) | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPGARDGDHCPLGPGRCCRLHTVRASLEDLGWAD |
| | | WVLSPREVQVTMCIGACPSQFRAANMHAQIKTSLHRLKPDTVPAPCCVPASYNPMVLIQK |
| | | TDTGVSLQTYDDLLAKDCHCI |
| Dh2CpmFc(+) | 260 | PSVFLFPPKPKDTLMISRTPEVTCVVVDVSHEDPEVKFNWYVDGVEVHNCKTKPREEQYG |
| (N297G)(L306C) | | STYRVVSVCTVLHQDWLNGKEYKCKVSNKALPAPIEKTISKAKGQPREPQVYTLPPCRKE |
| (S354C) | | MTKNQVSLTCLVKGFYPSDIAVEWESNGQPENNYKTTPPVLKSDGSFFLYSKLTVDKSRW |
| | | QQGNVFSCSVMHEALHNHYTQKSLSLSPGK |
| Dh2CpmFc(−) | 261 | PSVFLFPPKPKDTLMISRTPEVTCVVVDVSHEDPEVKFNWYVDGVEVHNCKTKPREEQYG |
| (N297G)(A287C) | | STYRVVSVCTVLHQDWLNGKEYKCKVSNKALPAPIEKTISKAKGQPREPQVCTLPPSREE |
| (Y349C)- | | MTKNQVSLTCLVKGFYPSDIAVEWESNGQPENNYDTTPPVLDSDGSFFLYSDLTVDKSRW |
| GDF15(Ndel3) | | QQGNVFSCSVMHEALHNHYTQKSLSLSPGGDHCPLGPGRCCRLHTVRASLEDLGWADWVL |
| | | SPREVQVTMCIGACPSQFRAANMHAQIKTSLHRLKPDTVPAPCCVPASYNPMVLIQKTDT |
| | | GVSLQTYDDLLAKDCHCI |
| Dh2CpmFc(−) | 262 | PSVFLFPPKPKDTLMISRTPEVTCVVVDVSHEDPEVKFNWYVDGVEVHNCKTKPREEQYG |
| (N297G)(A287C) | | STYRVVSVCTVLHQDWLNGKEYKCKVSNKALPAPIEKTISKAKGQPREPQVCTLPPSREE |
| (Y349C) | | MTKNQVSLTCLVKGFYPSDIAVEWESNGQPENNYDTTPPVLDSDGSFFLYSDLTVDKSRW |
| | | QQGNVFSCSVMHEALHNHYTQKSLSLSPG |
| Dh2CpmFc(+) | 263 | PSVFLFPPKPKDTLMISRTPEVTCVVVDVSHEDPEVKFNWYVDGVEVHNCKTKPREEQYG |
| (N297G)(L306C) | | STYRVVSVCTVLHQDWLNGKEYKCKVSNKALPAPIEKTISKAKGQPREPQVYTLPPCRKE |
| (S354C) | | MTKNQVSLTCLVKGFYPSDIAVEWESNGQPENNYKTTPPVLKSDGSFFLYSKLTVDKSRW |
| | | QQGNVFSCSVMHEALHNHYTQKSLSLSPG |
| Dh2CpmFc(−) | 264 | PSVFLFPPKPKDTLMISRTPEVTCVVVDVSHEDPEVKFNWYVDGVEVHNCKTKPREEQYG |
| (N297G)(A287C) | | STYRVVSVCTVLHQDWLNGKEYKCKVSNKALPAPIEKTISKAKGQPREPQVCTLPPSREE |
| (Y349C)- | | MTKNQVSLTCLVKGFYPSDIAVEWESNGQPENNYDTTPPVLDSDGSFFLYSDLTVDKSRW |
| GDF15(N3D) | | QQGNVFSCSVMHEALHNHYTQKSLSLSPGARDGDHCPLGPGRCCRLHTVRASLEDLGWAD |
| | | WVLSPREVQVTMCIGACPSQFRAANMHAQIKTSLHRLKPDTVPAPCCVPASYNPMVLIQK |
| | | TDTGVSLQTYDDLLAKDCHCI |
| GG- | 265 | GGPSVFLFPPKPKDTLMISRTPEVTCVVVDVSHEDPEVKFNWYVDGVEVHNAKTKPREEQ |
| Dh2CpmFc(+) | | YNSTYRVVSVLTVLHQDWLNGKEYKCKVSNKALPAPIEKTISKAKGQPREPQVYTLPPSR |
| | | KEMTKNQVSLTCLVKGFYPSDIAVEWESNGQPENNYKTTPPVLKSDGSFFLYSKLTVDKS |
| | | RWQQGNVFSCSVMHEALHNHYTQKSLSLSPGK |
| GG- | 266 | MEWSWVFLFFLSVTTGVHSGGPSVFLFPPKPKDTLMISRTPEVTCVVVDVSHEDPEVKFN |
| Dh2CpmFc(+) | | WYVDGVEVHNAKTKPREEQYNSTYRVVSVLTVLHQDWLNGKEYKCKVSNKALPAPIEKTI |
| with VH21 | | SKAKGQPREPQVYTLPPSRKEMTKNQVSLTCLVKGFYPSDIAVEWESNGQPENNYKTTPP |
| signal | | VLKSDGSFFLYSKLTVDKSRWQQGNVFSCSVMHEALHNHYTQKSLSLSPGK |
| sequence | | |
| GG- | 267 | GGPSVFLFPPKPKDTLMISRTPEVTCVVVDVSHEDPEVKFNWYVDGVEVHNAKTKPREEQ |
| Dh2CpmFc(−)- | | YNSTYRVVSVLTVLHQDWLNGKEYKCKVSNKALPAPIEKTISKAKGQPREPQVYTLPPSR |
| GDF15(Ndel3) | | EEMTKNQVSLTCLVKGFYPSDIAVEWESNGQPENNYDTTPPVLDSDGSFFLYSDLTVDKS |
| | | RWQQGNVFSCSVMHEALHNHYTQKSLSLSPGGDHCPLGPGRCCRLHTVRASLEDLGWADW |
| | | VLSPREVQVTMCIGACPSQFRAANMHAQIKTSLHRLKPDTVPAPCCVPASYNPMVLIQKT |
| | | DTGVSLQTYDDLLAKDCHCI |
| GG- | 268 | MEWSWVFLFFLSVTTGVHSGGPSVFLFPPKPKDTLMISRTPEVTCVVVDVSHEDPEVKFN |
| Dh2CpmFc(−)- | | WYVDGVEVHNAKTKPREEQYNSTYRVVSVLTVLHQDWLNGKEYKCKVSNKALPAPIEKTI |
| GDF15(Ndel3) | | SKAKGQPREPQVYTLPPSREEMTKNQVSLTCLVKGFYPSDIAVEWESNGQPENNYDTTPP |
| with VH21 | | VLDSDGSFFLYSDLTVDKSRWQQGNVFSCSVMHEALHNHYTQKSLSLSPGGDHCPLGPGR |
| signal | | CCRLHTVRASLEDLGWADWVLSPREVQVTMCIGACPSQFRAANMHAQIKTSLHRLKPDTV |
| sequence | | PAPCCVPASYNPMVLIQKTDTGVSLQTYDDLLAKDCHCI |
| GG- | 269 | GGPSVFLFPPKPKDTLMISRTPEVTCVVVDVSHEDPEVKFNWYVDGVEVHNAKTKPREEQ |
| Dh2CpmFc(−) | | YNSTYRVVSVLTVLHQDWLNGKEYKCKVSNKALPAPIEKTISKAKGQPREPQVYTLPPSR |
| | | EEMTKNQVSLTCLVKGFYPSDIAVEWESNGQPENNYDTTPPVLDSDGSFFLYSDLTVDKS |
| | | RWQQGNVFSCSVMHEALHNHYTQKSLSLSPG |
| GG- | 270 | GGPSVFLFPPKPKDTLMISRTPEVTCVVVDVSHEDPEVKFNWYVDGVEVHNAKTKPREEQ |
| Dh2CpmFc(+) | | YNSTYRVVSVLTVLHQDWLNGKEYKCKVSNKALPAPIEKTISKAKGQPREPQVYTLPPSR |
| | | KEMTKNQVSLTCLVKGFYPSDIAVEWESNGQPENNYKTTPPVLKSDGSFFLYSKLTVDKS |
| | | RWQQGNVFSCSVMHEALHNHYTQKSLSLSPG |
| GG- | 271 | GGPSVFLFPPKPKDTLMISRTPEVTCVVVDVSHEDPEVKFNWYVDGVEVHNAKTKPREEQ |
| Dh2CpmFc(−)- | | YNSTYRVVSVLTVLHQDWLNGKEYKCKVSNKALPAPIEKTISKAKGQPREPQVYTLPPSR |
| GDF15(N3D) | | EEMTKNQVSLTCLVKGFYPSDIAVEWESNGQPENNYDTTPPVLDSDGSFFLYSDLTVDKS |
| | | RWQQGNVFSCSVMHEALHNHYTQKSLSLSPGARDGDHCPLGPGRCCRLHTVRASLEDLGW |
| | | ADWVLSPREVQVTMCIGACPSQFRAANMHAQIKTSLHRLKPDTVPAPCCVPASYNPMVLI |
| | | QKTDTGVSLQTYDDLLAKDCHCI |
| GG- | 272 | MEWSWVFLFFLSVTTGVHSGGPSVFLFPPKPKDTLMISRTPEVTCVVVDVSHEDPEVKFN |
| Dh2CpmFc(−)- | | WYVDGVEVHNAKTKPREEQYNSTYRVVSVLTVLHQDWLNGKEYKCKVSNKALPAPIEKTI |
| GDF15(N3D) | | SKAKGQPREPQVYTLPPSREEMTKNQVSLTCLVKGFYPSDIAVEWESNGQPENNYDTTPP |
| with VH21 | | VLDSDGSFFLYSDLTVDKSRWQQGNVFSCSVMHEALHNHYTQKSLSLSPGARDGDHCPLG |
| signal | | PGRCCRLHTVRASLEDLGWADWVLSPREVQVTMCIGACPSQFRAANMHAQIKTSLHRLKP |
| sequence | | DTVPAPCCVPASYNPMVLIQKTDTGVSLQTYDDLLAKDCHCI |
| GG- | 273 | GGPSVFLFPPKPKDTLMISRTPEVTCVVVDVSHEDPEVKFNWYVDGVEVHNAKTKPREEQ |
| Dh2CpmFc(+) | | YNSTYRVVSVLTVLHQDWLNGKEYKCKVSNKALPAPIEKTISKAKGQPREPQVYTLPPCR |
| (S354C) | | KEMTKNQVSLTCLVKGFYPSDIAVEWESNGQPENNYKTTPPVLKSDGSFFLYSKLTVDKS |
| | | RWQQGNVFSCSVMHEALHNHYTQKSLSLSPGK |
| GG- | 274 | MEWSWVFLFFLSVTTGVHSGGPSVFLFPPKPKDTLMISRTPEVTCVVVDVSHEDPEVKFN |
| Dh2CpmFc(+) | | WYVDGVEVHNAKTKPREEQYNSTYRVVSVLTVLHQDWLNGKEYKCKVSNKALPAPIEKTI |
| (S354C) with | | SKAKGQPREPQVYTLPPCRKEMTKNQVSLTCLVKGFYPSDIAVEWESNGQPENNYKTTPP |
| VH21 signal | | VLKSDGSFFLYSKLTVDKSRWQQGNVFSCSVMHEALHNHYTQKSLSLSPGK |
| sequence | | |
| GG- | 275 | GGPSVFLFPPKPKDTLMISRTPEVTCVVVDVSHEDPEVKFNWYVDGVEVHNAKTKPREEQ |
| Dh2CpmFc(−) | | YNSTYRVVSVLTVLHQDWLNGKEYKCKVSNKALPAPIEKTISKAKGQPREPQVCTLPPSR |
| (Y349C)- | | EEMTKNQVSLTCLVKGFYPSDIAVEWESNGQPENNYDTTPPVLDSDGSFFLYSDLTVDKS |
| GDF15(Ndel3) | | RWQQGNVFSCSVMHEALHNHYTQKSLSLSPGGDHCPLGPGRCCRLHTVRASLEDLGWADW |
| | | VLSPREVQVTMCIGACPSQFRAANMHAQIKTSLHRLKPDTVPAPCCVPASYNPMVLIQKT |
| | | DTGVSLQTYDDLLAKDCHCI |
| GG- | 276 | MEWSWVFLFFLSVTTGVHSGGPSVFLFPPKPKDTLMISRTPEVTCVVVDVSHEDPEVKFN |
| Dh2CpmFc(−) | | WYVDGVEVHNAKTKPREEQYNSTYRVVSVLTVLHQDWLNGKEYKCKVSNKALPAPIEKTI |
| (Y349C)- | | SKAKGQPREPQVCTLPPSREEMTKNQVSLTCLVKGFYPSDIAVEWESNGQPENNYDTTPP |
| GDF15(Ndel3) | | VLDSDGSFFLYSDLTVDKSRWQQGNVFSCSVMHEALHNHYTQKSLSLSPGGDHCPLGPGR |
| with VH21 | | CCRLHTVRASLEDLGWADWVLSPREVQVTMCIGACPSQFRAANMHAQIKTSLHRLKPDTV |
| signal | | PAPCCVPASYNPMVLIQKTDTGVSLQTYDDLLAKDCHCI |
| sequence | | |
| GG- | 277 | GGPSVFLFPPKPKDTLMISRTPEVTCVVVDVSHEDPEVKFNWYVDGVEVHNAKTKPREEQ |
| Dh2CpmFc(−) | | YNSTYRVVSVLTVLHQDWLNGKEYKCKVSNKALPAPIEKTISKAKGQPREPQVCTLPPSR |
| (Y349C) | | EEMTKNQVSLTCLVKGFYPSDIAVEWESNGQPENNYDTTPPVLDSDGSFFLYSDLTVDKS |
| | | RWQQGNVFSCSVMHEALHNHYTQKSLSLSPG |
| GG- | 278 | GGPSVFLFPPKPKDTLMISRTPEVTCVVVDVSHEDPEVKFNWYVDGVEVHNAKTKPREEQ |
| Dh2CpmFc(+) | | YNSTYRVVSVLTVLHQDWLNGKEYKCKVSNKALPAPIEKTISKAKGQPREPQVYTLPPCR |
| (S354C) | | KEMTKNQVSLTCLVKGFYPSDIAVEWESNGQPENNYKTTPPVLKSDGSFFLYSKLTVDKS |
| | | RWQQGNVFSCSVMHEALHNHYTQKSLSLSPG |
| GG- | 279 | GGPSVFLFPPKPKDTLMISRTPEVTCVVVDVSHEDPEVKFNWYVDGVEVHNAKTKPREEQ |
| Dh2CpmFc(−) | | YNSTYRVVSVLTVLHQDWLNGKEYKCKVSNKALPAPIEKTISKAKGQPREPQVCTLPPSR |
| (Y349C)- | | EEMTKNQVSLTCLVKGFYPSDIAVEWESNGQPENNYDTTPPVLDSDGSFFLYSDLTVDKS |
| GDF15(N3D) | | RWQQGNVFSCSVMHEALHNHYTQKSLSLSPGARDGDHCPLGPGRCCRLHTVRASLEDLGW |
| | | ADWVLSPREVQVTMCIGACPSQFRAANMHAQIKTSLHRLKPDTVPAPCCVPASYNPMVLI |
| | | QKTDTGVSLQTYDDLLAKDCHCI |
| GG- | 280 | MEWSWVFLFFLSVTTGVHSGGPSVFLFPPKPKDTLMISRTPEVTCVVVDVSHEDPEVKFN |
| Dh2CpmFc(−) | | WYVDGVEVHNAKTKPREEQYNSTYRVVSVLTVLHQDWLNGKEYKCKVSNKALPAPIEKTI |
| (Y349C)- | | SKAKGQPREPQVCTLPPSREEMTKNQVSLTCLVKGFYPSDIAVEWESNGQPENNYDTTPP |
| GDF15(N3D) | | VLDSDGSFFLYSDLTVDKSRWQQGNVFSCSVMHEALHNHYTQKSLSLSPGARDGDHCPLG |
| with VH21 | | PGRCCRLHTVRASLEDLGWADWVLSPREVQVTMCIGACPSQFRAANMHAQIKTSLHRLKP |
| signal | | DTVPAPCCVPASYNPMVLIQKTDTGVSLQTYDDLLAKDCHCI |
| sequence | | |
| Dh3CpmFc(+) | 281 | GPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRK |
| | | EMTKNQVSLTCLVKGFYPSDIAVEWESNGQPENNYKTTPPVLKSDGSFFLYSKLTVDKSR |
| | | WQQGNVFSCSVMHEALHNHYTQKSLSLSPGK |
| Dh3CpmFc(+) | 282 | MDMRVPAQLLGLLLLWLRGARCGPSVFLFPPKPKDTLMISRTPEVTCVVVDVSHEDPEVK |
| with VH21 | | FNWYVDGVEVHNAKTKPREEQYNSTYRVVSVLTVLHQDWLNGKEYKCKVSNKALPAPIEK |
| signal | | TISKAKGQPREPQVYTLPPSRKEMTKNQVSLTCLVKGFYPSDIAVEWESNGQPENNYKTT |
| sequence | | PPVLKSDGSFFLYSKLTVDKSRWQQGNVFSCSVMHEALHNHYTQKSLSLSPGK |
| Dh3CpmFc(−)- | 283 | GPSVFLFPPKPKDTLMISRTPEVTCVVVDVSHEDPEVKFNWYVDGVEVHNAKTKPREEQY |
| GDF15(Ndel3) | | NSTYRVVSVLTVLHQDWLNGKEYKCKVSNKALPAPIEKTISKAKGQPREPQVYTLPPSRE |
| | | EMTKNQVSLTCLVKGFYPSDIAVEWESNGQPENNYDTTPPVLDSDGSFFLYSDLTVDKSR |
| | | WQQGNVFSCSVMHEALHNHYTQKSLSLSPGGDHCPLGPGRCCRLHTVRASLEDLGWADWV |
| | | LSPREVQVTMCIGACPSQFRAANMHAQIKTSLHRLKPDTVPAPCCVPASYNPMVLIQKTD |
| | | TGVSLQTYDDLLAKDCHCI |
| Dh3CpmFc(−)- | 284 | MDMRVPAQLLGLLLLWLRGARCGPSVFLFPPKPKDTLMISRTPEVTCVVVDVSHEDPEVK |
| GDF15(Ndel3) | | FNWYVDGVEVHNAKTKPREEQYNSTYRVVSVLTVLHQDWLNGKEYKCKVSNKALPAPIEK |
| with VH21 | | TISKAKGQPREPQVYTLPPSREEMTKNQVSLTCLVKGFYPSDIAVEWESNGQPENNYDTT |
| signal | | PPVLDSDGSFFLYSDLTVDKSRWQQGNVFSCSVMHEALHNHYTQKSLSLSPGGDHCPLGP |
| sequence | | GRCCRLHTVRASLEDLGWADWVLSPREVQVTMCIGACPSQFRAANMHAQIKTSLHRLKPD |
| | | TVPAPCCVPASYNPMVLIQKTDTGVSLQTYDDLLAKDCHCI |
| Dh3CpmFc(−) | 285 | GPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRE |
| | | EMTKNQVSLTCLVKGFYPSDIAVEWESNGQPENNYDTTPPVLDSDGSFFLYSDLTVDKSR |
| | | WQQGNVFSCSVMHEALHNHYTQKSLSLSPG |
| Dh3CpmFc(+) | 286 | GPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRK |
| | | EMTKNQVSLTCLVKGFYPSDIAVEWESNGQPENNYKTTPPVLKSDGSFFLYSKLTVDKSR |
| | | WQQGNVFSCSVMHEALHNHYTQKSLSLSPG |
| Dh3CpmFc(−)- | 287 | GPSVFLFPPKPKDTLMISRTPEVTCVVVDVSHEDPEVKFNWYVDGVEVHNAKTKPREEQY |
| GDF15(N3D) | | NSTYRVVSVLTVLHQDWLNGKEYKCKVSNKALPAPIEKTISKAKGQPREPQVYTLPPSRE |
| | | EMTKNQVSLTCLVKGFYPSDIAVEWESNGQPENNYDTTPPVLDSDGSFFLYSDLTVDKSR |
| | | WQQGNVFSCSVMHEALHNHYTQKSLSLSPGARDGDHCPLGPGRCCRLHTVRASLEDLGWA |
| | | DWVLSPREVQVTMCIGACPSQFRAANMHAQIKTSLHRLKPDTVPAPCCVPASYNPMVLIQ |
| | | KTDTGVSLQTYDDLLAKDCHCI |
| Dh3CpmFc(−)- | 288 | MDMRVPAQLLGLLLLWLRGARCGPSVFLFPPKPKDTLMISRTPEVTCVVVDVSHEDPEVK |
| GDF15(N3D) | | FNWYVDGVEVHNAKTKPREEQYNSTYRVVSVLTVLHQDWLNGKEYKCKVSNKALPAPIEK |
| with VH21 | | TISKAKGQPREPQVYTLPPSREEMTKNQVSLTCLVKGFYPSDIAVEWESNGQPENNYDTT |
| signal | | PPVLDSDGSFFLYSDLTVDKSRWQQGNVFSCSVMHEALHNHYTQKSLSLSPGARDGDHCP |
| sequence | | LGPGRCCRLHTVRASLEDLGWADWVLSPREVQVTMCIGACPSQFRAANMHAQIKTSLHRL |
| | | KPDTVPAPCCVPASYNPMVLIQKTDTGVSLQTYDDLLAKDCHCI |
| Dh3CpmFc(+) | 289 | GPSVFLFPPKPKDTLMISRTPEVTCVVVDVSHEDPEVKFNWYVDGVEVHNAKTKPREEQY |
| (S354C) | | NSTYRVVSVLTVLHQDWLNGKEYKCKVSNKALPAPIEKTISKAKGQPREPQVYTLPPCRK |
| | | EMTKNQVSLTCLVKGFYPSDIAVEWESNGQPENNYKTTPPVLKSDGSFFLYSKLTVDKSR |
| | | WQQGNVFSCSVMHEALHNHYTQKSLSLSPGK |
| Dh3CpmFc(+) | 290 | MDMRVPAQLLGLLLLWLRGARCGPSVFLFPPKPKDTLMISRTPEVTCVVVDVSHEDPEVK |
| (S354C) with | | FNWYVDGVEVHNAKTKPREEQYNSTYRVVSVLTVLHQDWLNGKEYKCKVSNKALPAPIEK |
| VH21 signal | | TISKAKGQPREPQVYTLPPCRKEMTKNQVSLTCLVKGFYPSDIAVEWESNGQPENNYKTT |
| sequence | | PPVLKSDGSFFLYSKLTVDKSRWQQGNVFSCSVMHEALHNHYTQKSLSLSPGK |
| Dh3CpmFc(−) | 291 | GPSVFLFPPKPKDTLMISRTPEVTCVVVDVSHEDPEVKFNWYVDGVEVHNAKTKPREEQY |
| (Y349C)- | | NSTYRVVSVLTVLHQDWLNGKEYKCKVSNKALPAPIEKTISKAKGQPREPQVCTLPPSRE |
| GDF15(Ndel3) | | EMTKNQVSLTCLVKGFYPSDIAVEWESNGQPENNYDTTPPVLDSDGSFFLYSDLTVDKSR |
| | | WQQGNVFSCSVMHEALHNHYTQKSLSLSPGGDHCPLGPGRCCRLHTVRASLEDLGWADWV |
| | | LSPREVQVTMCIGACPSQFRAANMHAQIKTSLHRLKPDTVPAPCCVPASYNPMVLIQKTD |
| | | TGVSLQTYDDLLAKDCHCI |
| Dh3CpmFc(−) | 292 | MDMRVPAQLLGLLLLWLRGARCGPSVFLFPPKPKDTLMISRTPEVTCVVVDVSHEDPEVK |
| (Y349C)- | | FNWYVDGVEVHNAKTKPREEQYNSTYRVVSVLTVLHQDWLNGKEYKCKVSNKALPAPIEK |
| GDF15(Ndel3) | | TISKAKGQPREPQVCTLPPSREEMTKNQVSLTCLVKGFYPSDIAVEWESNGQPENNYDTT |
| with VH21 | | PPVLDSDGSFFLYSDLTVDKSRWQQGNVFSCSVMHEALHNHYTQKSLSLSPGGDHCPLGP |
| signal | | GRCCRLHTVRASLEDLGWADWVLSPREVQVTMCIGACPSQFRAANMHAQIKTSLHRLKPD |
| sequence | | TVPAPCCVPASYNPMVLIQKTDTGVSLQTYDDLLAKDCHCI |
| Dh3CpmFc(−) | 293 | GPSVFLFPPKPKDTLMISRTPEVTCVVVDVSHEDPEVKFNWYVDGVEVHNAKTKPREEQY |
| (Y349C) | | NSTYRVVSVLTVLHQDWLNGKEYKCKVSNKALPAPIEKTISKAKGQPREPQVCTLPPSRE |
| | | EMTKNQVSLTCLVKGFYPSDIAVEWESNGQPENNYDTTPPVLDSDGSFFLYSDLTVDKSR |
| | | WQQGNVFSCSVMHEALHNHYTQKSLSLSPG |
| Dh3CpmFc(+) | 294 | GPSVFLFPPKPKDTLMISRTPEVTCVVVDVSHEDPEVKFNWYVDGVEVHNAKTKPREEQY |
| (S354C) | | NSTYRVVSVLTVLHQDWLNGKEYKCKVSNKALPAPIEKTISKAKGQPREPQVYTLPPCRK |
| | | EMTKNQVSLTCLVKGFYPSDIAVEWESNGQPENNYKTTPPVLKSDGSFFLYSKLTVDKSR |
| | | WQQGNVFSCSVMHEALHNHYTQKSLSLSPG |
| Dh3CpmFc(−) | 295 | GPSVFLFPPKPKDTLMISRTPEVTCVVVDVSHEDPEVKFNWYVDGVEVHNAKTKPREEQY |
| (Y349C)- | | NSTYRVVSVLTVLHQDWLNGKEYKCKVSNKALPAPIEKTISKAKGQPREPQVCTLPPSRE |
| GDF15(N3D) | | EMTKNQVSLTCLVKGFYPSDIAVEWESNGQPENNYDTTPPVLDSDGSFFLYSDLTVDKSR |
| | | WQQGNVFSCSVMHEALHNHYTQKSLSLSPGARDGDHCPLGPGRCCRLHTVRASLEDLGWA |
| | | DWVLSPREVQVTMCIGACPSQFRAANMHAQIKTSLHRLKPDTVPAPCCVPASYNPMVLIQ |
| | | KTDTGVSLQTYDDLLAKDCHCI |
| Dh3CpmFc(−) | 296 | MDMRVPAQLLGLLLLWLRGARCGPSVFLFPPKPKDTLMISRTPEVTCVVVDVSHEDPEVK |
| (Y349C)- | | FNWYVDGVEVHNAKTKPREEQYNSTYRVVSVLTVLHQDWLNGKEYKCKVSNKALPAPIEK |
| GDF15(N3D) | | TISKAKGQPREPQVCTLPPSREEMTKNQVSLTCLVKGFYPSDIAVEWESNGQPENNYDTT |
| with VH21 | | PPVLDSDGSFFLYSDLTVDKSRWQQGNVFSCSVMHEALHNHYTQKSLSLSPGARDGDHCP |
| signal | | LGPGRCCRLHTVRASLEDLGWADWVLSPREVQVTMCIGACPSQFRAANMHAQIKTSLHRL |
| sequence | | KPDTVPAPCCVPASYNPMVLIQKTDTGVSLQTYDDLLAKDCHCI |
| Sequence for | 297 | APELLGGPSVFLFPPKPKDTLMISRTPEVTCVVVDVSHEDPEVKFNWYVDGVEVHNAKTK |
| dimer of | | PREEQYGSTYRVVSVLTVLHQDWLNGKEYKCKVSNKALPAPIEKTISKAKGQPREPQVTT |
| DhMonoFc(N297G)- | | LPPSREEMTKNQVSLTCLVKGFYPSDIAVEWESNGQPENNYDTTPPVLDSDGSFFLYSDL |
| GDF15 | | TVDKSRWQQGNVFSCSVMHEALHNHYTQKSLSLSPGARNGDHCPLGPGRCCRLHTVRASL |
| | | EDLGWADWVLSPREVQVTMCIGACPSQFRAANMHAQIKTSLHRLKPDTVPAPCCVPASYN |
| | | PMVLIQKTDTGVSLQTYDDLLAKDCHCI |
| Sequence for | 298 | APELLGGPSVFLFPPKPKDTLMISRTPEVTCVVVDVSHEDPEVKFNWYVDGVEVHNAKTK |
| dimer of | | PREEQYGSTYRVVSVLTVLHQDWLNGKEYKCKVSNKALPAPIEKTISKAKGQPREPQVTT |
| DhMonoFc(N297G)- | | LPPSREEMTKNQVSLTCLVKGFYPSDIAVEWESNGQPENNYDTTPPVLDSDGSFFLYSDL |
| (G4S)4- | | TVDKSRWQQGNVFSCSVMHEALHNHYTQKSLSLSPGGGGGSGGGGSGGGGSGGGGSARNG |
| GDF15 | | DHCPLGPGRCCRLHTVRASLEDLGWADWVLSPREVQVTMCIGACPSQFRAANMHAQIKTS |
| | | LHRLKPDTVPAPCCVPASYNPMVLIQKTDTGVSLQTYDDLLAKDCHCI |
| Sequence for | 299 | APELLGGPSVFLFPPKPKDTLMISRTPEVTCVVVDVSHEDPEVKFNWYVDGVEVHNAKTK |
| dimer of | | PREEQYGSTYRVVSVLTVLHQDWLNGKEYKCKVSNKALPAPIEKTISKAKGQPREPQVTT |
| DhMonoFc(N297G)- | | LPPSREEMTKNQVSLTCLVKGFYPSDIAVEWESNGQPENNYDTTPPVLDSDGSFFLYSDL |
| G4-GDF15 | | TVDKSRWQQGNVFSCSVMHEALHNHYTQKSLSLSPGGGGGARNGDHCPLGPGRCCRLHTV |
| | | RASLEDLGWADWVLSPREVQVTMCIGACPSQFRAANMHAQIKTSLHRLKPDTVPAPCCVP |
| | | ASYNPMVLIQKTDTGVSLQTYDDLLAKDCHCI |
| hGDF15-AHA- | 320 | AHAGDHCPLGPGRCCRLHTVRASLEDLGWADWVLSPREVQVTMCIGACPSQFRAANMHAQI |
| [C(O)PEG2NH] | | KTSLHRLKPDTVPAPCCVPASYNPMVLIQKTDTGVSLQTYDDLLAKDCHCI |
| 2-FA | | |

In some embodiments, the methods of the invention comprise GDF15 fusion proteins, such as Fc fusions or albumin fusions. Said fusions can comprise wild type GDF15 or variants thereof. In some embodiments, the methods of the present invention comprise polypeptides which can be fused to a heterologous amino acid sequence, optionally via a linker, such as GS (SEQ ID NO: 313) or (GGGGS)n (SEQ ID NO:303), wherein n is one to about 20, and preferably 1, 2, 3 or 4.

The heterologous amino acid sequence can be an IgG constant domain or fragment thereof (e.g., the Fc region), Human Serum Albumin (HSA), or albumin-binding polypeptides. In some embodiments, the heterologous amino acid sequence is derived from the human IgG4 Fc region because of its reduced ability to bind Fcγ receptors and complement factors compared to other IgG sub-types. Such methods can comprise multimers of said fusion polypeptides. In some embodiments, the methods of the present invention comprise fusion proteins in which the heterologous amino acid sequence (e.g., HSA, Fc, etc.) is fused to the amino-terminal of the GDF15 protein or variants as described herein; in other embodiments, the fusion occurs at the carboxyl-terminal of the GDF15 protein or variants.

In some embodiments, the methods of the invention comprise GDF15 conjugates, such as GDF15 fatty acid (FA) conjugates, e.g., GDF15 wild type protein (full length, mature, or fragment or truncation thereof) or variant covalently attached to a fatty acid moiety via a linker. In specific embodiments, the methods provided herein comprises administering a GDF15 conjugate or a GDF15 variant conjugate which is not a fatty acid conjugate. In specific embodiments, the methods provided herein comprises administering a GDF15 fatty acid conjugate or a GDF15 variant fatty acid conjugate wherein the fatty acid moiety is not myristic acid and is not a fatty acid according to Formula A1, A2 and A3 as described herein.

In some embodiments, the methods of the invention comprise GDF15 fusion proteins or conjugates which are covalently linked to one or more polymers, such as polyethylene glycol (PEG) or polysialic acid. The PEG group is attached in such a way so as enhance, and/or not to interfere with, the biological function of the constituent portions of the fusion proteins or conjugates of the invention.

The invention also provides methods of treatment with a pharmaceutical composition comprising the GDF15 fusion proteins or GDF15 conjugates disclosed herein and a pharmaceutically acceptable formulation agent. Such pharmaceutical compositions can be used in a method for treating one or more of non-alcoholic fatty liver disease (NAFLD) and non-alcoholic steatohepatitis (NASH), as well as end-stage liver disease, hepatic steatosis (fatty liver), liver fibrosis, liver inflammation, liver cirrhosis, primary biliary cirrhosis (PBC), and hepatocellular carcinoma (HCC), and the methods comprise administering to a human patient in need thereof a pharmaceutical composition of the invention.

The invention also provides methods of treatment with a pharmaceutical composition comprising the GDF15 fusion proteins or GDF15 conjugates disclosed herein and a pharmaceutically acceptable formulation agent. Such pharmaceutical compositions can be used in a method for treating one or more of non-alcoholic fatty liver disease (NAFLD) and non-alcoholic steatohepatitis (NASH), as well as end-stage liver disease, hepatic steatosis (fatty liver), liver fibrosis, liver inflammation, liver cirrhosis, primary biliary cirrhosis (PBC), and hepatocellular carcinoma (HCC), and the methods comprise administering to a human patient in need thereof a pharmaceutical composition of the invention.

The invention also provides GDF15 fusion proteins or GDF15 conjugates disclosed herein for the treatment of one or more of non-alcoholic fatty liver disease (NAFLD) and non-alcoholic steatohepatitis (NASH), as well as end-stage liver disease, hepatic steatosis (fatty liver), liver fibrosis, liver inflammation, liver cirrhosis, primary biliary cirrhosis (PBC), and hepatocellular carcinoma (HCC). The invention also provides pharmaceutical compositions comprising GDF15 fusion proteins or GDF15 conjugates disclosed herein for the treatment of one or more of non-alcoholic fatty liver disease (NAFLD) and non-alcoholic steatohepatitis (NASH), as well as end-stage liver disease, hepatic steatosis (fatty liver), liver fibrosis, liver inflammation, liver cirrhosis, primary biliary cirrhosis (PBC), and hepatocellular carcinoma (HCC).

Non-limiting embodiments of the disclosure are described in the following aspects:

1. A method of treating non-alcoholic fatty liver disease (NAFLD), non-alcoholic steatohepatitis (NASH), end-stage liver disease, hepatic steatosis (fatty liver), liver fibrosis, liver inflammation, liver cirrhosis, primary biliary cirrhosis (PBC), or hepatocellular carcinoma (HCC) by administering a therapeutically effective amount of a GDF15 therapeutic agent comprising one or more of a GDF15 variant, GDF15 fusion protein, or GDF15 conjugate.

2. The method of aspect 1 wherein the GDF15 therapeutic agent is GDF15 conjugate.

3. The method of aspect 1 wherein the GDF15 therapeutic agent is an HSA-GDF15 fusion protein or an Fc-GDF15 fusion protein.

4. The method of aspect 1 wherein the GDF15 therapeutic agent is selected from Table 1.

5. A method of treating non-alcoholic fatty liver disease (NAFLD) or non-alcoholic steatohepatitis (NASH) by administering a therapeutically effective amount of a GDF15 therapeutic agent comprising one or more of a GDF15 protein, variant, mutant, fusion, or conjugate.

6. The method of aspect 5 wherein the GDF15 therapeutic agent is a fatty acid-GDF15 conjugate or a PEG-GDF15 conjugate.

7. The method of aspect 5 wherein the GDF15 therapeutic agent is an HSA-GDF15 fusion protein or an Fc-GDF15 fusion protein.

8. The method of aspect 5 wherein the GDF15 therapeutic agent is selected from Table 1.

9. A method of treating non-alcoholic fatty liver disease (NAFLD), non-alcoholic steatohepatitis (NASH), end-stage liver disease, hepatic steatosis (fatty liver), liver fibrosis, liver inflammation, liver cirrhosis, primary biliary cirrhosis (PBC), or hepatocellular carcinoma (HCC) by administering a therapeutically effective amount of a pharmaceutical composition comprising GDF15 therapeutic agent comprising one or more of a GDF15 protein, variant, mutant, fusion, or conjugate.

10. The method of aspect 9 wherein the GDF15 therapeutic agent is a fatty acid-GDF15 conjugate or a PEG-GDF15 conjugate.

11. The method of aspect 9 wherein the GDF15 therapeutic agent is an HSA-GDF15 fusion protein or an Fc-GDF15 fusion protein.

12. The method of aspect 9 wherein the GDF15 therapeutic agent is selected from Table 1.

13. A method of treating non-alcoholic fatty liver disease (NAFLD) or non-alcoholic steatohepatitis (NASH) by administering a therapeutically effective amount of a pharmaceutical composition comprising GDF15 therapeutic agent comprising one or more of a GDF15 protein, variant, mutant, fusion, or conjugate.

14. The method of aspect 13 wherein the GDF15 therapeutic agent is a fatty acid-GDF15 conjugate or a PEG-GDF15 conjugate.

15. The method of aspect 13 wherein the GDF15 therapeutic agent is an HSA-GDF15 fusion protein or an Fc-GDF15 fusion protein.

16. The method of aspect 13 wherein the GDF15 therapeutic agent is selected from Table 1.

17. The method of any one of aspect 1-16, wherein the GDF15 therapeutic agent does not comprise a GDF15 polypeptide comprising the amino acid sequence of SEQ ID NO: 41.

18. The method of any one of aspects 1-17, wherein the GDF15 therapeutic agent is not a fatty acid-GDF15 conjugate comprising the amino acid sequence of SEQ ID NO: 41.

19. The method of any one of aspects 1, 2, 4, 5, 6, 8, 9, 10, 12-14 and 16 wherein the GDF15 therapeutic is a fatty acid conjugate which does not comprise the amino sequence of:

(i)
SEQ ID NO: 41;
(ii)

(SEQ ID NO: 321)

MHHHH HHAR NGDHC PLGPG RCCRL HTVRA SLEDL GWADW
VLSPR EVQVT MCIGA CPSQF RAANM HAQIK TSLHR LKPDT
VPAPC CVPAS YNPMV LIQKT DTGVS LQTYD DLLAK DCHCI
(M-(his)₆-hGDF15 (197-308)),
(iii)

(SEQ ID NO: 322)

MHHHHHHMARNGDHCPLGPGRCCRLHTVRASLEDLGWADWVLSPREVQV
TMCIGACPSQFRAANMHAQIKTSLHRLKPDTVPAPCCVPASYNPMVLIQ
KTDTGVSLQTYDDLLAKDCHCI (M-(his)₆-M-hGDF15
(197-308)),
(iv)

(SEQ ID NO: 323)

MHHHHHHAHARDGCPLGEGRCCRLQSLRASLQDLGWANWVVAPRELDVR
MCVGACPSQFRSANTHAQMQARLHGLNPDAAPAPCCVPASYEPVVLMHQ
DSDGRVSLTPFDDLVAKDCHCV (M-(his)₆-dGDF15),
(v)

(SEQ ID NO: 324)

MHNGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (MH-hGDF15(199-308)),
(vi)

(SEQ ID NO: 325)

MHAGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (MHA-hGDF15(200-308)),
or
(vii)

(SEQ ID NO: 326)

AHNGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (AH-hGDF15(199-308)).

20. The method of any one of aspects 1-17, wherein the GDF15 therapeutic agent is not albumin-GDF15 fusion comprising the amino acid sequence of SEQ ID NO: 41, such as a human serum albumin-GDF15 fusion.

21. The method of any one of aspects 1-16, wherein the GDF15 therapeutic agent comprises the amino acid sequence of any one of the following: SEQ ID NO: 5, SEQ ID NO: 6, SEQ ID NO: 7, SEQ ID NO: 8, SEQ ID NOs: 42-63, SEQ ID NO: 69-107, SEQ ID NO: 148, SEQ ID NO: 149, and SEQ ID NO: 320; or any one of the following: SEQ ID NOs: 42-63, SEQ ID NO: 69-107, SEQ ID NO: 148, SEQ ID NO: 149, and SEQ ID NO: 320.

22. The method of any one of aspects 1-16, wherein the GDF15 therapeutic agent does not comprise one of the following amino acid sequences:

(i)

(SEQ ID NO: 321)

MHHHH HHAR NGDHC PLGPG RCCRL HTVRA SLEDL GWADW
VLSPR EVQVT MCIGA CPSQF RAANM HAQIK TSLHR LKPDT
VPAPC CVPAS YNPMV LIQKT DTGVS LQTYD DLLAK DCHCI
(M-(his)₆-hGDF15 (197-308)),
(ii)
SEQ ID NO: 6,
(iii)
SEQ ID NO: 7,
(iv)

(SEQ ID NO: 322)

MHHHHHHMARNGDHCPLGPGRCCRLHTVRASLEDLGWADWVLSPREVQV
TMCIGACPSQFRAANMHAQIKTSLHRLKPDTVPAPCCVPASYNPMVLIQ
KTDTGVSLQTYDDLLAKDCHCI (M-(his)₆-M-hGDF15
(197-308)),
(v)

(SEQ ID NO: 323)

MHHHHHHAHARDGCPLGEGRCCRLQSLRASLQDLGWANWVVAPRELDVR
MCVGACPSQFRSANTHAQMQARLHGLNPDAAPAPCCVPASYEPVVLMHQ
DSDGRVSLTPFDDLVAKDCHCV (M-(his)₆-dGDF15),
(vi)

(SEQ ID NO: 324)

MHNGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (MH-hGDF15(199-308)),
(vii)

(SEQ ID NO: 325)

MHAGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (MHA-hGDF15(200-308)),
(viii)
SEQ ID NO: 41,
and
(ix)

(SEQ ID NO: 326)

AHNGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (AH-hGDF15(199-308)).

23. The method of any one of aspects 1, 2, 4, 5, 6, 8, 9, 10, 12-14 and 16, wherein the GDF15 therapeutic agent is a fatty acid-GDF15 conjugate which does not comprise a fatty acid according to any one of Formula A1, A2, and A3:

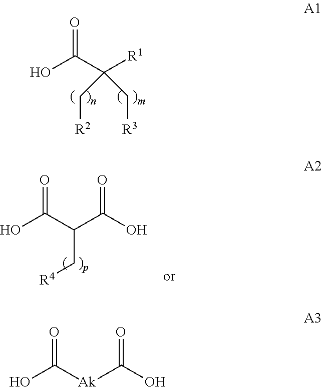

R¹ is CO₂H or H;
R², R³ and R⁴ are independently of each other H, OH, CO₂H, —CH═CH₂ or —C≡CH;
Ak is a branched C₆-C₃₀alkylene;
n, m and p are independently of each other an integer between 6 and 30; and which does not comprise tetradecanoic acid.

24. The method of any one of aspects 1, 2, 4, 5, 6, 8, 9, 10, 12-14, 16 and 22, wherein the GDF15 therapeutic agent is a fatty acid-GDF15 conjugate which does not comprise one or more of the following fatty acids:

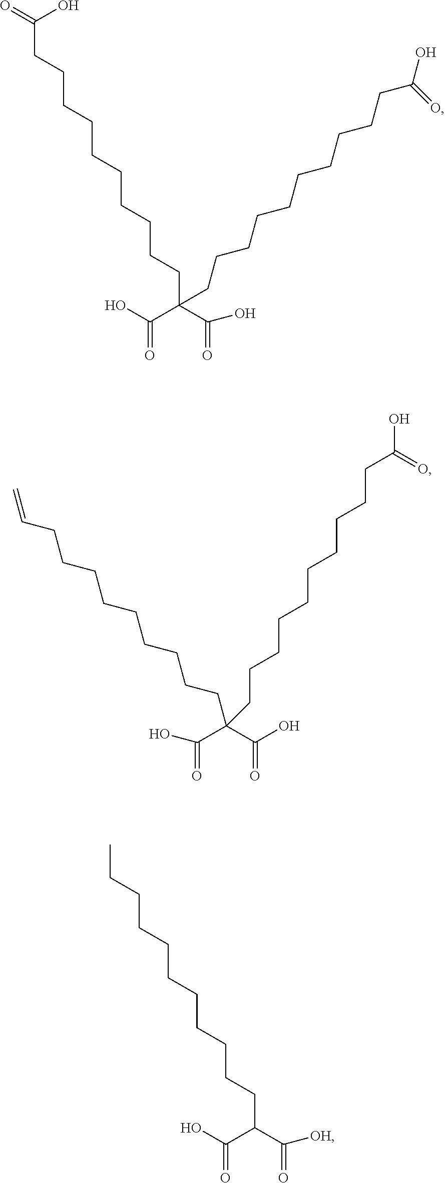 [SMILES: O=C(O)CCCCCCCCCCC(CCCCCCCCCCC(=O)O)(C(=O)O)C(=O)O ; C=CCCCCCCCCCC(CCCCCCCCCCC(=O)O)(C(=O)O)C(=O)O ; CCCCCCCCCCCC(C(=O)O)C(=O)O]

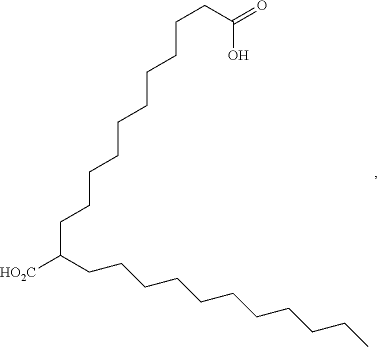 [SMILES: CCCCCCCCCCCC(CCCCCCCCCCC(=O)O)C(=O)O]

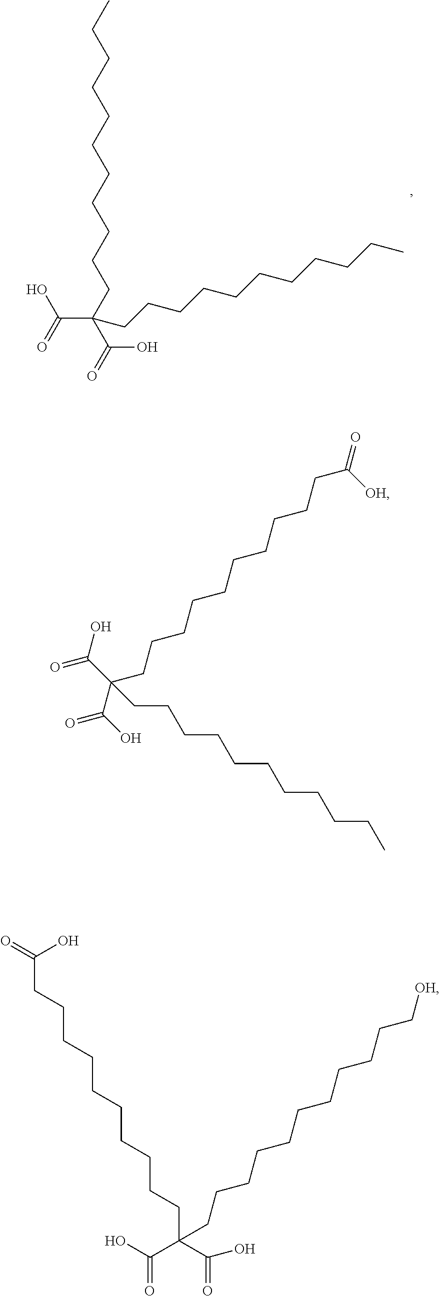 [SMILES: CCCCCCCCCCCC(CCCCCCCCCCC)(C(=O)O)C(=O)O ; CCCCCCCCCCCC(CCCCCCCCCCC(=O)O)(C(=O)O)C(=O)O ; O=C(O)CCCCCCCCCCC(CCCCCCCCCCCO)(C(=O)O)C(=O)O]

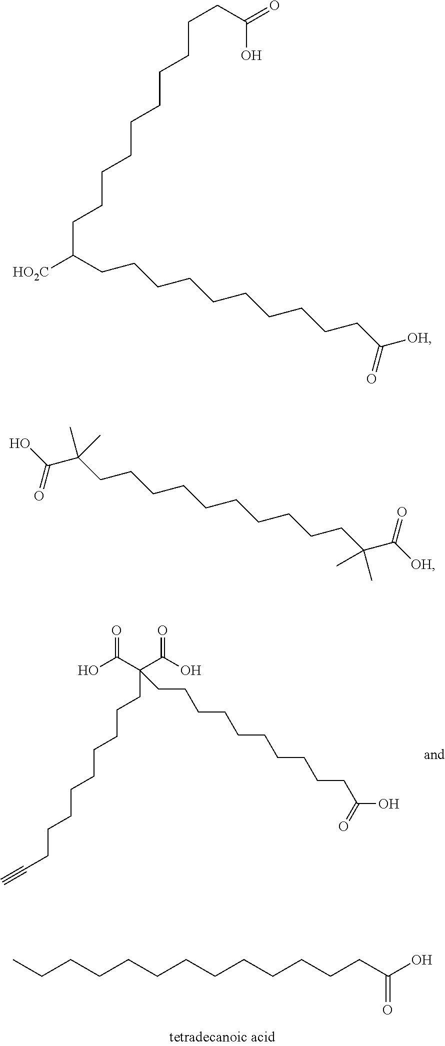 [SMILES: O=C(O)CCCCCCCCCCC(CCCCCCCCCCC(=O)O)C(=O)O ; CC(C)(CCCCCCCCCCC(C)(C)C(=O)O)C(=O)O ; C#CCCCCCCCCCC(CCCCCCCCCCC(=O)O)(C(=O)O)C(=O)O ; CCCCCCCCCCCCCC(=O)O]

These and other aspects of the invention will be elucidated in the following detailed description of the invention.

### BRIEF DESCRIPTION OF THE DRAWINGS

FIGS. 1A-B depict % change in body weight and cumulative food intake, respectively, following the administration of 0.0125 and 0.5 mg/kg of a fatty acid-GDF15 conjugate (6 week study).

FIGS. 2A-B depict changes in liver weight and hepatic steatosis following the administration of 0.0125 and 0.5 mg/kg of a fatty acid-GDF15 conjugate (6 week study).

FIG. 3 depicts % change in body weight following the administration of 0.0125 and 0.5 mg/kg of a fatty acid-GDF15 conjugate (16 week study).

FIGS. 4A-B depict changes in liver weight and heaptic steatosis following the administration of 0.0125 and 0.5 mg/kg of a fatty acid-GDF15 conjugate (16 week study).

### DETAILED DESCRIPTION

This invention relates to the treatment of non-alcoholic fatty liver disease (NAFLD) and non-alcoholic steatohepatitis (NASH), as well as related conditions that include but are not limited to alcoholic steatohepatitis (ASH), end-stage liver disease, hepatic steatosis (fatty liver), liver fibrosis, liver inflammation, liver cirrhosis, primary biliary cirrhosis (PBC), and hepatocellular carcinoma (HCC), by the administration of GDF15, e.g., variants, conjugates, or fusions of GDF15.

Growth differentiation factor 15 (GDF15) is a divergent member of the TGFβ superfamily. It is also called macrophage inhibitory cytokine 1 (MIC1) (Bootcov M R, 1997, Proc Natl Acad Sci 94: 11514-9), placental bone morphogenetic factor (PLAB) (Hromas R 1997, Biochim Biophys Acta. 1354:40-4), placental transforming growth factor beta (PTGFB) (Lawton L N 1997, Gene. 203: 17-26), prostate derived factor (PDF) (Paralkar V M 1998, J Biol Chem. 273: 13760-7), and nonsteroidal antiinflammatory drug-activated gene (NAG-1) (Baek S J 2001, J Biol Chem. 276: 33384-92).

Human GDF15 gene is located on chromosome 19p 13.2-13.1; rat GDF15 gene is located on chromosome 16; and mouse GDF15 gene is located on chromosome 8. The GDF15 open reading frames span two exons (Bottner M 1999, Gene. 237: 105-11 and NCBI). The mature GDF15 peptide shares low homology with other family members (Katoh M 2006, IntJMol Med. 17:951-5.).

GDF15 is synthesized as a large precursor protein that is cleaved at the dibasic cleavage site to release the carboxyterminal mature peptide. The mouse and rat GDF15 prepro-peptides both contain 303 amino acids. Human full-length precursor contains 308 amino acids. The rodent mature peptides contain 115 amino acids after processing at the RGRR (SEQ ID NO: 1) cleavage site. The human mature peptide contains 112 amino acids after processing at the RGRRRAR (SEQ ID NO:302) cleavage site. Human mature GDF15 peptide shares 66.1 percent and 68.1 percent sequence similarity with rat and mouse mature GDF15 peptides (Bottner M 1999, Gene. 237: 105-11; Bauskin A R 2000, EMBO J. 19:2212-20; NCBI). There is no glycosylation site in the mature GDF15 peptide.

The mature GDF15 peptide contains the seven conserved cysteine residues required for the formation of the cysteine knot motif (having three intrachain disulfide bonds) and the single interchain disulfide bond that are typical for TGF superfamily members. The mature GDF15 peptide further contains two additional cysteine residues that form a fourth intrachain disulfide bond. Biologically active GDF15 is a 25 KD homodimer of the mature peptide covalently linked by one interchain disulfide bond.

GDF15 circulating levels have been reported to be elevated in multiple pathological and physiological conditions, most notably pregnancy (Moore A G 2000. J Clin Endocrinol Metab 85: 4781-4788), beta-thalassemia (Tanno T 2007, Nat Med 13: 1096-101) (Zimmermann M B, 2008 Am J Clin Nutr 88: 1026-31), and congenital dyserythropoietic anemia (Tamary H 2008, Blood. 112:5241-4). GDF15 has also been linked to multiple biological activities in literature reports. Studies of GDF15 knockout and transgenic mice suggested that GDF15 may be protective against ischemic/reperfusion- or overload-induced heart injury (Kempf T, 2006, Circ Res.98:351-60) (Xu J, 2006, Circ Res. 98:342-50), protective against aging-associated motor neuron and sensory neuron loss (Strelau J, 2009, J Neurosci. 29: 13640-8), mildly protective against metabolic acidosis in kidney, and may cause cachexia in cancer patients (Johnen H 2007 Nat Med. 11: 1333-40). Many groups also studied the role of GDF15 in cell apoptosis and proliferation and reported controversial results using different cell culture and xenograft models. Studies on transgenic mice showed that GDF15 is protective against carcinogen or Apc mutation induced neoplasia in intestine and lung (Baek S J 2006, Gastroenterology. 131: 1553-60; Cekanova M 2009, Cancer Prev Res 2:450-8).

The X-ray crystal structure of the human mature GDF15 protein reveals a disulfide-linked dimeric structure. Each GDF15 monomer adopts a fold similar to other TGFbeta superfamily cysteine knot proteins with a significant difference seen at the N-terminal. The mature GDF15 protein contains a total of nine cysteines all of which are disulfide bonded with Cys273, forming the inter-chain disulfide across the dimer interface. The disulfide bonding pattern of the first four Cysteines is unique to GDF15 when compared with TGFbeta and BMP family members. Cys203 and Cys210 (the first two cysteines in the mature protein) form a disulfide with each other to make a small loop structure protruding from the protein.

The remaining disulfides are structurally similar to the TGFbeta family but are formed by Cys211-Cys274 (third and seventh cysteines), Cys240-Cys305 (fourth and eighth cysteines) and Cys244-Cys307 (fifth and ninth cysteines). The crystal structure further revealed that there is an extensive peptide-peptide interface in the human GDF-15 homodimer, with ˜1300 square Angstroms of buried surface area and involvement of 37 amino acids.

The crystal structure shows that the following amino acids are involved in the peptide-peptide interface: Val216, Asp222, Leu223, Trp225, Val237, Met239, Ile241, Asn252, Met253, His254, Ile257, Lys258, Ser260, Leu261, Leu264, Lys265, Thr268, Val269, Pro270, Cys273, Val275, Pro276, Tyr279, Tyr297, Asp299, Leu300 and Ile308. The last amino-acid of the mature peptide, Ile308, is positioned fewer than 10 angstroms away from its dimer partner. Unusually for the superfamily, the electron density is consistent with the side-chain pointing toward the interior of the protein structure to form a hydrophobic pocket with Val275 and Pro276. Other family members have the carboxylic acid pointing toward the inside of the structure and the sidechain solvent exposed (ref TGFb3 (2PJY), BMP6(2R52), BMP7(1LX5), GDFS(3EVS), GDF2(4FAO)). This suggests that GDF15 might be unique in its ability to accommodate longer peptide sequences at the COOH-termini without perturbation of its protein fold.

In one embodiment, the methods of the invention comprise GDF15 fusion proteins as described herein, e.g., the serum albumin fusions. In some embodiments, said fusions can contain any suitable SA moiety, any suitable GDF15 moiety, and if desired, any suitable linker. Generally, the SA moiety, GDF15 moiety and, if present, linker, are selected to provide a fusion polypeptide that would be predicted to have therapeutic efficacy in NASH, NAFLD, or the other disorders described herein, and to be immunologically compatible with the species to which it is intended to be administered. For example, when the fusion polypeptide is intended to be administered to humans the SA moiety can be HSA or a functional variant thereof, and the GDF15 moiety can be human GDF15 or a functional variant thereof. Similarly, SA and functional variants thereof and GDF15 and functional variants thereof that are derived from other species (e.g., pet or livestock animals) can be used when the fusion protein is intended for use in such species.

In a particular embodiment, GDF15 fusions for use in the methods of the present invention do not comprise GDF15 fusions (e.g., SA-GDF15 fusions or HSA-GDF15 fusions) described in PCT Publication No. WO2015/198199, which is incorporated by reference herein in its entirety.

In a particular embodiment, GDF15 conjugates for use in the methods of the present invention do not comprise GDF15 conjugates (e.g., fatty acid-GDF15 conjugates) described in PCT Publication No. WO2015/200078, which is incorporated by reference herein in its entirety.

### GDF15 Moiety

The GDF15 moiety used in the present methods of the invention, e.g., in any GDF15 fusion protein or conjugate, such as fatty acid conjugate, can be any suitable GDF15 polypeptide or functional variant thereof, for example a GDF15 variant described in Table 1. Preferably, the GDF15 moiety is human GDF15 or a functional variant thereof. Human GDF15 is synthesized as a 308 amino acid preproprotein (SEQ ID NO:1) that includes a signal peptide (amino acids 1-29), a propeptide (amino acids 30-196), and the 112 amino acid mature GDF15 peptide (amino acids 197-308 (SEQ ID NO:5)). The propeptide and mature peptide have been reported as amino acids 30-194 and 195-308 of SEQ ID NO:2, respectively. (See, Uniprot sequence Q99988.) Sequence variations have been reported. For example, amino acids 202, 269 and 288 (in SEQ ID NO:2) have been reported to be Asp, Glu and Ala, respectively. (Hromas R, et al., Biochem. Biophys. Acta 1354:40-44 (1997), Lawton L. N. et al, Gene 203:17-26 (1997).)

Fusion proteins used in the present methods of the invention that contain a human GDF15 moiety generally contain the 112 amino acid mature GDF15 peptide (e.g., amino acids 197-308 of SEQ ID NO:1, SEQ ID NO:5) or a functional variant thereof. The functional variant can include one or more amino acid deletions, additions or replacements in any desired combination, for example, a GDF15 variant in Table 1. The amount of amino acid sequence variation (e.g., through amino acid deletions, additions or replacements) is limited to preserve weight loss activity of the mature GDF15 peptide. In some embodiments, the functional variant of a mature GDF15 peptide has from 1 to about 20, 1 to about 18, 1 to about 17, 1 to about 16, 1 to about 15, 1 to about 14, 1 to about 13, 1 to about 12, 1 to about 11, 1 to about 10, 1 to about 9, 1 to about 8, 1 to about 7, 1 to about 6, or 1 to about 5 amino acid deletions, additions or replacements, in any desired combination, relative to SEQ ID NO:5. Alternatively or in addition, the functional variant can have an amino acid sequence that has at least about 80%, at least about 85%, at least about 90%, or at least about 95%, 96%, 97%, 98%, or 99% amino acid sequence identity with SEQ ID NO:5, preferably when measured over the full length of SEQ ID NO:5. In a specific embodiment, a GDF15 functional variant can have an amino acid sequence that has at least 90%, at least 95%, or at least 98% amino acid sequence identity with SEQ ID NO:5, preferably when measured over the full length of SEQ ID NO:5.

Without wishing to be bound by any particular theory, it may be that GDF15's therapeutic efficacy in NASH, NAFLD, and related conditions is mediated either through cellular signaling initiated by the binding of GDF15 (and the fusion proteins and variants described herein) to one or more receptors and/or soluble co-factors, or by regulation of signaling pathways utilized by other factors via direct competition or allosteric modulation. Amino acid substitutions, deletions, or additions are preferably at positions that are not involved with receptor or co-factor binding, nor involved in maintaining overall protein conformation via intr-peptide interactions.

For example, the amino acids at positions 216, 222, 223, 225, 237, 239, 241, 252, 253, 254, 257, 258, 260, 261, 264, 265, 268, 269, 270, 273, 275, 276, 279, 297, 299, 300 and 308 are involved in the peptide-peptide interface. In specific embodiments, any amino acid replacements at these positions are generally disfavored, and any replacements should be conservative replacements. Amino acids that are surface exposed but are not conserved among species can generally be replaced with other amino acids without disrupting the folding of the peptide or its weight loss activity. The inventors have determined the crystal structure of the human mature GDF15 peptide and identified the amino acids at positions 217, 219, 226, 234, 243, 246, 247, 263, 265, 268, 277, 280, 287, 290, 303 and 304 as surface exposed residues that are not conserved in other species.

The inventors have determined the crystal structure of the human mature GDF15 peptide and identified the amino acids at positions 217, 219, 226, 234, 243, 246, 247, 263, 265, 268, 277, 280, 287, 290, 303 and 304 as surface exposed residues that are not conserved in other species. In addition, the amino terminal of mature human GDF15 (amino acids 197-210 of SEQ ID NO:1) and Cys203, Cys 210 and Cys273, which are not essential for weight loss activity, can generally be replaced with another amino acid and/or omitted. In a specific embodiment, the first 1-8 or the first 1-6 N-terminal amino acids of mature human GDF15 can be removed or substituted. In a specific embodiment, the first 1-5 or the first 1-4 N-terminal amino acids of mature human GDF15 can be removed or substituted. In a specific embodiment, the first 2 or the first 3 N-terminal amino acids of mature human GDF15 can be removed or substituted. In a specific embodiment, the first 3 or the first 6 N-terminal amino acids of mature human GDF15 can be removed or substituted.

Exemplary variants of human mature GDF15 peptide that are suitable for use in the fusion polypeptides include SEQ ID NO:5 in which one or more of the residues from position 1 to about 25 are replaced or deleted. For example, the variant can have the sequence of SEQ ID NO:44 in which the first 25, the first 15, the first 14, the first 13, the first 12, the first 11, the first 10, the first 9, the first 8, the first 7, the first 6, the first 5, the first 4, the first 3, the first 2, or the first 1 amino acid is deleted.

Additional exemplary variants of human mature GDF15 peptide that are suitable for use in the fusion polypeptides of the present invention include amino acids 197-308 of SEQ ID NO:1 (SEQ ID NO:5) in which the Arg at position 198, Asn at position 199, or Arg at position 198 and Asn at position 199 are replaced with one or more other amino acids. When amino acids are replaced, conservative amino acid replacements are preferred. In particular embodiments, Arg at position 198 is replaced with His or Asn at position 199 is replaced with Ala or Glu. In more particular embodiments Arg at position 198 is replaced with His and Asn at position 199 is replaced with Ala.

In a particular embodiment, exemplary variants of human mature GDF15 peptide that are suitable for use in the conjugates and fusion polypeptides of the present invention include amino acids 197-308 of SEQ ID NO:1 (SEQ ID NO:5) in which the Arg at position 198 is not replaced with His and Asn at position 199 is not replaced with Ala. In a specific embodiment, exemplary variants of human mature GDF15 peptide that are suitable for use in the conjugates and fusion polypeptides of the present invention do not comprise the GDF15 variant of SEQ ID NO: 41, or SEQ ID NO: 320, or

(SEQ ID NO: 321)

MHHHH HHAR NGDHC PLGPG RCCRL HTVRA SLEDL GWADW
VLSPR EVQVT MCIGA CPSQF RAANM HAQIK TSLHR LKPDT
VPAPC CVPAS YNPMV LIQKT DTGVS LQTYD DLLAK DCHCI
(M-(his)6-hGDF15),
or

(SEQ ID NO: 322)

MHHHHHHMARNGDHCPLGPGRCCRLHTVRASLEDLGWADWVLSPREVQV
TMCIGACPSQFRAANMHAQIKTSLHRLKPDTVPAPCCVPASYNPMVLIQ
KTDTGVSLQTYDDLLAKDCHCI (M-(his)6-M-hGDF15).

In a specific embodiment, exemplary variants of human mature GDF15 peptide that are suitable for use in fatty acid-GDF15 conjugates of the present invention do not comprise the GDF15 variant of SEQ ID NO: 41, or SEQ ID NO: 320, or

(SEQ ID NO: 321)

MHHHH HHAR NGDHC PLGPG RCCRL HTVRA SLEDL GWADW
VLSPR EVQVT MCIGA CPSQF RAANM HAQIK TSLHR LKPDT
VPAPC CVPAS YNPMV LIQKT DTGVS LQTYD DLLAK DCHCI
(M-(his)6-hGDF15),
or

(SEQ ID NO: 322)

MHHHHHHMARNGDHCPLGPGRCCRLHTVRASLEDLGWADWVLSPREVQV
TMCIGACPSQFRAANMHAQIKTSLHRLKPDTVPAPCCVPASYNPMVLIQ
KTDTGVSLQTYDDLLAKDCHCI (M-(his)6-M-hGDF15).

In a specific embodiment, exemplary variants of human mature GDF15 peptide that are suitable for use in fatty acid-GDF15 conjugates of the present invention do not comprise the GDF15 variant of SEQ ID NO: 41, or SEQ ID NO: 320, or

(SEQ ID NO: 321)

MHHHH HHAR NGDHC PLGPG RCCRL HTVRA SLEDL GWADW
VLSPR EVQVT MCIGA CPSQF RAANM HAQIK TSLHR LKPDT
VPAPC CVPAS YNPMV LIQKT DTGVS LQTYD DLLAK DCHCI
(M-(his)6-hGDF15),
or

(SEQ ID NO: 322)

MHHHHHHMARNGDHCPLGPGRCCRLHTVRASLEDLGWADWVLSPREVQV
TMCIGACPSQFRAANMHAQIKTSLHRLKPDTVPAPCCVPASYNPMVLIQ
KTDTGVSLQTYDDLLAKDCHCI (M-(his)6-M-hGDF15).

In a specific embodiment, exemplary variants of human mature GDF15 peptide that are suitable for use in a GDF15 fusion polypeptide, such as an SA-GDF15 fusion, of the present invention do not comprise the GDF15 variant of SEQ ID NO: 41, or SEQ ID NO: 320, or

(SEQ ID NO: 321)

MHHHH HHAR NGDHC PLGPG RCCRL HTVRA SLEDL GWADW
VLSPR EVQVT MCIGA CPSQF RAANM HAQIK TSLHR LKPDT
VPAPC CVPAS YNPMV LIQKT DTGVS LQTYD DLLAK DCHCI
(M-(his)6-hGDF15),
or

(SEQ ID NO: 322)

MHHHHHHMARNGDHCPLGPGRCCRLHTVRASLEDLGWADWVLSPREVQV
TMCIGACPSQFRAANMHAQIKTSLHRLKPDTVPAPCCVPASYNPMVLIQ
KTDTGVSLQTYDDLLAKDCHCI (M-(his)6-M-hGDF15).

In a particular embodiment, variants of human mature GDF15 peptide that are suitable for use in the conjugates and fusion polypeptides of the present invention include an amino acid sequence which is at 95% identical to SEQ ID NO:5, wherein the Arg at position 198 is not replaced with His and Asn at position 199 is not replaced with Ala, or wherein GDF15 variant is not SEQ ID NO: 41 or SEQ ID NO: 320. In a certain embodiment, variants of human mature GDF15 peptide that are suitable for use in the conjugates and fusion polypeptides of the present invention include those described in Table 1, wherein the Arg at position 198 is not replaced with His and Asn at position 199 is not replaced with Ala, or wherein GDF15 variant is not SEQ ID NO: 41 or SEQ ID NO: 320. In a certain embodiment, variants of human mature GDF15 peptide that are suitable for use in the conjugates (e.g., fatty acid-GDF15 conjugate) and fusion polypeptides (e.g., SA-GDF15 fusion polypeptide) of the present invention include those described in Table 1, wherein the variants of human mature GDF15 peptide do not comprise the GDF15 variant of SEQ ID NO: 41 or SEQ ID NO: 320, or

(SEQ ID NO: 321)

MHHHH HHAR NGDHC PLGPG RCCRL HTVRA SLEDL GWADW
VLSPR EVQVT MCIGA CPSQF RAANM HAQIK TSLHR LKPDT
VPAPC CVPAS YNPMV LIQKT DTGVS LQTYD DLLAK DCHCI
(M-(his)6-hGDF15),
or

(SEQ ID NO: 322)

MHHHHHHMARNGDHCPLGPGRCCRLHTVRASLEDLGWADWVLSPREVQV
TMCIGACPSQFRAANMHAQIKTSLHRLKPDTVPAPCCVPASYNPMVLIQ
KTDTGVSLQTYDDLLAKDCHCI (M-(his)6-M-hGDF15).

In a particular embodiment, variants of human mature GDF15 peptide that are suitable for use in the conjugates and fusion polypeptides of the present invention do not comprise GDF15 variants described in PCT Publication No. WO2015/198199, which is incorporated by reference herein in its entirety, for example SEQ ID NOs: SEQ ID NOS: 20, 26, 28, 30, 32, 38, 40 and 42 provided therein. In a particular embodiment, variants of human mature GDF15 peptide that are suitable for use in the conjugates and fusion polypeptides of the present invention do not comprise GDF15 variants comprising an amino acid replacement or deletion of one or more surface exposed residues (e.g., Arg217, Ser219, Ala226, Glu234, Ala243, Ser246, Gln247, Arg263, Lys265, Thr268, A3a277, Asn280, Lys287, Thr290, Lys303 and Asp3G4), one or more N-terminal amino acids (ammo acids 197-210), Cys 203, Cys 210 and/or Cys273. In a particular embodiment, variants of human mature GDF15 peptide that are suitable for use in the fusion polypeptides, such as albumin-GDF15 fusions (e.g., HSA-GDF15 fusions), of the present invention do not comprise GDF15 variants comprising an amino acid replacement or deletion of one or more surface exposed residues (e.g., Arg217, Ser219, Ala226, Glu234, Ala243, Ser246, Gln247, Arg263, Lys265, Thr268, A3a277, Asn280, Lys287, Thr290, Lys303 and Asp3G4), one or more N-terminal amino acids (ammo acids 197-210), Cys 203, Cys 210 and/or Cys273.

In a particular embodiment, variants of human mature GDF15 peptide that are suitable for use in the conjugates (e.g., fatty acid-GDF15 conjugates) and fusion polypeptides (e.g., SA-GDF15 fusion polypeptides such as HSA-GDF15 fusion polypeptides) of the present invention do not comprise the following GDF15 variants:

(i)
His6-hGDF15(197-308):

(SEQ ID NO: 6)

HHHHHHARNG DHCPLGPGRC CRLHTVRASL EDLGWADWVL
SPREVQVTMC IGACPSQFRA ANMHAQIKTS LHRLKPDTVP
APCCVPASYN PMVLIQKTDT GVSLQTYDDL LAKDCHCI;
(ii)
His8-TEV-hGDF15(197-308):

(SEQ ID NO: 7)

HHHHHHHHGG SENLYFQGAR NGDHCPLGPG RCCRLHTVRA
SLEDLGWADW VLSPREVQVT MCIGACPSQF RAANMHAQIK
TSLHRLKPDT VPAPCCVPAS YNPMVLIQKT DTGVSLQTYD
DLLAKDCHCI;
and
(iii)
hGDF15(197-308):

(SEQ ID NO: 5)

ARNGDHCPLG PGRCCRLHTV RASLEDLGWA DWVLSPREVQ
VTMCIGACPS QFRAANMHAQ IKTSLHRLKP DTVPAPCCVP
ASYNPMVLIQ KTDTGVSLQT YDDLLAKDCH CI.

Mature human GDF15 includes 9 cysteine residues, eight of which form intra-chain disulfide bonds in a pattern that is unique among TGFbeta superfamily members. In one embodiment, Cys203, 210 and 273 can be replaced with other amino acids or omitted if desired.

### Serum Albumin (SA) Moiety

The SA moiety is any suitable serum albumin (e.g., human serum albumin (HSA), or serum albumin from another species) or a functional variant thereof. Preferably, the SA moiety is an HSA or a functional variant thereof. The SA moiety prolongs the serum half-life of the fusion polypeptides to which it is added, in comparison to wild type GDF15. Methods for pharmacokinetic analysis and determination of serum half-life will be familiar to those skilled in the art. Details may be found in Kenneth, A et al: Chemical Stability of Pharmaceuticals: A Handbook for Pharmacists and in Peters et al, Pharmacokinetc analysis: A Practical Approach (1996). Reference is also made to “Pharmacokinetics,” M Gibaldi & D Perron, published by Marcel Dekker, 2^{nd} Rev. ex edition (1982), which describes pharmacokinetic parameters such as t alpha and t beta half-lives and area under the curve (AUC).

Human Serum Albumin (HSA) is a plasma protein of about 66,500 KDa and is comprised of 585 amino acids, including at least 17 disulfide bridges. (Peters, T., Jr. (1996), All about Albumin: Biochemistry, Genetics and Medical, Applications, pp 10, Academic Press, Inc., Orlando (ISBN 0-12-552110-3). HSA has a long half-life and is cleared very slowly by the liver. The plasma half-life of HSA is reported to be approximately 19 days (Peters, T., Jr. (1985) Adv. Protein Chem. 37, 161-245; Peters, T., Jr. (1996) All about Albumin, Academic Press, Inc., San Diego, Calif. (page 245-246)); Benotti P, Blackburn G L: Crit Care Med (1979) 7:520-525).

HSA has been used to produce fusion proteins that have improved shelf and half-lifes. For example, PCT Publications WO01/79271 A and WO03/59934 A disclose (i) albumin fusion proteins comprising a variety of therapeutic protein (e.g., growth factors, scFvs); and (ii) HSAs that are reported to have longer shelf and half-lives than their therapeutic proteins alone.

HSA may comprise the full length sequence of 585 amino acids of mature naturally occurring HSA (following processing and removal of the signal and propeptides (SEQ ID NO:4)) or naturally occurring variants thereof, including allelic variants. Naturally occurring HSA and variants thereof are well-known in the art. (See, e.g., Meloun, et al., FEBS Letters 58:136 (1975); Behrens, et al., Fed. Proc. 34:591 (1975); Lawn, et al., Nucleic Acids Research 9:6102-6114 (1981); Minghetti, et al., J. Biol. Chem. 261:6747 (1986)); and Weitkamp, et al., Ann. Hum. Genet. 37:219 (1973).)

Fusion proteins that contain a human serum albumin moiety generally contain the 585 amino acid HSA (amino acids 25-609 of SEQ ID NO:3, SEQ ID NO:4) or a functional variant thereof. The functional variant can include one or more amino acid deletions, additions or replacement in any desired combination, and includes functional fragments of HSA. The amount of amino acid sequence variation (e.g., through amino acid deletions, additions or replacements) is limited to preserve the serum half-life extending properties of HSA.

In some embodiments, the functional variant of HSA for use in the fusion proteins disclosed herein can have an amino acid sequence that has at least about 80%, at least about 85%, at least about 90%, or at least about 95% amino acid sequence identity with SEQ ID NO:4, preferably when measured over the full length sequence of SEQ ID NO:4. Alternatively or in addition, the functional variant of HSA can have from 1 to about 20, 1 to about 18, 1 to about 17, 1 to about 16, 1 to about 15, 1 to about 14, 1 to about 13, 1 to about 12, 1 to about 11, 1 to about 10, 1 to about 9, 1 to about 8, 1 to about 7, 1 to about 6, or 1 to about 5 amino acid deletions, additions or replacement, in any desired combination. In a specific embodiment, a functional variant of HSA for use in the fusion proteins disclosed herein comprises a C34A mutation.

Some functional variants of HSA for use in the fusion proteins disclosed herein may be at least 100 amino acids long, or at least 150 amino acids long, and may contain or consist of all or part of a domain of HSA, for example domain I (amino acids 1-194 of SEQ ID NO:4), II (amino acids 195-387 of SEQ ID NO:4), or III (amino acids 388-585 of SEQ ID NO:4). If desired, a functional variant of HSA may consist of or alternatively comprise any desired HSA domain combination, such as, domains I+II (amino acids 1-387 of SEQ ID NO:4), domains II+III (amino acids 195-585 of SEQ ID NO:4) or domains I+III (amino acids 1-194 of SEQ ID NO:4+amino acids 388-585 of SEQ ID NO:4). As is well-known in the art, each domain of HSA is made up of two homologous subdomains, namely amino acids 1-105 and 120-194, 195-291 and 316-387, and 388-491 and 512-585 of domains I, II, and III respectively, with flexible inter-subdomain linker regions comprising residues Lys106 to Glu119, Glu292 to Val315 and Glu492 to Ala511. In certain embodiments, the SA moiety of the fusions proteins of the present invention contains at least one subdomain or domain of HSA.

Functional fragments of HSA suitable for use in the fusion proteins disclosed herein will contain at least about 5 or more contiguous amino acids of HSA, preferably at least about 10, at least about 15, at least about 20, at least about 25, at least about 30, at least about 50, or more contiguous amino acids of HSA sequence or may include part or all of specific domains of HSA.

In some embodiments, the functional variant (e.g., fragment) of HSA for use in the fusion proteins disclosed herein includes an N-terminal deletion, a C-terminal deletions or a combination of N-terminal and C-terminal deletions. Such variants are conveniently referred to using the amino acid number of the first and last amino acid in the sequence of the functional variant. For example, a functional variant with a C-terminal truncation can be amino acids 1-387 of HSA (SEQ ID NO:4).

Examples of HSA and HSA variants (including fragments) that are suitable for use in the GDF15 fusion polypeptides described herein are known in the art. Suitable HSA and HSA variants include, for example full length mature HSA (SEQ ID NO:4) and fragments, such as amino acids 1-387, amino acids 54 to 61, amino acids 76 to 89, amino acids 92 to 100, amino acids 170 to 176, amino acids 247 to 252, amino acids 266 to 277, amino acids 280 to 288, amino acids 362 to 368, amino acids 439 to 447, amino acids 462 to 475, amino acids 478 to 486, and amino acids 560 to 566 of mature HSA. Such HSA polypeptides and functional variants are disclosed in PCT Publication WO 2005/077042A2, which is incorporated herein by reference in its entirety. Further variants of HSA, such as amino acids 1-373, 1-388, 1-389, 1-369, 1-419 and fragments that contain amino acid 1 through amino acid 369 to 419 of HSA are disclosed in European Published Application EP322094A1, and fragments that contain 1-177, 1-200 and amino acid 1 through amino acid 178 to 199 are disclosed in European Published Application EP399666A1.

In a particular embodiment, HSA-GDF15 fusion polypeptides that are suitable for use of the present invention do not comprise the following fusions:

(i)
HSA(25-609),C34S,N503Q-hGDF15(211-308):

(SEQ ID NO: 327)

DAHKSEVAHR FKDLGEENFK ALVLIAFAQY LQQSPFEDHV
KLVNEVTEFA KTCVADESAE NCDKSLHTLF GDKLCTVATL
RETYGEMADC CAKQEPERNE CFLQHKDDNP NLPRLVRPEV
DVMCTAFHDN EETFLKKYLY EIARRHPYFY APELLFFAKR
YKAAFTECCQ AADKAACLLP KLDELRDEGK ASSAKQRLKC
ASLQKFGERA FKAWAVARLS QRFPKAEFAE VSKLVTDLTK
VHTECCHGDL LECADDRADL AKYICENQDS ISSKLKECCE
KPLLEKSHCI AEVENDEMPA DLPSLAADFV ESKDVCKNYA
EAKDVFLGMF LYEYARRHPD YSVVLLLRLA KTYETTLEKC
CAAADPHECY AKVFDEFKPL VEEPQNLIKQ NCELFEQLGE
YKFQNALLVR YTKKVPQVST PTLVEVSRNL GKVGSKCCKH
PEAKRMPCAE DYLSVVLNQL CVLHEKTPVS DRVTKCCTES
LVNRRPCFSA LEVDETYVPK EFQAETFTFH ADICTLSEKE
RQIKKQTALV ELVKHKPKAT KEQLKAVMDD FAAFVEKCCK
ADDKETCFAE EGKKLVAASQ AALGLGGGGS GGGGSGGGGS
CRLHTVRASL EDLGWADWVL SPREVQVTMC IGACPSQFRA
ANMHAQIKTS LHRLKPDTVP APCCVPASYN PMVLIQKTDT
GVSLQTYDDL LAKDCHCI;
(ii)
HSA-3x4GS-hGDF15(197-308):

(SEQ ID NO: 328)

DAHKSEVAHR FKDLGEENFK ALVLIAFAQY LQQCPFEDHV
KLVNEVTEFA KTCVADESAE NCDKSLHTLF GDKLCTVATL
RETYGEMADC CAKQEPERNE CFLQHKDDNP NLPRLVRPEV
DVMCTAFHDN EETFLKKYLY EIARRHPYFY APELLFFAKR
YKAAFTECCQ AADKAACLLP KLDELRDEGK ASSAKQRLKC
ASLQKFGERA FKAWAVARLS QRFPKAEFAE VSKLVTDLTK
VHTECCHGDL LECADDRADL AKYICENQDS ISSKLKECCE
KPLLEKSHCI AEVENDEMPA DLPSLAADFV ESKDVCKNYA
EAKDVFLGMF LYEYARRHPD YSVVLLLRLA KTYETTLEKC
CAAADPHECY AKVFDEFKPL VEEPQNLIKQ NCELFEQLGE
YKFQNALLVR YTKKVPQVST PTLVEVSRNL GKVGSKCCKH
PEAKRMPCAE DYLSVVLNQL CVLHEKTPVS DRVTKCCTES
LVNRRPCFSA LEVDETYVPK EFNAETFTFH ADICTLSEKE
RQIKKQTALV ELVKHKPKAT KEQLKAVMDD FAAFVEKCCK
ADDKETCFAE EGKKLVAASQ AALGLGGGGS GGGGSGGGGS
ARNGDHCPLG PGRCCRLHTV RASLEDLGWA DWVLSPREVQ
VTMCIGACPS QFRAANMHAQ IKTSLHRLKP DTVPAPCCVP
ASYNPMVLIQ KTDTGVSLQT YDDLLAKDCH CI;
(iii)
HSA-GGGGS-hGDF15(197-308):

(SEQ ID NO: 329)

DAHKSEVAHR FKDLGEENFK ALVLIAFAQY LQQCPFEDHV
KLVNEVTEFA KTCVADESAE NCDKSLHTLF GDKLCTVATL
RETYGEMADC CAKQEPERNE CFLQHKDDNP NLPRLVRPEV
DVMCTAFHDN EETFLKKYLY EIARRHPYFY APELLFFAKR
YKAAFTECCQ AADKAACLLP KLDELRDEGK ASSAKQRLKC
ASLQKFGERA FKAWAVARLS QRFPKAEFAE VSKLVTDLTK
VHTECCHGDL LECADDRADL AKYICENQDS ISSKLKECCE
KPLLEKSHCI AEVENDEMPA DLPSLAADFV ESKDVCKNYA
EAKDVFLGMF LYEYARRHPD YSVVLLLRLA KTYETTLEKC
CAAADPHECY AKVFDEFKPL VEEPQNLIKQ NCELFEQLGE
YKFQNALLVR YTKKVPQVST PTLVEVSRNL GKVGSKCCKH
PEAKRMPCAE DYLSVVLNQL CVLHEKTPVS DRVTKCCTES
LVNRRPCFSA LEVDETYVPK EFNAETFTFH ADICTLSEKE
RQIKKQTALV ELVKHKPKAT KEQLKAVMDD FAAFVEKCCK
ADDKETCFAE EGKKLVAASQ AALGLGGGGS ARNGDHCPLG
PGRCCRLHTV RASLEDLGWA DWVLSPREVQ VTMCIGACPS
QFRAANMHAQ IKTSLHRLKP DTVPAPCCVP ASYNPMVLIQ
KTDTGVSLQT YDDLLAKDCH CI;
(iv)
HSA-GPPGS-hGDF15(197-308):

(SEQ ID NO: 330)

DAHKSEVAHR FKDLGEENFK ALVLIAFAQY LQQCPFEDHV
KLVNEVTEFA KTCVADESAE NCDKSLHTLF GDKLCTVATL
RETYGEMADC CAKQEPERNE CFLQHKDDNP NLPRLVRPEV
DVMCTAFHDN EETFLKKYLY EIARRHPYFY APELLFFAKR
YKAAFTECCQ AADKAACLLP KLDELRDEGK ASSAKQRLKC
ASLQKFGERA FKAWAVARLS QRFPKAEFAE VSKLVTDLTK
VHTECCHGDL LECADDRADL AKYICENQDS ISSKLKECCE
KPLLEKSHCI AEVENDEMPA DLPSLAADFV ESKDVCKNYA
EAKDVFLGMF LYEYARRHPD YSVVLLLRLA KTYETTLEKC
CAAADPHECY AKVFDEFKPL VEEPQNLIKQ NCELFEQLGE
YKFQNALLVR YTKKVPQVST PTLVEVSRNL GKVGSKCCKH
PEAKRMPCAE DYLSVVLNQL CVLHEKTPVS DRVTKCCTES
LVNRRPCFSA LEVDETYVPK EFNAETFTFH ADICTLSEKE
RQIKKQTALV ELVKHKPKAT KEQLKAVMDD FAAFVEKCCK
ADDKETCFAE EGKKLVAASQ AALGLGPPGS ARNGDHCPLG
PGRCCRLHTV RASLEDLGWA DWVLSPREVQ VTMCIGACPS
QFRAANMHAQ IKTSLHRLKP DTVPAPCCVP ASYNPMVLIQ
KTDTGVSLQT YDDLLAKDCH CI;
(v)
HSA-hGDF15(197-308)(no linker):

(SEQ ID NO: 331)

DAHKSEVAHR FKDLGEENFK ALVLIAFAQY LQQCPFEDHV
KLVNEVTEFA KTCVADESAE NCDKSLHTLF GDKLCTVATL
RETYGEMADC CAKQEPERNE CFLQHKDDNP NLPRLVRPEV
DVMCTAFHDN EETFLKKYLY EIARRHPYFY APELLFFAKR
YKAAFTECCQ AADKAACLLP KLDELRDEGK ASSAKQRLKC
ASLQKFGERA FKAWAVARLS QRFPKAEFAE VSKLVTDLTK
VHTECCHGDL LECADDRADL AKYICENQDS ISSKLKECCE
KPLLEKSHCI AEVENDEMPA DLPSLAADFV ESKDVCKNYA
EAKDVFLGMF LYEYARRHPD YSVVLLLRLA KTYETTLEKC
CAAADPHECY AKVFDEFKPL VEEPQNLIKQ NCELFEQLGE
YKFQNALLVR YTKKVPQVST PTLVEVSRNL GKVGSKCCKH
PEAKRMPCAE DYLSVVLNQL CVLHEKTPVS DRVTKCCTES
LVNRRPCFSA LEVDETYVPK EFNAETFTFH ADICTLSEKE
RQIKKQTALV ELVKHKPKAT KEQLKAVMDD FAAFVEKCCK
ADDKETCFAE EGKKLVAASQ AALGLARNGD HCPLGPGRCC
RLHTVRASLE DLGWADWVLS PREVQVTMCI GACPSQFRAA
NMHAQIKTSL HRLKPDTVPA PCCVPASYNP MVLIQKTDTG
VSLQTYDDLL AKDCHCI;
(vi)
HSA-hGDF15(197-308), R198H:

(SEQ ID NO: 332)

DAHKSEVAHR FKDLGEENFK ALVLIAFAQY LQQCPFEDHV
KLVNEVTEFA KTCVADESAE NCDKSLHTLF GDKLCTVATL
RETYGEMADC CAKQEPERNE CFLQHKDDNP NLPRLVRPEV
DVMCTAFHDN EETFLKKYLY EIARRHPYFY APELLFFAKR
YKAAFTECCQ AADKAACLLP KLDELRDEGK ASSAKQRLKC
ASLQKFGERA FKAWAVARLS QRFPKAEFAE VSKLVTDLTK
VHTECCHGDL LECADDRADL AKYICENQDS ISSKLKECCE
KPLLEKSHCI AEVENDEMPA DLPSLAADFV ESKDVCKNYA
EAKDVFLGMF LYEYARRHPD YSVVLLLRLA KTYETTLEKC
CAAADPHECY AKVFDEFKPL VEEPQNLIKQ NCELFEQLGE
YKFQNALLVR YTKKVPQVST PTLVEVSRNL GKVGSKCCKH
PEAKRMPCAE DYLSVVLNQL CVLHEKTPVS DRVTKCCTES
LVNRRPCFSA LEVDETYVPK EFNAETFTFH ADICTLSEKE
RQIKKQTALV ELVKHKPKAT KEQLKAVMDD FAAFVEKCCK
ADDKETCFAE EGKKLVAASQ AALGLGGGGS GGGGSGGGGS
AHNGDHCPLG PGRCCRLHTV RASLEDLGWA DWVLSPREVQ
VTMCIGACPS QFRAANMHAQ IKTSLHRLKP DTVPAPCCVP
ASYNPMVLIQ KTDTGVSLQT YDDLLAKDCH CI;
(vii)
HSA-hGDF15(197-308), R198H, N199A:

(SEQ ID NO: 333)

DAHKSEVAHR FKDLGEENFK ALVLIAFAQY LQQCPFEDHV
KLVNEVTEFA KTCVADESAE NCDKSLHTLF GDKLCTVATL
RETYGEMADC CAKQEPERNE CFLQHKDDNP NLPRLVRPEV
DVMCTAFHDN EETFLKKYLY EIARRHPYFY APELLFFAKR
YKAAFTECCQ AADKAACLLP KLDELRDEGK ASSAKQRLKC
ASLQKFGERA FKAWAVARLS QRFPKAEFAE VSKLVTDLTK
VHTECCHGDL LECADDRADL AKYICENQDS ISSKLKECCE
KPLLEKSHCI AEVENDEMPA DLPSLAADFV ESKDVCKNYA
EAKDVFLGMF LYEYARRHPD YSVVLLLRLA KTYETTLEKC
CAAADPHECY AKVFDEFKPL VEEPQNLIKQ NCELFEQLGE
YKFQNALLVR YTKKVPQVST PTLVEVSRNL GKVGSKCCKH
PEAKRMPCAE DYLSVVLNQL CVLHEKTPVS DRVTKCCTES
LVNRRPCFSA LEVDETYVPK EFNAETFTFH ADICTLSEKE
RQIKKQTALV ELVKHKPKAT KEQLKAVMDD FAAFVEKCCK
ADDKETCFAE EGKKLVAASQ AALGLGGGGS GGGGSGGGGS
AHAGDHCPLG PGRCCRLHTV RASLEDLGWA DWVLSPREVQ
VTMCIGACPS QFRAANMHAQ IKTSLHRLKP DTVPAPCCVP
ASYNPMVLIQ KTDTGVSLQT YDDLLAKDCH CI;
and
(viii)
HSA-hGDF15(197-308), N199E:

(SEQ ID NO: 334)

DAHKSEVAHR FKDLGEENFK ALVLIAFAQY LQQCPFEDHV
KLVNEVTEFA KTCVADESAE NCDKSLHTLF GDKLCTVATL
RETYGEMADC CAKQEPERNE CFLQHKDDNP NLPRLVRPEV
DVMCTAFHDN EETFLKKYLY EIARRHPYFY APELLFFAKR
YKAAFTECCQ AADKAACLLP KLDELRDEGK ASSAKQRLKC
ASLQKFGERA FKAWAVARLS QRFPKAEFAE VSKLVTDLTK
VHTECCHGDL LECADDRADL AKYICENQDS ISSKLKECCE
KPLLEKSHCI AEVENDEMPA DLPSLAADFV ESKDVCKNYA
EAKDVFLGMF LYEYARRHPD YSVVLLLRLA KTYETTLEKC
CAAADPHECY AKVFDEFKPL VEEPQNLIKQ NCELFEQLGE
YKFQNALLVR YTKKVPQVST PTLVEVSRNL GKVGSKCCKH
PEAKRMPCAE DYLSVVLNQL CVLHEKTPVS DRVTKCCTES
LVNRRPCFSA LEVDETYVPK EFNAETFTFH ADICTLSEKE
RQIKKQTALV ELVKHKPKAT KEQLKAVMDD FAAFVEKCCK
ADDKETCFAE EGKKLVAASQ AALGLGGGGS GGGGSGGGGS
AREGDHCPLG PGRCCRLHTV RASLEDLGWA DWVLSPREVQ
VTMCIGACPS QFRAANMHAQ IKTSLHRLKP DTVPAPCCVP
ASYNPMVLIQ KTDTGVSLQT YDDLLAKDCH CI.

### Linkers

Regarding the GDF15 fusion proteins (e.g., the serum albumin GDF15 fusion proteins) used in the present methods of the invention, the heterologous protein/peptide, e.g., SA, and GDF15 moieties can be directly bonded to each other in the contiguous polypeptide chain, or preferably indirectly bonded to each other through a suitable linker. The linker is preferably a peptide linker. Peptide linkers are commonly used in fusion polypeptides and methods for selecting or designing linkers are well-known. (See, e.g., Chen X et al. Adv. Drug Deliv. Rev. 65(10):135701369 (2013) and Wriggers W et al., Biopolymers 80:736-746 (2005).)

Peptide linkers generally are categorized as i) flexible linkers, ii) helix forming linkers, and iii) cleavable linkers, and examples of each type are known in the art. Preferably, a flexible linker is included in the fusion polypeptides described herein. Flexible linkers may contain a majority of amino acids that are sterically unhindered, such as glycine and alanine. The hydrophilic amino acid Ser is also conventionally used in flexible linkers. Examples of flexible linkers include, polyglycines (e.g., (Gly)₄ (SEQ ID NO: 335) and (Gly)₅) (SEQ ID NO: 336), polyalanines poly(Gly-Ala), and poly(Gly-Ser) (e.g., (Glyₙ-Serₙ)ₙ or (Serₙ-Glyₙ)ₙ, wherein each n is independent an integer equal to or greater than 1).

Peptide linkers can be of a suitable length. The peptide linker sequence may be at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues in length. For example, a peptide linker can be from about 5 to about 50 amino acids in length; from about 10 to about 40 amino acids in length; from about 15 to about 30 amino acids in length; or from about 15 to about 20 amino acids in length. Variation in peptide linker length may retain or enhance activity, giving rise to superior efficacy in activity studies. The peptide linker sequence may be comprised of a naturally, or non-naturally, occurring amino acids.

In some aspects, the amino acids glycine and serine comprise the amino acids within the linker sequence. In certain aspects, the linker region comprises sets of glycine repeats (GSG₃)ₙ, where n is a positive integer equal to or greater than 1 (preferably 1 to about 20) (SEQ ID NO:305). More specifically, the linker sequence may be GSGGG (SEQ ID NO:306). The linker sequence may be GSGG (SEQ ID NO:307). In certain other aspects, the linker region orientation comprises sets of glycine repeats (SerGly₃)ₙ, where n is a positive integer equal to or greater than 1 (preferably 1 to about 20) (SEQ ID NO:308).

In more embodiments, a linker may contain glycine (G) and serine (S) in a random or preferably a repeated pattern. For example, the linker can be (GGGGS)ₙ (SEQ ID NO:303), wherein n is an integer ranging from 1 to 20, preferably 1 to 4. In a particular example, n is 3 and the linker is GGGGSGGGGSGGGGS (SEQ ID NO:300).

In other embodiments, a linker may contain glycine (G), serine (S) and proline (P) in a random or preferably repeated pattern. For example, the linker can be (GPPGS)ₙ (SEQ ID NO:304), wherein n is an integer ranging from 1 to 20, preferably 1-4. In a particular example, n is 1 and the linker is GPPGS (SEQ ID NO:309).

In general, the linker is not immunogenic when administered in a patient, such as a human. Thus linkers may be chosen such that they have low immunogenicity or are thought to have low immunogenicity.

The linkers described herein are exemplary, and the linker can include other amino acids, such as Glu and Lys, if desired. The peptide linkers may include multiple repeats of, for example, (G₄S) (SEQ ID NO:310), (G₃S) (SEQ ID NO:311), (G₂S) (SEQ ID NO:312) and/or (GlySer) (SEQ ID NO:313), if desired. In certain aspects, the peptide linkers may include multiple repeats of, for example, (SW (SEQ ID NO:314), (SG₃) (SEQ ID NO:315), (SG₂) (SEQ ID NO:316) or (SerGly) (SEQ ID NO:317). In other aspects, the peptide linkers may include combinations and multiples of repeating amino acid sequence units, such as (G₃S)+(G₄S)+(GlySer) (SEQ ID NO:311+SEQ ID NO:310+SEQ ID NO:313). In other aspects, Ser can be replaced with Ala e.g., (G₄A) (SEQ ID NO:318) or (G₃A) (SEQ ID NO: 301). In yet other aspects, the linker comprises the motif (EAAAK)ₙ, where n is a positive integer equal to or greater than 1, preferably 1 to about 20 (SEQ ID NO:319). In certain aspects, peptide linkers may also include cleavable linkers.

In a particular embodiment, a GDF15 fusion or conjugate used in the present methods of the invention comprises a GDF15 moiety (e.g., a GDF15 polyptide comprising an amino acid sequence that is at least 95% identical to SEQ ID NO: 5) linked to a heterologous protein/peptide (e.g., HSA or Fc) or a conjugate moiety with a linker, wherein the linker has the amino acid sequence GGSSEAAEAAEAAEAAEAAEAAE (SEQ ID NO: 337). Additional non-limiting examples of linkers are described in PCT Publication No. WO2015/197446, which is incorporated herein by reference in its entirety, such as SEQ ID NOs: 4-13 and 24-38.

Regarding the GDF15 conjugates (e.g., the GDF15 FA conjugates) used in the present methods of the invention, the GDF15 moiety and conjugate moiety, e.g., fatty acid moiety, can be joined by a linker as follows:

The linker separates the GDF15 moiety and the conjugate moiety, e.g., fatty acid moiety. In particular embodiments, its chemical structure is not critical, since it serves primarily as a spacer.

In a specific embodiment, the linker is a chemical moiety that contains two reactive groups/functional groups, one of which can react with the GDF15 moiety and the other with the conjugate moiety, e.g., fatty acid moiety. The two reactive/functional groups of the linker are linked via a linking moiety or spacer, structure of which is not critical as long as it does not interfere with the coupling of the linker to the GDF15 moiety and the conjugate moiety, e.g., fatty acid moiety, such as for example fatty acid moieties of Formula A1, A2 or A3.

The linker can be made up of amino acids linked together by peptide bonds. The amino acids can be natural or non-natural amino acids. In some embodiments of the present invention, the linker is made up of from 1 to 20 amino acids linked by peptide bonds, wherein the amino acids are selected from the 20 naturally occurring amino acids. In various embodiments, the 1 to 20 amino acids are selected from the amino acids glycine, serine, alanine, methionine, asparagine, glutamine, cysteine, glutamic acid and lysine, or amide derivatives thereof such as lysine amide. In some embodiments, a linker is made up of a majority of amino acids that are sterically unhindered, such as glycine and alanine. In some embodiments, linkers are polyglycines, polyalanines, combinations of glycine and alanine (such as poly(Gly-Ala)), or combinations of glycine and serine (such as poly(Gly-Ser)). In some embodiments, a linker is made up of a majority of amino acids selected from histidine, alanine, methionine, glutamine, asparagine and glycine. In some embodiments, the linker contains a poly-histidine moiety. In other embodiments, the linker contains glutamic acid, glutamine, lysine or lysine amide or combination thereof.

In some embodiment, the linker may have more than two available reactive functional groups and can therefore serve as a way to link more than one fatty acid moiety. For example, amino acids such as Glutamine, Glutamic acid, Serine or Lysine can provide several points of attachment for a fatty acid moiety: the side chain of the amino acid and the functionality at the N-terminus or the C-terminus.

In some embodiments, the linker comprises 1 to 20 amino acids which are selected from non-natural amino acids. While a linker of 1-10 amino acid residues is preferred for conjugation with the fatty acid moiety, the present invention contemplates linkers of any length or composition. An example of non-natural amino acid linker is 8-Amino-3,6-dioxaoctanoic acid having the following formula:

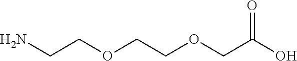 [SMILES: NCCOCCOCC(=O)O]

or its repeating units.

The linkers described herein are exemplary, and linkers that are much longer and which include other residues are contemplated by the present invention. Non-peptide linkers are also contemplated by the present invention.

In other embodiments, the linker comprise one or more alkyl groups, alkenyl groups, cycloalkyl groups, aryl groups, heteroaryl groups, heterocyclic groups, polyethylene glycol and/or one or more natural or unatural amino acids, or combination thereof, wherein each of the alkyl, alkenyl, cycloalkyl, aryl, heteroaryl, heterocyclyl, polyethylene glycol and/or the natural or unatural amino acids are optionally combined and linked together, or linked to the GDF15 moiety and/or to the fatty acid moiety, via a chemical group selected from —C(O)O—, —OC(O)—, —NHC(O)—, —C(O)NH—, —O—, —NH—, —S—, —C(O)—, —OC(O)NH—, —NHC(O)—O—, ═NH—O—, ═NH—NH— or ═NH—N(alkyl)-.

Linkers containing alkyl spacer are for example —NH—(CH₂)_{z}—C(O)— or —S—(CH₂)_{z}—C(O)— or —O—(CH₂)_{z}—C(O)—, —NH—(CH₂)_{z}—NH—, —O—C(O)—(CH₂)_{z}—C(O)—O—, —C(O)—(CH₂)_{z}—O—, —NHC(O)—(CH₂)_{z}—C(O)—NH— and the like wherein z is 2-20 can be used. These alkyl linkers can further be substituted by any non-sterically hindering group, including, but not limited to, a lower alkyl (e.g., C₁-C₆), lower acyl, halogen (e.g., Cl, Br), CN, NH₂, or phenyl.

The linker can also be of polymeric nature. The linker may include polymer chains or units that are biostable or biodegradable. Polymers with repeat linkage may have varying degrees of stability under physiological conditions depending on bond lability. Polymers may contain bonds such as polycarbonates (—O—C(O)—O—), polyesters (—C(O)—O—), polyurethanes (—NH—C(O)—O—), polyamide (—C(O)—NH—). These bonds are provided by way of examples, and are not intended to limit the type of bonds employable in the polymer chains or linkers of the invention. Suitable polymers include, for example, polyethylene glycol (PEG), polyvinyl pyrrolidone, polyvinyl alcohol, polyamino acids, divinylether maleic anhydride, N-(2-hydroxypropyl)-methacrylicamide, dextran, dextran derivatives, polypropylene glycol, polyoxyethylated polyol, heparin, heparin fragments, polysaccharides, cellulose and cellulose derivatives, starch and starch derivatives, polyalkylene glycol and derivatives thereof, copolymers of polyalkylene glycols and derivatives thereof, polyvinyl ethyl ether, and the like and mixtures thereof. A polymer linker is for example polyethylene glycol (PEG). The PEG linker can be linear or branched. A molecular weight of the PEG linker in the present invention is not restricted to any particular size, but certain embodiments have a molecular weight between 100 to 5000 Dalton for example 500 to 1500 Dalton.

The linking moiety (or spacer) contains appropriate functional-reactive groups at both terminals that form a bridge between an amino group of the peptide or polypeptide/protein (e.g. N-terminus or side chain of a lysine) and a functional/reactive group on the fatty acid moiety (e.g the carboxylic acid functionality of the fatty acid moiety). Alternatively, the linking moiety (or spacer) contains appropriate functional-reactive groups at both terminals that form a bridge between an acid carboxylic group of the peptide or polypeptide/protein (e.g. C-terminus) and a functional/reactive group on the fatty acid moiety (e.g the carboxylic acid functionality of the fatty acid moiety of formula A1, A2 and A3).

The linker may comprise several linking moieties (or spacer) of different nature (for example a combination of amino acids, heterocyclyl moiety, PEG and/or alkyl moieties). In this instance, each linking moiety contains appropriate functional-reactive groups at both terminals that form a bridge between an amino group of the peptide or polypeptide/protein (e.g. the N-terminus or the side chain of a lysine) and the next linking moiety of different nature and/or contains appropriate functional-reactive groups that form a bridge between the prior linking moiety of different nature and the fatty acid moiety. In other instance, each linking moiety contains appropriate functional-reactive groups at both terminals that form a bridge between an acid carboxylic group of the peptide or polypeptide/protein (e.g. the C-terminus) and the next linking moiety of different nature and/or contains appropriate functional-reactive groups that form a bridge between the prior linking moiety of different nature and the fatty acid moiety.

Additionally, a linking moiety may have more than 2 terminal functional groups and can therefore be linked to more than one fatty acid moiety. Example of these multi-functional groups moieties are glutamic acid, lysine or serine. The side chain of the amino acid can also serve as a point of attachment for another fatty acid moiety.

The modified peptides or polypeptides and/or peptide-polypeptide partial construct (i.e. peptide/polypeptide attached to a partial linker) include reactive groups which can react with available reactive functionalities on the fatty acid moiety (or modified fatty acid moiety: i.e. already attached a partial linker) to form a covalent bond. Reactive groups are chemical groups capable of forming a covalent bond. Reactive groups are located at one site of conjugation and can generally be carboxy, phosphoryl, acyl group, ester or mixed anhydride, maleimide, N-hydroxysuccinimide, tetrazine, alkyne, imidate, pyridine-2-yl-disulfanyl, thereby capable of forming a covalent bond with functionalities like amino group, hydroxyl group, alkene group, hydrazine group, hydroxylamine group, an azide group or a thiol group at the other site of conjugation.

Reactive groups of particular interest for conjugating a GDF15 moiety to a linker and/or a linker to the fatty acid moiety and/or to conjugate various linking moieties of different nature together are N-hydroxysuccinimide, alkyne (more particularly cyclooctyne).

Functionalities include: 1. thiol groups for reacting with maleimides, tosyl sulfone or pyridine-2-yldisulfanyl; 2. amino groups (for example amino functionality of an amino acid) for bonding to carboxylic acid or activated carboxylic acid (e.g. amide bond formation via N-hydroxysuccinamide chemistry), phosphoryl groups, acyl group or mixed anhydride; 3. Azide to undergo a Huisgen cycloaddition with a terminal alkyne and more particularly cyclooctyne (more commonly known as click chemistry); 4. carbonyl group to react with hydroxylamine or hydrazine to form oxime or hydrazine respectively; 5. Alkene and more particularly strained alkene to react with tetrazine in an aza [4+2] addition. While several examples of linkers and functionalities/reactive group are described herein, the methods of the present invention contemplate linkers of any length and composition.

### GDF15 Fusion Polypeptides

In specific aspects, GDF15 fusion polypeptides described herein as useful for administration for the present methods of treatment of the invention may contain a GDF15 moiety and a heterologous moiety, and optionally a linker. In a particular embodiment, a GDF15 fusion polypeptide described herein as useful for administration for the present methods of treatment of the invention may contain a GDF15 moiety and a heterologous moiety which is alpha-1-antitrypsin (A1AT) or a variant thereof, and optionally a linker. For example, GDF15-A1AT fusion polypeptides are described in PCT Publication No. WO2016/102580, which is incorporated by reference herein in its entirety.

In specific aspects, GDF15 fusion polypeptides described herein as useful for administration for the present methods of treatment of the invention may contain a GDF15 moiety and a serum albumin (SA) moiety, and optionally a linker. In one embodiment, the fusion polypeptide is a contiguous amino acid chain in which the SA moiety is located N-terminally to the GDF15 moiety. The C-terminus of the SA moiety can be directly bonded to the N-terminus of the GDF15 moiety. Preferably, the C-terminus of the SA moiety is indirectly bonded to the N-terminus of the GDF15 moiety through a peptide linker.

The SA moiety and GDF15 moiety can be from any desired species. For example, the fusion protein can contain SA and GDF15 moieties that are from human, mouse, rat, dog, cat, horse or any other desired species. The SA and GDF15 moieties are generally from the same species, but fusion peptides in which the SA moiety is from one species and the GDF15 moiety is from another species (e.g., mouse SA and human GDF15) are also encompassed by this disclosure.

In some embodiments, the fusion polypeptide comprises mouse serum albumin or functional variant thereof and mature human GDF15 peptide or functional variant thereof. For example, the fusion protein can have the amino acid sequence of any of SEQ ID NOS: 9, 10, 12, 13, and 18.

In preferred embodiments, the SA moiety is an HSA or a functional variant thereof and the GDF15 moiety is the mature human GDF peptide or a functional variant thereof. When present, the optional linker is preferably a flexible peptide linker. In particular embodiments, the fusion polypeptide comprises

A) an SA moiety selected from the group consisting of HSA(25-609) (SEQ ID NO: 4), and HSA(25-609) in which Cys34 is replaced with Ser and Asn503 is replaced with Gln; and

B) a GDF15 moiety selected from the group consisting of:
human GDF15(197-308) (SEQ ID NO:5);
human GDF15(211-308) (amino acids 211-308 of SEQ ID NO:2);
human GDF15(197-308) (SEQ ID NO:5) in which Cys203 is replaced with Ser (C2035) and Cys210 is replaced with Ser (C210S); and
human GDF15(197-308) (SEQ ID NO:5) in which Cys273 is replaced with Ser (C273S).

If desired, the fusion polypeptide can further comprise a linker that links the C-terminus of the SA moiety to the N-terminus of the GDF15 moiety. Preferably, the linker is selected from (GGGGS)n (SEQ ID NO:303) and (GPPGS)n (SEQ ID NO:304), wherein n is one to about 20. Preferred linkers include ((GGGGS)n (SEQ ID NO:303) and (GPPGS)n (SEQ ID NO:304), wherein n is 1, 2, 3 or 4.

In more particular embodiments, the fusion polypeptide comprises HSA or a functional variant thereof, a linker, and mature human GDF15 polypeptide or a functional variant thereof and has an amino acid sequence that has at least about 90%, at least about 95%, at least about 96%, at least about 97%, at least about 98%, or at least about 99% amino acid sequence identity to any of SEQ ID NOs: 11, 18, 19, 15.

In even more particular embodiments, the fusion polypeptide has the amino acid sequence of SEQ ID NOs: 11, 14, 15, 16, 17, 20, 21, and 22.

If desired, the fusion polypeptide can contain additional amino acid sequence. For example, an affinity tag can be included to facilitate detecting and/or purifying the fusion polypeptide.

### GDF15 Conjugates

Various embodiments of the GDF15 conjugates, e.g., GDF15 fatty acid conjugates, that can be used in the present methods of treatment of the invention are described herein. It will be recognized that features specified in each embodiment may be combined with other specified features to provide further embodiments.

In a specific embodiment, a GDF15 conjugate for the methods provided here comprises a GDF15 polypeptide or a functional variant thereof conjugated to a moiety, such as a fatty acid moiety, optionally comprising a linker. In some embodiment of the invention, the fatty acid residue is a lipophilic residue.

In another embodiment the fatty acid residue is negatively charged at physiological pH. In another embodiment the fatty acid residue comprises a group which can be negatively charged. One preferred group which can be negatively charged is a carboxylic acid group.

In another embodiment of the invention, the fatty acid residue binds non-covalently to albumin or other plasma proteins. In yet another embodiment of the invention the fatty acid residue is selected from a straight chain alkyl group, a branched alkyl group, a group which has an ω-carboxylic acid group, a partially or completely hydrogenated cyclopentanophenanthrene skeleton.

In another embodiment the fatty acid residue is a cibacronyl residue.

In another embodiment the fatty acid residue has from 6 to 40 carbon atoms, from 8 to 26 carbon atoms or from 8 to 20 carbon atoms.

In another embodiment, the fatty acid residue is an acyl group selected from the group comprising R—C(O)— wherein R is a C₄₋₃₈ linear or branched alkyl or a C₄₋₃₈ linear or branched alkenyl where each said alkyl and alkenyl are optionally substituted with one ore more substituents selected from —CO₂H, hydroxyl, —SO₃H, halo and —NHC(O)C(O)OH. The acyl group (R—C(O)—) derives from the reaction of the corresponding carboxylic acid R—C(O)OH with an amino group on the GDF15 polypetide.

In another embodiment the fatty acid residue is an acyl group selected from the group comprising CH₃(CH₂)ᵣ—CO, wherein r is an integer from 4 to 38, preferably an integer from 4 to 24, more preferred selected from the group comprising CH₃(CH₂)₆CO—, CH₃(CH₂)₈—CO—, CH₃(CH₂)₁₀—CO—, CH₃(CH₂)₁₂—CO—, CH₃(CH₂)₁₄—CO—, CH₃(CH₂)₁₆—CO—, CH₃(CH₂)₁₈—CO—, CH₃(CH₂)₂₀—CO and CH₃(CH₂)₂₂—CO—.

In another embodiment the fatty acid residue is an acyl group of a straight-chain or branched alkane α, ω dicarboxylic acid.

In another embodiment the fatty acid residue is an acyl group selected from the group comprising HOOC—(CH₂)ₛCO—, wherein s is an integer from 4 to 38, preferably an integer from 4 to 24, more preferred selected from the group comprising HOOC(CH₂)₁₄—CO—, HOOC(CH₂)₁₆—CO—, HOOC(CH₂)₁₈—CO—, HOOC(CH₂)₂₀—CO— and HOOC(CH₂)₂₂—CO—.

In another embodiment the fatty acid residue is a group of the formula CH₃—(CH₂)ₓ—CO—NH—CH(CH₂CO₂H)—C(O)— wherein x is an integer of from 8 to 24.

In yet another embodiment the fatty acid residue is selected from the group consisting of:

CH₃—(CH₂)₆₋₂₄—CO₂H; CF₃—(CF₂)₄₋₉—CH₂CH₂—CO₂H; CF₃—(CF₂)₄₋₉—CH₂CH₂—O—CH₂—CO₂H; CO₂H—(CH₂)₆₋₂₄—CO₂H; SO₂H—(CH₂)₆₋₂₄—CO₂H; wherein the fatty acid is linked to an amino group on GDF15 polypeptide (N-terminus or side chain of a lysine) or to an amino group on a linker via one of its carboxylic functionalities.

Specific examples of fatty acid are:

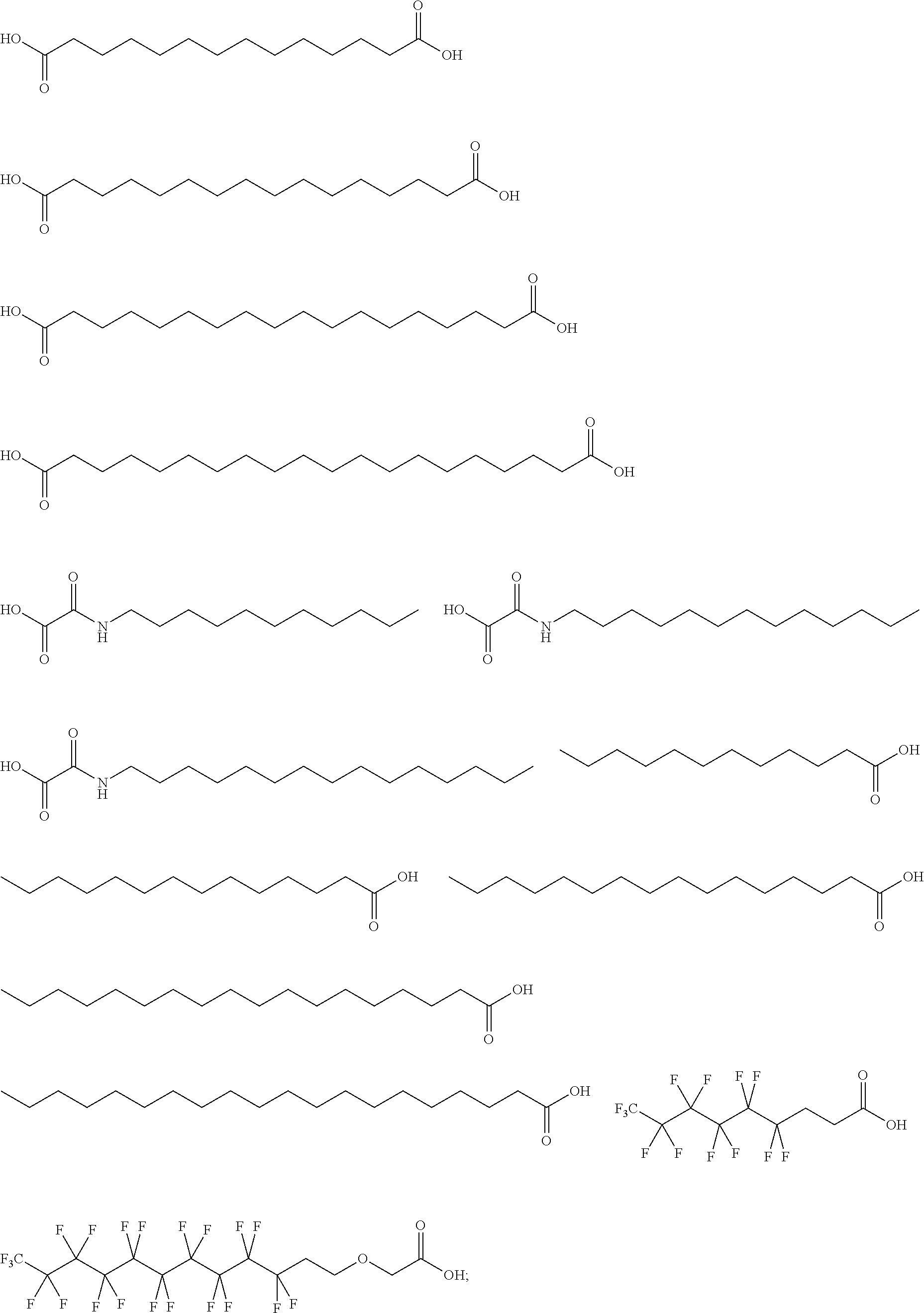

wherein the fatty acid is linked to the N-terminus of GDF15 or to an amino group on the side chain of GDF15 or to an amino group on a linker via one of its carboxylic acid functionalities.

Of particular interest, the linker between the above mentioned fatty acids and the GDF15 comprises lysine, glutamic acid, repeating units of:

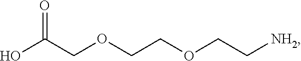

preferably 1 to 3; or mixture thereof.

More preferably, the linker comprises one or more glutaminc acid amino acids and one or more repeating unit of CO₂H—CH₂—O—CH₂—CH₂—O—CH₂—CH₂—NH₂.

Examples of fatty acid linked to one or two glutamic acid amino acids are:

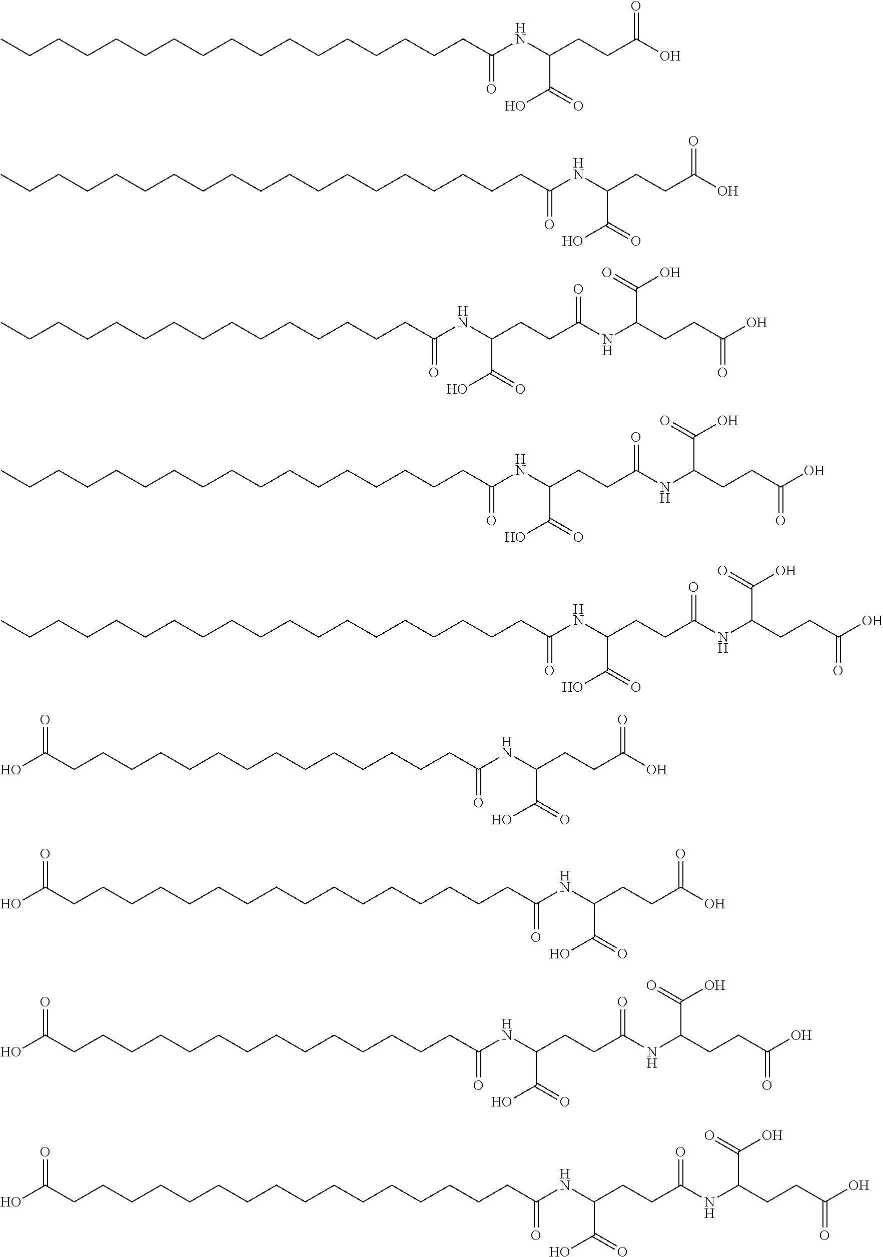

wherein the chiral carbon atoms independently are either R or S and wherein the fatty acid-linker moiety is linked to the N-terminus of GDF15 or to an amino group on the side chain of GDF15 or to an amino group on another linking moiety via one of the Glutamic acid's carboxylic acid functionalities.

Also of particular interest, the linker comprises one or more Lysine or Lysine amide amino acids, and one or more repeating unit of CO₂H—CH₂—O—CH₂—CH₂—O—CH₂—CH₂—NH₂.

Example of fatty acid moity(ies) linked to a Lysine or/and a Lysine amide amino acids are:

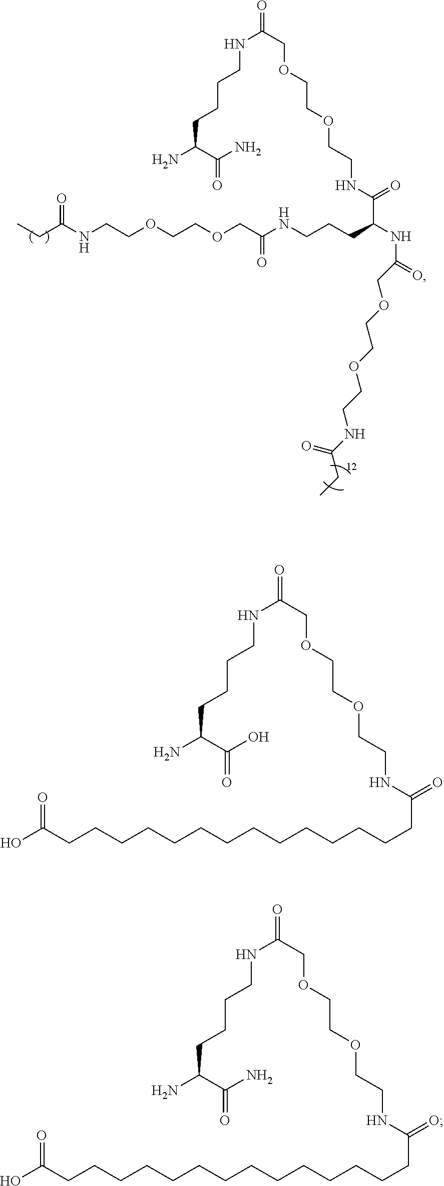

wherein the primary amino group of the lysine is attached the C-terminus of GDF15 or to a carboxylic acid functionality on a side chain of GDF15; or to a carboxylic acid functionality on another linking moiety.

Another specific example of linkers to be used with above fatty acids is 4-sulfamoylbutanoic acid:

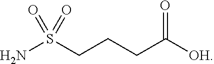

Examples of fatty acids linked to the above linker are:

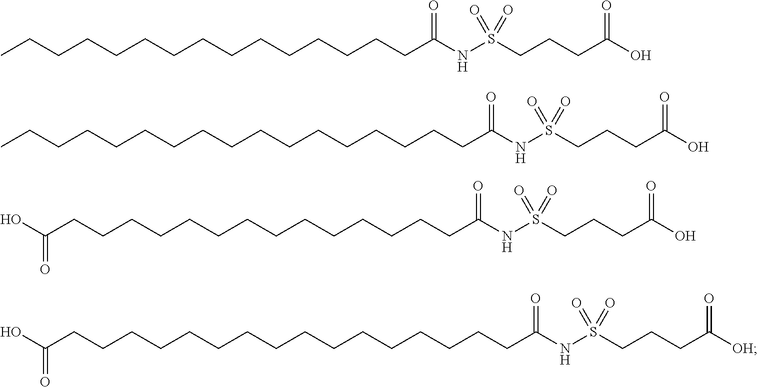

wherein the fatty acid-linker moiety is linked to the N-terminus of GDF15 or to an amino group on the side chain of GDF15 or to an amino group on another linking moiety via the carboxylic acid functionality on the sulfamoyl butanoic acid moiety.

Additionally, such fatty acid linker construct can further comprise repeating units of:

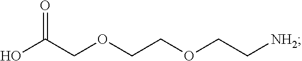

preferably 1 to 4.

Other examples of fatty acid-linker constructs are further disclosed in US 2013/0040884, Albumin-binding conjugates comprising fatty acid and PEG (Novo Nordisk) which is incorporated by reference.

Such constructs are preferably linked to the N-terminus of GDF15 via a carboxylic acid functionality.

In embodiment 1, the invention pertains to a conjugate comprising a GDF15 moiety linked to a fatty acid moiety via a linker wherein the fatty acid moiety has the following Formulae A1, A2 or A3:

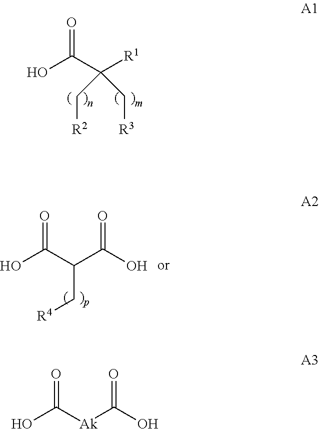

R¹ is CO₂H, H;
R², R³ and R⁴ are independently of each other H, OH, CO₂H, —CH═CH₂ or —C≡CH;
Ak is a branched C₆-C₃₀alkylene;
n, m and p are independently of each other an integer between 6 and 30; or an amide, an ester or a pharmaceutically acceptable salt thereof.

In embodiment 1A, the invention pertains to a conjugate according to embodiment 1 wherein the fatty acid moiety is of Formula A1. In a particular aspect of this embodiment, the conjugate comprises a fatty acid moiety of Formula A1 wherein n and m are independently 8 to 20, preferably 10 to 16. In another aspect of this embodiment, the invention pertains to a conjugate according to embodiment 1 or 1A wherein the fatty acid moiety is of Formula A1 and wherein at least one of R2 and R3 is CO2H.

In embodiment 2, the invention pertains to a conjugate according to embodiment 1 or 1A, wherein the fatty acid moiety is selected from the following Formulae:

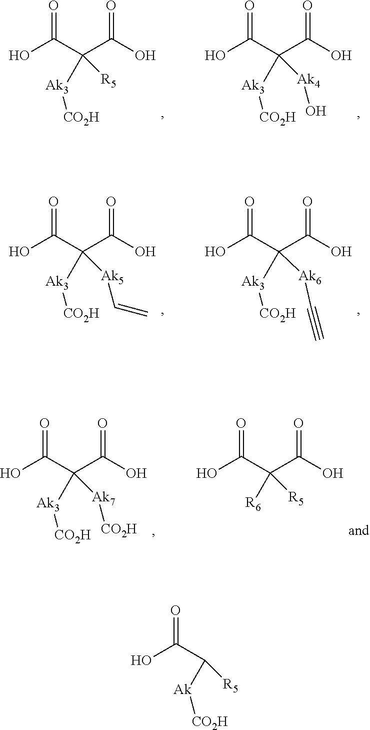

wherein Ak3, Ak4, Ak5, Ak6 and Ak7 are independently a (C₈₋₂₀)alkylene, R5 and R6 are independently (C₈₋₂₀)alkyl.

In embodiment 3, the invention pertains to a conjugate according to embodiment 1, 1A or 2 wherein the fatty acid moiety is selected from the following Formulae:

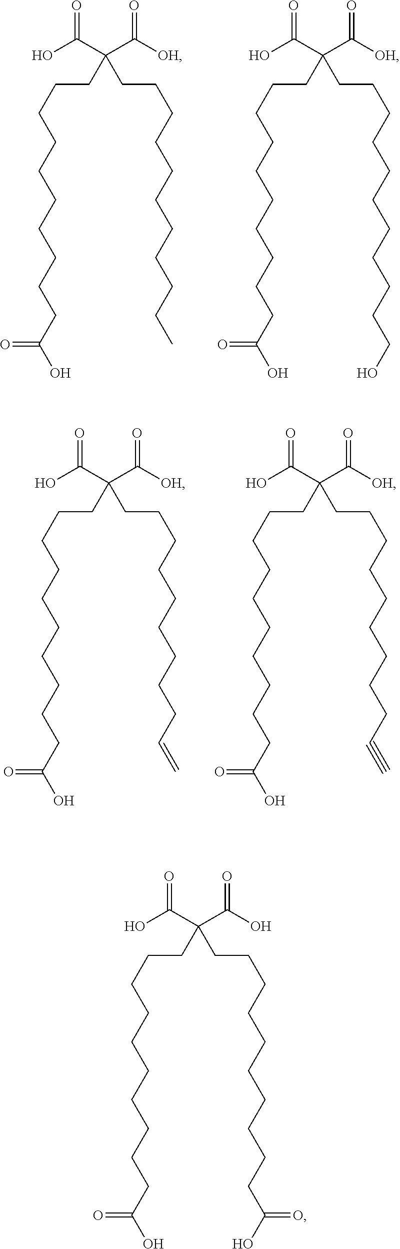

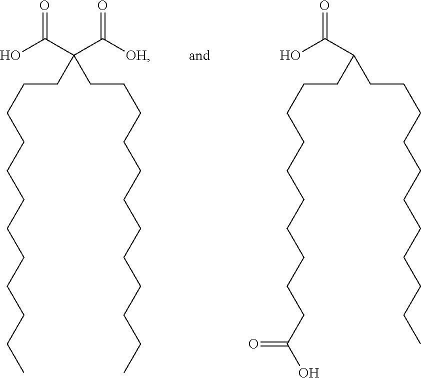 [SMILES: CCCCCCCCCCCCC(CCCCCCCCCCCC)(C(=O)O)C(=O)O ; "and" ; CCCCCCCCCCCC(CCCCCCCCCCC(=O)O)C(=O)O]

In embodiment 3A, the invention pertains to a conjugate according to embodiment 1, 1A or 2 wherein the fatty acid moiety is selected from the following Formulae:

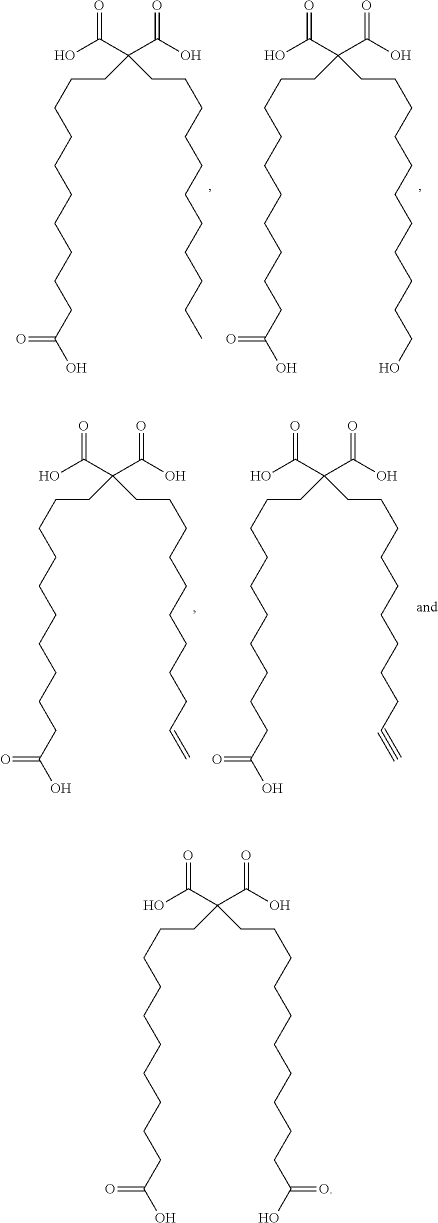

In embodiment 3B, the invention pertains to a conjugate according to embodiment 1 wherein the fatty acid moiety is of Formula A2 or A3. In a particular aspect of this embodiment, the conjugate comprises a fatty acid moiety of Formula A2 wherein p is 8 to 20, or a fatty acid moiety of Formula A3 wherein Ak is C₈₋₂₀alkylene.

In embodiment 3C, the invention pertains to a conjugate according to embodiment 1 or 3B wherein the fatty acid moiety is selected from the following Formulae:

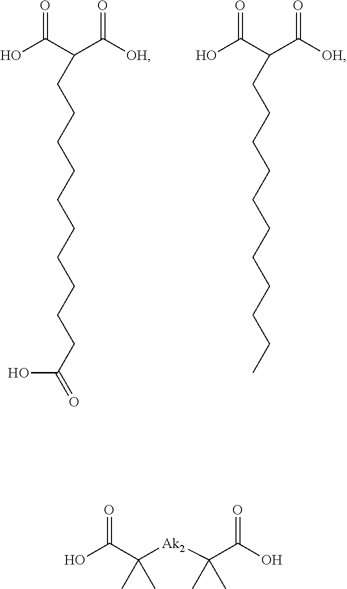

wherein Ak₂ is C₈₋₂₀alkylene.

In embodiment 4, the invention pertains to a conjugate according to any of the preceding conjugate's embodiments wherein the linker comprise one or more alkyl groups, alkenyl groups, cycloalkyl groups, aryl groups, heteroaryl groups, heterocyclic groups, polyethylene glycol, one or more natural or unatural amino acids, or combination thereof, wherein each of the alkyl, alkenyl, cycloalkyl, aryl, heteroaryl, heterocyclyl, polyethylene glycol and/or the natural or unatural amino acids are optionally combined and linked together or linked to the GDF15 moiety and/or to the fatty acid moiety via a chemical group selected from —C(O)O—, —OC(O)—, —NHC(O)—, —C(O)NH—, —O—, —NH—, —S—, —C(O)—, —OC(O)NH—, —NHC(O)—O—, ═NH—O—, ═NH—NH— or ═NH—N(alkyl)-.

In embodiment 5, the fatty acid conjugates that can be used in the present methods of treatment of the invention pertain to a conjugate according to any of the preceding conjugate's embodiments, wherein the linker comprises an unbranched oligo ethylene glycol moiety of Formula:

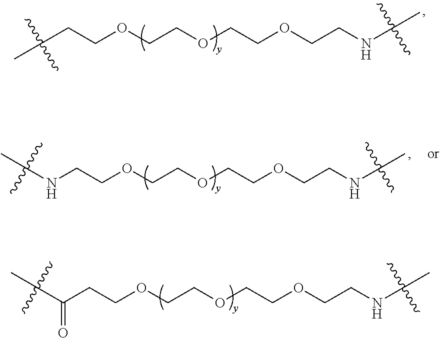

wherein y is 0 to 34.

In embodiment 6, the invention pertains to conjugate according to any of the preceding conjugate's embodiments wherein the linker comprises (or further comprises) a heterocyclic moiety selected from the following Formulae:

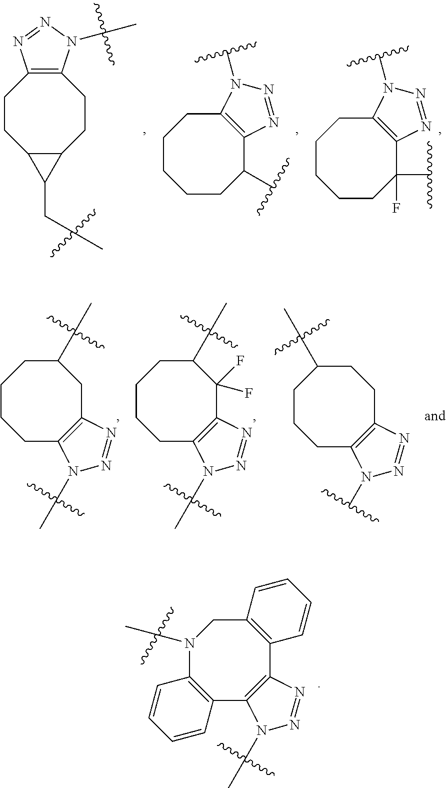

Such heterocyclyl containing linkers are obtained for example by azide-alkyne Huisgen cycloaddition, which more commonly known as click chemistry. More particularly, some of the heterocyclyl depicted supra result from the reaction of a cycloalkyne with an azide-containing moiety.

Cycloalkyne are readily available from commercial sources and can therefore be functionalized via cycloaddition with a moiety containing an azide functionality (e.g. a linker containing a terminal azide functionality). Examples of the use of cyclic alkyne click chemistry in protein labeling has been described in US 2009/0068738 which is herein incorporated by reference.

Non-limiting examples of cycloakyne agents which can be used in Huisgen cycloaddition are:

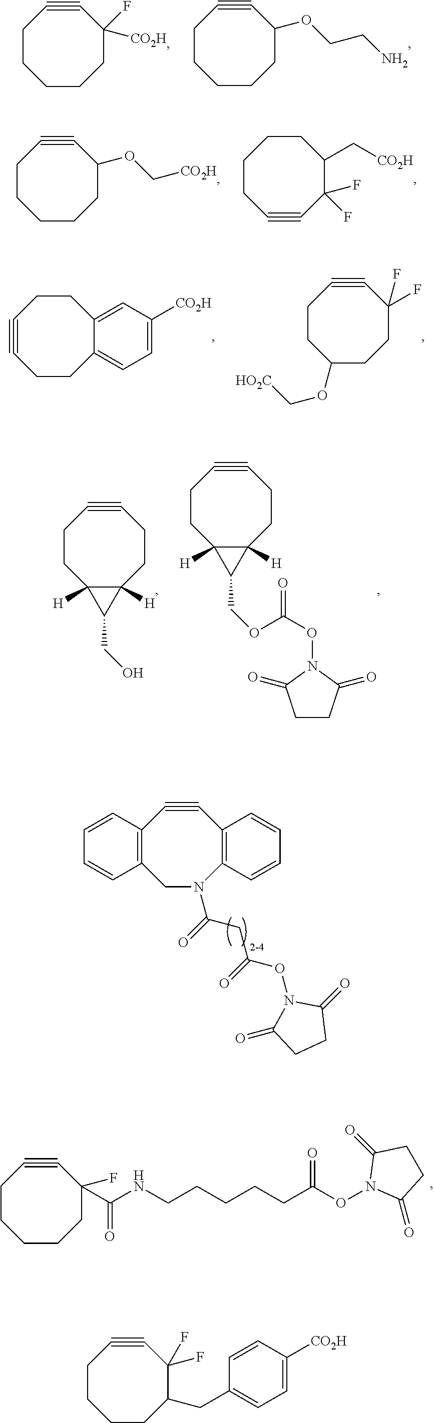

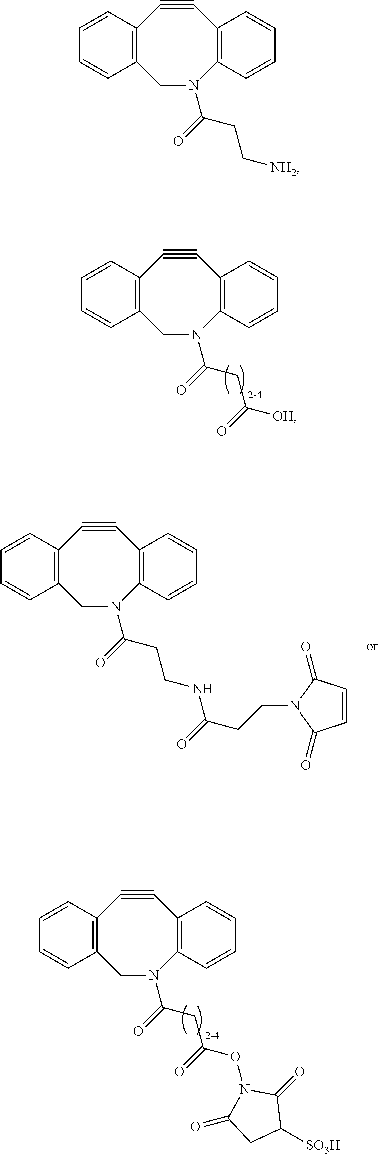

In embodiment 6A, the invention pertains to a fatty acid conjugate wherein the linker comprises (or further comprises) a heterocyclyl selected from the following Formulae:

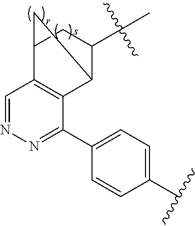

wherein r is an integer of 0 to 2 and s is an integer of 0 to 3.

Such heterocyclic linkers can be obtained via an aza [4+2] cycloadditon of an alkene, or preferably a strained alkene such as cycloalkane, with the following moiety:

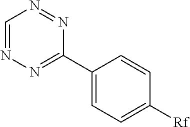

wherein Rf is for example —CH₂NH₂, —OH, —CH₂—CO₂H, —S—CH₂—CO₂H, —(O—CH₂)₄₋₆—C(O)—OH— or

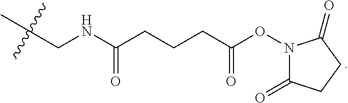 [SMILES: *CNC(=O)CCCC(=O)ON1C(=O)CCC1=O]

Such tetrazine moieties are readily available from commercial sources and can react with an alkene-containing moiety, for example a linker containing terminal alkene functionality.

In embodiment 6B, the invention pertains to a fatty acid conjugate wherein the linker comprises (or further comprises) a heterocyclyl of Formula:

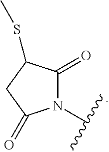 [SMILES: *N1C(=O)CC(SC)C1=O]

Such heterocyclic moiety can be obtained by reacting a maleimide with a thiol containing moiety, such as for example a linker containing a terminal thiol functionality.

These reagents which are readily available and/or commercially available are attached directly or via a linker as described supra to the peptide or polypeptide of interest. The alkyne, maleimide or tetrazine reactive groups are reacted with a functional group (azide, thiol and alkene respectively) which is present on the fatty acid moiety or on a linker-fatty acid construct (such as for example a PEG-fatty acid construct).

In embodiment 7, the invention pertains to a conjugate according to any of the preceding conjugate's embodiments wherein the linker comprises or further comprises one or more amino acids independently selected from histidine, methionine, alanine, glutamine, asparagine and glycine. In one particular aspect of this embodiment, the linker comprises 1 to 6 amino acid selected from histidine, alanine and methionine.

In embodiment 8, the invention pertains to a conjugate according to any one of the preceding conjugate's embodiments wherein the GDF15 moiety is human Growth Differentiation Factor 15 (GDF15), or related proteins and homologs, variants, fragments and other modified forms thereof. In embodiment 8A, the invention pertains to a conjugate according to any one of the preceding conjugate's embodiments wherein the GDF15 moiety is human Growth Differentiation Factor 15 (GDF15) variant.

In embodiment 8B, the invention contemplates a conjugate according to embodiment 8A wherein the human GDF15 variant is obtained by replacement of one or more amino acid residues of the mature polypeptide with another residue. In one particular aspect of this embodiment, the last two amino acid residues at the N-terminal of human GDF15 (i.e Arginine 198 and Alanine 197) have been replaced with an amino acid sequence XH— wherein H is histidine and X is an amino acid selected from methionine, alanine, glutamine, asparagine and glycine. In a preferred aspect of this embodiment, the hGDF15 variant is MH(199-308)hGDF15 or AH(199-308)hGDF15.

In embodiment 8C, the last three amino acid residues at the N-terminal of human GDF15 (i.e. Asparagine 199, Arginine 198 and Alanine 197) have been replaced with an amino acid sequence XHX′— wherein H is histidine and X′ and X are amino acids independently selected from selected from methionine, alanine, glutamine, asparagine and glycine. In another aspect of this embodiment, the last three amino acid residues at the N-terminal of human GDF15 (i.e. Asparagine 199, Arginine 198 and Alanine 197) have been replaced with an amino acid sequence AHX′— wherein H is histidine and X′ is an amino acids independently selected from selected from methionine, alanine, glutamine, asparagine and glycine. In a preferred aspect of this embodiment, the modified GDF15 protein is MHA(200-308)hGDF15 or AHA(200-308)hGDF15.

In one embodiment, the invention is directed toward a GDF15 fatty acid conjugates comprising a fatty acid is of the Formula R—CO2H wherein R is a C₄₋₃₈ linear or branched alkyl or a C₄₋₃₈ linear or branched alkenyl where each said alkyl and alkenyl are optionally substituted with one ore more substituents selected from —CO₂H, hydroxyl, —SO₃H, halo and —NHC(O)C(O)OH but wherein the fatty acid residue is not a fatty acid according to Formulae A1, A2, A3 and wherein the fatty acid is not myristic acid.

In another embodiment, the invention pertains to a GDF15 fatty acid conjugates according to any one of embodiments 7, 8, 8B, 8C wherein the GDF15 fatty acid conjugates do not comprise a fatty acid of Formula A1, A2 or A3 and do not comprise myristic acid.

In another embodiment, the invention is directed toward a GDF15-fatty acid conjugates according to embodiment 1-8 wherein the GDF15 fatty acid conjugate does not comprise the amino sequence of:

(i)
SEQ ID NO: 41;
(ii)

(SEQ ID NO: 321)

MHHHH HHAR NGDHC PLGPG RCCRL HTVRA SLEDL GWADW
VLSPR EVQVT MCIGA CPSQF RAANM HAQIK TSLHR LKPDT
VPAPC CVPAS YNPMV LIQKT DTGVS LQTYD DLLAK DCHCI
(M-(his)₆-hGDF15 (197-308)),
(iii)

(SEQ ID NO: 322)

MHHHHHHMARNGDHCPLGPGRCCRLHTVRASLEDLGWADWVLSPREVQV
TMCIGACPSQFRAANMHAQIKTSLHRLKPDTVPAPCCVPASYNPMVLIQ
KTDTGVSLQTYDDLLAKDCHCI (M-(his)₆-M-hGDF15
(197-308)),
(iv)

(SEQ ID NO: 323)

MHHHHHHAHARDGCPLGEGRCCRLQSLRASLQDLGWANWVVAPRELDVR
MCVGACPSQFRSANTHAQMQARLHGLNPDAAPAPCCVPASYEPVVLMHQ
DSDGRVSLTPFDDLVAKDCHCV (M-(his)₆-dGDF15),
(v)

(SEQ ID NO: 324)

MHNGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (MH-hGDF15(199-308)),
(vi)

(SEQ ID NO: 325)

MHAGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (MHA-hGDF15(200-308)),
and
(vii)

(SEQ ID NO: 326)

AHNGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (AH-hGDF15(199-308).

Examples of Fatty acid GDF15 conjugates comprising fatty acid of Formula A1, A2, A3 have been described in PCT application No. WO2015/200080.

Compared to the native GDF15, the GDF15 variant enables the selective labeling of the protein at the N-terminus (i.e. conjugation of the fatty acid at the preferred N-terminus of the GDF15). The selective labeling of peptide and protein is described in further details in PCT application No. WO2015/200078.

### Nucleic Acids and Host Cells

The invention also relates to nucleic acids that encode the fusion polypeptides described herein as useful for administration for the present methods of treatment of the invention, including vectors that can be used to produce the fusion polypeptides. The nucleic acids are isolated and/or recombinant. In certain embodiments, the nucleic acid encodes a fusion polypeptide in which HSA or a functional variant thereof is located N-terminally to human mature GDF15 or a functional variant thereof. If desired the nucleic acid can further encode a linker (e.g., a flexible peptide linker) that bonds the C-terminus of the HSA or a functional variant thereof to the N-terminus of human mature GDF15 or a functional variant thereof. If desired, the nucleic acid can also encode a leader, or signal, sequence to direct cellular processing and secretion of the fusion polypeptide.

In preferred embodiments, the nucleic acid encodes a fusion polypeptide in which the SA moiety is HSA or a functional variant thereof and the GDF15 moiety is the mature human GDF peptide or a functional variant thereof. When present, the optional linker is preferably a flexible peptide linker. In particular embodiments, the nucleic acid encodes a fusion polypeptide that comprises A) an SA moiety selected from the group consisting of HSA(25-609) (SEQ ID NO:4), and HSA(25-609) in which Cys34 is replaced with Ser and Asn503 is replaced with Gln; and

B) a GDF15 moiety selected from the group consisting of:
human GDF15(197-308) (SEQ ID NO:5);
human GDF15(211-308) (amino acids 211-308 of SEQ ID NO:2);
human GDF15(197-308) (SEQ ID NO:5) in which Cys203 is replaced with Ser (C2035) and Cys210 is replaced with Ser (C210S); and
human GDF15(197-308) (SEQ ID NO:5) in which Cys273 is replaced with Ser (C273S).

If desired, the encoded fusion polypeptide can further comprise a linker that links the C-terminus of the SA moiety to the N-terminus of the GDF15 moiety. Preferably, the linker is selected from (GGGGS)n (SEQ ID NO: 303) and (GPPGS)n (SEQ ID NO:304) and (GPPGS)n (SEQ ID NO:304), wherein n is one to about 20. Preferred linkers include ((GGGGS)n (SEQ ID NO:303) and (GPPGS)n (SEQ ID NO:304), wherein n is 1, 2, 3 or 4.

For expression in host cells, the nucleic acid encoding a fusion polypeptide can be present in a suitable vector and after introduction into a suitable host, the sequence can be expressed to produce the encoded fusion polypeptide according to standard cloning and expression techniques, which are known in the art (e.g., as described in Sambrook, J., Fritsh, E. F., and Maniatis, T. Molecular Cloning: A Laboratory Manual 2^{nd}, ed., Cold Spring Harbor Laboratory, Cold Spring Harbor Laboratory Press, Cold Spring Harbor, N.Y., 1989). The invention also relates to such vectors comprising a nucleic acid sequence according to the invention.

A recombinant expression vector can be designed for expression of a GDF15 fusion polypeptide in prokaryotic (e.g., E. coli) or eukaryotic cells (e.g., insect cells, yeast cells, or mammalian cells). Representative host cells include many E. coli strains, mammalian cell lines, such as CHO, CHO-K1, and HEK293; insect cells, such as Sf9 cells; and yeast cells, such as S. cerevisiae and P. pastoris. Alternatively, the recombinant expression vector can be transcribed and translated in vitro, for example using T7 promoter regulatory sequences and T7 polymerase and an in vitro translation system. Vectors suitable for expression in host cells and cell-free in vitro systems are well known in the art. Generally such a vector contains one or more expression control elements that are operably linked to the sequence encoding the fusion polypeptide. Expression control elements include, for example, promoters, enhancers, splice sites, poly adenylation signals and the like. Usually a promoter is located upstream and operably linked to the nucleic acid sequence encoding the fusion polypeptide. The vector can comprise or be associated with any suitable promoter, enhancer, and other expression-control elements. Examples of such elements include strong expression promoters (e.g., a human CMV IE promoter/enhancer, an RSV promoter, SV40 promoter, SL3-3 promoter, MMTV promoter, or HIV LTR promoter, EF1 alpha promoter, CAG promoter) and effective poly (A) termination sequences. Additional elements that can be present in a vector to facilitate cloning and propagation include, for example, an origin of replication for plasmid product in E. coli, an antibiotic resistance gene as a selectable marker, and/or a convenient cloning site (e.g., a polylinker).

In another aspect of the instant disclosure, host cells comprising the nucleic acids and vectors disclosed herein are provided. In various embodiments, the vector or nucleic acid is integrated into the host cell genome, which in other embodiments the vector or nucleic acid is extra-chromosomal. If desired the host cells can be isolated.

Recombinant cells, such as yeast, bacterial (e.g., E. coli), and mammalian cells (e.g., immortalized mammalian cells) comprising such a nucleic acid, vector, or combinations of either or both thereof are provided. In various embodiments, cells comprising a non-integrated nucleic acid, such as a plasmid, cosmid, phagemid, or linear expression element, which comprises a sequence coding for expression of a fusion polypeptide comprising the human serum albumin or the functional variant thereof and human GDF15 protein or a functional variant thereof, are provided.

A vector comprising a nucleic acid sequence encoding a GDF15 fusion polypeptide provided herein can be introduced into a host cell using any suitable method, such as by transformation, transfection or transduction. Suitable methods are well known in the art. In one example, a nucleic acid encoding a fusion polypeptide comprising the human serum albumin or the functional variant thereof and human GDF15 protein or the functional variant thereof can be positioned in and/or delivered to a host cell or host animal via a viral vector. Any suitable viral vector can be used in this capacity.

The invention also provides a method for producing a fusion polypeptide as described herein, comprising maintaining a recombinant host cell comprising a recombinant nucleic acid of the invention under conditions suitable for expression of the recombinant nucleic acid, whereby the recombinant nucleic acid is expressed and a fusion polypeptide is produced. In some embodiments, the method further comprises isolating the fusion polypeptide.

### Therapeutic Methods and Pharmaceutical Compositions

The invention relates to methods for treating non-alcoholic fatty liver disease (NAFLD) and non-alcoholic steatohepatitis (NASH), as well as end-stage liver disease, hepatic steatosis (fatty liver), liver fibrosis, liver inflammation, liver cirrhosis, primary biliary cirrhosis (PBC), and hepatocellular carcinoma (HCC) in a subject in need thereof, said method comprising administering to the subject in need thereof an effective amount of, e.g, a GDF15 polypeptide, a GDF15 variant, GDF15 fusion polypeptide, or GDF15 FA conjugate (usually in the form of a pharmaceutical composition) as described herein.

Non-alcoholic fatty liver disease (NAFLD) is a disorder affecting as many as 1 in 3-5 adults and 1 in 10 children in the United States, and refers to conditions where there is an accumulation of excess fat in the liver of people who drink little or no alcohol. The most common form of NAFLD is a non-serious condition called hepatic steatosis (fatty liver), in which fat accumulates in the liver cells: although this is not normal, by itself it probably does not damage the liver. NAFLD most often presents itself in individuals with a constellation of risk factors called the metabolic syndrome, which is characterized by elevated fasting plasma glucose (FPG) with or without intolerance to post-prandial glucose, being overweight or obese, high blood lipids such as cholesterol and triglycerides (TGs) and low high-density lipoprotein cholesterol (HDL-C) levels, and high blood pressure; but not all patients have all the manifestations of the metabolic syndrome. Obesity is thought to be the most common cause of NAFLD; and some experts estimate that about two-thirds of obese adults and one-half of obese children may have fatty liver. The majority of individuals with NAFLD have no symptoms and a normal physical examination (although the liver may be slightly enlarged); children may exhibit symptoms such as abdominal pain and fatigue, and may show patchy dark skin discoloration (acanthosis nigricans). The diagnosis of NAFLD is usually first suspected in an overweight or obese person who is found to have mild elevations in their liver blood tests during routine testing, though NAFLD can be present with normal liver blood tests, or incidentally detected on imaging investigations such as abdominal ultrasound or CT scan. It is confirmed by imaging studies, most commonly a liver ultrasound or magnetic resonance imaging (MRI), and exclusion of other causes.

Some people with NAFLD may develop a more serious condition called non-alcoholic steatohepatitis (NASH): about 2-5 percent of adult Americans and up to 20 percent of those who are obese may suffer from NASH. In NASH, fat accumulation in the liver is associated with inflammation and different degrees of scarring. NASH is a potentially serious condition that carries a substantial risk of progression to end-stage liver disease, cirrhosis and hepatocellular carcinoma. Some patients who develop cirrhosis are at risk of liver failure and may eventually require a liver transplant.

NAFLD may be differentiated from NASH by the NAFLD Activity Score (NAS), the sum of the histopathology scores of a liver biopsy for steatosis (0 to 3), lobular inflammation (0 to 2), and hepatocellular ballooning (0 to 2). A NAS of <3 corresponds to NAFLD, 3-4 corresponds to borderline NASH, and >5 corresponds to NASH. The biopsy is also scored for fibrosis (0 to 4).

Non-alcoholic fatty liver disease (NAFLD) is a condition ranging from benign lipid accumulation in the liver (steatosis) to steatosis combined with inflammation. The latter is referred to as non-alcoholic steatohepatitis (NASH). NASH is viewed as the hepatic component of metabolic syndrome. Estimates from the USA are that 5.7% to 17% of all adults have NASH, while 17% to 33% of Americans have NAFLD [1, 2]. As obesity and insulin resistance reach epidemic proportions in industrialized countries, the prevalence of both NAFLD and NASH is increasing and is therefore considered to be a major health hazard [3]. Steatosis alone is considered a relatively benign condition for the liver itself and is also a reversible condition However, the transition towards NASH represents a key step in the pathogenesis, as it sets the stage for further damage to the liver, such as fibrosis, cirrhosis and liver cancer. While the mechanisms leading to steatosis are well described, little is known about the actual risk factors that drive hepatic inflammation during the progression to NASH. Consequently, therapeutic options are poor.

NASH is a leading cause of end-stage liver disease; while NAFLD, and to an even greater degree NASH, are intimately related to states of the metabolic syndrome, including insulin resistance (pre-diabetes) and type 2 diabetes mellitus (T2DM), and abdominal obesity. T2DM has been the most prominent predictor for a poor prognosis in NAFLD, whereas elevated liver enzymes are considered unreliable. NASH develops much more frequently in the presence of longstanding T2DM, and the majority of patients with cryptogenic cirrhosis are obese and/or diabetic. Studies have demonstrated that 60 percent of patients with T2DM and NAFLD had biopsy-proven NASH, and that advanced hepatic fibrosis was present in 75 percent of those with diabetes and hypertension compared to only 7 percent without either condition. Haukeland, “Abnormal glucose tolerance is a predictor of nonalcoholic steatohepatitis and fibrosis in patients with non-alcoholic fatty liver disease”, Scand J. Gastroenterol., 40, 1469-1477 (2005), reported that impaired glucose tolerance (IGT) and T2DM were the only independent risk factors for severe NAFLD and NASH, increasing the odds ratio almost 4-fold. Mofrad, “Clinical and histological spectrum of nonalcoholic fatty liver disease associated with normal ALT levels”, Hepatology, 37, 1286-1292 (2003), reported a study that demonstrated the lack of predictive value for elevated liver transaminases to diagnose NASH in patients with NAFLD and found T2DM to be the only factor independently associated with an increased risk of advanced fibrosis.

Thus, NASH is an overlooked complication of T2DM that is frequently associated with fibrosis and in approximately 10 percent of patients results in cirrhosis; while the risk of hepatocellular carcinoma is also increased in patients with T2DM and NASH. Patients with NAFLD and NASH usually demonstrate mixed dyslipidemia and the other metabolic derangements described above, including an atherogenic low-density lipoprotein (LDL) phenotype consisting of predominantly of small dense particles. Both metabolic syndrome and NAFLD/NASH are characterized by increased cardiovascular inflammation as measured by elevations in high sensitivity C-reactive protein (hsCRP) and other inflammatory cytokines.

“Non-alcoholic steatohepatitis” or NASH is a common liver disease, which resembles alcoholic liver disease, but occurs in people who drink little or no alcohol. The major feature in NASH is fat in the liver, along with inflammation and damage. NASH can lead to cirrhosis, in which the liver is permanently damaged and scarred and is no longer able to work properly. NASH affects 2 to 5 percent of the U.S. population. Currently, no specific therapies for NASH exist. An additional 10 to 20 percent of Americans have fat in their liver, but no substantial inflammation or liver damage, a condition called “non-alcoholic fatty liver disease” (NAFLD). Although having fat in the liver is not normal, by itself it probably causes little harm or permanent damage. If fat is suspected based on blood test results or scans of the liver, this problem is referred to as NAFLD. If a liver biopsy is performed in this case, it will show that some people have NASH while others have NAFLD.

NASH is usually first suspected in a person who is found to have elevations in liver tests that are included in routine blood test panels, such as alanine aminotransferase (ALT) or aspartate aminotransferase (AST). When further evaluation shows no apparent reason for liver disease (such as medications, viral hepatitis, or excessive use of alcohol) and when x rays or imaging studies of the liver show fat, NASH is suspected. NASH is diagnosed and separated from NAFLD by a liver biopsy. For a liver biopsy, a needle is inserted through the skin to remove a small piece of the liver. NASH is diagnosed when examination of the tissue with a microscope shows fat along with inflammation and damage to liver cells. If the tissue shows fat without inflammation and damage, NAFLD is diagnosed. An important piece of information learned from the biopsy is whether scar tissue has developed in the liver.

NASH can slowly worsen, causing scarring or fibrosis to appear and accumulate in the liver. As fibrosis worsens, cirrhosis develops; the liver becomes severely scarred, hardened, and unable to function normally. Once serious scarring or cirrhosis is present, few treatments can halt the progression. A person with cirrhosis experiences fluid retention, muscle wasting, bleeding from the intestines, and liver failure. Liver transplantation is the only treatment for advanced cirrhosis with liver failure, and transplantation is increasingly performed in people with NASH. For example, NASH ranks as one of the major causes of cirrhosis in the U.S.A., behind hepatitis C and alcoholic liver disease.

There are no drugs currently approved to prevent or treat NAFLD or NASH. A number of pharmacological interventions have been tried in NAFLD/NASH but with overall limited benefit. Antioxidant agents may arrest lipid peroxidation and cytoprotective agents stabilize phospholipid membranes, but agents tried unsuccessfully or with only modest benefit so far include ursodeoxycholic acid, vitamins E (a-tocopherol) and C, and pentoxifylline, among others. Weight-loss agents such as orlistat have had no significant benefit compared to just the use of diet and exercise to achieve weight loss (“weight loss alone”). Most weight-loss studies in NAFLD/NASH have been pilot studies of short duration and limited success, reporting only a modest improvement in necroinflammation or fibrosis. A randomized, double-blind, placebo-controlled 6-month trial (Belfort, “A placebo-controlled trial of pioglitazone in subjects with nonalcoholic steatohepatitis”, N. Engl. J. Med., 355, 2297-2307 (2006)) of weight loss alone against pioglitazone, a thiazolidinedione peroxisome proliferator-activated receptor-gamma (PPARgamma) agonist and insulin sensitizer, failed to demonstrate any improvement for weight loss alone, but treatment with pioglitazone improved glycemic control, insulin sensitivity, indicators of systemic inflammation (including hsCRP, tumor necrosis factor-a, and transforming growth factor-beta), and liver histology in patients with NASH and IGT or T2DM. Treatment with pioglitazone also ameliorated adipose, hepatic, and muscle IR, and was associated with an approximately 50 percent decrease in necroinflammation (p<0.002) and a 37 percent reduction in fibrosis (p=0.08) Improvement in hepatocellular injury and fibrosis has been recently reported in another controlled trial with pioglitazone of 12 months duration.

In contrast, while the first randomized clinical study with rosiglitazone, the other thiazolidinedione approved for diabetes treatment, in NASH demonstrated a reduction in IR, plasma alanine aminotransferase (ALT) levels and steatosis, rosiglitazone treatment had no significant effect on necrosis, inflammation, or fibrosis. A preliminary report of the 2-year, open-label follow-up of this trial was also disappointing, with no significant benefit from rosiglitazone treatment. Thus, the pharmacological agent with the most robust efficacy in NASH is pioglitazone. Unfortunately, pioglitazone is also associated with a significantly increased risk of weight gain, edema, congestive heart failure, and osteoporotic fractures in both women and men.

An effective amount of the fusion polypeptide, usually in the form of a pharmaceutical composition, is administered to a subject in need thereof. The fusion polypeptide can be administered in a single dose or multiple doses, and the amount administered and dosing regimen will depend upon the particular fusion protein selected, the severity of the subject's condition and other factors. A clinician of ordinary skill can determined appropriate dosing and dosage regimen based on a number of other factors, for example, the individual's age, sensitivity, tolerance and overall well-being.

The administration can be performed by any suitable route using suitable methods, such as parenterally (e.g., intravenous, subcutaneous, intraperitoneal, intramuscular, intrathecal injections or infusion), orally, topically, intranasally or by inhalation. Parental administration is generally preferred. Subcutaneous administration is preferred.

GDF15 conjugates or GDF15 fusion polypeptides of the present invention can be administered to the subject in need thereof alone or with one or more other agents. When the fusion polypeptide is administered with another agent, the agents can be administered concurrently or sequentially to provide overlap in the therapeutic effects of the agents. Examples of other agents that can be administered in combination with the fusion polypeptide include:

1. Antidiabetic agents, such as insulin, insulin derivatives and mimetics; insulin secretagogues such as the sulfonylureas (e.g., chlorpropamide, tolazamide, acetohexamide, tolbutamide, glyburide, glimepiride, glipizide); glyburide and Amaryl; insulinotropic sulfonylurea receptor ligands such as meglitinides, e.g. nateglinide and repaglinide; thiazolidinediones (e.g., rosiglitazone (AVANDIA), troglitazone (REZULIN), pioglitazone (ACTOS), balaglitazone, rivoglitazone, netoglitazone, troglitazone, englitazone, ciglitazone, adaglitazone, darglitazone that enhance insulin action (e.g., by insulin sensitization), thus promoting glucose utilization in peripheral tissues; protein tyrosine phosphatase-1B (PTP-1B) inhibitors such as PTP-112; Cholesteryl ester transfer protein (CETP) inhibitors such as torcetrapib, GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose cotransporter inhibitors such as T-1095; glycogen phosphorylase A inhibitors such as BAY R3401; biguanides such as metformin and other agents that act by promoting glucose utilization, reducing hepatic glucose production and/or diminishing intestinal glucose output; alpha-glucosidase inhibitors such as acarbose and migiitoi) and other agents that slow down carbohydrate digestion and consequently absorption from the gut and reduce postprandial hyperglycemia; GLP-1 (glucagon like peptide-1), GLP-1 analogs such as Exendin-4 and GLP-1 mimetics; and DPPIV (dipeptidyl peptidase IV) inhibitors such as vildagliptin;

2. Hypolipidemic agents such as 3-hydroxy-3-methyl-glutaryl coenzyme A (HMG-CoA) reductase inhibitors, e.g. lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; bile acid sequenstrants, such as cholestyramine and colesevelam; fibrates; nicotinic acid and aspirin;

3. Anti-obesity agents such as orlistat, rimonabant, phentermine, topiramate, qnexa, and locaserin;

4. Anti-hypertensive agents, e.g. loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibitors such as sacubitril; ACE/NEP inhibitors such as omapatrilat, sampatrilat and fasidotril; angiotensin II antagonists such as candesartan, eprosartan, irbesartan, losartan, telmisartan and valsartan, in particular valsartan; combinantions of NEP inhibitors and angiotensin II antagonists such as sacubitril and valsartan (i.e. Entresto); renin inhibitors such as ditekiren, zankiren, terlakiren, aliskiren, RO 66-1132 and RO-66-1168; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors;

5. Agonists of peroxisome proliferator-activator receptors, such as fenofibrate, pioglitazone, rosiglitazone, tesaglitazar, BMS-298585, L-796449, the compounds specifically described in the patent application WO 2004/103995 i.e. compounds of examples 1 to 35 or compounds specifically listed in claim 21, or the compounds specifically described in the patent application WO 03/043985 i.e. compounds of examples 1 to 7 or compounds specifically listed in claim 19 and especially (R)-1-{4-[5-methyl-2-(4-trifluoromethyl-phenyl)-oxazol-4-ylmethoxy]-benzenesulfonyl}-2,3-dihydro-1H-indole-2-carboxylic or a salt thereof; and

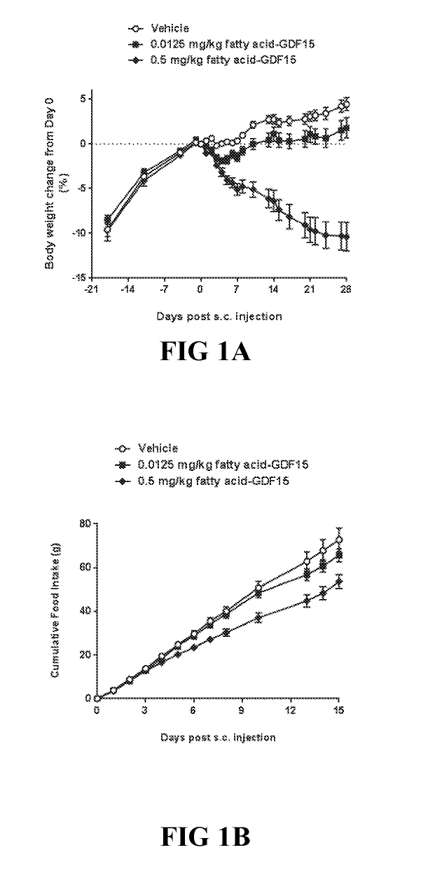

6. The specific anti-diabetic compounds described in Expert Opin Investig Drugs 2003, 12(4): 623-633, FIGS. 1 to 7.

The invention also relates to pharmaceutical compositions comprising a GDF15 conjugate or a GDF15 fusion polypeptide as described herein (e.g., comprising a fusion polypeptide comprising human serum albumin or a functional variant thereof and human GDF15 protein or a functional variant thereof). Such pharmaceutical compositions can comprise a therapeutically effective amount of the fusion polypeptide and a pharmaceutically or physiologically acceptable carrier. The carrier is generally selected to be suitable for the intended mode of administration and can include agents for modifying, maintaining, or preserving, for example, the pH, osmolarity, viscosity, clarity, color, isotonicity, odor, sterility, stability, rate of dissolution or release, adsorption, or penetration of the composition. Typically, these carriers include aqueous or alcoholic/aqueous solutions, emulsions or suspensions, including saline and/or buffered media.

Suitable agents for inclusion in the pharmaceutical compositions include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as free serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as Polysorbate 20 or Polysorbate 80; Triton; tromethamine; lecithin; cholesterol or tyloxapal), stability enhancing agents (such as sucrose or sorbitol), tonicity enhancing agents (such as alkali metal halides, such as sodium or potassium chloride, or mannitol sorbitol), delivery vehicles, diluents, excipients and/or pharmaceutical adjuvants

Parenteral vehicles include sodium chloride solution, Ringer's dextrose, dextrose and sodium chloride and lactated Ringer's. Suitable physiologically-acceptable thickeners such as carboxymethylcellulose, polyvinylpyrrolidone, gelatin and alginates may be included. Intravenous vehicles include fluid and nutrient replenishers and electrolyte replenishers, such as those based on Ringer's dextrose. In some cases it will be preferable to include agents to adjust tonicity of the composition, for example, sugars, polyalcohols such as mannitol, sorbitol, or sodium chloride in a pharmaceutical composition. For example, in many cases it is desirable that the composition is substantially isotonic. Preservatives and other additives, such as antimicrobials, antioxidants, chelating agents and inert gases, may also be present. The precise formulation will depend on the route of administration. Additional relevant principle, methods and components for pharmaceutical formulations are well known. (See, e.g., Allen, Loyd V. Ed, (2012) Remington's Pharmaceutical Sciences, 22th Edition)

When parenteral administration is contemplated, the pharmaceutical compositions are usually in the form of a sterile, pyrogen-free, parenterally acceptable composition. A particularly suitable vehicle for parenteral injection is a sterile, isotonic solution, properly preserved. The pharmaceutical composition can be in the form of a lyophilizate, such as a lyophilized cake.

In certain embodiments, the pharmaceutical composition is for subcutaneous administration. Suitable formulation components and methods for subcutaneous administration of polypeptide therapeutics (e.g., antibodies, fusion proteins and the like) are known in the art. See, e.g., Published United States Patent Application No 2011/0044977 and U.S. Pat. No. 8,465,739 and U.S. Pat. No. 8,476,239. Typically, the pharmaceutical compositions for subcutaneous administration contain suitable stabilizers (e.g, amino acids, such as methionine, and or saccharides such as sucrose), buffering agents and tonicifying agents.

### Definitions

The term “amino acid mimetic,” as used herein, refers to chemical compounds that have a structure that is different from the general chemical structure of an amino acid, but functions in a manner similar to a naturally occurring amino acid.

“Conservative” amino acid replacements or substitutions refer to replacing one amino acid with another that has a side chain with similar size, shape and/or chemical characteristics. Examples of conservative amino acid replacements include replacing one amino acid with another amino acid within the following groups: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

The term alkyl refers to a fully saturated branched or unbranched (or straight chain or linear) hydrocarbon moiety, comprising 1 to 50 carbon atoms. Preferably the alkyl comprises 5 to 20 carbon atoms, and more preferably 10 to 15 carbon atoms. For example C₁₀₋₁₅alkyl refers to an alkyl chain comprising 10 to 15 carbons. The term “alkylene” refer to a divalent alkyl as defined supra.

The term “alkenyl” refers to a branched or unbranched hydrocarbon having at least one carbon-carbon double bond. For example, the term “C₂₋₃₈-alkenyl” refers to a hydrocarbon having two to 38 carbon atoms and comprising at least one carbon-carbon triple

The term cycloalkyl refers to saturated or unsaturated but non-aromatic monocyclic, bicyclic or tricyclic hydrocarbon groups of 3-12 carbon atoms, preferably 3-8, or 3-7 carbon atoms. For bicyclic, and tricyclic cycloalkyl system, all rings are non-aromatic. For example, cycloalkyl encompasses cycloalkenyl and cycloalkynyl. The term “cycloalkenyl” refers to a bicyclic or tricyclic hydrocarbon group of 3-12 carbon atoms, having at least one carbon-carbon double bond. The term “cycloalkynyl” refers to a bicyclic or tricyclic hydrocarbon group of 3-12 carbon atoms, having at least one carbon-carbon triple bond.

The term heteroaryl includes monocyclic or bicyclic heteroaryl, containing from 5-10 ring members selected from carbon atoms and 1 to 5 heteroatoms, and each heteroatoms is independently selected from O, N or S wherein S and N may be oxidized to various oxidation states. For bicyclic heteroaryl system, the system is fully aromatic (i.e. all rings are aromatic).

The term heterocyclyl refers to a saturated or unsaturated non-aromatic (partially unsaturated but non-aromatic) monocyclic, bicyclic or tricyclic ring system which contains at least one heteroatom selected from O, S and N, where the N and S can also optionally be oxidized to various oxidation states. In one embodiment, heterocyclyl moiety represents a saturated monocyclic ring containing from 5-7 ring atoms and optionally containing a further heteroatom, selected from 0, S or N. The heterocyclic ring may be substituted with alkyl, halo, oxo, alkoxy, haloalkyl, haloalkoxy. In other embodiment, heterocyclyl is di- or tricyclic. For polycyclic system, some ring may be aromatic and fused to saturated or partially saturated ring or rings. The overall fused system is not fully aromatic. For example, a heterocyclic ring system can be an aromatic heteroaryl ring fused with saturated or partially saturated cycloalkyl ring system.

The term aryl refers to monocyclic or bicyclic aromatic hydrocarbon groups having 6-10 carbon atoms in the ring portion. Representative examples of aryl are phenyl or naphthyl.

The term “effective amount” refers to an amount sufficient to achieve the desired therapeutic effect, under the conditions of administration, such as an amount sufficient to lower fasting plasma glucose (FPG), reduce weight, reduce blood lipids such as cholesterol and triglycerides (TGs), reduce liver enzymes, raise high-density lipoprotein cholesterol (HDL-C) levels, and lower blood pressure. For example, a “therapeutically-effective amount” of a GDF15 therapeutic agent administered to a patient exhibiting, suffering, or prone to suffer from NASH or NAFLD is such an amount which causes an improvement in the pathological symptoms, disease progression, physiological conditions associated with or induces resistance to succumbing to the afore mentioned disorders.

“Functional variant” and “biologically active variant” refer to a polypeptide that contains an amino acid sequence that differs from a reference polypeptide (e.g., HSA, human IgFc, human wild type mature GDF15 peptide) by sequence replacement, deletion, or addition (e.g. HSA or IgFc fusion polypeptide), and/or addition of non-polypeptide moieties (e.g. PEG, fatty acids) but retains desired functional activity of the reference polypeptide. The amino acid sequence of a functional variant can include one or more amino acid replacements, additions or deletions relative to the reference polypeptide, and include fragments of the reference polypeptide that retain the desired activity.

For example, a functional variant of SA (e.g., HSA) prolongs the serum half-life of the fusion polypeptides described herein in comparison to the half-life of GDF15, while retaining the reference GDF15 (e.g., human GDF15) polypeptide's activity (e.g., weight loss, appetite suppressing, insulin release, insulin sensitivity, and/or fat mass reduction) activity. Polypeptide variants possessing a somewhat decreased level of activity relative to their wild-type versions can nonetheless be considered to be functional or biologically active polypeptide variants, although ideally a biologically active polypeptide possesses similar or enhanced biological properties relative to its wild-type protein counterpart (a protein that contains the reference amino acid sequence).

“Identity” means, in relation to nucleotide or amino acid sequence of a nucleic acid or polypeptide molecule, the overall relatedness between two such molecules. Calculation of the percent sequence identity (nucleotide or amino acid sequence identity) of two sequences, for example, can be performed by aligning the two sequences for optimal comparison purposes (e.g., gaps can be introduced in one or both of a first and a second nucleic acid or amino acid sequence for optimal alignment). The nucleotides or amino acids at corresponding positions are then compared. When a position in the first sequence is occupied by the same nucleotide or amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two sequences can be determined using methods such as those described by the National Center for Biotechnology Information (http://www.ncbi.nlm.nih.gov/). For example, the percent identity between two sequences can be determined using Clustal 2.0 multiple sequence alignment program and default parameters. Larkin M A et al. (2007) “Clustal W and Clustal X version 2.0.” Bioinformatics 23(21): 2947-2948.

The term “moiety,” as used herein, refers to a portion of a fusion polypeptide (e.g., HSA-GDF15) or fatty acid-conjugate described herein (e.g., AHA-(200-308)-hGDF15). The fusion proteins used in the methods of the present invention include, e.g., a GDF15 moiety, which contains an amino acid sequence derived from GDF15, and an SA moiety, which contains an amino acid sequence derived from SA. The fatty acid conjugates used in the methods of the present invention include, e.g., a GDF15 moiety, which contains an amino acid sequence derived from GDF15, and an fatty acid moiety, e.g., a fatty acid comprising one of the Formulae further described herein. The term “moiety” can also refer to a linker or functional molecule (e.g., PEG) comprising a fatty acid conjugate or fusion protein used in the methods of the present invention. The fusion protein optionally contains a linker moiety, which links the GDF15 moiety and the SA moiety, in the fusion polypeptide.

Without wishing to be bound by any particular theory, it is believed that the GDF15 moiety confers biological function of decreasing appetite, promoting weight loss and treating obesity and other metabolic diseases, while the SA moiety prolongs the serum half-life, improves expression and stability of the fusion polypeptides described herein.

The term “naturally occurring” when used in connection with biological materials such as nucleic acid molecules, polypeptides, host cells, and the like, refers to materials that are found in nature and are not manipulated by man. Similarly, “non-naturally occurring” as used herein refers to a material that is not found in nature or that has been structurally modified or synthesized by man. When used in connection with nucleotides, the term “naturally occurring” refers to the bases adenine (A), cytosine (C), guanine (G), thymine (T), and uracil (U). When used in connection with amino acids, the term “naturally occurring” refers to the 20 conventional amino acids (i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y)), as well as selenocysteine, pyrrolysine (PYL), and pyrroline-carboxy-lysine (PCL).

“Nonalcoholic steatohepatitis (NASH)” is a liver disease, not associated with alcohol consumption, characterized by fatty change of hepatocytes, accompanied by intralobular inflammation and fibrosis.

NASH is a common, often “silent”, liver disease. It resembles alcoholic liver disease, but occurs in people who drink little or no alcohol. The major feature in NASH is fat in the liver, along with inflammation and damage. Most people with NASH feel well and are not aware that they have a liver problem. Nevertheless, NASH can be severe and can lead to cirrhosis in which the liver is permanently damaged and scarred and no longer able to function properly.

NAFLD is a fatty liver disease common in chronic liver disease subjects. Excess liver fat can lead to liver complications. While not alcohol-related, these conditions can be related to obesity, diet, and other health-related issues.

Individuals with elevated liver enzymes and/or one having a fatty liver (e.g. determined by ultrasound or fatty liver index) are considered to have NASH or NAFLD. A reduction in enzymes, fat, or fatty liver index is an indicator of an improving or corrected condition.

“Alcoholic steatohepatitis (ASH)” is Alcoholic steatohepatitis (ASH) is a serious complication of alcoholic liver disease. The diagnosis of ASH requires the association of steatosis, evidence of hepatocellular injury with ballooning degeneration, and polynuclear neutrophil infiltration on liver biopsy.

NASH and ASH are advanced stages of non-alcoholic fatty liver disease (NAFLD) and alcoholic fatty liver disease (AFLD). NAFLD is characterized by excessive fat accumulation in the liver (steatosis), without any other evident causes of chronic liver diseases (viral, autoimmune, genetic, etc.), and with an alcohol consumption <20-30 g/day. On the contrary, AFLD is defined as the presence of steatosis and alcohol consumption >20-30 g/day. The most common phenotypic manifestations of primary NAFLD/NASH are overweight/obesity, visceral adiposity, type 2 diabetes, hypertriglyceridemia and hypertension.

As used herein, the terms “variant,” “mutant,” as well as any like terms, when used in reference to GDF15 or SA or specific versions thereof (e.g., “GDF15 variant,” “human GDF15 variant,” etc.) define protein or polypeptide sequences that comprise modifications, truncations, deletions, or other variants of naturally occurring (i.e., wild-type) protein or polypeptide counterparts or corresponding native sequences. “GDF15 variant,” for instance, is described relative to the wild-type (i.e., naturally occurring) GDF15 protein as described herein and known in the literature.

A “subject” is an individual to whom a GDF15 fusion polypeptide or GDF15 conjugate (e.g., usually in the form of a pharmaceutical composition) is administered. The subject is preferably a human, but “subject” includes pet and livestock animals, such as cows, sheep, goats, horses, dogs, cats, rabbits, guinea pigs, rats, mice or other bovine, ovine, equine, canine, feline, rodent or murine species, poultry and fish.

The term “GDF15 therapeutic agent” as used herein means a GDF15 polypeptide, GDF15 variant, GDF15 fusion protein, or GDF15 conjugate (e.g., a GDF15 fatty acid conjugate), or a pharmaceutical composition comprising one or more of the same, that is administered to a subject in order to treat non-alcoholic fatty liver disease (NAFLD) and non-alcoholic steatohepatitis (NASH), as well as related conditions that include but are not limited to alcoholic steatohepatitis (ASH), end-stage liver disease, hepatic steatosis (fatty liver), liver fibrosis, liver inflammation, liver cirrhosis, primary biliary cirrhosis (PBC), and hepatocellular carcinoma (HCC), or similar condition.

The terms “conjugate” and “fatty acid conjugate” are used interchangeably and are intended to refer to the entity formed as a result of a covalent attachment of a polypeptide or protein (or fragment and/or variant thereof) and a fatty acid moiety, optionally via a linker.

A “ribonucleic acid” (RNA) is a polymer of nucleotides linked by a phosphodiester bond, where each nucleotide contains ribose or a modification thereof as the sugar component. Each nucleotide contains an adenine (A), a guanine (G), a cytosine (C), a uracil (U) or a modification thereof as the base. The genetic information in a mRNA molecule is encoded in the sequence of the nucleotide bases of the mRNA molecule, which are arranged into codons consisting of three nucleotide bases each. Each codon encodes for a specific amino acid of the polypeptide, except for the stop codons, which terminate translation (protein synthesis). Within a living cell, mRNA is transported to a ribosome, the site of protein synthesis, where it provides the genetic information for protein synthesis synthesis (translation). For a fuller description, see, Alberts B et al. (2007) Molecular Biology of the Cell, Fifth Edition, Garland Science.

As used herein, the term “polypeptide” refers to a polymer of amino acid residues linked by peptide bonds, whether produced naturally or synthetically. Polypeptides of less than about 10 amino acid residues are commonly referred to as “peptides.” The term “peptide” is intended to indicate a sequence of two or more amino acids linked by peptide bonds, wherein said amino acids may be natural or unnatural. The term encompasses the terms polypeptides and proteins, which may consist of two or more peptides held together by covalent interactions, such as for instance cysteine bridges, or non-covalent interactions. The art-recognized three letter or one letter abbreviations are used to represent amino acid residues that constitute the peptides and polypeptides of the invention. Except when preceded with “D”, the amino acid is an L-amino acid. When the one letter abbreviation is a capital letter, it refers to the D-amino acid. When the one letter abbreviation is a lower case letter, it refers to the L-amino acid. Groups or strings or amino acid abbreviations are used to represent peptides. Peptides are indicated with the N-terminus on the left and the sequence is written from the N-terminus to the C-terminus.

Peptides of the invention contain non-natural amino acids (i.e., compounds that do not occur in nature) and other amino acid analogs as are known in the art may alternatively be employed.

Certain non-natural amino acids can be introduced by the technology described in Deiters et al., J Am Chem Soc 125:11782-11783, 2003; Wang and Schultz, Science 301:964-967, 2003; Wang et al., Science 292:498-500, 2001; Zhang et al., Science 303:371-373, 2004 or in U.S. Pat. No. 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the non-natural amino acid of choice. Particular non-natural amino acids that are beneficial for purpose of conjugating moieties to the polypeptides of the invention include those with acetylene and azido side chains.

A “protein” is a macromolecule comprising one or more polypeptide chains. A protein may also comprise non-peptidic components, such as carbohydrate groups. Carbohydrates and other nonpeptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless. A protein or polypeptide encoded by a non-host DNA molecule is a “heterologous” protein or polypeptide.

An “isolated polypeptide or isolated protein” is a polypeptide or protein (for example GDF15) that is essentially free from cellular components, such as carbohydrate, lipid, or other proteinaceous impurities associated with the polypeptide in nature. Typically, a preparation of isolated polypeptide or protein contains the polypeptide or protein in a highly purified form, i.e., at least about 80% pure, at least about 90% pure, at least about 95% pure, greater than 95% pure, such as 96%, 97%, or 98% or more pure, or greater than 99% pure. One way to show that a particular protein preparation contains an isolated polypeptide or protein is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide or protein in alternative physical forms, such as dimers or alternatively glycosylated or derivatized forms. Preferably, the isolated polypeptide is substantially free from any other contaminating polypeptides or other contaminants that are found in its natural environment that would interfere with its therapeutic, diagnostic, prophylactic or research use.

One of ordinary skill in the art will appreciate that various amino acid substitutions, e.g, conservative amino acid substitutions, may be made in the sequence of any of the polypeptide or protein described herein, without necessarily decreasing its activity. As used herein, “amino acid commonly used as a substitute thereof” includes conservative substitutions (i.e., substitutions with amino acids of comparable chemical characteristics). For the purposes of conservative substitution, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Examples of amino acid substitutions include substituting an L-amino acid for its corresponding D-amino acid, substituting cysteine for homocysteine or other non-natural amino acids having a thiol-containing side chain, substituting a lysine for homolysine, diaminobutyric acid, diaminopropionic acid, ornithine or other non-natural amino acids having an amino containing side chain, or substituting an alanine for norvaline or the like.

The term “amino acid,” as used herein, refers to naturally occurring amino acids, unnatural amino acids, amino acid analogues and amino acid mimetics that function in a manner similar to the naturally occurring amino acids, all in their D and L stereoisomers if their structure allows such stereoisomeric forms Amino acids are referred to herein by either their name, their commonly known three letter symbols or by the one-letter symbols recommended by the IUPAC-IUB Biochemical Nomenclature Commission.

The term “naturally occurring” refers to materials which are found in nature and are not manipulated by man. Similarly, “non-naturally occurring,” “un-natural,” and the like, as used herein, refers to a material that is not found in nature or that has been structurally modified or synthesized by man. When used in connection with amino acids, the term “naturally occurring” refers to the 20 conventional amino acids (i.e., alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr)).

The terms “non-natural amino acid” and “unnatural amino acid,” as used herein, are interchangeably intended to represent amino acid structures that cannot be generated biosynthetically in any organism using unmodified or modified genes from any organism, whether the same or different. The terms refer to an amino acid residue that is not present in the naturally occurring (wild-type) protein sequence or the sequences of the present invention. These include, but are not limited to, modified amino acids and/or amino acid analogues that are not one of the 20 naturally occurring amino acids, selenocysteine, pyrrolysine (Pyl), or pyrroline-carboxy-lysine (Pcl, e.g., as described in PCT patent publication WO2010/48582). Such non-natural amino acid residues can be introduced by substitution of naturally occurring amino acids, and/or by insertion of non-natural amino acids into the naturally occurring (wild-type) protein sequence or the sequences of the invention. The non-natural amino acid residue also can be incorporated such that a desired functionality is imparted to the molecule, for example, the ability to link a functional moiety (e.g., PEG). When used in connection with amino acids, the symbol “U” shall mean “non-natural amino acid” and “unnatural amino acid,” as used herein.

The term “analogue” as used herein referring to a polypeptide or protein means a modified peptide or protein wherein one or more amino acid residues of the peptide/protein have been substituted by other amino acid residues and/or wherein one or more amino acid residues have been deleted from the peptide/protein and/or wherein one or more amino acid residues have been added the peptide/protein. Such addition or deletion of amino acid residues can take place at the N-terminal of the peptide and/or at the C-terminal of the peptide.

The terms “GDF15 polypeptide” and “GDF15 protein” are used interchangeably and mean a naturally-occurring wild-type polypeptide expressed in a mammal, such as a human or a mouse. For purposes of this disclosure, the term “GDF15 protein” can be used interchangeably to refer to any full-length GDF15 polypeptide, which consists of 308 amino acid residues; (NCBI Ref. Seq. NP_004855.2) containing a 20 amino acid signal peptide, a 167 amino acid pro-domain, and a mature domain of 112 amino acids which is excised from the prodomain by furin-like proteases. A 308-amino acid GDF15 polypeptide is referred to as “full-length” GDF15 polypeptide; a 112 amino acids GDF15 polypeptide (e.g. amino acids 197-308) is a “mature” GDF15 polypeptide. The mature GDF15 peptide contains the seven conserved cysteine residues required for the formation of the cysteine knot motif (having three intrachain disulfide bonds) and the single interchain disulfide bond that are typical for TGFβ superfamily members. The mature GDF15 peptide contains two additional cysteine residues that form a fourth intrachain disulfide bond and N-terminal loop. A GDF15 protein or polypeptide therefore also includes multimer, more particularly dimer of the protein.

The term “GDF15 variant” encompasses a GDF15 polypeptide in which a naturally occurring GDF15 polypeptide sequence has been modified. Such modifications include, but are not limited to, one or more amino acid substitutions, including substitutions with non-naturally occurring amino acids non-naturally-occurring amino acid analogs and amino acid mimetics.

In one aspect, the term “GDF15 variant” refers to a GDF15 protein sequence in which at least one residue normally found at a given position of a native GDF15 polypeptide is deleted or is replaced by a residue not normally found at that position in the native GDF15 sequence. In some cases it will be desirable to replace a single residue normally found at a given position of a native GDF15 polypeptide with more than one residue that is not normally found at the position; in still other cases it may be desirable to maintain the native GDF15 polypeptide sequence and insert one or more residues at a given position in the protein; in still other cases it may be desirable to delete a given residue entirely; all of these constructs are encompassed by the term “GDF15 variant. The methods of the present invention also encompass nucleic acid molecules encoding such GDF15 variant polypeptide sequences.

In various embodiments, a GDF15 variant comprises an amino acid sequence that is at least about 85 percent identical to a naturally-occurring GDF15 protein. In other embodiments, a GDF15 polypeptide comprises an amino acid sequence that is at least about 90%, or about 95%, 96%, 97%, 98%, or 99% identical to a naturally-occurring GDF15 polypeptide amino acid sequence. Such GDF15 mutant polypeptides preferably, but need not, possess at least one activity of a wild-type GDF15 mutant polypeptide, such as the ability to lower blood glucose, insulin, triglyceride, or cholesterol levels; the ability to reduce body weight; or the ability to improve glucose tolerance, energy expenditure, or insulin sensitivity; the ability to treat, prevent, or ameliorate non-alcoholic fatty liver disease (NAFLD) and non-alcoholic steatohepatitis (NASH), as well as end-stage liver disease, hepatic steatosis (fatty liver), liver fibrosis, liver inflammation, liver cirrhosis, primary biliary cirrhosis (PBC), and hepatocellular carcinoma (HCC).

Although the GDF15 polypeptides and GDF15 mutant polypeptides, and the constructs comprising such polypeptides are primarily disclosed in terms of human GDF15, the invention is not so limited and extends to GDF15 polypeptides and GDF15 mutant polypeptides and the constructs comprising such polypeptides where the GDF15 polypeptides and GDF15 mutant polypeptides are derived from other species (e.g., cynomolgous monkeys, mice and rats). In some instances, a GDF15 polypeptide or a GDF15 mutant polypeptide can be used to treat or ameliorate a metabolic disorder in a subject in a mature form of a GDF15 mutant polypeptide that is derived from the same species as the subject.

A GDF15 mutant polypeptide is preferably biologically active. In various respective embodiments, a GDF15 polypeptide or a GDF15 mutant polypeptide has a biological activity that is equivalent to, greater to or less than that of the naturally occurring form of the mature GDF15 protein. Examples of biological activities include the ability to lower blood glucose, insulin, triglyceride, or cholesterol levels; the ability to reduce body weight; or the ability to improve glucose tolerance, lipid tolerance, or insulin sensitivity; the ability to lower urine glucose and protein excretion; the ability to treat non-alcoholic fatty liver disease (NAFLD) and non-alcoholic steatohepatitis (NASH), as well as end-stage liver disease, hepatic steatosis (fatty liver), liver fibrosis, liver inflammation, liver cirrhosis, primary biliary cirrhosis (PBC), and hepatocellular carcinoma (HCC).

As used herein in the context of the structure of a polypeptide or protein, the term “N-terminus” (or “amino terminus”) and “C-terminus” (or “carboxyl terminus”) refer to the extreme amino and carboxyl ends of the polypeptide, respectively.

The term “therapeutic polypeptide” or “therapeutic protein” as used herein means a polypeptide or protein which is being developed for therapeutic use, or which has been developed for therapeutic use.

### EXAMPLES

The following examples, including the experiments conducted and results achieved, are provided for illustrative purposes only and are not to be construed as limiting the present invention.

### Example 1: GDF15-Conjugate Improves Measures of Metabolic Disease Including Diabetes and Fatty Liver Disease in Obese Mice

Diet-induced obese mice were dosed once weekly with vehicle or fatty acid-GDF15 (0.5 mg/kg/s.c.) for 4 weeks. Non-fasted glucose and insulin were measured 2 weeks after the first dose, and overnight fasted blood glucose and insulin were measured 4 weeks after the first dose. Fatty acid-GDF15 reduced non-fasted glucose by 23% (207.1 mg/dl vehicle treated vs. 160.4 mg/dl fatty acid-GDF15; p<0.05). Fatty acid-GDF15 reduced non-fasted insulin levels by 75% compared to vehicle treated mice (2.1 vs 8.7 ng/ml; p<0.05). Four weeks after the initial dose, fatty acid-GDF15 reduced fasting blood glucose by 28% (142.7 vs. 199.5 mg/dl; p<0.05) and fasting insulin by 78% (0.77 vs. 3.5 ng/ml; p<0.05). Markers of fatty liver disease were also improved by four, once-weekly doses of fatty acid-GDF15. Fatty acid-GDF15 reduced hepatic steatosis by 57.5% (11.36 vs. 26.73% liver fat; p<0.05) and serum levels of a marker of hepatocyte damage, alanine aminotransferase (ALT), by 58% (46.2 vs. 110.5 U/L; p<0.05). In addition, fatty acid-GDF15 was shown to decrease the hepatic expression of PNPLA3, a causative gene in progressive liver diseases, by 77% (p<0.05), and to decrease the hepatic expression of COL1A1, or type I collagen, whose production correlates with liver fibrosis, by 57% (p<0.05). Treatment of diet-induced obese mice with a mouse serum albumin-GDF15 fusion protein (SEQ ID NO: 9) gave similar results for all of the study endpoints noted above for fatty acid-GDF15.

### Example 2: Fatty Acid-GDF15 Conjugate Improves Measures of Metabolic Disease Including Fatty Liver in Leptin-Deficient Ob/Ob Mice

Leptin-deficient obese (ob/ob) mice are predisposed to hepatic steatosis on a normal chow diet. However, on a diet high in trans-fat, fructose, and cholesterol (e.g. D09100301, Research Diets, Inc., New Brunswick, N.J.) the hepatic steatosis is exacerbated and liver fibrosis develops. Body weight as well as circulating cholesterol and liver enzymes also increase when ob/ob mice are fed the high trans-fat, fructose, and cholesterol diet. As such, this mouse strain and diet combination has been studied as a model of human NAFLD and NASH that is responsive to pharmacological intervention (Trevaskis J L, et al. (2012) Am J Physiol Gastrointest Liver Physiol 302:G762-G772). Exogenous GDF15 induces hypophagia and body weight loss in ob/ob mice (Johnen H, et al. (2007) Nat Med 13:1333-1340). We therefore tested the effect of GDF15 protein administration on measures of metabolic disease including fatty liver in ob/ob mice on the NAFLD and NASH-inducing high trans-fat, fructose, and cholesterol diet.

Six Week Study of Fatty Acid-GDF15 in Leptin-Deficient Ob/Ob Mice on a High Trans-Fat, High Fructose, and High Cholesterol Diet.

Leptin-deficient obese (ob/ob) mice were fed a diet high in trans-fat, fructose and cholesterol (D09100301, Research Diets, Inc., New Brunswick, N.J.) for 6 weeks. Following two weeks on diet, mice were injected subcutaneously once a week with either vehicle (30 mM NaOAc, pH 4.0), 0.0125 mg/kg fatty acid-GDF15 conjugate or 0.5 mg/k fatty acid-GDF15 conjugate for 4 weeks. Body weight and food intake were measured once weekly. Serum was collected two and four weeks post-treatment for serum chemistry analysis. Animals were subjected to a 4-hour fast prior to serum collection. Four weeks after the first injection, treatment with 0.0125 mg/kg fatty acid-GDF15 conjugate resulted in a 3% vehicle-adjusted body weight loss while treatment with 0.5 mg/kg fatty acid-GDF15 conjugated resulted in a 15% vehicle-adjusted weight loss (FIG. 1A). Body weight loss was consistent with a drop in cumulative food intake compared to vehicle controls during the first two weeks of treatment (10% in 0.0125 mg/kg group, 30% in 0.5 mg/kg group) (FIG. 1B).

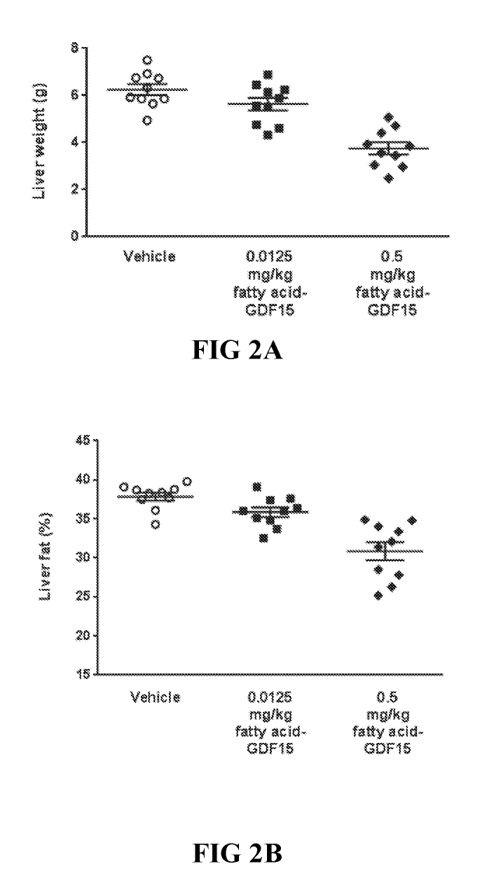

Compared to vehicle-treated mice, treatment with 0.0125 mg/kg fatty acid-GDF15 conjugate reduced total liver weight and hepatic steatosis (measured on a Bruker minispec body composition analyzer) 10% and 5%, respectively while treatment with 0.5 mg/kg fatty acid-GDF15 conjugate reduced total liver weight and hepatic steatosis by 40% and 20%, respectively (FIGS. 2A-B). Treatment with 0.0125 mg/kg or 0.5 mg/kg fatty acid-GDF15 conjugate reduced serum markers of liver damage (ALT (30% and 45%, respectively), AST (29% and 31%, respectively) and ALP (12% and 28%, respectively)) compared to vehicle treated animals (measured on a Roche Diagnostics Cobas 6000 series analyzer) (Table 2). Treatment with 0.0125 mg/kg or 0.5 mg/kg fatty acid-GDF15 conjugate also reduced plasma triglyceride (13% and 36%, respectively) and cholesterol levels (4% and 18%, respectively) compared to vehicle treated animals (Table 3). Glucose levels were reduced by 17% in the 0.5 mg/kg group compared to vehicle treated animals.

TABLE 2
Terminal plasma liver enzyme levels

| | ALT (U/L) | AST (U/L) | ALP (U/L) |
|---|---|---|---|
| Treatment | mean (SEM) | mean (SEM) | mean (SEM) |
| Vehicle | 2264.4 (498.7) | 1180.0 (217.3) | 427.1 (15.8) |
| 0.0125 | 1578.0 (145.4) | 834.0 (51.8) | 377.3 (15.2) |
| mg/kg fatty | | | |
| acid-GDF15 | | | |
| 0.5 mg/kg | 1242.0 (71.6) | 809.8 (52.0) | 308.0 (13.8) |
| fatty acid- | | | |
| GDF15 | | | |

TABLE 3
Terminal plasma glucose, triglyceride, and cholesterol levels

| | Glucose | Triglycerides | Cholesterol |
|---|---|---|---|
| | (mmol/L) | (mmol/L) | (mmol/L) |
| Treatment | mean (SEM) | mean (SEM) | mean (SEM) |
| Vehicle | 13.59 (0.58) | 1.03 (0.05) | 11.31 (0.37) |
| 0.0125 | 13.83 (0.94) | 0.90 (0.05) | 10.89 (0.37) |
| mg/kg fatty | | | |
| acid-GDF15 | | | |
| 0.5 mg/kg | 11.24 (0.69) | 0.66 (0.02) | 9.30 (0.55) |
| fatty acid- | | | |
| GDF15 | | | |

Sixteen Week Study of Fatty Acid-GDF15 in Leptin-Deficient Ob/Ob Mice on a High Trans-Fat, High Fructose, and High Cholesterol Diet.

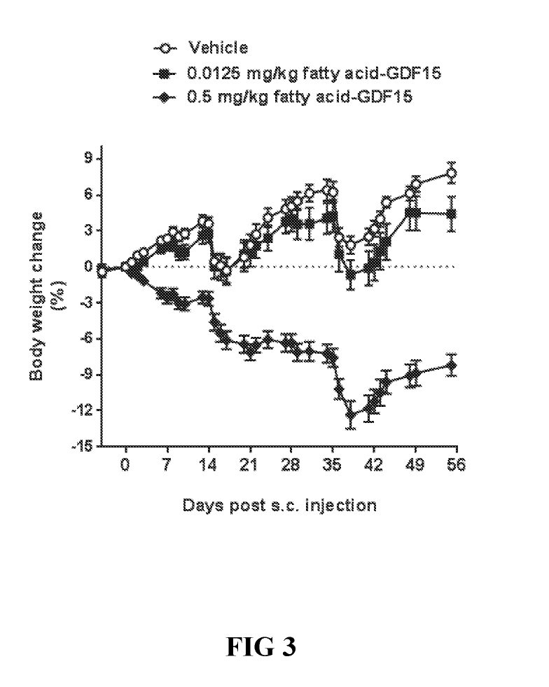

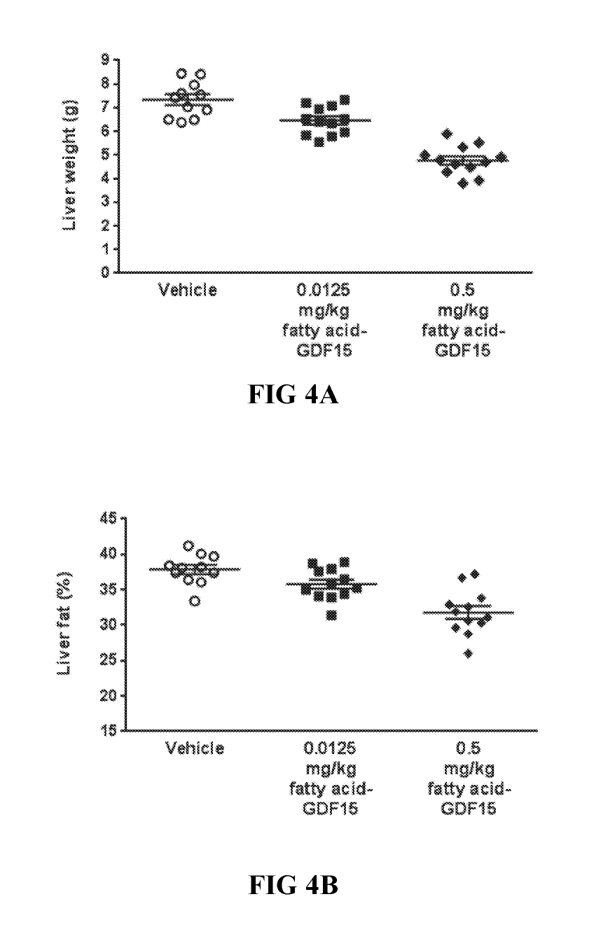

Leptin-deficient obese (ob/ob) mice were fed a diet high in trans-fat, fructose and cholesterol (D09100301, Research Diets, Inc., New Brunswick, N.J.) for 16 weeks. Following eight weeks on diet, mice were injected subcutaneously once a week with either vehicle (30 mM NaOAc, pH 4.0), 0.0125 mg/kg fatty acid-GDF15 conjugate or 0.5 mg/k fatty acid-GDF15 conjugate for eight weeks. Body weight and food intake were measured once weekly. Serum was collected two, five and 8 weeks post-treatment for serum chemistry analysis. Animals were subjected to a 4-hour fast prior to serum and tissue collection at eight weeks post-treatment. Eight weeks after the first injection, treatment with 0.0125 mg/kg fatty acid-GDF15 conjugate resulted in a 3% vehicle-adjusted body weight loss while treatment with 0.5 mg/kg fatty acid-GDF15 conjugated resulted in a 15% vehicle-adjusted weight loss (FIG. 3). Treatment with 0.0125 mg/kg or 0.5 mg/kg fatty acid-GDF15 conjugate reduced total liver weight (12% and 35%, respectively) compared to vehicle-treated mice (FIG. 4A). Hepatic steatosis was reduced by 16% in the 0.5 mg/kg group compared to vehicle treated controls (FIG. 4B). As seen in Table 4, ALP levels were also reduced by 10% in the 0.5 mg/kg group compared to vehicle treated controls.

TABLE 4
Terminal plasma alkaline phosphatase (ALP) levels

| Treatment | ALP (U/L) mean (SEM) |
|---|---|
| Vehicle | 361.1 (12.4) |
| 0.0125 | 337.8 (11.5) |
| mg/kg fatty | |
| acid-GDF15 | |
| 0.5 mg/kg | 325.3 (9.5) |
| fatty acid- | |
| GDF15 | |

### Example 3: Expression and Purification of HSA Fusion Polypeptides

A. Mammalian Cell Expression and Purification

Constructs of human GDF15 were expressed in transiently transfected HEK293F cells. Briefly, a liter of HEK293F cells 1 mg of DNA and 3 mg of linear 25 kDa polyethylenimine were mixed in 100 mL of medium, incubated at room temperature for 10 minutes, and then added to the cells. The cells were incubated for 5 days post transfection at 37° C. at 125 rpm (50 mm throw) at 8% CO₂ at 80% humidity. The cells were removed by centrifugation for 20 minutes at 6,000×g at 4° C. The supernatant was filtered through a 0.8/0.2 μm membrane and buffer exchanged into 100 mM TRIS pH 8.0 by TFF. The GDF15 constructs were captured on a Q Sepharose anion exchange column and eluted in a 10 column volume gradient from 0-400 mM NaCl in 100 mM TRIS pH 8.0. The fractions containing GDF15 were further purified by size exclusion chromatography in 1×DPBS, 1.47 mM KH₂PO₄, 8.06 mM Na₂HPO₄-7H₂O, 137.9 mM NaCl, 2.67 mM KCl. The fractions containing GDF15 were flask frozen in liquid nitrogen and stored at −80° C.

Mammalian Cell Expression and Purification of HSA-GDF15 Fusion

Constructs of His-human GDF15 fusion proteins were expressed in transiently transfected HEK293F cells. Briefly, per 2.5 liters of HEK293F cells 2.5 mg of DNA and 7.5 mg of linear 25 kDa polyethylenimine were mixed in 250 mL of medium, incubated at room temperature for 10 minutes, and then added to the cells. The cells were incubated for 4 days post transfection at 37° C. at 125 rpm (50 mm throw) at 8% CO₂ at 80% humidity. The cells were removed by centrifugation for 20 minutes at 6,000×g at 4° C. The supernatant was filtered through a 0.8/0.2 μm membrane. 1 M citric acid pH 3 was added to the filtered supernatant to a final concentration of 135 mM, solid sodium chloride was added to a final concentration of 2 M, and the supernatant was filtered through a 0.22 μm membrane. 5 mL of phenyl sepharose resin were equilibrated in 100 mM citric acid, 2 M NaCl, pH 3 and added to the supernatant. The resin was incubated with the supernatant for 2 hours at room temperature and packed into a 5 cm gravity column. The resin was washed with 20 mL of 100 mM citric acid, 2 M NaCl, pH 3; 20 mL of 100 mM citric acid, 1.5 M NaCl, pH 3; 100 mM citric acid, 1 M NaCl, pH 3; 100 mM citric acid, 0.5 M NaCl, pH 3; 100 mM citric acid, pH 3; 100 mM citric acid, 20% ethanol, pH 3; and 100 mM citric acid, 50% ethanol, pH 3. The washes containing no NaCl were pooled. 2 M TRIS base added to the phenyl sepharose pool to a final concentration of 180 mM yielding a final pH of 7.5. 5 M NaCl was added to a final concentration of 150 mM. 160 μL of Ni Sepharose HP resin were equilibrated in PBS, added to the phenyl sepharose pool, and incubated for 1 hour at room temperature. The resin was packed into a 1 cm gravity column and washed with 20 mL of PBS followed by 1 mL of PBS+100 mM imidazole. The bound protein was eluted in 1 mL of PBS+500 mM imidazole. The fractions containing GDF15 were flash frozen in liquid nitrogen and stored at −80° C.

B. Yeast Expression and Purification

Constructs of human GDF15 were expressed in Pichia pastoris utilizing methanol induction. Plasmid DNA was linearized with SacI for use in transformation. The linearized DNA was transformed into Pichia pastoris strain SMD1168 and expressed in BMMY medium at pH 6 with 1% (v/v) methanol at 30° C. at 200 rpm (1 inch throw) for 4 days. Methanol was added to a final concentration of 1% (v/v) each day during expression. The cells were removed by centrifugation for 20 minutes at 5,000×g at 4° C. and the supernatant was filtered through a 0.22 μm membrane. An equal volume of 1 M citric acid, 3 M NaCl pH 2.75 was added to the filtered supernatant. Phenyl Sepharose 6 was added to the supernatant and the GDF15 was bound by incubation for 1 hour at room temperature while stiffing. The resin was packed into a gravity column and the flow-through was removed. The resin was washed with 25 column volumes of 0.5 M citric acid, 1.5 M NaCl pH 3, 5 column volumes of 100 mM citric acid pH 3, and 5 column volumes of 100 mM citric acid, 20% ethanol pH 3. The bound protein was eluted in 5×1 column volume of 100 mM citric acid, 50% ethanol, pH 3. The elution fractions containing GDF15 were combined, diluted 1:10 into 25 mM bis-TRIS pH 5, and filtered through a 0.22 μm membrane. SP Sepharose cation exchange resin was added to the GDF15 and incubated for 1 hour at room temperature. The resin was packed into a gravity column and the flow-through was removed. The column was washed with 50 column volumes of 25 mM bis-TRIS pH 5 and eluted in 10 column volumes of 50 mM sodium phosphate, 150 mM NaCl pH 6.2.

C. E. coli Expression

E coli produced GDF15 was fused to a modified autoprotease P20 from Classical swine fever virus and expressed in inclusion bodies. E. coli transformed with GDF15 plasmid DNA were grown for 60 hours at 30° C. in ZYP-5052 auto induction medium (Studier F. W., Protein Expression and Purification 41 (2005) 207-234). The cell pellet was harvested by centrifugation for 30 minutes at 5,000×g at 18° C. Per liter of culture, the pellet was resuspended in 250 mL of 100 mM TRIS pH 8, 150 mM NaCl, 3 mM EDTA, 0.01% (v/v) Triton X-100, 1 mg/mL lysozyme and incubated for 20 minutes at room temperature, rotating. 250 mL of 100 mM TRIS pH 8, 150 mM NaCl, 20 mM CaCl₂, 20 mM MgCl₂, 0.25 mg/mL DNase I was added followed by an incubation for 20 minutes at room temperature, stiffing.

The pellet was centrifuged for 15 minutes at 5,000×g at 18° C. and the supernatant was discarded. The pellet was resuspended in 500 mL of 2% (v/v) Triton X-100 and incubated for 20 minutes at room temperature, rotating. The pellet was centrifuged for 15 minutes at 5,000×g at 18° C. and the supernatant was discarded. The pellet was resuspended in 500 mL of 500 mM NaCl and incubated for 20 minutes at room temperature, rotating. The pellet was centrifuged for 20 minutes at 5,000×g at 18° C. and the supernatant was discarded. The pellet was resuspended in 500 mL of 100 mM TRIS pH 8, 150 mM NaCl, 20 mM CaCl₂, 20 mM MgCl₂, 0.25 mg/mL DNase I and incubated for 20 minutes at room temperature, rotating. The pellet was centrifuged for 20 minutes at 5,000×g at 18° C. and the supernatant was discarded. The pellet was resuspended in 500 mL of 80% (v/v) ethanol and incubated for 20 minutes at room temperature, rotating. The pellet was centrifuged for 20 minutes at 5,000×g at 18° C. and the supernatant was discarded. The pellet was resuspended in 500 mL 100 mM TRIS pH 8, 500 mM NaCl, 8 M urea and incubated for 1 hour at room temperature, rotating. 10 mL of Ni Sepharose High Performance resin were added and incubated at room temperature for 1 hour, rotating.

The resin was packed into a gravity column and the flow-through was discarded. The resin was washed with 25 column volumes of 100 mM TRIS pH 8, 500 mM NaCl, 8 M urea the 25 column volumes of 100 mM TRIS pH 8, 1 M NaCl, 2 M urea. The bound protein was eluted in 2×5 column volumes of 100 mM TRIS pH 8, 1 M NaCl, 2 M urea, 0.5 M imidazole. The eluted protein was diluted 1:10 into 1 M TRIS-base, 1 M NaCl, 0.2 M histidine, 10 mM TCEP, pH 8.5. The sample was stirred briefly to mix and incubated overnight at room temperature with no agitation. The sample was loaded over a 6 gram HLB cartridge, washed in 100 mL of 0.1% (v/v) formic acid in water, and eluted in 50 mL of 0.1% (v/v) formic acid in isopropanol. The HLB elution was diluted 1:20 into 1 liter of 50 mM HEPES, 500 mM NaCl, 2 mM TCEP, 8 M urea, pH 7.6. 10 mL of Ni Sepharose High Performance resin were added and incubated at room temperature for 1 hour, stiffing. The resin was packed into a gravity column and the flow-though was saved.

The Ni flow-through was loaded over a 6 gram HLB cartridge, washed in 100 mL of 0.1% (v/v) formic acid in water, and eluted in 50 mL of 0.1% (v/v) formic acid in isopropanol. The second HLB elution was diluted 1:20 into 1 liter of 100 mM TRIS pH 8, 0.5 M urea, 2 mM oxidized glutathione, 2 mM reduced glutathione. The sample was stirred briefly to mix and incubated overnight at room temperature with no agitation. 100 mL of 5 M NaCl were added to make a final concentration of 500 mM and the sample was loaded over a 6 gram HLB cartridge. The cartridge was washed with 100 mL of 0.1% (v/v) formic acid in water and eluted in 25 mL of 0.1% (v/v) formic acid in ethanol. The HLB elution was diluted 1:4 by the addition of 75 mL of 50 mM bis-TRIS pH 4.8 and 1 mL of SP Sepharose resin was added. The resin was incubated with the GDF15 for 1 hour at room temperature and the packed into a gravity column. The resin was washed with 1 mL of 50 mM bis-TRIS pH 4.8 and eluted in 3×1 mL of PBS pH 6.4. Fractions 1 and 2 were combined, flash frozen in liquid nitrogen, and stored at −80° C.

### Animal Studies

Animal Studies: All animal studies described in this document were approved by the Novartis Institutes for Biomedical Research Animal Care and Use Committee in accordance with local and federal regulations and guidelines. Male mice (C57BL/6NTac) fed either a standard laboratory chow diet or a 60% fat diet (Research Diets D12492i) from 6-weeks of age onward were purchased from Taconic. Upon arrival, mice were housed one animal per cage typically under a 12h:12h reverse light-dark cycle. Animals all received a minimum of 1 week acclimation prior to any use. Mice were typically studied between 3-5 months of age. Prior to being studied, mice were randomized (typically 1-day prior to the experimental period) based on body weight such that each group had a similar average body weight.

Hydrodynamic DNA injections: On the day of study, mice were placed in fresh cages, and the old food removed. Each study animal (diet-induced obese male mice) received a single hydrodynamic injection of plasmid DNA via tail vein. DNA (typically 3 micrograms/mouse) was diluted in sterile saline at a volume ˜6.5% of the animal's body weight and rapidly injected within ˜5-10 seconds Immediately after injection, pre-weighed fresh high-fat diet was added to each cage at the end of the procedures above. Food intake and body weight were measured at the indicated time points.

Recombinant GDF15 analogs: On the day of study, mice were placed in fresh cages, and the old food removed. Approximately 1h later and just prior to the dark cycle, mice received a subcutaneous dose of either vehicle (1×PBS) or a GDF15 analog at the indicated times. After all injections are completed, the mice were reweighed and a defined amount of food returned (˜50 g per mouse of standard chow or high-fat diet). Food intake and body weight were measured over the course of the study at the times indicated.

Plasma GDF15 exposure: In surrogate animals treated as described above, plasma was collected into EDTA coated tubes at the indicated times, and human GDF15 levels were measured by ELISA as per the manufacturer's instructions (R&D Systems Quantikine Human GDF15 Immunoassay; DGD150). This assay does not recognize endogenous mouse GDF15.

Body composition: In some animals, body composition was assessed by NMR (Bruker MiniSpec Model LF90ii) as per the manufacturer's instructions. The mass of fat tissue, lean tissue and free fluid was calculated using MiniSpec software V.2.59.rev.6.

### Results

All mammalian cell expressed constructs were secreted using the mouse Igκ chain V-III region MOPC 63 signal peptide with the exception of the mouse albumin domain 1 fusion and the non 3×4GS linkers (SEQ ID NO: 300) which were secreted using the human CD8A signal peptide. Yeast expressed constructs were secreted using a modified mating factor alpha-1 signal peptide.

GDF15 can cause or promote weight loss agent in mice. However, characteristics of GDF15 make the naturally occurring peptide unsuitable for use as a therapeutic in humans, such as the short lived plasma half-life (˜1h) of the wild-type human peptide and poor expression levels in mammalian cells (Fairlie W D, et. al. Gene (2000) 254:67-76). To help understand whether GDF15 can be modified to improve its properties, e.g., extend its plasma half-life, the inventors solved the crystal structure of the protein. The GDF15 crystal structure revealed a unique disulfide pattern for GDF15 compared to other members of the TGFbeta superfamily that contain the 9 conserved cysteine residues, such as TGFB1-3 and inhibin beta (Galat A Cell. Mol. Life Sci. (2011) 68:3437-3451). To test the functional importance of these disulfide bonds, mammalian expression vectors were constructed that encoded proteins where each of the conserved cysteine residues that make up the disulfide bonds were individually mutated to serine residues. The expression constructs were delivered by hydrodynamic DNA injection to diet-induced obese mice as described in the Material and Methods section. Mice injected with the expression vector encoding naturally occurring GDF15 ate 31.1% less food and were 31.3% lighter 3 weeks post treatment compared to mice injected with the empty vector. Mice receiving the expression vector encoding mutations at C203S, C210S, or C273S ate 27.9, 28.0, and 33.9% less food and weighed 25.5, 20.4, and 30.3% less, respectively, than the control mice receiving the empty vector. Mice receiving the expression vector encoding C203S, C210S, and C273S ate 27.9, 28.0, and 33.9% less food and weighed 25.5, 20.4, and 30.3% less, respectively, than the control mice receiving the empty vector. Food intake and body weight were similar among empty vector treated mice and mice treated with an expression vector encoding C2115, C240S, C244S, C274S, C3055, or C3075. These data demonstrate that the first disulfide bond between C203 and C210 is not required for efficacy and suggest the amino-terminus of mature GDF15 can be manipulated. Interestingly, C273, which forms the interchain disulfide bond, is also not required for efficacy of GDF15.

The structural data combined with the functional data from the cysteine mutagenesis studies suggested that the amino terminus of GDF15 and potentially the carboxy terminus could be modified to extend the half-life of GDF15. To test this, mammalian expression vectors were constructed that encoded N-terminal Fc-GDF15 and C-terminal fusion proteins as well as mature GDF15 protein. Mice receiving a single hydrodynamic injection of an expression vector encoding mature GDF15 consistently ate approximately 25% less food than mice receiving a hydrodynamic injection of empty vector (Table 5). By the end of 4 weeks these mice weighed 28.9% less than the control mice (Table 6). Mice injected with a vector encoding an N-terminal Fc-GDF15 fusion protein ate about 25% less food over the first two weeks than the empty vector treated mice; however, by week 3 Fc-GDF15 treated mice were eating similar amounts of food as controls.

Body weights of Fc-GDF15 treated mice also initially decreased but then started to rebound such that by 4 weeks post injection, the Fc-GDF15 mice only weighed 9.8 percent less than empty vector treated mice. In contrast, mice injected with a vector encoding a C-terminal GDF15-Fc fusion protein consumed similar levels of food and gained weight exactly like empty vector treated mice throughout the duration of the experiment. High plasma GDF15 levels were detected at 1 and 3 weeks post injection for the mature GDF15 treated group (2.6 and 1.8 nM, respectively). Plasma GDF15 levels were 2.8 nM one week post dose but were undetectable 3 weeks post injection of the vector encoding Fc-GDF15. No GDF15 was detected at any time in mice treated with the GDF15-Fc expression vector. In summary, these data indicate that the C-terminal fusion of GDF15 was inactive, while N-terminal fusion of GDF15 was active. However, the loss of expression of GDF15 in the Fc-GDF15 fusion group suggests that Fc fusions to GDF15 may not be suitable therapeutics.

TABLE 5
Weekly Food Consumption (gram)

| | Empty Vector | Mature GDF15 | Fc-GDF15 | GDF15-Fc |
|---|---|---|---|---|
| Week 1 | 15.1 ± 0.62 | 11.6 ± 0.34 (−22.3) | 11.7 ± 0.52 (−22.9) | 15.7 ± 0.69 (3.8) |
| Week 2 | 17.4 ± 0.73 | 13.1 ± 0.47 (−24.7) | 13.1 ± 2.64 (−24.8) | 17.5 ± 0.72 (0.2) |
| Week 3 | 18.0 ± 0.56 | 13.7 ± 0.51 (−24.1) | 16.8 ± 0.49 (−6.4) | 18.6 ± 0.54 (3.4) |
| Week 4 | 18.4 ± 0.6 | 14.1 ± 0.62 (−23.4) | 17.6 ± 0.18 (−4.3) | 18.1 ± 0.52 (−1.5) |

Mean ± SEM (Percent Change Relative to Empty Vector)

TABLE 6
Body Weight (grams)

| | Empty Vector | Mature GDF15 | Fc-GDF15 | GDF15-Fc |
|---|---|---|---|---|
| Baseline | 31.1 ± 1.1 | 31.7 ± 0.8 | 31.0 ± 0.7 | 31.6 ± 0.8 |
| Week 1 | 30.7 ± 1.1 | 28.9 ± 0.7 | 28.3 ± 0.8 | 31.7 ± 1.1 |
| Week 2 | 32.5 ± 1.5 | 27.7 ± 0.5 | 29.4 ± 0.6 | 33.3 ± 1.1 |
| Week 3 | 34.2 ± 1.7 | 26.6 ± 0.5 | 30.9 ± 0.5 | 35.5 ± 1.2 |
| Week 4 | 36.7 ± 1.8 | 26.1 ± 0.6 | 33.1 ± 0.6 | 37.3 ± 1.4 |

Mean ± SEM

Based upon the opposing dimerization orientations of Fc and GDF15 and the loss of detectable plasma GDF15 in the Fc-GDF15 group, we suspected that Fc-GDF15 fusion proteins would be prone to aggregation, likely resulting in animals mounting an immune response against the Fc-GDF15 fusion protein. To determine if Fc-GDF15 fusion proteins are prone to aggregation, an Fc-GDF15 fusion protein was expressed in HEK293 cells. While the Fc-GDF15 fusion protein was expressed, a large proportion of the protein migrated close to the origin when analyzed under non-reducing conditions on a polyacrylamide gel, consistent with aggregation of the protein. (FIG. 1a) Further analysis by size exclusion chromatography confirmed the protein was aggregated.

In studies to identify GDF15 fusion proteins that were active but did not aggregate mammalian expression vectors encoding an N-terminal human serum albumin-[GGGGS]₃-GDF15 (HSA-GDF15) fusion protein and a mouse serum albumin-[GGGGS]₃-GDF15 (MSA-GDF15) were transfected into HEK293 cells. Unlike the Fc-GDF15 fusion protein, both HSA-GDF15 and MSA-GDF15 migrated at the expected molecular weight when analyzed under non-reducing conditions on a polyacrylamide gel and by size exclusion chromatography. (FIG. 1b) Unexpectedly, expression of both albumin-GDF15 fusion proteins in mammalian cells was also about 1000× greater than that for the mature GDF15 protein.

To determine if fusion of albumin to the N-terminus of GDF15 resulted in an active protein, lean mice were dosed with a single subcutaneous injection of vehicle or 99 micrograms (˜0.6 nmol of dimer) of MSA-GDF15 (197-308), MSA-GDF15 (197-308, C203S, C210S), MSA-GDF15 (211-308), or MSA-GDF15 (197-308, C273S). Compared to vehicle treated animals, food intake was reduced by 34, 34, 42, and 25 percent in animals receiving MSA-GDF15 (197-308), MSA-GDF15 (197-308, C203S, C210S), MSA-GDF15 (197-308, C273S), and MSA-GDF15 (211-308), respectively. These data clearly demonstrate that fusion of albumin to the N-terminus of GDF15 results in biologically active protein.

Fusion of albumin to the N-terminus of GDF15 also greatly increased the plasma half-life compared to the mature GDF15. The plasma half-life of mature GDF15 was ˜1h while the plasma half-life of the N-terminal serum albumin-GDF15 fusion proteins was ˜50h. Once weekly administration of MSA-GDF15 for 3 consecutive weeks greatly enhanced weight loss in obese mice compared to mature GDF15 at equivalent doses (0.6 nmol dimer/mouse, s.c.). Twenty eight days after the first dose and 2-weeks after the previous dose, MSA-GDF15 treated mice lost 12.8 percent of their starting body weight while, over the same duration, vehicle treated and GDF15 treated mice increased their starting body weight by an additional 10.9% and 5.6%, respectively. Analysis of body composition indicated that the weight loss induced by MSA-GDF15 is largely from fat mass with sparing of lean mass. On day 23 post initiation of dosing, the fat mass of MSA-GDF15 treated mice was 18.3% compared to 25.2% and 24.5% for vehicle and GDF15 treated mice, respectively. Lean mass in MSA-GDF15 treated mice was 55.6% of their body weight compared 51.5% and 52% for vehicle treated and GDF15 treated mice, respectively.

The HSA-GDF15 fusion was also biologically active. Obese mice receiving a single subcutaneous dose (3 mg/kg s.c.) of HSA-GDF15 ate 31% less food over 24h than vehicle-treated controls while MSA-GDF15 treated mice ate 27% less than vehicle controls. HSA-GDF15 fusions with different peptide linkers between albumin and GDF15 were also biologically active. Obese mice which were treated with a single subcutaneous dose (3 mg/kg s.c.) of HSA-no linker-GDF15, HSA-GGGGS-GDF15, HSA-GPPGS ate 22, 27, and 21% less food over 24 hours than vehicle treated mice. In summary, these data indicate that fusion of albumin to the N-terminus of GDF15 with various linkers are biologically active.

The amino terminus of GDF15 contains potential proteolytic (R198) and deamidation sites (N199) that may adversely impact development (e.g., stability) of a therapeutic albumin-GDF15 fusion protein. To determine if these sites are required for GDF15 activity, a series of albumin-GDF15 mutants were produced and tested for in vivo activity. Obese mice were treated with a single subcutaneous dose (3 mg/kg s.c.) of HSA-GDF15, HSA-GDF15 (R198H), HSA-GDF15 (N199E) or HSA-GDF15 (R198H, N199A). Cumulative food intake over the course of 6 days was reduced by 29% in mice treated with HSA-GDF15 compared to vehicle controls. Food intake over the same time period was reduced by 35, 28, and 25% in obese mice treated with HSA-GDF15 (R198H), HSA-GDF15 (N199E) or HSA-GDF15 (R198H, N199A) relative to controls. Over the 6 days, the body weight of vehicle treated animals increased by 6.1%, while body weight was reduced by 4.7% in HSA-GDF15 treated mice. Body weight was reduced by 5.2, 4.4, and 3.2 in obese mice treated with HSA-GDF15 (R198H), HSA-GDF15 (N199E) or HSA-GDF15 (R198H, N199A), respectively. Thus, fusion proteins containing mutation of these potential post-translational modification sites in the amino terminus of GDF15 retain biological activity.

As the receptor(s) for GDF15 is unknown, a series of structure-guided site-directed mutants were designed to elucidate domains and residues essential for function and those amenable to modification. GDF15 contains the fingers domain, knuckle domain, wrist domain, the newly discovered N-terminal loop domain, and back-of-hand domain. GDF15 analogs that disrupt the newly discovered amino-terminus region of GDF15, e.g. MSA-GDF15(211-308) and MSA-GDF15 (C203S, C210S), still retain biological activity demonstrating that this loop is not required for activity. The knuckle, finger, and wrist region of TGFbeta superfamily members are known to be important for receptor binding and signaling. To determine if these regions of GDF15 are critical for activity, key surface residues were mutated to a large side-chain containing amino acid, arginine, to attempt to induce a loss of function. MSA-GDF15 fusion proteins containing mutations in GDF15 residues leucine 294 (knuckle), aspartic acid 289 (fingers), glutamine 247 (wrist), and serine 278 (back of hand) were produced and then dosed subcutaneously to obese mice (3 mg/kg s.c.).

A single subcutaneous injection of MSA-GDF15 reduced food intake over the course of 7 days by 30% compared to vehicle control. Food intake was also reduced relative to control by the finger region mutant (D289R), the wrist mutant (Q247R), and the back of the hand mutant (S278R) by 22, 14, and 24%, respectively. In contrast, the knuckle region mutant (L294R) increased food intake by 17% relative to control. Over the course of the 7 days, body weight increased in the vehicle and L294R treated mice (2.2 and 6.3% respectively) while body weight decreased in by 6.6, 5.7, 5.7, and 5.4% in the MSA-GDF15, MSA-GDF15 (D289R), MSA-GDF15 (Q247R), and MSA-GDF15 (S278R) treated mice, respectively. These data indicate that L294 and the knuckle region of GDF15 are critical for activity, and likely interact with the GDF15 receptor. Mutations in the other regions of GDF15 are tolerated.

### Example 4: Variant of hGDF15 (AHA-hGDF15) Conjugated to a Fatty Acid

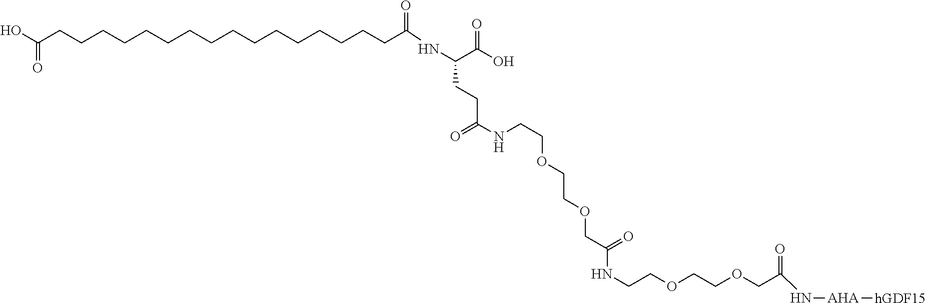

### Intermediate 1: AHA-(200-308)-hGDF15

(SEQ ID NO: 41)

AHAGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI.

LCMS: Calculated mass(dimer): 24430: Observed mass (dimer): 24432

Expression of Human GDF-15 Proteins in E. coli Cells

E. coli strains BL21 (DE3) Gold (Stratagene) and Rosetta (DE3) pLysS cells (Novagen) were transformed with constructs 51 to 56 and construct MAHA-(200-308)-hGDF15 respectively, cloned into pET26b vectors. Transformed cells were grown under antibiotic selection first in 3 ml and then in 50 ml Luria Broth (Bacto-Tryptone 10 g/L, yeast extract 5 g/L, NaCl 5/L, glucose 6 g/L) until an OD600 of 1.5 was reached. The pre-cultures were used to inoculate two 1-L fermenters filled with Terrific Broth medium (NH4SO4 1.2 g/L, H2PO4 0.041 g/L, K2HPO4 0.052 g/L, Bacto-Tryptone 12 g/L, Yeast Extract 24 g/L). The cultures were induced by automatic addition of 1 mM isopropyl-beta-D-thiogalactopyranoside (IPTG) when pH increased above 7.1. Other fermentation parameters were: temp=37° C.; pH 7.0+/−0.2 adjusted by addition of 2N NaOH/H2SO4; pO2>30% with cascades of stirrer speed, air flow and oxygen addition. Five hours post induction the cultures were cooled to 10° C. and cells were harvested by centrifugation.

Purification and Refolding of GDF15 Variant

Inclusion Bodies

Recombinant coli pellets expressing the protein of interest were resuspended (5% w/v) in 50 mM NaH₂PO₄/150 mM NaCl/5 mM benzamidine.HCl/5 mM DTT, pH 8.0 at 4° C., homogenized and lysed by 2 passages through a French press (800 and 80 bar). Inclusion bodies (IBs) were isolated by centrifugation at 12′000 rpm for 60 min at 4° C.

Purification of Crude Unfolded Protein

IBs were solubilized (5% w/v) in 6 M guanidine/100 mM NaH₂PO₄/10 mM Tris/20 mM beta-mercaptoethanol, pH 8.0 and stirred for 2 hours at room temperature. Debris was removed by centrifugation at 12′000 rpm. The solubilized IBs were further purified on Ni-NTA-Superflow (the construct without His tag binds as well to this resin due to the high histidine content). After base-line washing with 6 M guanidine/100 mM NaH₂PO₄/10 mM Tris/5 mM beta-mercaptoethanol, pH 8.0, bound material was eluted with the same buffer adjusted to pH 4.5. The eluate was adjusted to pH 8.0, 100 mM DTT was added and the solution stirred over night at 4° C. The pH was then adjusted to 2 by addition of trifluoroacetic acid (TFA, 10% stock in water) and the solution further diluted 1:1 with 0.1% TFA in water. The crude protein solution was further purified by RP-HPLC (Poros) using a gradient of 0-50% acetonitrile in 50 min. The GDF-15 containing fractions were pooled and lyophilized.

Protein Folding

Method 1: Lyophilized material was dissolved at 2 mg/ml in 100 mM acetic acid, diluted 15-20 folds in folding buffer (100 mM CHES/1 M NaCl/30 mM CHAPS/5 mM GSH/0.5 mM GSSG/20% DMSO, pH 9.5, 4° C.) and the solution gently stirred during 3 days at 4° C. After 3 days 3 volumes of 100 mM acetic acid was added and the solution concentrated by ultrafiltration (5 kDa cut-off) to about 100-200 ml, diluted 10 fold with 100 mM acetic acid and re-concentrated. The refolded material was further purified by preparative RP-HPLC on a Vydac C4 column run at 50° C. (buffer A: 0.1% TFA in water; buffer B: 0.05% TFA in acetonitrile). After loading the column was washed with 15% buffer B and eluted with a gradient of 15% B to 65% B in 50 min Collected fractions containing the protein of interest were diluted with an equal volume of buffer A and lyophilized. Refolding yields were about 25% for both proteins.

Method 2: Protocol followed as in method 1 with folding buffer: 100 mM CHES, pH 9.4, 0.9 M arginine, 0.5 M NaCl, 1 mM EDTA, 2.5 mM GSH, 1 mM GSSG (final concentration).

Intermediate 2: Synthesis of the Fatty Acid Construct

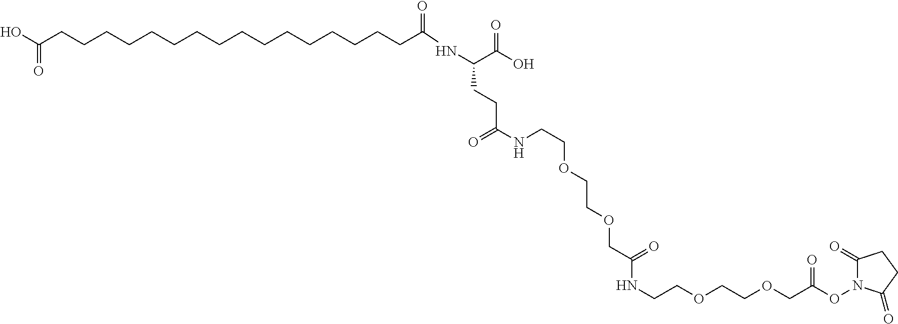 [SMILES: O=C(O)CCCCCCCCCCCCCCCCC(=O)N[C@@H](CCC(=O)NCCOCCOCC(=O)NCCOCCOCC(=O)ON1C(=O)CCC1=O)C(=O)O]

### Step 1: 18-(benzyloxy)-18-oxooctadecanoic acid, Dibenzyl octadecanedioate

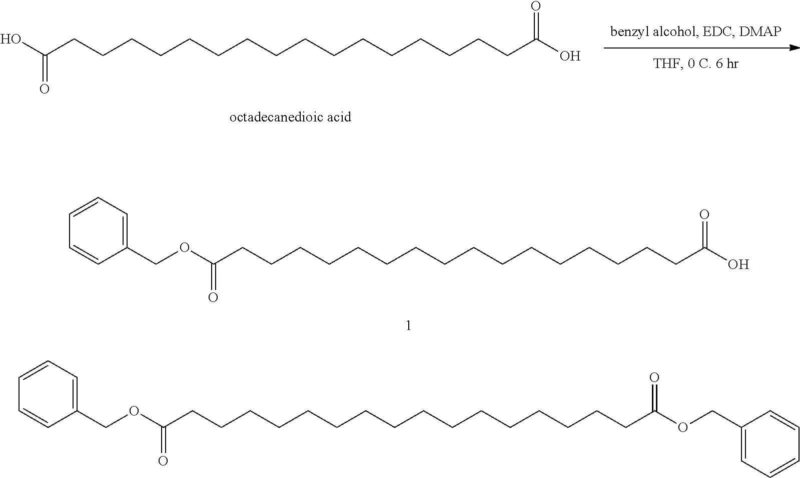 [SMILES: O=C(O)CCCCCCCCCCCCCCCCC(=O)O ; O=C(O)CCCCCCCCCCCCCCCCC(=O)OCc1ccccc1 ; O=C(CCCCCCCCCCCCCCCCC(=O)OCc1ccccc1)OCc1ccccc1]

A solution of octadecanedioic acid (100 mg, 0.318 mmol) in THF (Volume: 5 mL) was cooled to 0° and EDC (0.084 mL, 0.477 mmol) and DMAP (3.89 mg, 0.032 mmol) were added. Benzyl alcohol (0.030 mL, 0.286 mmol) was added slowly dropwise and the reaction was slowly warmed to r.t. and stirred for 16 hours. LCMS analysis showed 25% desired product and 38%+2 by ELSD (Method A (see Table 7, below, for this and all Methods cited in this example)), Rₜ prod=1.18 min, M+H 405.1; Rₜ+2prod=1.49 min, M+H₂O 512.4). Reaction mixture solvent was removed and crude material was taken up in DCM. The organics were washed with 1M HCl (aq) (25 mL×3), giving a white solid. The organic layer was then washed with a saturated aqueous solution of sodium carbonate (25 mL×3), using brine to aid the separation. The organics were dried over Na₂SO₄, filtered and concentrated to a white film.

The crude material was dissolved in a minimal amount of 2:1 ACN/DMSO and loaded onto a 20 g C18 15 uM column for reverse phase chromatography. Purified over 0-100% ACN/H₂O (0.1% TFA) 24 min gradient. Fractions with desired product were pooled, concentrated, frozen and lyophilized to give 38.3 mg of white powder (28%). The material was identified as the desired product 18-(benzyloxy)-18-oxooctadecanoic acid (1). LCMS Method B, Rₜ=2.39 min, M+H 405.4). 1H NMR (400 MHz, Chloroform-d) δ 7.39-7.32 (m, 5H), 5.11 (s, 2H), 2.35 (t, J=7.6 Hz, 4H), 1.67-1.61 (m, 4H), 1.36-1.21 (m, 24H).

### Step 2: 1-benzyl 18-(2,5-dioxopyrrolidin-1-yl) octadecanedioate

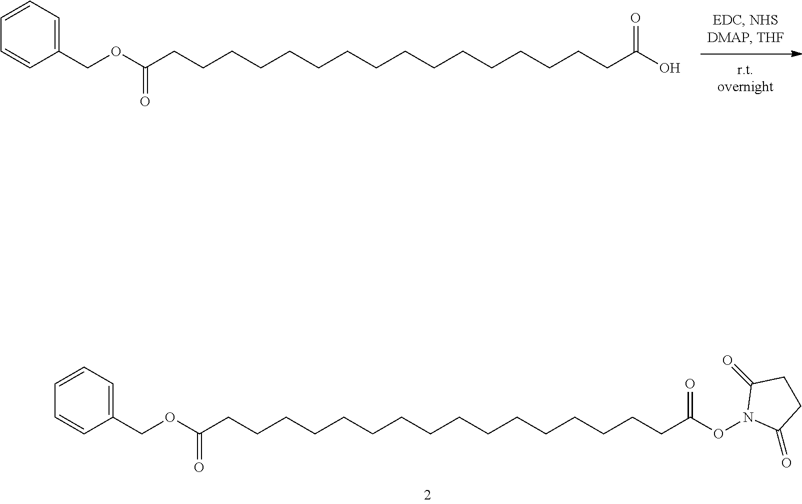 [SMILES: O=C(O)CCCCCCCCCCCCCCCCC(=O)OCc1ccccc1 ; O=C(CCCCCCCCCCCCCCCCC(=O)ON1C(=O)CCC1=O)OCc1ccccc1]

To a solution (1, 38.8 mg, 0.096 mmol) in THF (Volume: 2 mL) was added N-hydroxysuccinimide (13.24 mg, 0.115 mmol), EDC (0.025 mL, 0.144 mmol) and DMAP (1.172 mg, 9.59 μmol). The reaction was stirred at r.t. under N₂ for 16 hours. LCMS analysis showed full consumption of starting material and formation of desired product (Method A, Rₜ=1.25 min, M+H 502.4). The solvent was removed and reaction mixture taken up in a minimal amount of ACN and loaded onto a 20 g C18 15 uM column for reverse phase chromatography. Purified over 0-100% ACN/H₂O (0.1% TFA) 24 min gradient. Fractions with desired product were pooled, concentrated, frozen and lyophilized, giving a fine white solid (35.9 mg, 71%). LCMS Method B, Rₜ=2.51 min, M+H 502.5, M+H₂O 519.5. 1H NMR (400 MHz, Chloroform-d) δ 7.41-7.32 (m, 5H), 5.11 (s, 2H), 2.83 (s, 4H), 2.60 (t, J=7.4 Hz, 2H), 2.35 (t, J=7.5 Hz, 2H), 1.73 (m, J=7.6 Hz, 4H), 1.25 (d, J=3.6 Hz, 24H).

### Step 3: (S)-5-((benzyloxy)carbonyl)-1-(9H-fluoren-9-yl)-3,8-dioxo-2,12,15-trioxa-4,9-diazaheptadecan-17-oic Acid

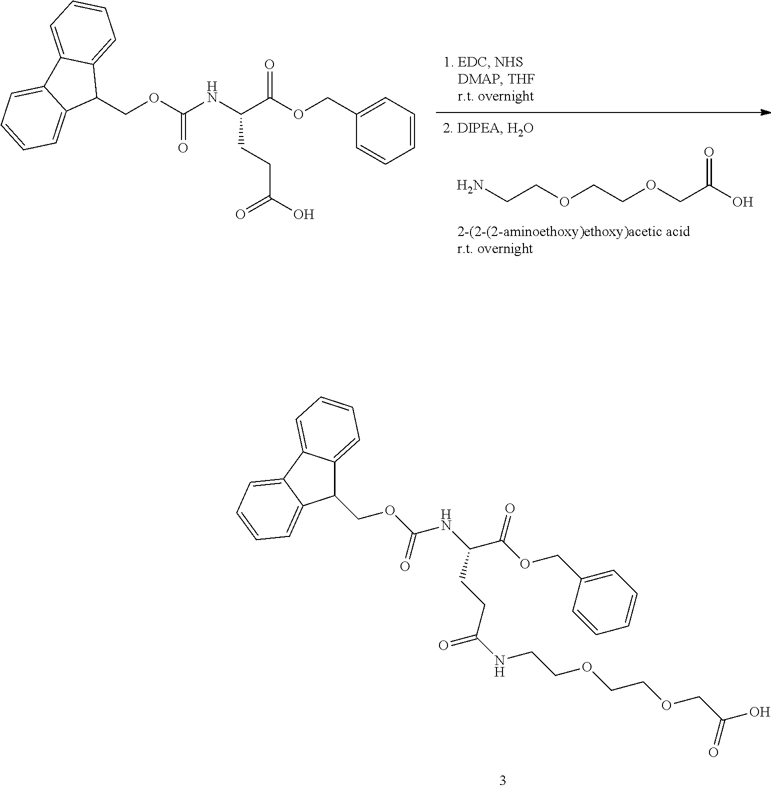 [SMILES: O=C(O)CC[C@H](NC(=O)OCC1c2ccccc2-c2ccccc21)C(=O)OCc1ccccc1 ; NCCOCCOCC(=O)O ; O=C(O)COCCOCCNC(=O)CC[C@H](NC(=O)OCC1c2ccccc2-c2ccccc21)C(=O)OCc1ccccc1]

To a solution of N-alpha-Fmoc-L-glutamic acid alpha-benzyl ester (250 mg, 0.544 mmol) in THF (Volume: 9 mL, Ratio: 9) was added N-hydroxy succinimide (75 mg, 0.653 mmol), EDC (0.144 mL, 0.816 mmol) and DMAP (6.65 mg, 0.054 mmol). The reaction was stirred at r.t. under N₂ for 3 hours. LCMS analysis showed presence of starting material (Method C, SM Rₜ=1.23 min, M+H 460.2; prod Rₜ=1.30 min, M+H 557.2) and so a 0.25 eq of N-hydroxysuccinimide was added and the reaction stirred at r.t. under N₂ for 16 hours. LCMS analysis showed full conversion to NHS ester product (Method C, Rₜ=1.30 min, M+H 557.2). 2-(2-(2-aminoethoxy)ethoxy)acetic acid (98 mg, 0.598 mmol) was dissolved in water (Volume: 1.000 mL, Ratio: 1.000) and added to the reaction mixture followed by DIPEA (0.442 mL, 2.72 mmol). The reaction mixture was stirred at r.t. under N₂ for 16 hours. LCMS analysis showed formation of desired product with some hydrolyzed glutamic acid (Method C, Rₜ=1.15 min, M+H 605.3). The reaction mixture (189 mg) was taken up in 5% MeOH/EtOAc and transferred to a separatory funnel, then washed 3× with 50 mL portions of 0.1N HCl. The combined aqueous layers were extracted once with 50 mL 5% MeOH/EtOAc and organic layers combined, dried over Na₂SO₄, filtered and concentrated to a colorless film (130 mg, 69%). LCMS analysis showed majority product in material (Method D, Rₜ=2.43 min, M+H 605.3). Material was carried on to next step without further purification.

### Step 4: (S)-4-amino-3,7-dioxo-1-phenyl-2,11,14-trioxa-8-azahexadecan-16-oic acid

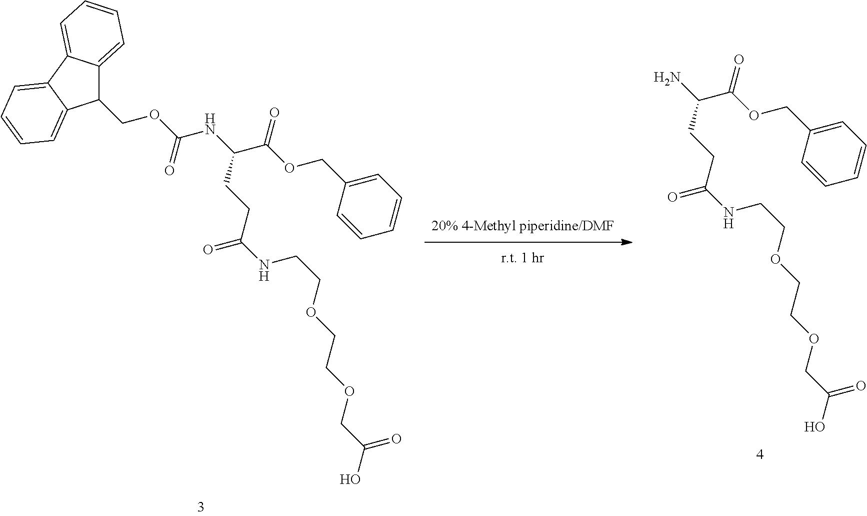 [SMILES: O=C(O)COCCOCCNC(=O)CC[C@H](NC(=O)OCC1c2ccccc2-c2ccccc21)C(=O)OCc1ccccc1 ; N[C@@H](CCC(=O)NCCOCCOCC(=O)O)C(=O)OCc1ccccc1]

3 is dissolved in 20% 4-Methyl piperidine/DMF solution (1 mL per 100 mg of material). The reaction is stirred at r.t. for one hour and then loaded onto 20 g C18 15 uM column for reverse phase chromatography. The crude material is purified over 0-100% ACN/H₂O (0.1% TFA) 24 min gradient. Fractions with desired product are pooled, concentrated, frozen and lyophilized.

### Step 5: (S)-22-((benzyloxy)carbonyl)-3,20,25-trioxo-1-phenyl-2,29,32-trioxa-21,26-diazatetratriacontan-34-oic acid

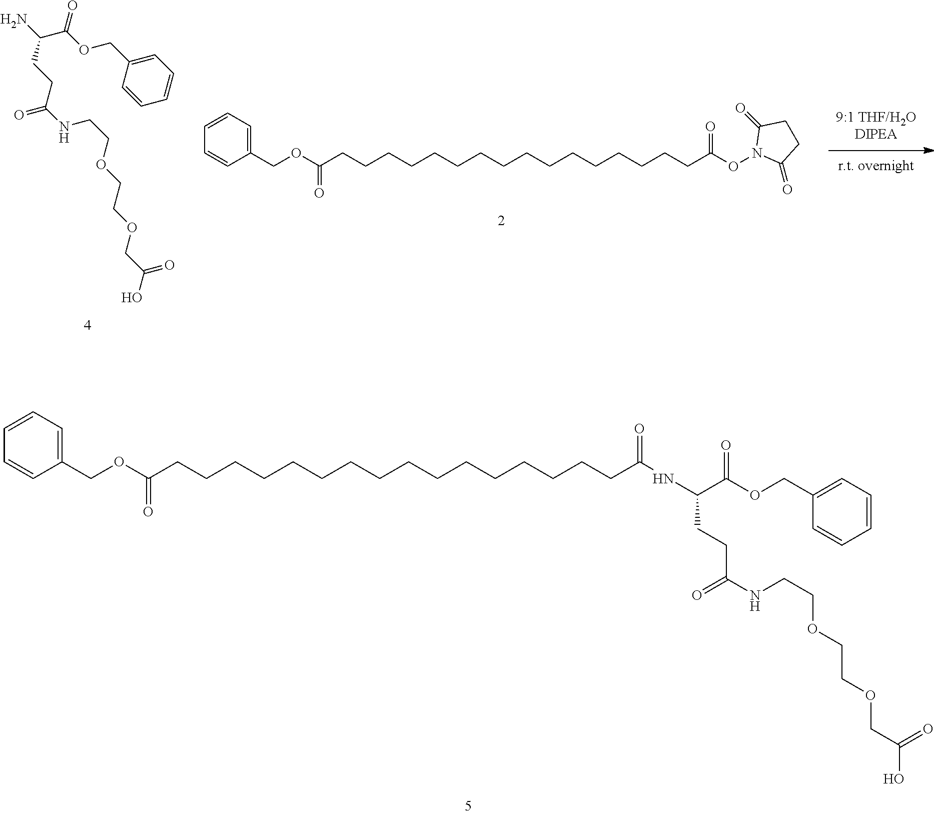 [SMILES: N[C@@H](CCC(=O)NCCOCCOCC(=O)O)C(=O)OCc1ccccc1 ; O=C(CCCCCCCCCCCCCCCCC(=O)ON1C(=O)CCC1=O)OCc1ccccc1 ; O=C(O)COCCOCCNC(=O)CC[C@H](NC(=O)CCCCCCCCCCCCCCCCC(=O)OCc1ccccc1)C(=O)OCc1ccccc1]

To a solution of 4 in THF (12 mmolar, 9:1 THF/H₂O) purged with N₂ is added a solution of 1-benzyl 18-(2,5-dioxopyrrolidin-1-yl) octadecanedioate (2, 1.2 eq.) in water. DIPEA (5 eq.) is added and the reaction is stirred under N₂ at r.t. for 16 hours. The solvent is removed and crude material taken up in a minimal amount of ACN and then loaded onto 20 g C18 15 uM column for reverse phase chromatography. The crude material is purified over 0-100% ACN/H₂O (0.1% TFA) 24 min gradient. Fractions with desired product are pooled, concentrated, frozen and lyophilized.

### Step 6: (S)-22-((benzyloxy)carbonyl)-3,20,25,34-tetraoxo-1-phenyl-2,29,32,38,41-pentaoxa-21,26,35-triazatritetracontan-43-oic acid

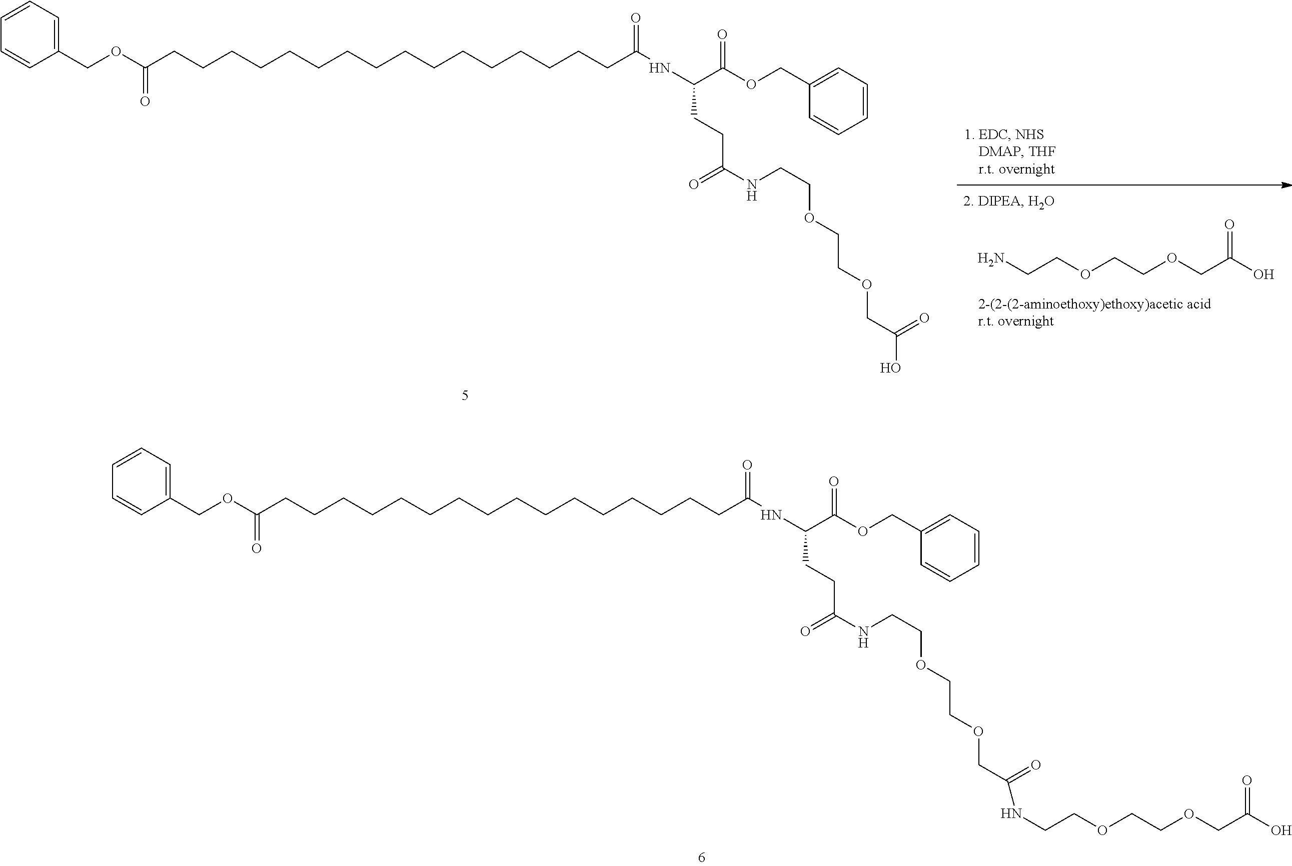 [SMILES: O=C(O)COCCOCCNC(=O)CC[C@H](NC(=O)CCCCCCCCCCCCCCCCC(=O)OCc1ccccc1)C(=O)OCc1ccccc1 ; NCCOCCOCC(=O)O ; O=C(O)COCCOCCNC(=O)COCCOCCNC(=O)CC[C@H](NC(=O)CCCCCCCCCCCCCCCCC(=O)OCc1ccccc1)C(=O)OCc1ccccc1]

5 is dissolved in THF (0.02 molar) under N₂ and N-hydroxysuccinimide (1.2 eq.), EDC (1.5 eq.) and DMAP (0.1 eq.) are added. The reaction is stirred under N₂ at r.t. for 16 hours to give the NHS-ester intermediate. A solution of 2-(2-(2-aminoethoxy)ethoxy)acetic acid (1.1 eq.) in water (9:1 THF/H₂O) and DIPEA (5 eq.) is added to the NHS-ester intermediate. The reaction is stirred at r.t. under N₂ for 16 hours. The solvent is removed and crude material taken up in a minimal amount of ACN and then loaded onto a 20 g C18 15 uM column for reverse phase chromatography. The crude material is purified over 0-100% ACN/H₂O (0.1% TFA) 24 min gradient. Fractions with desired product are pooled, concentrated, frozen and lyophilized.

### Step 7: 21,39-dibenzyl 1-(2,5-dioxopyrrolidin-1-yl) (S)-9,18,23-trioxo-2,5,11,14-tetraoxa-8,17,22-triazanonatriacontane-1,21,39-tricarboxylate

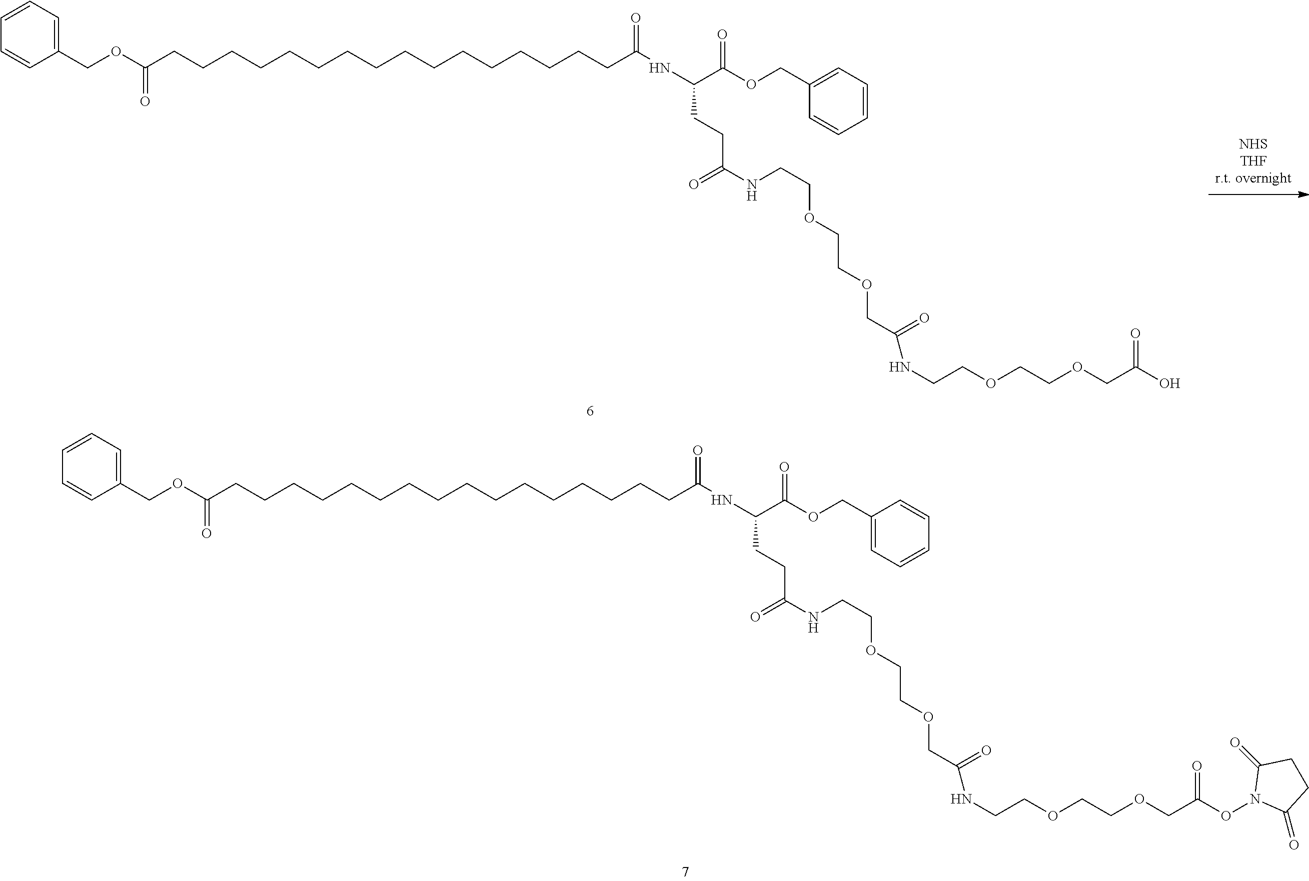 [SMILES: O=C(O)COCCOCCNC(=O)COCCOCCNC(=O)CC[C@H](NC(=O)CCCCCCCCCCCCCCCCC(=O)OCc1ccccc1)C(=O)OCc1ccccc1 ; O=C(CC[C@H](NC(=O)CCCCCCCCCCCCCCCCC(=O)OCc1ccccc1)C(=O)OCc1ccccc1)NCCOCCOCC(=O)NCCOCCOCC(=O)ON1C(=O)CCC1=O]

To a solution of 6 in dry THF (0.02 molar) under N₂ is added N-hydroxysuccinimide (1.2 eq.). The reaction is stirred under N₂ at r.t. for 16 hours. The reaction solvent is evaporated and crude material taken up in a minimal amount of ACN and then loaded onto 20 g C18 15 uM column for reverse phase chromatography. The crude material is purified over 0-100% ACN/H₂O (0.1% TFA) 24 min gradient. Fractions with desired product are pooled, concentrated, frozen and lyophilized.

### Step 8: (S)-22-carboxy-1-((2,5-dioxopyrrolidin-1-yl)oxy)-1,10,19,24-tetraoxo-3,6,12,15-tetraoxa-9,18,23-triazahentetracontan-41-oic acid

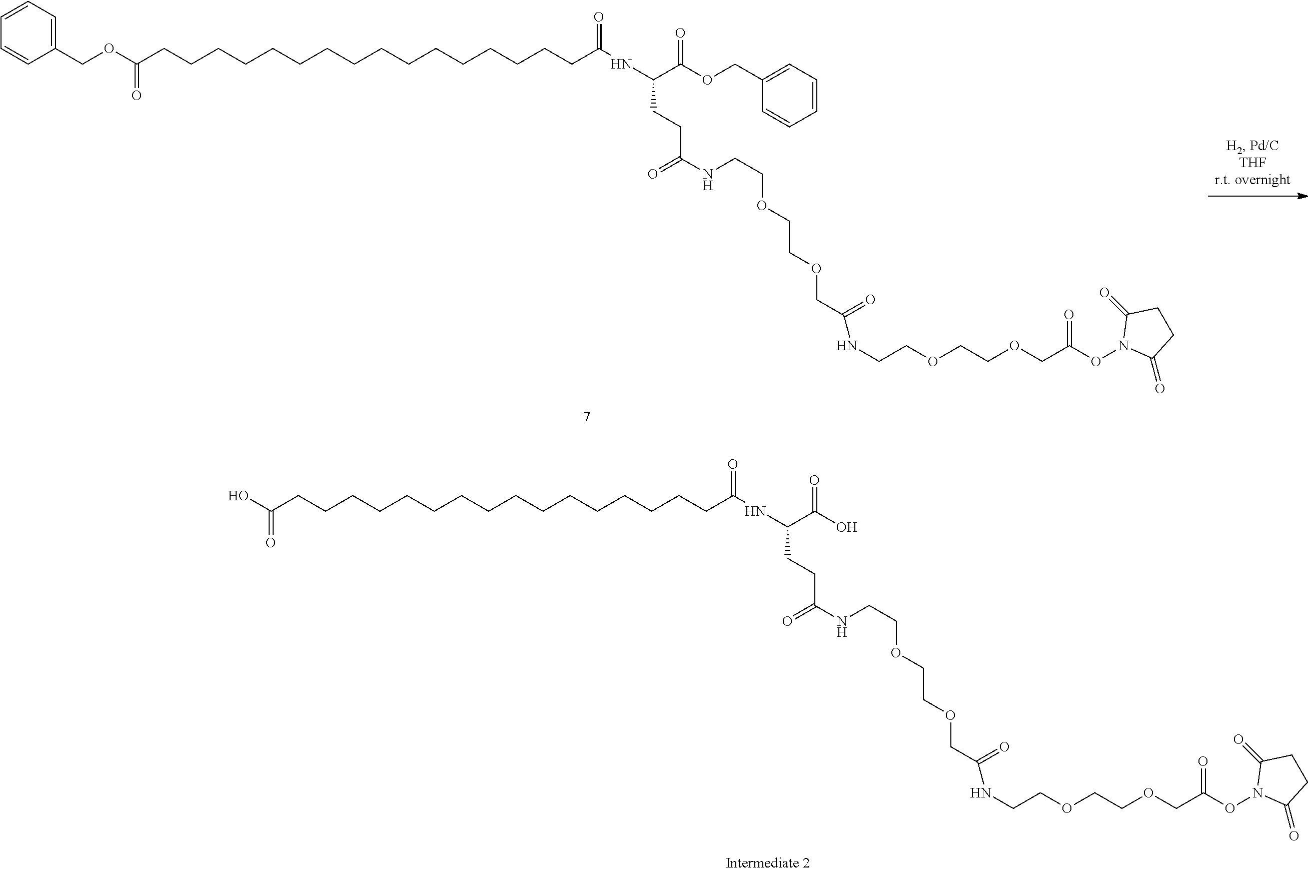 [SMILES: O=C(CC[C@H](NC(=O)CCCCCCCCCCCCCCCCC(=O)OCc1ccccc1)C(=O)OCc1ccccc1)NCCOCCOCC(=O)NCCOCCOCC(=O)ON1C(=O)CCC1=O ; O=C(O)CCCCCCCCCCCCCCCCC(=O)N[C@@H](CCC(=O)NCCOCCOCC(=O)NCCOCCOCC(=O)ON1C(=O)CCC1=O)C(=O)O]

To a solution of 7 in dry THF (0.2 molar) flushed with N₂ is added Pd/C (10% activated on charcoal, 0.1 eq.). The reaction is stirred at r.t. under N₂ for ten minutes. The N₂ source is then closed and hydrogen added to the reaction flask. The hydrogen source is then removed and N₂ flow returned. The reaction is stirred at r.t. for 16 hours. The reaction mixture is filtered through a solvent-wet pad of celite and rinsed 3× with excess THF and resulting filtrate concentrated. The crude material is dissolved in a minimal amount of ACN/water and loaded onto a 20 g C18 15 uM column for reverse phase chromatography. The crude material is purified over 0-100% ACN/H₂O (0.1% TFA) 24 min gradient. Fractions with desired product are pooled, concentrated, frozen and lyophilized.

### Example 4A: Protein Conjugation with Fatty Acid Construct (Intermediate 2)

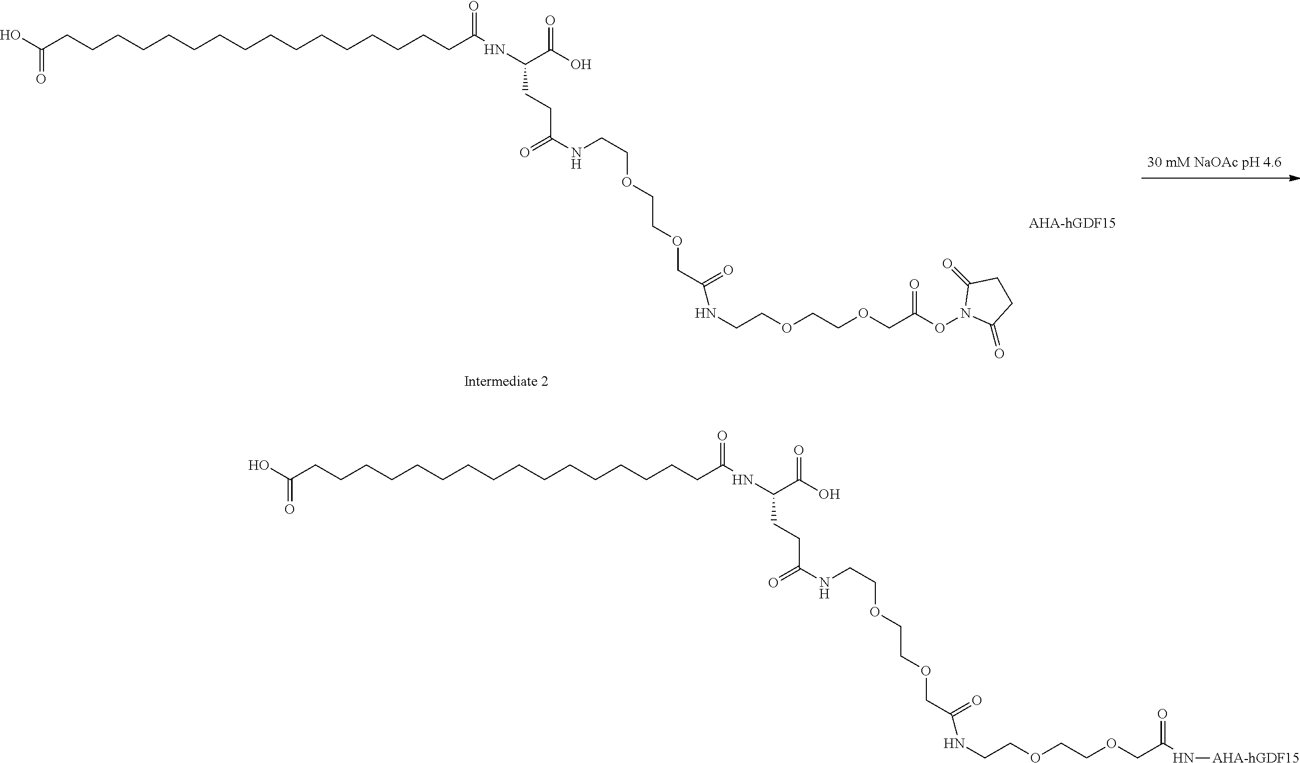

General Protein Conjugation Procedure:

A 10 mg/mL solution of fatty acid-NHS ester is prepared in water. A solution of AHA-hGDF15 (1.0 eq) in is diluted with 30 mM NaOAc pH 4.6 to give a final reaction concentration of 0.88 mg/mL. Fatty acid solution (10 eq. at 10 mg/mL) is added to the protein solution and reaction shaken at r.t. for 16 hours. The reaction mixture is purified.

In view of the homodimeric nature of hGDF15, a mixture of AHA-hGDF15+1 fatty acid and AHA-hGDF15+2 fatty acids can be obtained. The fatty acid construct (Intermediate 2) is linked at the N-terminus of one or of two of the monomeric units. Such mixture of conjugates could be represented as follow:

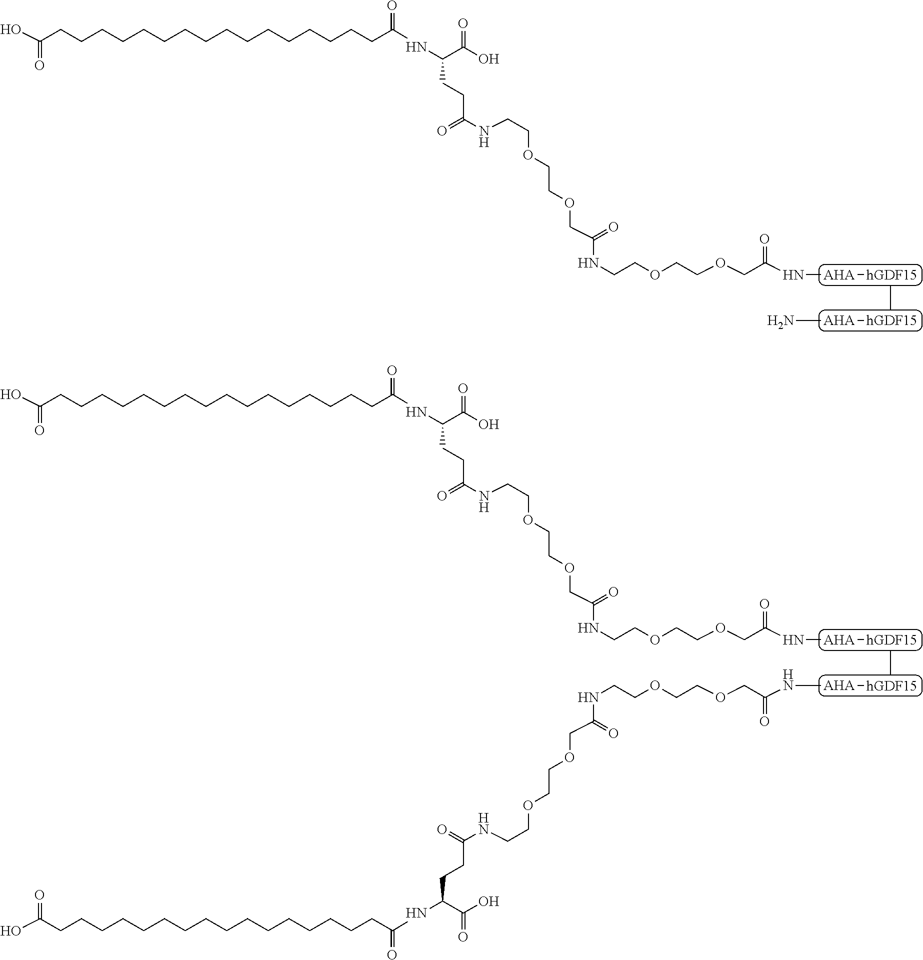

wherein the line between the 2 AHA-hGDF15 units is a disulfide bond.

TABLE 7
Methods for characterizing fatty acids of GDF15 conjugates

| | | |
|---|---|---|
| Method | Open Access | SQ2 RXNMON-Acidic-NonPolar |
| A | method Name | |
| | Column | ACQUITY UPLC ® BEH C18 2.1 × |
| | | 50 mm, 1.7 μm |
| | Column | 50 C. |
| | Temperature | |
| | Eluents | A: Water + 0.1% Formic Acid |
| | | B: ACN + 0.1% Formic Acid |
| | Flow Rate | 1 mL/min |
| | Gradient | 0-1.40 min 60% A; 1.40-2.06 min 2% A |
| | Mass | Single Quadrupole ESI scan range 120-1600 |
| | Spectrometer | |
| | HPLC | Waters Acquity |
| Method | Open Access | SQ2 ProductAnalysis-Acidic-NonPolar |
| B | method Name | |
| | Column | ACQUITY UPLC ® BEH C18 2.1 × |
| | | 50 mm, 1.7 μm |
| | Column | 50 C. |
| | Temperature | |
| | Eluents | A: Water + 0.1% Formic Acid |
| | | B: ACN + 0.1% Formic Acid |
| | Flow Rate | 1 mL/min |
| | Gradient | 0 min 60% A; 3.40 min 2% A; 5.15 min 2% A |
| | Mass | Single Quadrupole ESI scan range 120-1600 |
| | Spectrometer | |
| | HPLC | Waters Acquity |
| Method | Open Access | SQ2 RXNMON-Acidic |
| C | method Name | |
| | Column | ACQUITY UPLC ® BEH C18 2.1 × |
| | | 50 mm, 1.7 μm |
| | Column | 50 C. |
| | Temperature | |
| | Eluents | A: Water + 0.1% Formic Acid |
| | | B: ACN + 0.1% Formic Acid |
| | Flow Rate | 1 mL/min |
| | Gradient | 0 min 98% A; 1.76 min 2% A; 2.06 min 2% A |
| | Mass | Single Quadrupole ESI scan range 120-1600 |
| | Spectrometer | |
| | HPLC | Waters Acquity |
| Method | Open Access | SQ2 ProductAnalysis-Acidic |
| D | method Name | |
| | Column | ACQUITY UPLC ® BEH C18 2.1 × |
| | | 50 mm, 1.7 μm |
| | Column | 50 C. |
| | Temperature | |
| | Eluents | A: Water + 0.1% Formic Acid |
| | | B: ACN + 0.1% Formic Acid |
| | Flow Rate | 1 mL/min |
| | Gradient | 0 min 98% A; 1.76 min 2% A; 2.06 min 2% A |
| | Mass | Single Quadrupole ESI scan range 120-1600 |
| | Spectrometer | |
| | HPLC | Waters Acquity |

### Example 5A: His-hGDF15 (I-59) Conjugated to Intermediate 37

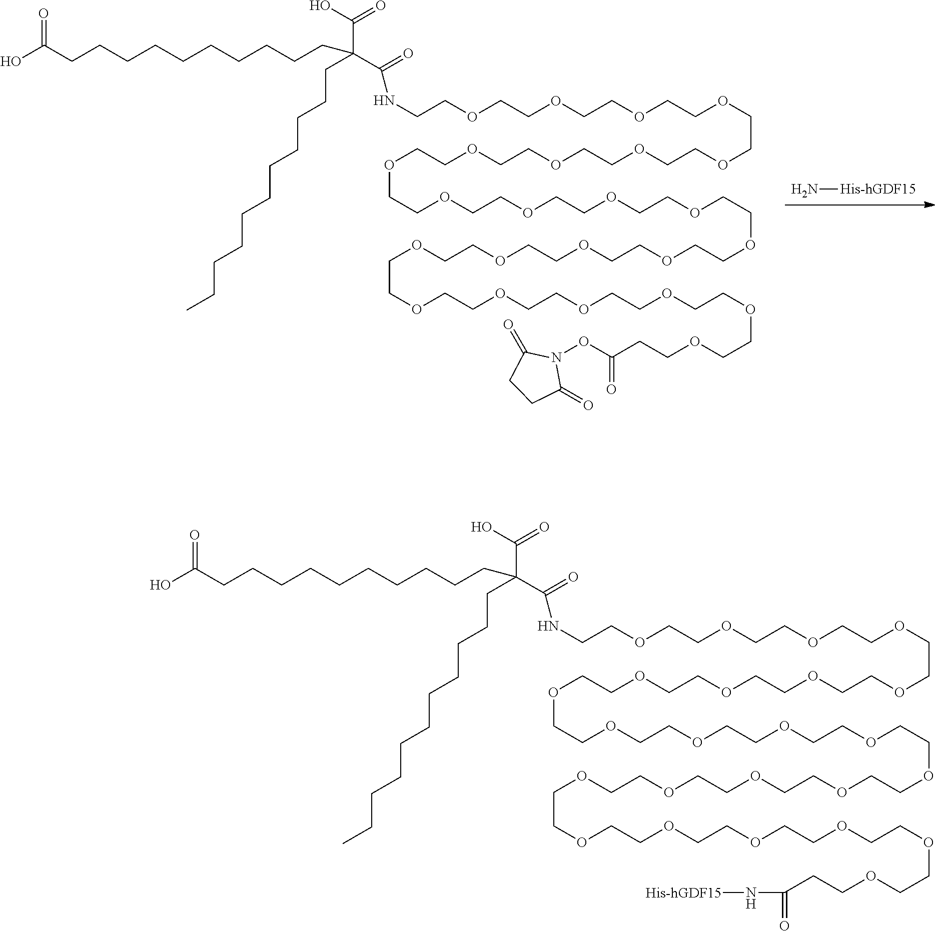

His-GDF15 (0.493 ml, 0.026 mol, 1.42 mg/ml) was added to 1.5 ml of 30 mM sodium acetate pH=4 buffer nhs fatty acid (0.221 mg, 0.132 umol, 10 mg/mL) was added to the solution. Overnight the reaction was not complete so 2.5 more equivalents of fatty acid NHS (0.110 mg, 0.066 umol, 10 mg/mL) were added and after 5 hrs Maldi showed+2 conjugate as major product. Product was purified by washing 5 times using amicon ultrafiltration 10 kD to give 565 ug of conjugate in 76% yield. MALDI: sm (18%), expected mass: 26468 observed mass: 26553; +1 fatty acid (38%) expected mass: 28022 observed mass: 28099; +2 fatty acid (40%) expected mass: 29576 observed mass: 29649; +3 fatty acid (4%) expected mass: 31130 observed mass: 31201.

### Example 5B: AHA-hGDF15 Conjugated with Intermediate 37

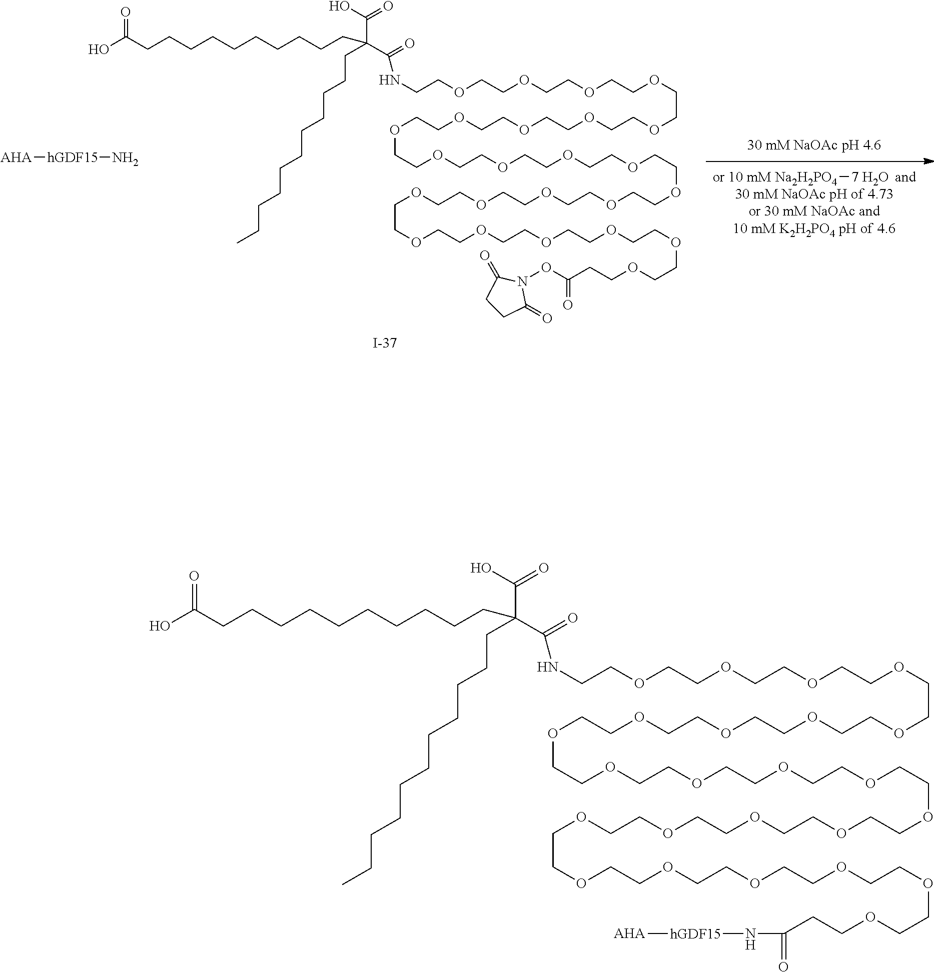

A 10 mg/mL solution of Intermediate 37 in molecular biology grade water was prepared. To AHA-hGDF15 (intermediate 57, 6.67 mg/mL in 30 mM NaOAc pH 4.0, 5.247 mL, 1.433 μmol) was added 30 mM NaOAc pH 4.6 (acceptable range 4.5-5.0) to give a final protein concentration of 0.88 mg/mL. Intermediate 37 (10 eq., 2.39 mL, 0.014 mmol) was added and the reaction was mixed at r.t. for 18 hours. Precipitate had formed in the reaction vial. The reaction mixture was split amongst 4×15 mL 10 kDa Amicon centrifugal filters and each was diluted to 15 mL with 30 mM NaOAc pH 4.0. The material was buffer exchanged 4× into 30 mM NaOAc pH 4.0 and samples were combined to a volume of 25.6 mL, agitating the precipitate in the filter with a pipette tip in between washes. Precipitate remained in solution so the mixture was let sit at 4° C. overnight. Concentration was measured by A280 (30040 cm⁻¹M⁻¹, 27538 g/mol) to be 1.62 mg/mL (100%). UPLC analysis showed 60% recovery of +1 and +2 products (Method J).

Example 5B crude mixture which was tested in vivo and reported in table 8:

TABLE 8

| Species | % observed |
|---|---|
| AHA-GDF15 | 29 |
| AHA-GDF15 + 1 FA | 27 |
| AHA-GDF15 + 2 FA | 33 |
| AHA-GDF15 + 3 FA | 11 |

AHA-hGDF15+1FA (Fatty acid) corresponds to a reaction at the N-terminus amino functionality on the one molecule of the GDF15 homodimer.

AHA-hGDF15+2FA (Fatty acid) corresponds to a reaction at the N-terminus amino functionality on the second molecule of the GDF15 homodimer.

AHA-hGDF15+3FA (Fatty acid) corresponds to a non-selective reaction at some other site of the GDF15 homodimer.

Purification:

The crude product was purified by reverse phase chromatography (Buffer A 0.1% TFA in water; Buffer B 0.1M TFA in ACN gradient; 99%-80% Buffer A) on a Waters BEH300 130 Å, 3.5 μm, 4.6 mm×100 mm flow rate 2.5 ml/min.

Fraction 1: Unreacted AHA-hGDF15: Rt=17.33 min

Fraction 2: (19B1): AHA-GDF15+1FA: Rt=20.2 min (approximately 15% yield)

Fraction 3: (19B2): AHA-GDF15+2FA: Rt=21.6 min (approximately 15% yield)

Fraction 4: (19B3): AHA-GDF15+3 FA: Rt=23.0 min (approximately 5% yield)

A 1:1 ratio mixture of 19B1 and 19B2 was prepared and tested (19Bm).

Alternatively the reaction may be carried out in 10 mM Na₂HPO₄-7H₂O and 30 mM NaOAc at a pH of 4.73: A 10 mg/mL solution of Intermediate 37 in molecular biology grade water was prepared. To AHA-hGDF15 (Intermediate 57, 12.04 mg/mL in 30 mM NaOAc pH 4.0, 4.15 μL, 0.002 μmol) was added 30 mM NaOAc 10 mM Na₂HPO₄-7H₂O pH 4.73 to give a final protein concentration of 0.88 mg/mL. Intermediate 37 (20 eq., 6.83 μL, 0.041 μmol) was added and the reaction was mixed at r.t. for 18 hours. The reaction mixture had turned cloudy with precipitate. UPLC analysis showed 58%+1 and +2 products (Method J).

TABLE 9

| Species | % observed |
|---|---|
| AHA-GDF15 | 0 |
| AHA-GDF15 + 1 FA | 11 |
| AHA-GDF15 + 2 FA | 47 |
| AHA-GDF15 + 3 FA | 34 |
| AHA-GDF15 + 4 FA | 7 |

The reaction may also be carried out in 30 mM NaOAc and 10 mM K₂HPO₄ at a pH of 4.6: A 10 mg/mL solution of intermediate 37 in molecular biology grade water was prepared. To AHA-hGDF15 (intermediate 57, 6.21 mg/mL in 30 mM NaOAc pH 4.0, 5.261 mL, 1.337 μmol) was added 30 mM NaOAc 10 mM K₂HPO₄ pH 4.6 (acceptable range 4.5-5.0) to give a final protein concentration of 0.88 mg/mL Intermediate 37 (10 eq., 68.3 μL, 0.409 μmop was added and the reaction was mixed at r.t. for 7 hours. The reaction mixture had turned cloudy with precipitate. The reaction mixture was split into four 9 mL portions in 15 mL 10 kDa Amicon centrifugal filter and diluted to 15 mL with 30 mM NaOAc pH 4.0. The material was buffer exchanged 4× into 30 mM NaOAc pH 4.0, agitating the precipitate between each wash with a pipette tip. The reaction mixture was concentrated to a volume of 75 mL. Precipitate remained so the material was stored at 4° C. for two days. Concentration was measured by A280 (30040 cm⁻¹M⁻¹, 27538 g/mol) to be 0.4 mg/mL (97%). UPLC analysis showed 61% recovery of +1 and +2 products.

TABLE 10

| Species | % observed |
|---|---|
| AHA-GDF15 | 34 |
| AHA-GDF15 + 1 FA | 34 |
| AHA-GDF15 + 2 FA | 27 |
| AHA-GDF15 + 3 FA | 5 |

### Reference Example 1: His-hGDF15 BCN (I-58) Conjugated to Intermediate PEG-Myristic Acid Construct

### Step 1

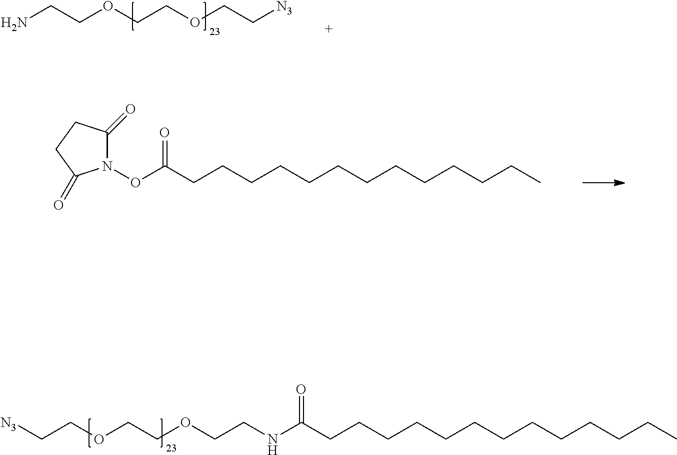

To a mixture of Azido-PEG23-Amine (30 mg, 0.027 mmol) and myristic NHS ester (Toronto Research Chemicals, cat # S69080) (12 mg, 0.037 mmol) was added DCM (1 mL) and DIPEA (13 uL), and the mixture was stirred at r.t. overnight. The mixture was purified by silica chromatography eluting with EtOAc/heptane (0-100%) then MeOH/DCM (0-10%) to give clean product at around 5% MeOH/DCM. LCMS: (Gradient: from 40 to 98% B in 1.4 min—flow 1 mL/min Eluent A: water+0.05% formic acid+3.75 mM ammonium acetate, Eluent B: acetonitrile+0.04% formic acid) LCMS: rt=2.20 (Method C) Mass +H calculated: 1354.71 Mass observed: 1354.4.

### Step 2

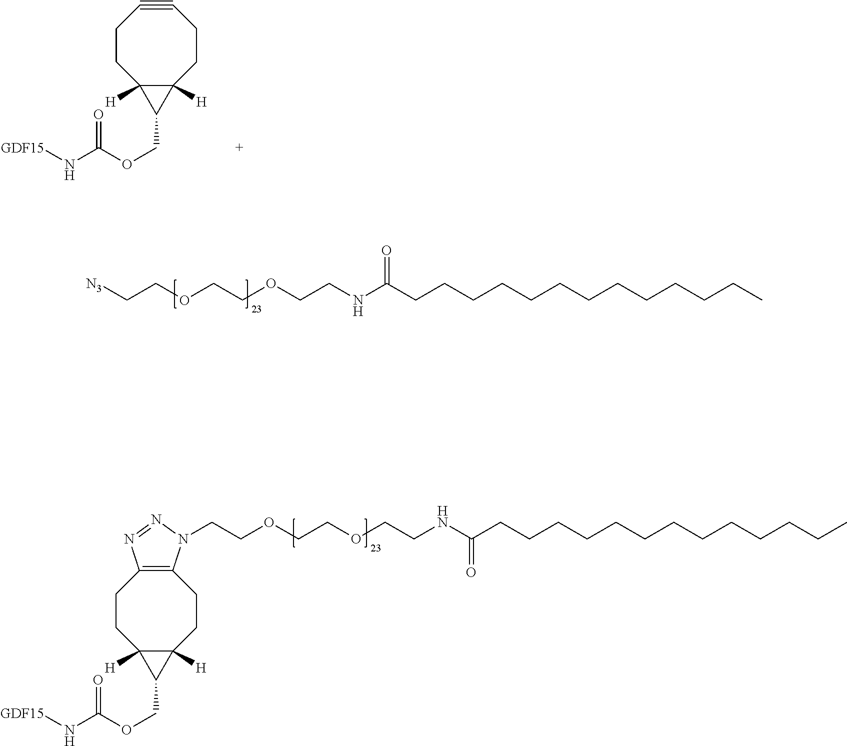

To a solution of BCN-hGDF15 (I-52: 800 uL, 0.25 mg/mL) was added a (2 mg/mL in DMSO, 6.3 uL, 10 eq), and the mixture was stirred at r.t. overnight. 1.1 mL 0.20 mg/mL in quantitative yield. (Maldi: +1 mass calculated: 28223 mass observed: 28640; +2 mass calculated: 29543; mass observed:29962, +3 mass calculated: 30863 mass observed:31426, +4 mass calculated: 32183 mass observed:32911).

### Reference Example 2: His-hGDF15-PEG23

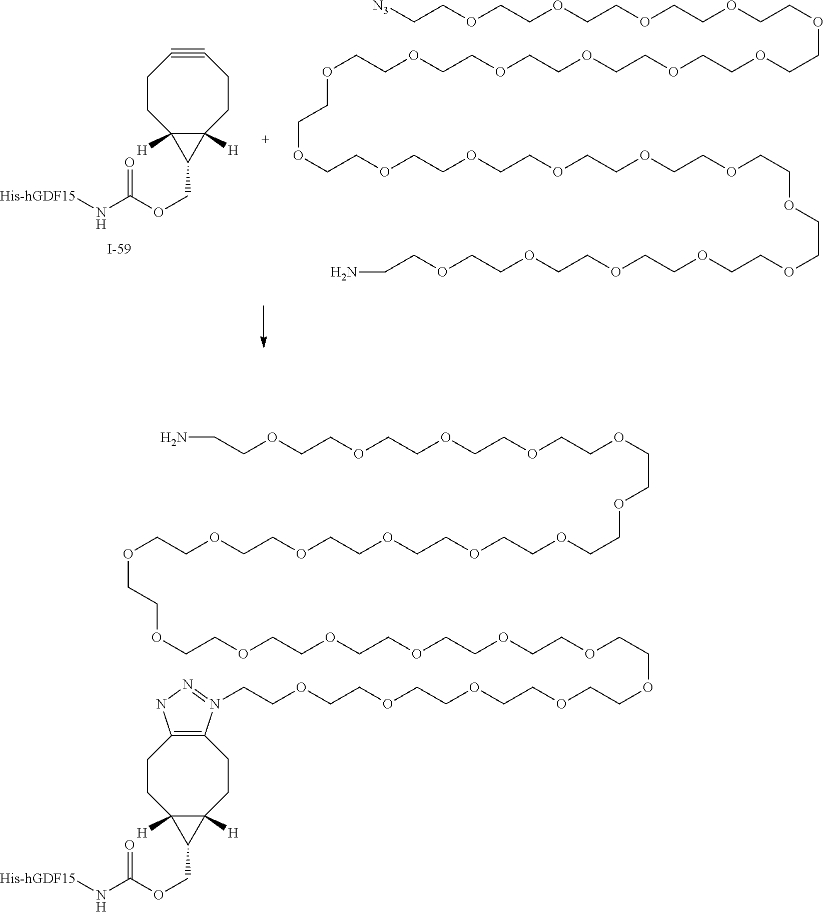

TABLE 11

| Degree of Labelling | Calculated | Observed | % |
|---|---|---|---|
| His-hGDF15 | 26468 | 26360.3 | 5 |
| His-hGDF15-BCN | 26644 | n/a | 0 |
| His-hGDF15 + 1 PEG23 | 27567 | 28178.6 | 15 |
| His-hGDF15 + 2 PEG23 | 28666 | 29385.1 | 46 |
| His-hGDF15 + 3 PEG23 | 29765 | 30547.2 | 28 |
| His-hGDF15 + 4 PEG23 | 30864 | 31731.8 | 5 |

To a solution of His-hGDF15 BCN (159: 427 μL, 1.17 mg/mL, 0.019 μmop in 30 mM NaOAc pH 4.0 (427 μL) was added azido-dPEG₂₃-amine (Quanta Biodesign, 104 μg, 0.094 μmol). The reaction was mixed at r.t. for 16 hours at which point the mixture was exchanged into 30 mM NaOAc pH 4.0 using 10 kDa MWCO Amicon centrifugal filter by diluting and concentrating the sample 5 times to a volume of 140 μL. MALDI analysis showed full conversion to +1 through +4 products. The concentration was measured by A₂₈₀ (29090 M−1 cm−1, 27600 g/mol) to be 2.099 mg/mL (57%).

1. A method of treating a disease or disorder selected from non-alcoholic fatty liver disease (NAFLD), non-alcoholic steatohepatitis (NASH), end-stage liver disease, hepatic steatosis (fatty liver), liver fibrosis, liver inflammation, liver cirrhosis, primary biliary cirrhosis (PBC), and hepatocellular carcinoma (HCC) by administering a therapeutically effective amount of a GDF15 therapeutic agent comprising one or more of a GDF15 protein, variant, mutant, fusion, or conjugate.

2. The method of claim 1, wherein the GDF15 therapeutic agent is GDF15 conjugate.

3. The method of claim 1, wherein the GDF15 therapeutic agent is an HSA-GDF15 fusion protein or an Fc-GDF15 fusion protein.

4. The method of claim 1, wherein the GDF15 therapeutic agent is selected from Table 1.

5. The method of claim 1, wherein the disease or disorder is selected from non-alcoholic fatty liver disease (NAFLD) and non-alcoholic steatohepatitis (NASH) by administering a therapeutically effective amount of a GDF15 therapeutic agent comprising one or more of a GDF15 protein, variant, mutant, fusion, or conjugate.

6. The method of claim 5, wherein the GDF15 therapeutic agent is a fatty acid-GDF15 conjugate or a PEG-GDF15 conjugate.

7. The method of claim 5, wherein the GDF15 therapeutic agent is an HSA-GDF15 fusion protein or an Fc-GDF15 fusion protein.

8. The method of claim 5, wherein the GDF15 therapeutic agent is selected from Table 1.

9. A method of treating a disease or disorder selected from non-alcoholic fatty liver disease (NAFLD), non-alcoholic steatohepatitis (NASH), end-stage liver disease, hepatic steatosis (fatty liver), liver fibrosis, liver inflammation, liver cirrhosis, primary biliary cirrhosis (PBC), and hepatocellular carcinoma (HCC) by administering a therapeutically effective amount of a pharmaceutical composition comprising GDF15 therapeutic agent comprising one or more of a GDF15 protein, variant, mutant, fusion, or conjugate.

10. The method of claim 9, wherein the GDF15 therapeutic agent is a fatty acid-GDF15 conjugate or a PEG-GDF15 conjugate.

11. The method of claim 9, wherein the GDF15 therapeutic agent is an HSA-GDF15 fusion protein or an Fc-GDF15 fusion protein.

12. The method of claim 9, wherein the GDF15 therapeutic agent is selected from Table 1.

13. The method of claim 9, wherein the disease or disorder is selected from non-alcoholic fatty liver disease (NAFLD) and non-alcoholic steatohepatitis (NASH) by administering a therapeutically effective amount of a pharmaceutical composition comprising GDF15 therapeutic agent comprising one or more of a GDF15 protein, variant, mutant, fusion, or conjugate.

14. The method of claim 13, wherein the GDF15 therapeutic agent is a fatty acid-GDF15 conjugate or a PEG-GDF15 conjugate.

15. The method of claim 13, wherein the GDF15 therapeutic agent is an HSA-GDF15 fusion protein or an Fc-GDF15 fusion protein.

16. The method of claim 13, wherein the GDF15 therapeutic agent is selected from Table 1.

17. The method of claim 1, wherein:
a) the GDF15 therapeutic agent does not comprise a GDF15 polypeptide comprising the amino acid sequence of SEQ ID NO: 41; or
b) the GDF15 therapeutic agent is not a fatty acid-GDF15 conjugate comprising the amino acid sequence of SEQ ID NO: 41, or
(c) the GDF15 therapeutic agent is not albumin-GDF15 fusion comprising the amino acid sequence of SEQ ID NO: 41, such as a human serum albumin-GDF15 fusion
(d) the GDF15 therapeutic is a fatty acid conjugate which does not comprise the amino sequence of:
(i)
SEQ ID NO: 41;
(ii)
(SEQ ID NO: 321)
MHHHH HHAR NGDHC PLGPG RCCRL HTVRA SLEDL GWADW
VLSPR EVQVT MCIGA CPSQF RAANM HAQIK TSLHR LKPDT
VPAPC CVPAS YNPMV LIQKT DTGVS LQTYD DLLAK DCHCI
(M-(his)₆-hGDF15 (197-308)),
(iii)
(SEQ ID NO: 322)
MHHHHHHMARNGDHCPLGPGRCCRLHTVRASLEDLGWADWVLSPREV
QVTMCIGACPSQFRAANMHAQIKTSLHRLKPDTVPAPCCVPASYNPMVL
IQKTDTGVSLQTYDDLLAKDCHCI (M-(his)₆-M-hGDF15
(197-308)),
(iv)
(SEQ ID NO: 323)
MHHHHHHAHARDGCPLGEGRCCRLQSLRASLQDLGWANWVVAPRELD
VRMCVGACPSQFRSANTHAQMQARLHGLNPDAAPAPCCVPASYEPVVL
MHQDSDGRVSLTPFDDLVAKDCHCV (M-(his)₆-dGDF15),
(v)
(SEQ ID NO: 324)
MHNGDHCPLGPGRCCRLHTVRASLEDLGWADWVLSPREVQVTMCIGAC
PSQFRAANMHAQIKTSLHRLKPDTVPAPCCVPASYNPMVLIQKTDTGVS
LQTYDDLLAKDCHCI (MH-hGDF15(199-308)),
(vi)
(SEQ ID NO: 325)
MHAGDHCPLGPGRCCRLHTVRASLEDLGWADWVLSPREVQVTMCIGAC
PSQFRAANMHAQIKTSLHRLKPDTVPAPCCVPASYNPMVLIQKTDTGVS
LQTYDDLLAKDCHCI (MHA-hGDF15(200-308)),
or
(vii)
(SEQ ID NO: 326)
AHNGDHCPLGPGRCCRLHTVRASLEDLGWADWVLSPREVQVTMCIGAC
PSQFRAANMHAQIKTSLHRLKPDTVPAPCCVPASYNPMVLIQKTDTGVS
LQTYDDLLAKDCHCI (AH-hGDF15(199-308);
or
(e) the GDF15 therapeutic agent does not comprise one of the following amino acid sequences:
(i)
(SEQ ID NO: 321)
MHHHH HHAR NGDHC PLGPG RCCRL HTVRA SLEDL GWADW
VLSPR EVQVT MCIGA CPSQF RAANM HAQIK TSLHR LKPDT
VPAPC CVPAS YNPMV LIQKT DTGVS LQTYD DLLAK DCHCI
(M-(his)₆-hGDF15 (197-308)),
(ii)
SEQ ID NO: 6,
(iii)
SEQ ID NO: 7,
(iv)
(SEQ ID NO: 322)
MHHHHHHMARNGDHCPLGPGRCCRLHTVRASLEDLGWADWVLSPREVQV
TMCIGACPSQFRAANMHAQIKTSLHRLKPDTVPAPCCVPASYNPMVLIQ
KTDTGVSLQTYDDLLAKDCHCI (M-(his)₆-M-hGDF15
(197-308)),
(v)
(SEQ ID NO: 323)
MHHHHHHAHARDGCPLGEGRCCRLQSLRASLQDLGWANWVVAPRELDVR
MCVGACPSQFRSANTHAQMQARLHGLNPDAAPAPCCVPASYEPVVLMHQ
DSDGRVSLTPFDDLVAKDCHCV (M-(his)₆-dGDF15),
(vi)
(SEQ ID NO: 324)
MHNGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (MH-hGDF15(199-308)),
(vii)
(SEQ ID NO: 325)
MHAGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (MHA-hGDF15(200-308)),
(viii)
SEQ ID NO: 41,
and
(ix)
(SEQ ID NO: 326)
AHNGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (AH-hGDF15(199-308);
or
(f) the GDF15 therapeutic agent is a fatty acid-GDF15 conjugate which does not comprise a fatty acid according to any one of Formula A1, A2, and A3: 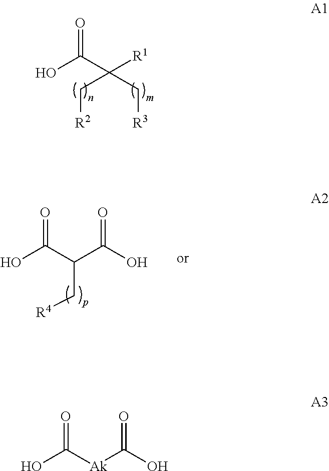
R¹ is CO₂H or H;
R², R³ and R⁴ are independently of each other H, OH, CO₂H, —CH═CH₂ or —C═CH;
Ak is a branched C₆-C₃₀alkylene;
n, m and p are independently of each other an integer between 6 and 30; and which does not comprise tetradecanoic acid; or
(g) the GDF15 therapeutic agent is a fatty acid-GDF15 conjugate which does not comprise one or more of the following fatty acids:

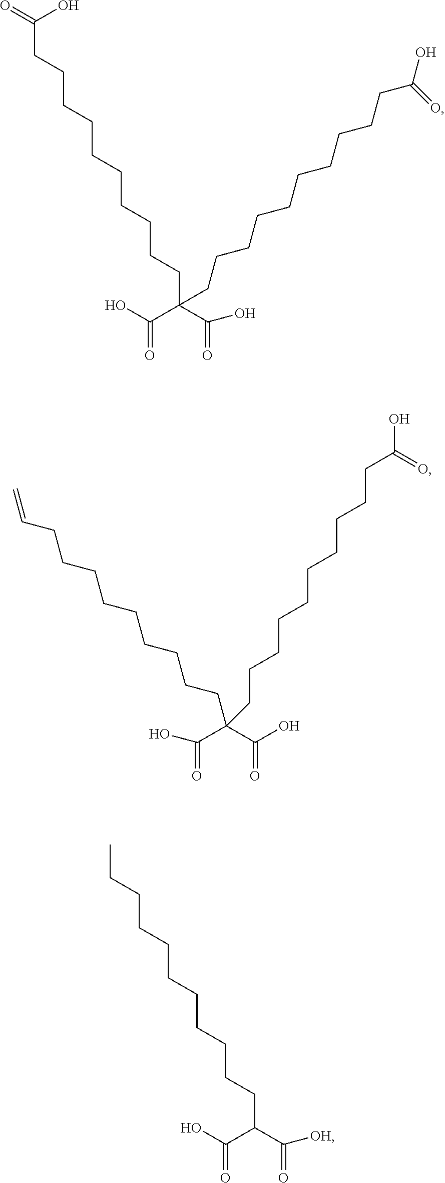 [SMILES: O=C(O)CCCCCCCCCCC(CCCCCCCCCCC(=O)O)(C(=O)O)C(=O)O ; C=CCCCCCCCCCC(CCCCCCCCCCC(=O)O)(C(=O)O)C(=O)O ; CCCCCCCCCCCC(C(=O)O)C(=O)O]

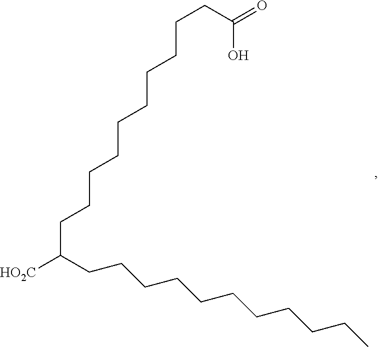 [SMILES: CCCCCCCCCCCC(CCCCCCCCCCC(=O)O)C(=O)O]

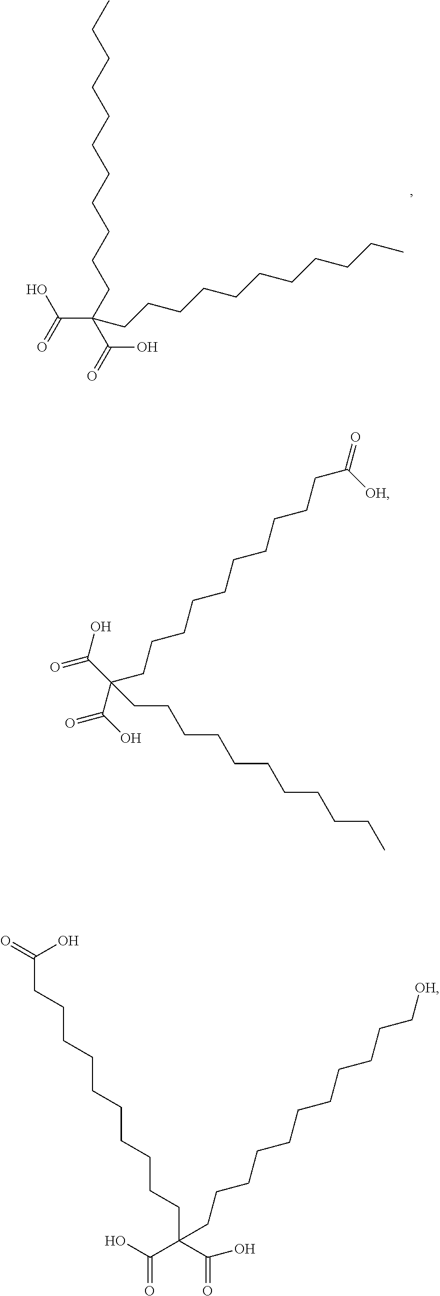 [SMILES: CCCCCCCCCCCC(CCCCCCCCCCC)(C(=O)O)C(=O)O ; CCCCCCCCCCCC(CCCCCCCCCCC(=O)O)(C(=O)O)C(=O)O ; O=C(O)CCCCCCCCCCC(CCCCCCCCCCCO)(C(=O)O)C(=O)O]

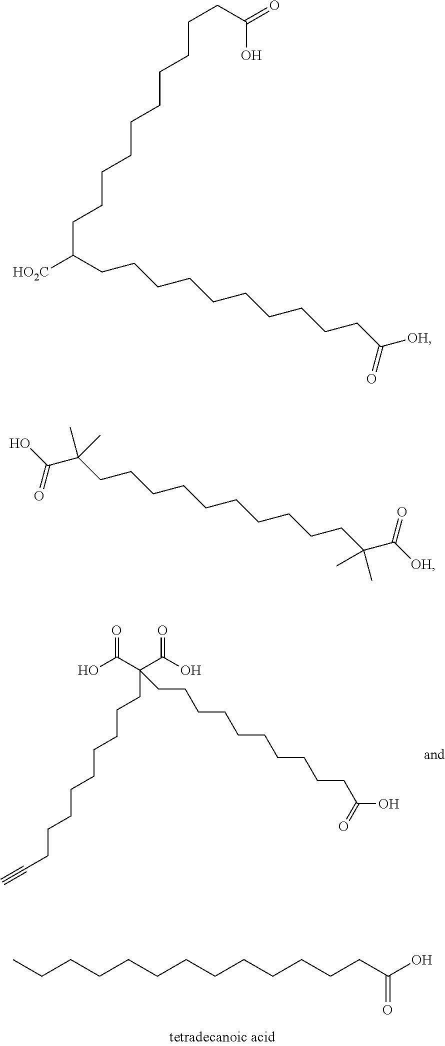 [SMILES: O=C(O)CCCCCCCCCCC(CCCCCCCCCCC(=O)O)C(=O)O ; CC(C)(CCCCCCCCCCC(C)(C)C(=O)O)C(=O)O ; C#CCCCCCCCCCC(CCCCCCCCCCC(=O)O)(C(=O)O)C(=O)O ; CCCCCCCCCCCCCC(=O)O]

18-20. (canceled)

21. The method of claim 1, wherein the GDF15 therapeutic agent comprises the amino acid sequence of any one of the following: SEQ ID NO: 5, SEQ ID NO: 6, SEQ ID NO: 7, SEQ ID NO: 8, SEQ ID NOs: 42-63, SEQ ID NO: 69-107, SEQ ID NO: 148, SEQ ID NO: 149, and SEQ ID NO: 320.

22-24. (canceled)

25. The method of claim 9, wherein:
a) the GDF15 therapeutic agent does not comprise a GDF15 polypeptide comprising the amino acid sequence of SEQ ID NO: 41; or
b) the GDF15 therapeutic agent is not a fatty acid-GDF15 conjugate comprising the amino acid sequence of SEQ ID NO: 41; or
(c) the GDF15 therapeutic agent is not albumin-GDF15 fusion comprising the amino acid sequence of SEQ ID NO: 41, such as a human serum albumin-GDF15 fusion
(d) the GDF15 therapeutic is a fatty acid conjugate which does not comprise the amino sequence of:
(i)
SEQ ID NO: 41;
(ii)
(SEQ ID NO: 321)
MHHHH HHAR NGDHC PLGPG RCCRL HTVRA SLEDL GWADW
VLSPR EVQVT MCIGA CPSQF RAANM HAQIK TSLHR LKPDT
VPAPC CVPAS YNPMV LIQKT DTGVS LQTYD DLLAK DCHCI
(M-(his)₆-hGDF15 (197-308)),
(iii)
(SEQ ID NO: 322)
MHHHHHHMARNGDHCPLGPGRCCRLHTVRASLEDLGWADWVLSPREV
QVTMCIGACPSQFRAANMHAQIKTSLHRLKPDTVPAPCCVPASYNPMVL
IQKTDTGVSLQTYDDLLAKDCHCI (M-(his)₆-M-hGDF15
(197-308)),
(iv)
(SEQ ID NO: 323)
MHHHHHHAHARDGCPLGEGRCCRLQSLRASLQDLGWANWVVAPRELD
VRMCVGACPSQFRSANTHAQMQARLHGLNPDAAPAPCCVPASYEPVVL
MHQDSDGRVSLTPFDDLVAKDCHCV (M-(his)₆-dGDF15),
(v)
(SEQ ID NO: 324)
MHNGDHCPLGPGRCCRLHTVRASLEDLGWADWVLSPREVQVTMCIGAC
PSQFRAANMEIAQIKTSLHRLKPDTVPAPCCVPASYNPMVLIQKTDTGV
SLQTYDDLLAKDCHCI (MH-hGDF15(199-308)),
(vi)
(SEQ ID NO: 325)
MHAGDHCPLGPGRCCRLHTVRASLEDLGWADWVLSPREVQVTMCIGAC
PSQFRAANMEIAQIKTSLHRLKPDTVPAPCCVPASYNPMVLIQKTDTGV
SLQTYDDLLAKDCHCI (MHA-hGDF15(200-308)),
or
(vii)
(SEQ ID NO: 326)
AHNGDHCPLGPGRCCRLHTVRASLEDLGWADWVLSPREVQVTMCIGAC
PSQFRAANMHAQIKTSLHRLKPDTVPAPCCVPASYNPMVLIQKTDTGVS
LQTYDDLLAKDCHCI (AH-hGDF15(199-308);
(e) the GDF15 therapeutic agent does not comprise one of the following amino acid sequences:
(i)
(SEQ ID NO: 321)
MHHHH HHAR NGDHC PLGPG RCCRL HTVRA SLEDL GWADW
VLSPR EVQVT MCIGA CPSQF RAANM HAQIK TSLHR LKPDT
VPAPC CVPAS YNPMV LIQKT DTGVS LQTYD DLLAK DCHCI
(M-(his)₆-hGDF15 (197-308)),
(ii)
SEQ ID NO: 6,
(iii)
SEQ ID NO: 7,
(iv)
(SEQ ID NO: 322)
MHHHHHHMARNGDHCPLGPGRCCRLHTVRASLEDLGWADWVLSPREVQV
TMCIGACPSQFRAANMHAQIKTSLHRLKPDTVPAPCCVPASYNPMVLIQK
TDTGVSLQTYDDLLAKDCHCI (M-(his)₆-M-hGDF15
(197-308)),
(v)
(SEQ ID NO: 323)
MHHHHHHAHARDGCPLGEGRCCRLQSLRASLQDLGWANWVVAPRELDVR
MCVGACPSQFRSANTHAQMQARLHGLNPDAAPAPCCVPASYEPVVLMHR
QDSDGRVSLTPFDDLVAKDCHCV (M-(his)₆-dGDF15),
(vi)
(SEQ ID NO: 324)
MHNGDHCPLGPGRCCRLHTVRASLEDLGWADWVLSPREVQVTMCIGACPS
QFRAANMHAQIKTSLHRLKPDTVPAPCCVPASYNPMVLIQKTDTGVSLQ
TYDDLLAKDCHCI (MH-hGDF15(199-308)),
(vii)
(SEQ ID NO: 325)
MHAGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (MHA-hGDF15(200-308)),
(viii)
SEQ ID NO: 41,
and
(ix)
(SEQ ID NO: 326)
AHNGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI (AH-hGDF15(199-308);
or
(f) the GDF15 therapeutic agent is a fatty acid-GDF15 conjugate which does not comprise a fatty acid according to any one of Formula A1, A2, and A3: 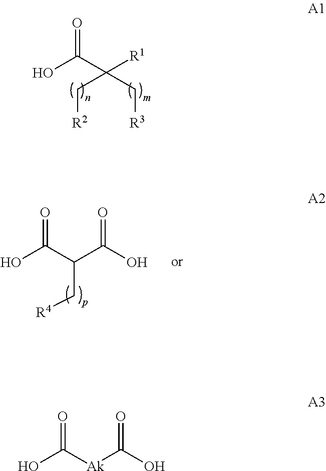
R¹ is CO₂H or H;
R², R³ and R⁴ are independently of each other H, OH, CO₂H, —CH═CH₂ or —C═CH;
Ak is a branched C₆-C₃₀alkylene;
n, m and p are independently of each other an integer between 6 and 30; and which does not comprise tetradecanoic acid; or
(g) the GDF15 therapeutic agent is a fatty acid-GDF15 conjugate which does not comprise one or more of the following fatty acids:

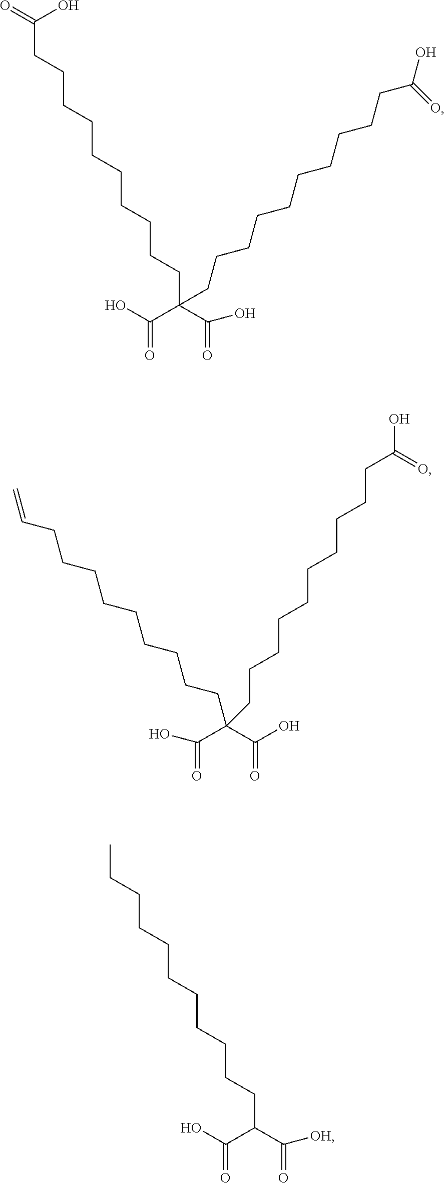 [SMILES: O=C(O)CCCCCCCCCCC(CCCCCCCCCCC(=O)O)(C(=O)O)C(=O)O ; C=CCCCCCCCCCC(CCCCCCCCCCC(=O)O)(C(=O)O)C(=O)O ; CCCCCCCCCCCC(C(=O)O)C(=O)O]

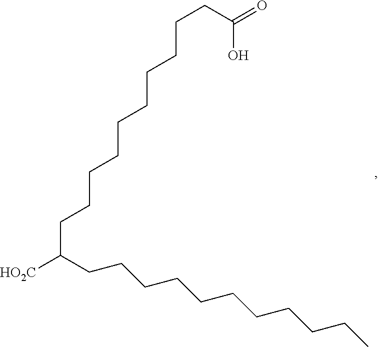 [SMILES: CCCCCCCCCCCC(CCCCCCCCCCC(=O)O)C(=O)O]

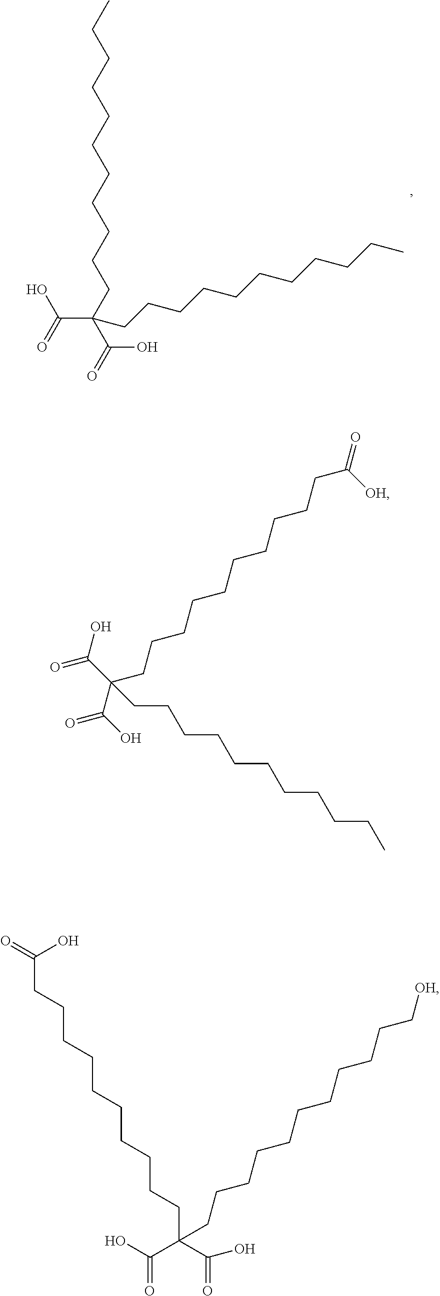 [SMILES: CCCCCCCCCCCC(CCCCCCCCCCC)(C(=O)O)C(=O)O ; CCCCCCCCCCCC(CCCCCCCCCCC(=O)O)(C(=O)O)C(=O)O ; O=C(O)CCCCCCCCCCC(CCCCCCCCCCCO)(C(=O)O)C(=O)O]

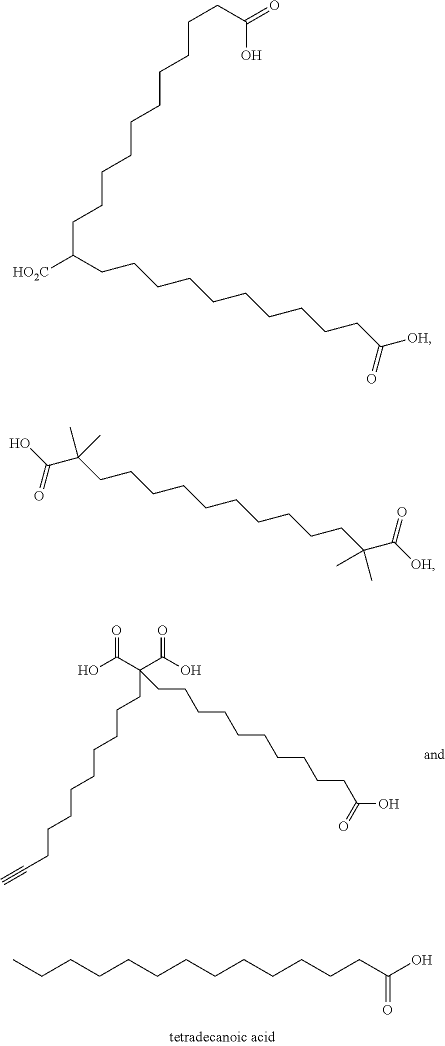 [SMILES: O=C(O)CCCCCCCCCCC(CCCCCCCCCCC(=O)O)C(=O)O ; CC(C)(CCCCCCCCCCC(C)(C)C(=O)O)C(=O)O ; C#CCCCCCCCCCC(CCCCCCCCCCC(=O)O)(C(=O)O)C(=O)O ; CCCCCCCCCCCCCC(=O)O]

26. The method of claim 9, wherein the GDF15 therapeutic agent comprises the amino acid sequence of any one of the following: SEQ ID NO: 5, SEQ ID NO: 6, SEQ ID NO: 7, SEQ ID NO: 8, SEQ ID NOs: 42-63, SEQ ID NO: 69-107, SEQ ID NO: 148, SEQ ID NO: 149, and SEQ ID NO: 320.
